(19) United States
(12) Patent Application Publication
(10) Pub. No.: US 2022/0175960 A1
(43) Pub. Date: Jun. 9, 2022

(54) FASL IMMUNOMODULATORY GENE THERAPY COMPOSITIONS AND METHODS FOR USE

(71) Applicant: Locanabio, Inc., San Diego, CA (US)

(72) Inventor: David A. NELLES, San Diego, CA (US)

(21) Appl. No.: 17/271,123

(22) PCT Filed: Aug. 26, 2019
(86) PCT No.: PCT/US2019/048148

§ 371 Date: Feb. 24, 2021

Related U.S. Application Data
(60) Provisional application No. 62/722,550, filed on Aug. 24, 2018.

(51) Int. Cl. A61K 48/00; C07K 14/705; C12N 9/22; C12N 7/00

(52) U.S. Cl. CPC A61K 48/005; C07K 14/70575; C12N 2750/14143; C12N 7/00; C12N 2750/14122; C12N 9/22

(57) ABSTRACT

Disclosed are compositions comprising a sequence encoding a non-self polypeptide of interest (POI), and a sequence encoding a non-cleavable FASL, wherein expression of the non-cleavable FASL in the presence of IL-6 or TNF-alpha eliminates WIC-mediated immunogenic peptides and helper T cells specific to the expression of the POI. Methods of making and methods of using compositions of the disclosure are also provided. For example, compositions of the disclosure may be used in the combined treatment of a disease or disorder in a subject and immune masking activity specific to the treatment. Exemplary disease or disorders of the disclosure include genetic and epigenetic diseases or disorders.

### RELATED APPLICATIONS

This application claims priority to U.S. Patent Application No. 62/722,550, filed Aug. 24, 2018, the contents of which are herein incorporated by reference in their entirety.

### FIELD OF THE DISCLOSURE

The disclosure is directed to molecular biology, gene therapy, and/or modifying expression and activity of RNA molecules, and more, specifically, to compositions and methods for attenuating the immune response to cells subjected to RNA modification and/or gene therapies via elimination of immune effector cells.

### INCORPORATION OF SEQUENCE LISTING

The contents of the text file named “LOCN_004_001WO_SeqList_ST25”, which was created on Aug. 24, 2019 and is 20.7 MB in size, are hereby incorporated by reference in their entirety.

### BACKGROUND

There has been a long-felt but unmet need in the art for attenuating the detrimental immune response to non-self gene therapies. The disclosure provides compositions and methods for promoting the elimination of immune effector cells specific to cells treated or modified by gene therapy techniques.

The importance of the role of FasL (Fas Ligand) in the pathway for immune regulation is well established. Activated T-cells upregulate Fas and become sensitive to FasL-mediated apoptosis in the process of activation-induced cell death and tolerance to self-antigens. Deficiencies in Fas or FasL often cause autoimmune pathologies or aberrant lymphoproliferation demonstrating the apparent lack of compensatory mechanisms in the pathway. While local presentation of mutated FasL has been shown to prevent rejection of transplanted cells in mice, ectopic expression of FASL in certain transplantation settings has had mixed results in achieving graft survival. In many instances, gene therapies delivering a non-self therapeutic transgene, such as a CRISPR/Cas complex, to a patient in need of such treatment can trigger an undesirable immune response to the therapeutic transgene and/or to the vector delivering the transgene. As such, there is a need to provide compositions and methods for masking immune activity and thereby promoting elimination of immune effector cells specific to cells treated and/or modified by gene therapy techniques.

### SUMMARY

The disclosure provides a composition comprising: a sequence encoding a non-self polypeptide of interest (POI), and a sequence encoding a non-cleavable FasL, wherein expression of the non-cleavable FasL eliminates MHC-mediated immunogenic peptides and helper T cells specific to the expression of the POI. In some embodiments, the POI is a CRISPR-Cas protein. In some embodiments, the POI is a viral capsid polypeptide such as an AAV viral capsid. In other embodiments, the POI is a heterologous non-self (foreign) protein antigen, fragment or variant thereof. In another embodiment, non-self proteins or POIs are selected from the group consisting of bacterial proteins, archaeal proteins, viral proteins, parasitic proteins, tumor proteins, mycoplasma proteins, yeast proteins or allergen proteins. In one embodiment, a non-self POI is a bacterially-derived CRISPR/Cas protein or an archaeal-derived CRISPR/Cas protein.

The disclosure also provides a composition comprising a sequence comprising: a guide RNA (gRNA) that specifically binds a target sequence within an RNA molecule, a sequence encoding an RNA-binding polypeptide, and a sequence encoding a non-cleavable FASL, wherein expression of the non-cleavable FASL eliminates MHC-mediated immunogenic peptides and helper T cells specific to the expression of the RNA-binding polypeptide.

In some embodiments of the compositions of the disclosure, the target sequence comprises at least one repeated sequence.

In some embodiments of the compositions of the disclosure, the sequences are within the same vector.

In some embodiments of the compositions of the disclosure, the vector is a viral vector. In some embodiments, the viral vector is an AAV vector, an adenoviral vector, or a retroviral vector such as a lentiviral vector.

In some embodiments of the compositions of the disclosure, the vector is an AAV vector and the vector comprises sequences encoding the AAV capsid.

In some embodiments of the compositions of the disclosure, the sequences comprise an IRES (Internal Ribosomal Entry Site) or a 2A ribosomal site.

In some embodiments of the compositions of the disclosure, the mutated non-cleavable FasL comprises at least one mutation or deletion in its metalloproteinase cleavage site. In some embodiments, the mutated non-cleavable FasL comprises at least one mutation or deletion in its protease recognition region. In another embodiment, the protease recognition region is at least amino acid residues 119 to 154 of wild-type human FasL.

In some embodiments, the metalloproteinase cleavage site comprises the amino acid sequence ELAELR. In another embodiment, the mutation comprises one or more of a substitution, an insertion, a deletion, a frameshift, an inversion, or a transposition of the amino acid sequence ELAELR.

In some embodiments, the non-cleavable FASL comprises the amino acid sequence of:

(SEQ ID NO: 210)
MQQPFNYPYPQIYWVDSSASSPWAPPGTVLPCPTSVPRRPGQRRPPPP
PPPPPLPPPPPPPPLPPLPLPPLKKRGNHSTGLCLLVMFFMVLVALVG
LGLGMFQLFHLQKX₁X₂X₃X₄X₅X₆ESTSQMHTASSLEKQIGHPSPPPEK
KELRKVAHLTGKSNSRSMPLEWEDTYGIVLLSGVKYKKGGLVINETGL
YFVYSKVYFRGQSCNNLPLSHKVYMRNSKYPQDLVMMEGKMMSYCTTG
QMWARSSYLGAVFNLTSADHLYVNVSELSLVNFEESQTFFGLYKL,

wherein X₁ is not a glutamic acid (E), X₂ is not an leucine (L), X₃ is not an alanine (A), X₄ is not an glutamic acid (E), X₅ is not an leucine (L) or X₆ is not an arginine (R).

In some embodiments, the non-cleavable FASL comprises the amino acid sequence of:

(SEQ ID NO: 210)
MQQPFNYPYPQIYWVDSSASSPWAPPGTVLPCPTSVPRRPGQRRPPPP
PPPPPLPPPPPPPPLPPLPLPPLKKRGNHSTGLCLLVMFFMVLVALVG
LGLGMFQLFHLQKX₁X₂X₃X₄X₅X₆ESTSQMHTASSLEKQIGHPSPPPEK
KELRKVAHLTGKSNSRSMPLEWEDTYGIVLLSGVKYKKGGLVINETGL
YFVYSKVYFRGQSCNNLPLSHKVYMRNSKYPQDLVMMEGKMMSYCTTG
QMWARSSYLGAVFNLTSADHLYVNVSELSLVNFEESQTFFGLYKL,

wherein X₁ is not a glutamic acid (E), X₂ is not an leucine (L), X₃ is not an alanine (A), X₄ is not an glutamic acid (E), X₅ is not an leucine (L) and X₆ is not an arginine (R).

In some embodiments, expression of the non-cleavable FASL selectively eliminates a T-cell that recognizes a MHC-peptide complex, wherein the peptide is derived from the non-self polypeptide, and wherein expression of FASL is in the presence of IL-6 or TNF-alpha.

In some embodiments, the non-cleavable FASL comprises an intron, wherein the intron blocks FASL splicing in the absence of IL-6 or TNF-alpha.

In some embodiments, the non-cleavable FASL comprises an intron, wherein the intron blocks FASL splicing in the absence of IL-6 or TNF-alpha. In a further embodiment, the composition comprises synthetic mRNA target sites which are expressed in the presence of IL-6 or TNF-alpha.

In some embodiments, the compositions comprise 1) a synthetic notch system, 2) microRNA target sites, or a 3) split intein and engineered IL-6 or TNF-alpha receptors for regulating expression of FASL in the presence of IL-6 or TNF-alpha.

In some embodiments of the compositions of the disclosure, the RNA-binding polypeptide or RNA-binding portion thereof is selected from the group consisting of Cas9, Cas13d, PUF, PUMBY, and PPR.

In some embodiments of the compositions of the disclosure, the sequences comprise a promoter or promoters.

In some embodiments, the promoter driving expression of FASL is regulated by the presence of IL-6 receptor or TNF-alpha receptor. In some embodiments, a promoter capable of driving FASL expression in the presence of IL-6 receptor or TNF-alpha receptor is a promoter listed in Table 1 or Table 2.

In some embodiments, the non-self POI is a nucleoprotein complex encoded by (i) a sequence comprising a guide RNA (gRNA) that specifically binds a target sequence within an RNA molecule, and (ii) a sequence encoding an RNA-binding polypeptide.

In some embodiments of the compositions of the disclosure, the sequence comprising the gRNA further comprises a sequence encoding a promoter capable of expressing the gRNA in a eukaryotic cell.

In some embodiments of the compositions of the disclosure, the eukaryotic cell is an animal cell. In some embodiments, the animal cell is a mammalian cell. In some embodiments, the animal cell is a human cell.

In some embodiments of the compositions of the disclosure, the promoter is a constitutively active promoter. In some embodiments, the promoter comprises a sequence isolated or derived from a promoter capable of diving expression of an RNA polymerase. In some embodiments, the promoter sequence comprises a sequence isolated or derived from a U6 promoter. In some embodiments, the promoter sequence comprises a sequence isolated or derived from a promoter capable of driving expression of a transfer RNA (tRNA). In some embodiments, the promoter sequence comprises a sequence isolated or derived from an alanine tRNA promoter, an arginine tRNA promoter, an asparagine tRNA promoter, an aspartic acid tRNA promoter, a cysteine tRNA promoter, a glutamine tRNA promoter, a glutamic acid tRNA promoter, a glycine tRNA promoter, a histidine tRNA promoter, an isoleucine tRNA promoter, a leucine tRNA promoter, a lysine tRNA promoter, a methionine tRNA promoter, a phenylalanine tRNA promoter, a proline tRNA promoter, a serine tRNA promoter, a threonine tRNA promoter, a tryptophan tRNA promoter, a tyrosine tRNA promoter, or a valine tRNA promoter. In some embodiments, the promoter comprises a sequence isolated or derived from a valine tRNA promoter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-B are schematic diagrams relevant to the compositions of the disclosure. (A) Depicts typical therapeutic non-self transgene delivery via AAV which result in presentation of non-self polypeptides that can activate T helper cells and potentiate a cytotoxic effect against treated tissue or cells. (B) Depicts various embodiments of the compositions of the disclosure by including sequences encoding mutated (metalloproteinase non-cleavable) versions of FasL in vector constructs comprising therapeutic transgenes (Tx genes), such as transgene components encoding a CRISPR/Cas9 complex, thereby resulting in the promotion of programmed death of T-cells that interrogate treated tissue or cells and preventing cytotoxic activity against the treated tissue or cells.

FIG. 2A-B are schematic diagrams relevant to the compositions of the disclosure. (A) Depicts repeated AAV administration in humans which results in formation of adaptive immunity against the AAV capsid in the form of both humoral and cellular responses. (B) Depicts compositions of the disclosure by including sequences encoding both mutated non-cleavable FasL and polypeptides from the AAV vector capsid in the vector constructs additionally comprising a therapeutic transgene (self or non-self). This results in elimination of T-cells specific to the viral capsid and prevention of the formation of adaptive immunity against the viral capsid which allows for efficient and safe redosing with the AAV vector.

FIG. 3A-F are schematic diagrams relevant to embodiments of the compositions disclosed herein that are capable of detecting the activity of T cells. (A) Depicts a construct configuration embodiment comprising FASL driven by a promoter that is regulated by IL-6 receptor or TNF-alpha receptor. (B) Depicts a construct configuration embodiment comprising a Cas13d RNA-targeting system and FASL. The FASL comprises an intron whose splicing is negatively regulated by Cas13d. Upon gene expression changes mediated by IL-6 or TNF-alpha, Cas13d is titrated away from the FASL construct so that splicing of FASL is allowed and FASL protein is produced. (C) Depicts a construct configuration embodiment similar to the construct configuration in (B) but with the addition of another component: engineered mRNA that is regulated by TNF-alpha receptor or IL-6 receptor that contains concatenated sites which titrate Cas13d away from the FASL pre-mRNA. (D) Depicts a construct configuration embodiment comprising an engineered receptor such as Syn-notch that detects IL-6 or TNF-alpha and subsequently releases a transcription factor such as GAL4 thereby promoting expression of a GAL4-regulated FASL gene. (E) Depicts a construct configuration embodiment comprising an engineered mRNA that codes for FASL and also contains concatenated target sites in the 3′UTR for a microRNA (miRNA) that is downregulated upon stimulation by TNF-alpha or IL-6. (F) Depicts a construct configuration embodiment comprising an engineered version of IL-6 receptor or TNF-alpha receptor that carries an intein on the intracellular domain along with a Cas13d-intein fusion present in the nucleus. This construct embodiment is similar to the embodiment of (B) in that the Cas13d regulates splicing of FASL but the release of the intein from the cell membrane and translocation to the nucleus upon IL-6 or TNF-alpha detection results in intein activity on Cas13d thereby releasing the splicing block on FASL.

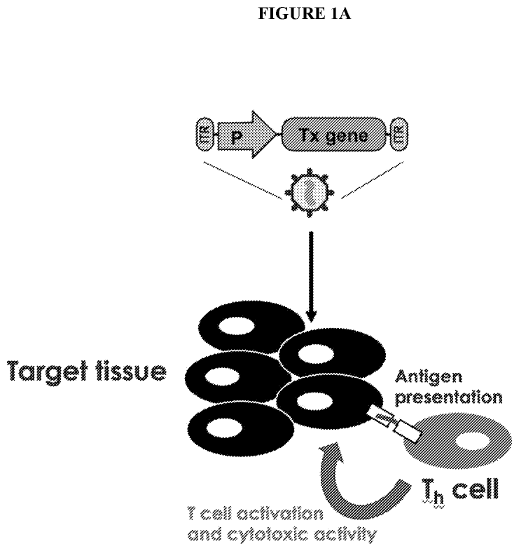

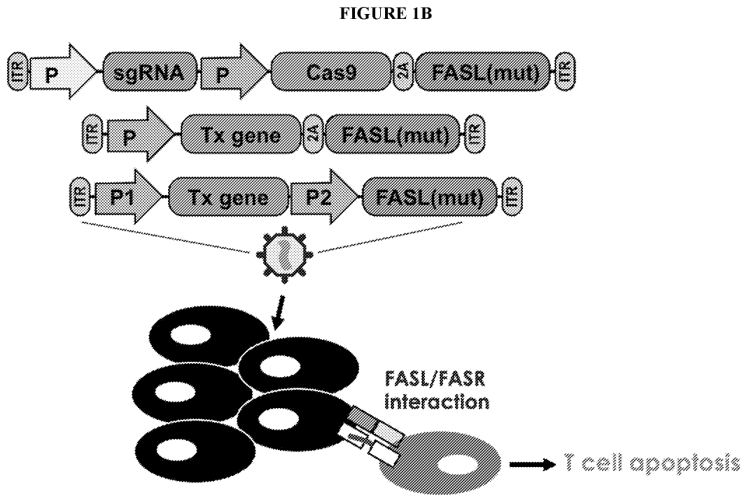

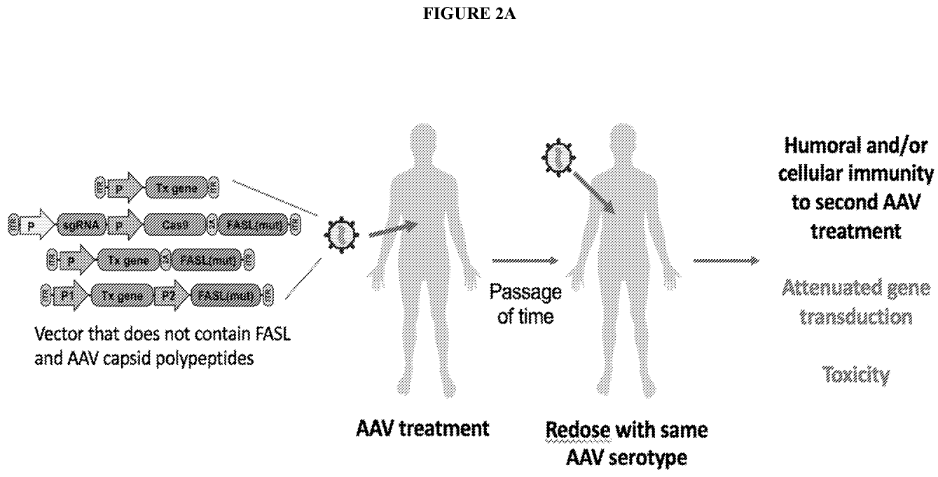

### DETAILED DESCRIPTION

The disclosure provides compositions and methods for combined therapeutic and immune masking activity. The immune masking activity eliminates MHC-mediated immunogenic peptides and helper T-cells specific to the expression of a non-self therapeutic activity, i.e., a non-self therapeutic protein such as a CRISPR/Cas ribonucleoprotein complex. The compositions comprise nucleic acid sequences which encode at least two functional components—a non-self protein of interest (POI) and a non-cleavable mutated FasL. In one embodiment of the compositions of the disclosure, the compositions comprise nucleic acid sequences comprising a gRNA that specifically binds a target sequence within an RNA molecule, a sequence encoding an RNA-binding polypeptide or RNA-binding portion thereof, and a sequence encoding a non-cleavable FasL. In another embodiment, the compositions comprise vector constructs. In other embodiments, the sequences comprise a promoter driving the functional components or separate promoters driving expression of each or certain of the functional components. Additional elements often used in the expression of multiple coding sequences such as 2A ribosomal skipping sites, or IRESs can be incorporated in the compositions comprising the vector constructs.

An important feature of the compositions and methods of the disclosure is controlling the timing and levels associated with FASL expression. Constitutive expression of FASL is associated with toxicity but by expressing FASL when cells are challenged by activated T cells, selective T cell elimination is achieved while avoiding these toxicity issues.

In one embodiment, temporal control of FASL expression is achieved by utilizing delivery modes that promote short-term expression of the FASL system. Specifically, nonviral delivery modes such as lipid nanoparticles carrying DNA or RNA encoding the FASL system promotes transient expression of the system in the target tissue.

In another embodiment, AAV vectors or other viral delivery or nonviral delivery modes comprise built-in temporal controls. One such approach involves promoters that cycle with circadian rhythms such as the clock gene. Another could involve the use of drug-inducible promoters such as, without limitation, tetracycline, cumate, galactose (GAL), alcohol oxidase (AOX), cellobiohydrolase, or glucoamylase.

In another embodiment, integrated sensors promote FASL expression only under controlled conditions. Specifically, a genetic circuit that recognizes expression of specific genes is used to identify the activity of cytotoxic T cells and subsequently promote FASL expression only in the presence of these activated T cells.

Accordingly, the disclosure provides compositions and methods for regulating and/or controlling expression of mutant (mFASL). In one embodiment, the composition produces mFASL only in the presence of activated T cells via detection of the cytokines, IL-6 or TNFalpha. This mFASL protein protects the therapeutic-treated cells via specific killing of the activated T cell. In the absence of the cytokines, the cells downregulate FASL which avoids safety issues associated with broad, constitutive expression of FASL.

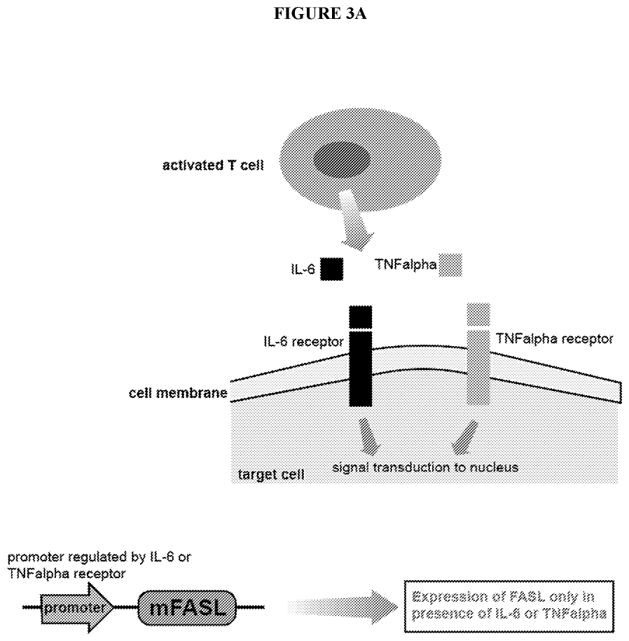

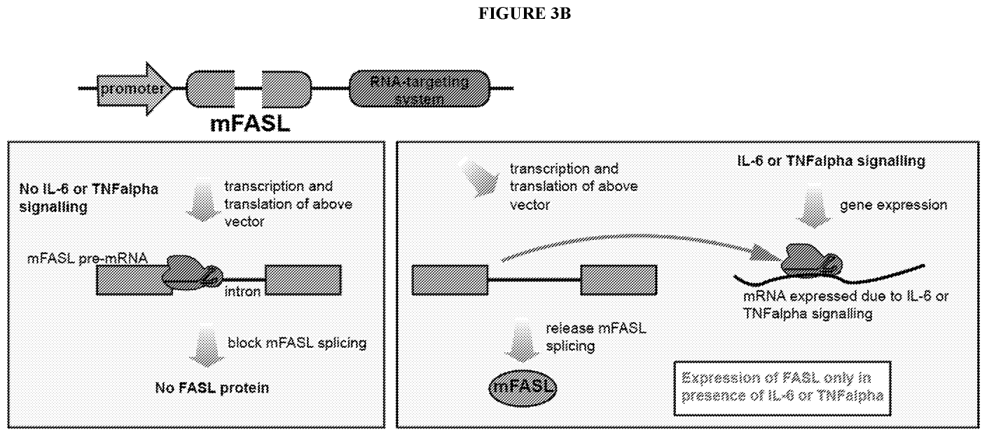

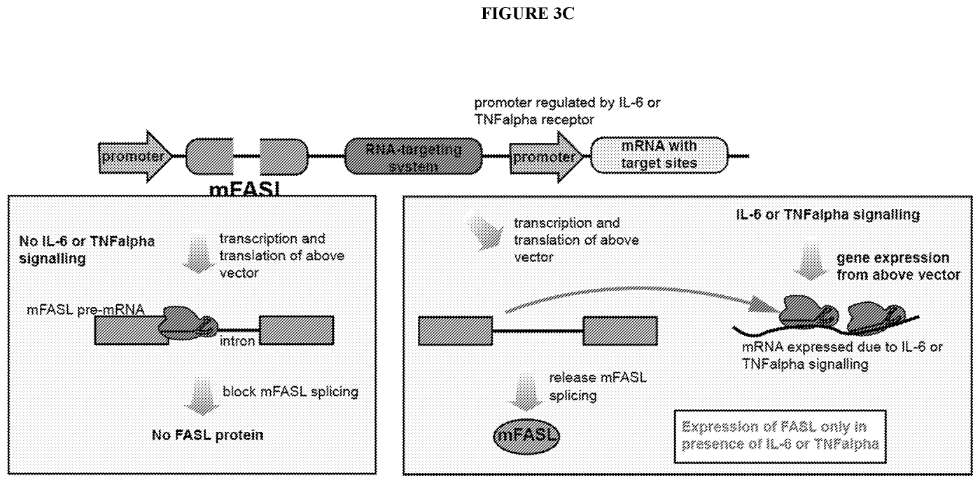

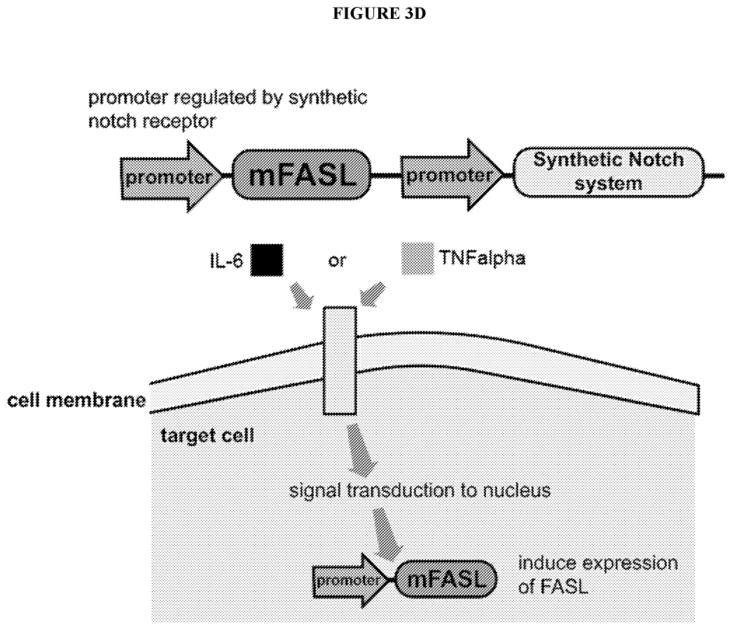

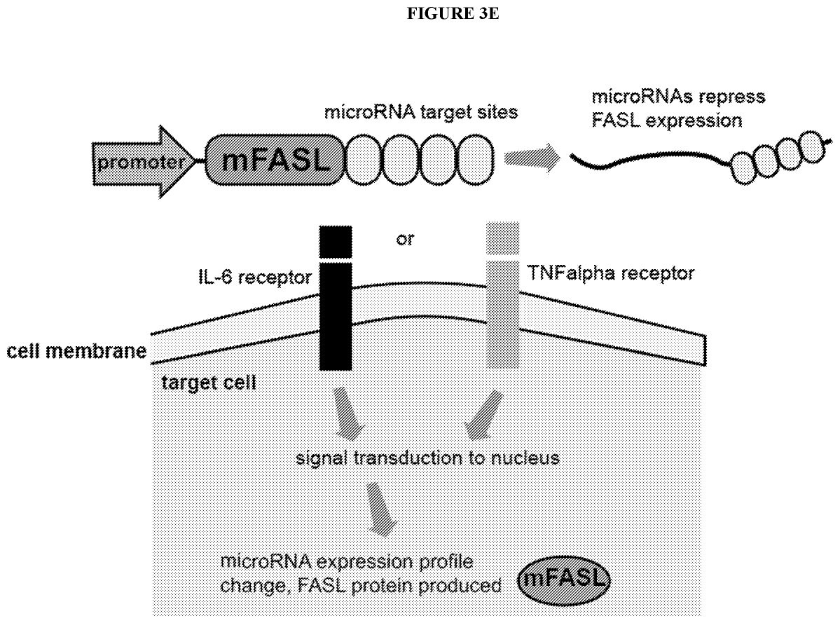

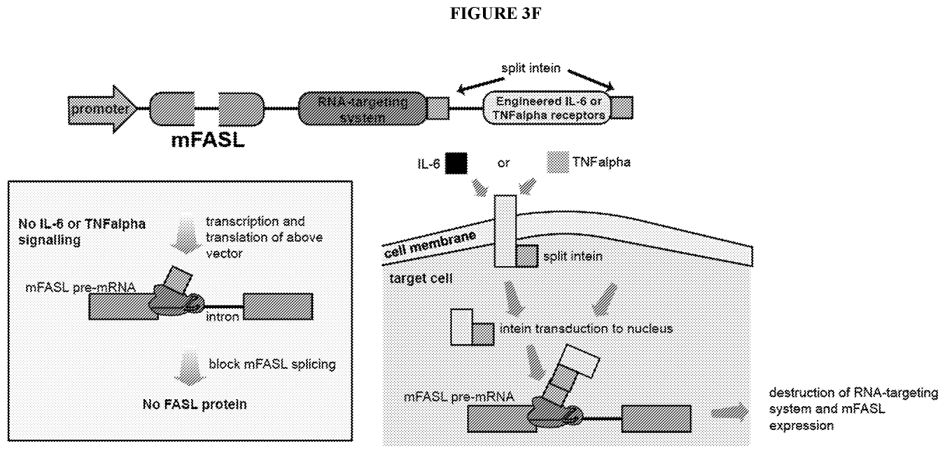

In one embodiment, the production of mFASL is only in the presence of activated T cells via use of a construct configuration, such as FIG. 3A, comprising a promoter which is specifically activated by one or both of IL-6 and/or TNF-alpha. Exemplary promoters which are specifically activated by one or both IL-6 and/or TNF-alpha include, without limitation, promoters listed in Table 1 and/or Table 2.

TABLE 1

```
Genes with promoters
regulated by TNF-alpha
  1. BLIMP1/PRDM1
  2. CCL5
  3. CCL15
  4. CCL17
  5. CCL19
  6. CCL20
  7. CCL22
  8. CCL23
  9. CCL28
  10. CXCL1
  11. CXCL11
  12. CXCL10
  13. CXCL3
  14. CXCL1
  15. GRO-beta
  16. GRO-gamma
  17. CXCL1
  18. ICOS
  19. IFNG
  20. IL1A
  21. IL1B
  22. IL1RN
  23. IL2
  24. IL6
  25. IL8
  26. IL9
  27. IL10
  28. IL11
  29. IL12B
  30. IL12A
  31. IL13
  32. IL15
  33. IL17
  34. IL23A
  35. IL27
  36. EBI3
  37. IFNB1
  38. CXCL5
  39. KC
  40. ligp1
  41. CXCL5
  42. CXCL6
  43. LTA
  44. LTB
  45. CCL2
  46. CXCL9
  47. CCL3
  48. CCL4
  49. CCL4
  50. CXCL3
  51. CCL20
  52. CXCL10
  53. CXCL5
  54. CCL5
  55. CCL1
  56. TNF
  57. LTA
  58. TNFSF10
  59. TFF3
  60. TNFSF15
  61. CD80
  62. BLR1
  63. CCR5
  64. CCR7
  65. IL8RA
  66. IL8RB
  67. TNFRSF9
  68. CD40LG
  69. CD3G
  70. CR2
  71. CD38
  72. CD40
  73. CD48
  74. CD83
  75. CD86
  76. SLC3A2
  77. TNFRSF4
  78. F11R
  79. FCGRT
  80. FCER2
  81. HLA-G
  82. ICOS
  83. IL2RA
  84. IGHG2
  85. IGHG1
  86. IGHG4
  87. IGHE
  88. IGKC
  89. BDKRB1
  90. HLA-B
  91. B2M
  92. NOD2
  93. pIgR
  94. PGLYRP1
  95. TCRB
  96. CD3G
  97. TLR2
  98. TLR9
  99. TNFRSF1B
100. TREM1
101. CFB
102. C3
103. CR2
104. PSMB9
105. TAP1
106. TAPBP
107. CD44
108. CD209
109. SELE
110. ENG
111. FN1
112. CD54
113. MADCAM1
114. NCAM
115. SELP
116. TNC
117. VCAM1
118. AGT
119. DEFB2
120. C4BPA
121. CFB
122. C4A
123. HAMP
124. LBP
125. PTX3
126. SAA1
127. SAA2
128. SAA3
129. F3
130. PLAU
131. CYP2E1
132. CYP2C11
133. CYP7B1
134. PTGS2
135. FTH1
136. GCLC
137. GCLM
138. HSP90AA1
139. ALOX5
140. ALOX12
141. NOS2A
142. MAP4K1
143. SENP2
144. SOD1
145. SOD1
146. SOD2
147. MX1
148. NQO1
149. PLA2
150. SELS
151. ABCA1
152. ABCC6
153. ADORA1
154. ADORA2A
155. ADAM19
156. SCNN1A
157. ADRA2B
158. BDKRB1
159. FCER2/CD23
160. C69
161. OPRD1
162. EGFR
163. ERBB2
164. KISS1
165. OLR1
166. KLRA1
167. ABCB4
168. OPRM1
169. GRM2
170. NPY1R
171. GRIN2A
172. GRIN1
173. OXTR
174. PTAFR
175. ABCB1
176. AGER
177. PYCARD
178. BAX
179. BCL2A1
180. BCL2L1
181. BCL2
182. BCL2L11
183. CD274
184. BNIP3
185. CASP4
186. CFLAR
187. FAS
188. CIDEA
189. PTPN13
190. FASLG
191. IER3
192. TRAF1
193. TRAF2
194. TIFA
195. XIAP
196. INHBA
197. ANGPT1
198. PI3KAP1
199. BDNF
200. TNFSF13B
201. BLNK
202. BMP2
203. BMP4
204. CALCB
205. FGF8
206. FSTL3
207. CSF3
208. CSF2
209. HGF
210. EPO
211. IGFBP1
212. IGFBP2
213. CSF1
214. MDK
215. NGFB
216. TACR1
217. NK4
218. NRG1
219. SPP1
220. PDGFB
221. PIGF
222. PENK
223. PRL
224. KITLG
225. THBS1
226. THBS2
227. VEGFC
228. WNT10B
229. TNFAIP2
230. EGR1
231. IER3
232. DCTN4
233. KLF10
234. TNFAIP3
235. TNIP3
236. AR
237. BCL3
238. BMI1
239. CDX1
240. FOS
241. MYB
242. MYC
243. REL
244. CEBPD
245. ZNF366
246. DMP1
247. E2F3
248. ELF3
249. AHCTF1
250. IER2
251. GATA3
252. NR3C1
253. HIF1A
254. HOXA9
255. IRF1
256. IRF2
257. IRF4
258. IRF7
259. NFKBIA
260. NFKBIE
261. JUNB
262. JMJD3
263. LEF1
264. CREB3
265. NFKBIZ
266. NFKB2
267. NFKB1
268. NLRP2
269. NR4A2
270. Osterix
271. TP53
272. PGR
273. SPI1
274. RELB
275. SNAI1
276. SOX9
277. STAT5A
278. TFEC
279. TWIST1
280. WT1
281. YY1
282. ABCB9
283. GCNT1
284. ADH1A
285. AICDA
286. AMACR
287. ARFRP1
288. ASS1
289. CYP19A1
290. ART1
291. SERPINA3
292. BACE1
293. BTK
294. CTSB
295. CTSL1
296. CDK6
297. UGCGL1
298. CHI3L1
299. Rdh1
300. Rdh7
301. MMP1
302. AKR1C1
303. DPYD
304. DNASE1L2
305. LIPG
306. ENO2
307. GAD1
308. ST8SIA1
309. NOX1
310. MMP9
311. GSTP1
312. GCLC
313. GCLC
314. GCLM
315. GCLC
316. G6PD
317. G6PC
318. GNRH2
319. GZMB
320. GUCY1A2
321. HPSE
322. HMOX1
323. HAS1
324. HSD11B2
325. HSD17B8
326. ATP1A2
327. DIO2
328. IDO1
329. PTGDS
330. LYZ
331. MTHFR
332. DUSP1
333. MMP3
334. MMP9
335. MYLK
336. NOS2A
337. NOS1
338. PDE7A
339. PIM1
340. PLK3
341. PIK3CA
342. PPP5C
343. PRKACA
344. PRKCD
345. PLCD1
346. PTGIS
347. PTGES
348. PTPN1
349. PTHLH
350. GNB2L1
351. REV3L
352. Slfn2
353. SERPINA2
354. ST6GAL1
355. NUAK2
356. SAT1
357. SUPV3L1
358. TERT
359. TGM1
360. TGM2
361. PAFAH2
362. UPP1
363. XDH
364. ABCG5
365. ABCG8
366. ASPH
367. ORM1
368. AFP
369. AMH
370. A4
371. APOBEC2
372. APOC3
373. APOD
374. APOE
375. AQP4
376. BGN
377. BRCA2
378. MYOZ1
379. CAV1
380. CDKN1A
381. CLDN2
382. COL1A2
383. GJB1
384. CCND1
385. CCND2
386. CCND3
387. IER3
388. SLC11A2
389. SKALP, PI3
390. EDN1
391. EPHA1
392. F8
393. FTH1
394. GADD45B
395. GNAI2
396. MT3
397. LGALS3
398. GBP1
399. HBE1
400. HBZ
401. IFI44L
402. KRT5
403. VPS53
404. HMGN1
405. FABP6
406. IGFBP2
407. KRT3
408. KRT6B
409. KRT15
410. LTF
411. LAMB2
412. LCN2
413. S100A4
414. SERPINB1
415. MUC2
416. MBP
417. SLC16A1
418. TNIP1
419. LCN2
420. FAM148A
421. S100A10
422. PSME1
423. PSME2
424. SERPINE1, PAI-1
425. PAX8
426. PTS
427. PRF1
428. PGK1
429. POMC
430. CGM3
431. PDYN
432. KLK3
433. PTEN
434. RAG1
435. RAG2
436. RBBP4
437. RIPK2
438. SERPINE2
439. S100A6
440. SH3BGRL
441. KCNN2
442. SKP2
443. SPATA19
444. OPN1SW
445. ERVWE1
446. SDC4
447. SLC6A6
448. KCNK5
449. TFPI2
450. TF
451. TICAM1
452. TRPC1
453. UBE2M
454. UCP2
455. UPK1B
456. CYP27B1
457. VIM
458. SERPINA1
459. CXCL1
```

TABLE 2

```
Promoters regulated
by IL6 (STAT3)
  1. BCAR3
  2. CALCB
  3. CCR6
  4. COL6A3
  5. CXCR5
  6. DHRS9
  7. FLT1
  8. FNBP1L
  9. FNDC9
10. GBP4
11. GPR87
12. GZMB
13. HOPX
14. HSD11B1
15. IFIT2
16. IFNL1
17. IGFBP6
18. IL12RB2
19. IL1R1
20. IL1R2
21. IL23R
22. IL24
23. KCNK18
24. MAF
25. NAPSA
26. PALLD
27. PRG4
28. PSD3
29. RORA
30. TNFSF1
31. TNFSF13B
32. TSHZ2
```

In another embodiment, mFASL expression is regulated by a construct configuration, such as FIG. 3B, comprising an RNA-targeting system (e.g., Cas13d) that prevents splicing of mFASL. Specifically, the FASL comprises an intron whose splicing is negatively regulated by the RNA-binding protein (e.g., Cas13d). Upon TNFalpha or IL-6 signaling, the RNA-targeting system is drawn to a stronger binding site in an RNA that is expressed upon TNFalpha or IL-6 signaling, that is, Cas13d is titrated away from the FASL construct. This releases the splicing block on mFASL (and splicing of FASL is permitted) and promotes production of the protein. In one embodiment, the Cas13d guide RNA (gRNA) is antisense to the mRNA of the regulated FASL construct configuration (such as in FIG. 3A). Spacer sequences for gRNAs targeting the IL6 or TNF-alpha-regulated mRNAs are listed in Table 3.

TABLE 3
Spacer sequences for
gRNAs targeting the
IL6 or TNF-alpha-
regulated mRNAs

| mRNA | SEQ ID NO: |
|---|---|
| FNDC9 | 250-2307 |
| PSD3 | 2308-13986 |
| FLT1 | 13987-21641 |
| TSHZ2 | 21642-33851 |
| RORA | 33852-44653 |
| KCNK18 | 44654-45783 |
| NAPSA | 45784-47664 |
| FNBP1L | 47665-53010 |
| CALCB | 53011-54027 |
| IL1R1 | 54028-59015 |
| COL6A3 | 59016-69589 |
| CCR6 | 69590-72780 |
| IL24 | 72781-74731 |
| HSD11B1 | 74732-76094 |
| IFNL1 | 76095-76844 |
| IL23R | 76945-79675 |
| MAF | 79676-82318 |
| PALLD | 82319-89021 |
| HOPX | 89022-90118 |
| IFIT2 | 90119-93485 |
| GPR87 | 93486-94953 |
| BCAR3 | 94954-98267 |
| IL1R2 | 98268-99704 |
| DHRS9 | 99705-101754 |
| IGFBP6 | 101755-102675 |
| PRG4 | 102676-107705 |
| CXCR5 | 107706-111973 |
| GZMB | 111974-112838 |
| IL12RB2 | 112839-116853 |
| GBP4 | 116854-122969 |
| TNFSF13B | 122970-125618 |
| TNFSF1 | 125619-127937 |

In a similar embodiment, a construct configuration, such as FIG. 3C, comprises an engineered RNA comprising concatenated sites that titrate Cas13d away from the FASL pre-mRNA and which is regulated by TNFalpha or IL-6 via use of an appropriate promoter (such as, without limitation, a promoter in Table 1 or Table 2). In this case, the engineered RNA contains multiple target sites for the RNA-targeting system. As such, expression of the engineered RNA releases the splicing block on the mFASL mRNA.

In another embodiment, a construct configuration, such as FIG. 3D, comprises an engineered receptor such as synthetic notch detects IL-6 or TNFalpha and regulates expression of a promoter that drives mFASL. In this manner, mFASL is only produced in the presence of TNFalpha or IL-6 signaling.

For example, such an engineered Syn-notch receptor would detect IL-6 or TNF-alpha and subsequently release a transcription factor such as GAL4 which promotes expression of a GAL4-regulated FASL gene. In one embodiment, the engineered receptor comprises three modules (from N- to C-terminus):

1) an IL-6 or TNF-alpha binding section such as, without limitation, an IL-6 scFV having an amino acid sequence as follows:

(SEQ ID NO: 227)
MSTVILSAAAPLSGVYAAMERGSHHHHHHGSGSGSGIEGRPYNGTGSA
CELGTQVQLKESGPGLVPSQSLSITCTVSDFSLTNYGVHWVRQSPGKG
LEWLGVIWSGGSTDYNAAFISRLSISKDNSKSQVFFEMNSLQADDTAI
YYCARNGNRYYGYALDYWGQGTSVTVSSGGGGSGGGGSGGGGSDVVMT
QTPLSLPVSLGDQASISCRSSQSIVHSNGNTYLEWYLQKPGQSPKLLI
YTVSNRLSGVPDRFSGSGSGTDFTLKISRVEAEDLGVYYCFQGSHGPY
TFGGGTKLEIKLQTCGRKLSLNQN

2) A synthetic notch such as, without limitation, having an amino acid sequence as follows:

(SEQ ID NO: 228)
ILDYSFTGGAGRDIPPPQIEEACELPECQVDAGNKVCNLQCNNHACGWDGG
DCSLNFNDPWKNCTQSLQCWKYFSDGHCDSQCNSAGCLFDGFDCQLTEGQC
NPLYDQYCKDHFSDGHCDQGCNSAECEWDGLDCAEHVPERLAAGTLVLVVL
LPPDQLRNNSFHFLRELSHVLHTNVVFKRDAQGQQMIFPYYGHEEELRKHP
IKRSTVGWATSSLLPGTSGGRQRRELDPMDIRGSIVYLEIDNRQCVQSSSQ
CFQSATDVAAFLGALASLGSLNIPYKIEAVKSEPVEPPLPSQLHLMYVAAA
AFVLLFFVGCGVLLSRKRRR

and 3) a transcription factor such as, without limitation, GAL4 having the amino acid sequence as follows:

(SEQ ID NO: 229)
MKLLSSIEQACDICRLKKLKCSKEKPKCAKCLKNNWECRYSPKTKRSPLTR
AHLTEVESRLERLEQLFLLIFPREDLDMILKMDSLQDIKALLTGLFVQDNV
NKDAVTDRLASVETDMPLTLRQHRISATSSSEESSNKGQRQLTVSAAAGGS
GGSGGSDALDDFDLDMLGSDALDDFDLDMLGSDALDDFDLDMLGSDALDDF
DLDMLGS

In another embodiment, in a construct configuration, such as FIG. 3E, mFASL is regulated via placement of microRNA (miRNA) binding sites of interest in the mRNA 3′UTR. The engineered mRNA comprises concatenated target sites (for an miRNA or miRNAs of interest) and are selected so that these microRNAs are expressed in cells that are not subjected to TNFalpha or IL-6. Cells that experience TNF-alpha or IL-6 reduce expression of the microRNA (i.e., the miRNA is downregulated upon stimulation by TNF-alpha or IL-6), resulting in mFASL expression only in the presence of cytokine signaling. In one embodiment, the engineered mRNA comprises target sites for miRNA, without limitation, selected from the group consisting of hsa-miR-934, hsa-miR-1269a, hsa-miR-671-5p, hsa-miR-663a, hsa-miR-1292, hsa-miR-615-5p, hsa-miR-2276, hsa-miR-1307-3p, hsa-miR-3654, hsa-miR-4741, hsa-miR-100-5p, hsa-miR-3189-3p, hsa-miR-548t-5p, hsa-miR-769-3p, hsa-miR-1307-5p, hsa-miR-3687, hsa-miR-324-5p, hsa-miR-449c-5p, hsa-miR-532-5p, hsa-miR-122-5p, hsa-miR-301b, hsa-miR-652-3p, hsa-miR-181a-5p, hsa-miR-140-3p, hsa-miR-331-3p, hsa-miR-10a-5p, hsa-miR-3656, hsa-miR-146a-5p, hsa-miR-1246, hsa-miR-143-3p, hsa-miR-23a-5p, hsa-miR-4508, hsa-miR-4488, hsa-miR-548o-3p, hsa-miR-29c-5p, hsa-miR-21-3p, hsa-miR-215, hsa-miR-139-3p, hsa-miR-720, hsa-miR-3141, hsa-miR-29b-1-5p, hsa-miR-141-5p, hsa-miR-25-5p, hsa-miR-197-5p, hsa-miR-1260b, hsa-miR-22-5p, and hsa-miR-628-5p.

In another embodiment, a construct configuration, such as FIG. 3F, comprises an engineered receptor that detects IL-6 or TNFalpha and comprises a split intein (e.g., an intein on the intracellular domain along with a Cas13d-intein fusion that is present in the nucleus). The RNA-targeting system (such as Cas13d) regulates the splicing of an mRNA encoding mFASL and releases the intein from the cell membrane. Accordingly, upon activation of the synthetic receptor, the fused split intein translocates to the nucleus where it interacts with the split intein fused to the RNA-targeting system. The result is the destruction of a functional RNA-targeting system, correct mFASL mRNA splicing, and the production of mFASL protein.

The disclosure provides vectors, compositions and cells comprising the therapeutic and FasL immune masking nucleic acid sequences. The disclosure provides methods of using the vectors, compositions and cells of the disclosure to treat a disease or disorder and at the same time eliminate the WIC-mediated immunogenic response specific to the vectors and/or compositions and treated cells.

### Preventing Adaptive Immune Response to a Non-Self Therapeutic Transgene

An AAV vector carrying a therapeutic, non-self transgene is packaged with mutant FALS (mFASL) so that both genes are expressed. After administration of the AAV vector, treated cells begin to express both the transgene and mFASL. Peptides derived from the transgene are displayed by WIC as part of the typical and typical process of antigen presentation conducted by many cell types. The formation of regulatory and effector T cells that target the non-self peptides occurs. These transgene-specific T cells interrogate infected (treated) cells that display the non-self peptides and simultaneously encounter mFASL. The presence of this non-self peptide display and mFASL results in apoptosis of the transgene-specific T cells. This eliminates this facet of adaptive immune response against the therapeutic transgene and the cells that harbor it.

### Treatment of Myotonic Dystrophy Type I (DM1)

Compositions of the disclosure are used for the treatment of myotonic dystrophy type I (DM1) wherein an RNA-targeting CRISPR system composed of a therapeutic transgene (Cas9) and single guide RNA targeting the CUG repeats that cause DM1 are delivered to patient muscle or the central nervous system. The presence of mFASL causes the elimination of T cells that are specific to Cas9 and potentially cytotoxic against treated cells.

### Treatment of Hemophilia

Compositions of the disclosure are used for the treatment of hemophilia. A secreted transgene such as Factor IX is used for the treatment of hemophilia. A vector carrying an expression cassette for factor IX along with mFASL reduces, eliminates, or prevents an adaptive immune response to Factor IX-expressing cells.

Preventing Adaptive Immune Response to a Non-Self Therapeutic Transgene while Simultaneously Preventing Immune Response to Repeated AAV Administrations

Compositions of the disclosure may comprise an AAV vector containing an expressed polypeptide composed of all or part of AAV viral capsid protein. The AAV capsid polypeptide is identical to the serotype used to deliver the system. Co-expression of this AAV capsid polypeptide causes the elimination of T cells that are specific to the AAV capsid in a manner described above. This causes depletion of T cells that can regulate both cellular and humoral immunity to the AAV capsid. This allows repeated dosing of the same AAV serotype. In the absence of the compositions of the disclosure, and using the standard of care prior to development of the compositions of the disclosure, an individual AAV serotype could not be used in more than once in a patient due to the formation of adaptive immune response against the viral capsid.

The compositions of the disclosure may be useful in situations wherein incomplete therapeutic transfer occurs during the first administration of a gene therapy or wherein a second dose is desired. In this case, the second dose of the gene therapy does not require the presence of the mFASL and AAV capsid polypeptide unless subsequent doses beyond the second dose are desired. One situation could be during the treatment of large organs such as skeletal muscle where the volume of virus required to transduce muscle in a single dose is prohibitively high. Another situation could be during treatment involving complicated administration methods in the brain or spine where initial treatments do not provide satisfactory infection of targeted cells.

### Non-Cleavable FasL

The Fas/FasL interaction is well established with regards to the immune system. The activation of T cells through the T cell receptor (TCR) upregulates both Fas and FasL. In circumstances of low to moderate TCR stimulation, T cells proliferate. Under conditions of repetitive or high levels of TCR stimulation, T cells are driven toward apoptosis. This phenomenon has been termed Antigen Induced Cell Death (AICD). The importance of AICD in regulating the immune system has been demonstrated in the LPR mouse. Nagata et al., Immunol. Today 16:39-43 (1995).

That the Fas/FasL interaction contributes to immune privilege is also well established. In particular, a number of studies demonstrate engineered immune privilege via the induction of FasL expression in transplantation settings. Bellgrau et al., Nature 377:630-632 (1995); Griffith et al., Science 270:1189-1192 (1995), Lau et al., Science 273:109-112 (1996).

FasL is proteolytically cleaved by matrix metalloproteases and bound to the cell membrane. Because soluble FasL is released into and circulated widely throughout the circulatory system, it is known to cause non-specific and widespread cell death. Ogasawara et al., Nature 364:806-809 (1993), published erratum, Nature 365:568 (1993), Tanaka et al., Nature Med. 2:317-322 (1996), Rodriguez et al., J. Exp. Med. 183:1031-1036 (1996). As such, selective modulation of Fas/FasL and the subsequent selective induction of apoptosis to specific target tissues and cells has been achieved by the mutation of the FasL protease recognition region. This is because it has been found that making at least one mutation or deletion in the wild-type FasL protease recognition region inhibits proteolytic cleavage of the FasL polypeptide from the cell membrane and minimizes the production of and the deleterious non-selective effects of soluble FasL. The sequence of the wild-type, full-length human FasL is known in the art. The extracellular domain of the wild-type, full-length human FasL is defined by amino acid residues 103 to 281, and the protease recognition region of wild-type human FasL comprises at least amino acid residues 119 to 154. Residues are numbered by reference to the known amino acid sequence of wild-type human FasL. See Takahashi et al., Int'l Immunol. 6:1567-1574 (1994). Moreover, non-cleavable mutated FasL polypeptides and methods of generating the same can be found, e.g., in WO 1999/036079, which is incorporated herein by reference in its entirety.

The terminology “FASL” and “mFasL” are used interchangeably herein to refer to non-cleavable mutated FasL.

In one embodiment, an exemplary mutated non-cleavable FasL (Mus musculus) (MMP cleavage site in bold) can be generated by making one or more mutations or deletions in the following amino acid sequence:

(SEQ ID NO: 209)
PGSVFPCPSCGPRGPDQRRPPPPPPPVSPLPPPSQPLPLPPLTPLKKKDHN
TNLWLPVVFFMVLVALVGMGLGMYQLFHLQKELAELREFTNQSLKVSSFEK
QIANPSTPSEKKEPRSVAHLTGNPHSRSIPLEWEDTYGTALISGVKYKKGG
LVINETGLYFVYSKVYFRGQSCNNQPLNHKVYMRNSKYPEDLVLMEEKRLN
YCTTGQIWAHSSYLGAVFNLTSADHLYVNISQLSLINFEESKTFFGLYKL.

In another embodiment, an exemplary mutated non-cleavable FasL (Homo sapiens) (MMP cleavage site in bold) can be generated by making one or more mutations or deletions in the following amino acid sequence:

(SEQ ID NO: 210)
MQQPFNYPYPQIYWVDSSASSPWAPPGTVLPCPTSVPRRPGQRRPPPPPPP
PPLPPPPPPPPLPPLPLPPLKKRGNHSTGLCLLVMFFMVLVALVGLGLGMF
QLFHLQKELAELRESTSQMHTASSLEKQIGHPSPPPEKKELRKVAHLTGKS
NSRSMPLEWEDTYGIVLLSGVKYKKGGLVINETGLYFVYSKVYFRGQSCNN
LPLSHKVYMRNSKYPQDLVMMEGKMMSYCTTGQMWARSSYLGAVFNLTSAD
HLYVNVSELSLVNFEESQTFFGLYKL.

### Non-Self POIs

With regard to an embodiment relating to one component of the compositions of the disclosure, a nucleic acid sequence of the composition encodes a non-self protein of interest (POI). In one embodiment, a non-self POI is a heterologous non-self (or foreign) protein antigen, fragment or variant thereof. Exemplary non-self proteins or POIs include, without limitation, bacterial proteins, archaeal proteins, viral proteins (e.g., viral capsids), parasitic proteins, tumor proteins, mycoplasma proteins, yeast proteins or allergen proteins. In one embodiment, a non-self POI is a bacterially-derived CRISPR/Cas protein or an archaeal-derived CRISPR/Cas protein. In another embodiment, a non-self POI is a viral capsid specific to the viral vector carrying a therapeutic transgene (self or non-self transgene).

### AAV Capsids—Repeated Administration of Self or Non-Self Gene Therapy

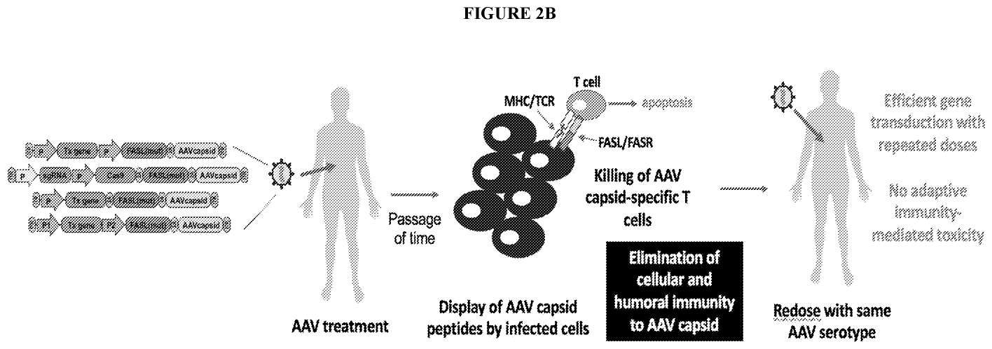

Repeated AAV administration in humans and animal models typically results in formation of adaptive immunity against the AAV capsid in the form of both humoral and cellular responses (FIG. 2A). As a result, repeated doses of AAV result in attenuated gene transduction after the initial dose with the potential for toxic effects. By include both FASL and polypeptides from the AAV capsid in the transgene payload (self or non-self transgenes) carried within the AAV vector used in the initial treatment, T-cells specific to the viral capsid can be eliminated (FIG. 2B). Elimination of these capsid-specific T-cells prevents the formation of adaptive immunity against the viral capsid and allows efficient and safe redosing. Specifically, the expression of the viral capsid polypeptide causes infected cells to display peptides specific to the viral capsid via WIC which will promote interaction among capsid-specific T cells (with TCRs for the viral capsid peptides) and infected cells. The co-expression of FASL on the infected cells will promote killing of these capsid-specific T-cells. As the T-cells are required for mounting of both cellular and humoral immunity against the capsid, subsequent treatments with the same AAV serotype will not be attenuated by the adaptive immune system.

AAV biology has been extensively studied and is well known in the art. AAV capsids for use in the compositions disclosed herein are derived from AAV serotypes which include, without limitation, AAV1, AAV2, AAV4, AAV5, AAV6 (a hybrid of AAV1 and AAV2), AAV7, AAV8, AAV9, AAV10, AAV11, AAV12, and synthetic AAV serotypes, such as, without limitation, Anc80 AAV (an ancestor of AAV 1, 2, 6, 8 and 9).

In one embodiment, the AAV capsid is derived from the AAV9 VP1 amino acid sequence which is:

(SEQ ID NO: 211)
MAADGYLPDWLEDNLSEGIREWWALKPGAPQPKANQQHQDNARGLVLPGYK
YLGPGNGLDKGEPVNAADAAALEHDKAYDQQLKAGDNPYLKYNHADAEFQE
RLKEDTSFGGNLGRAVFQAKKRLLEPLGLVEEAAKTAPGKKRPVEQSPQEP
DSSAGIGKSGAQPAKKRLNFGQTGDTESVPDPQPIGEPPAAPSGVGSLTMA
SGGGAPVADNNEGADGVGSSSGNWHCDSQWLGDRVITTSTRTWALPTYNNH
LYKQISNSTSGGSSNDNAYFGYSTPWGYFDFNRFHCHFSPRDWQRLINNNW
GFRPKRLNFKLFNIQVKEVTDNNGVKTIANNLTSTVQVFTDSDYQLPYVLG
SAHEGCLPPFPADVFMIPQYGYLTLNDGSQAVGRSSFYCLEYFPSQMLRTG
NNFQFSYEFENVPFHSSYAHSQSLDRLMNPLIDQYLYYLSKTINGSGQNQQ
TLKFSVAGPSNMAVQGRNYIPGPSYRQQRVSTTVTQNNNSEFAWPGASSWA
LNGRNSLMNPGPAMASHKEGEDRFFPLSGSLIFGKQGTGRDNVDADKVMIT
NEEEIKTTNPVATESYGQVATNHQSAQAQAQTGWVQNQGILPGMVWQDRDV
YLQGPIWAKIPHTDGNFHPSPLMGGFGMKHPPPQILIKNTPVPADPPTAFN
KDKLNSFITQYSTGQVSVEIEWELQKENSKRWNPEIQYTSNYYKSNNVEFA
VNTEGVYSEPRPIGTRYLTRNL.

In another embodiment, the predicted surface residues of AAV9 capsid (subset of VP1) is:

(SEQ ID NO: 212)
AKTAPGKKRPVEQSPQEPDSSAGIGKSGAQPAKKRLNFGQTGDTESVPDPQ
PIGEPPAAPSGVGSLTMASWHCDSQWLGDRVITTSTRTWALPTYNNHLYKQ
ISNSTSGGSSNDNAYFGYSTPWGYFDFNRFWHCDSQWLGDRVITTSTRTWA
LPTYNNHLYKQISNSTSGGSSNDNAYFGYSTPWGYFDFNRFDVFMIPQYGY
LTLNDGSQAVGRSSFYCLEYFPSQMLRTGNNFQFSYEFENVPFHSSYAHSQ
SLDRLMNPLIDQYLYYLSKTINGSGQNQQTLKFSVAGPSNMAVQGRNYIPG
PSYRQQRVSTTVTQNNNSEFAWPGASSWALNGRNSLMNPGPAMASHKEGED
RFFPLSGSLIFGKQGTGRDNVDADKVMITNEEEIKTTNPVATESYGQVATN
HQSAQAQAQTGWVQNQGILPGMVWIKNTPVPADPPTAFNKDKLNSFITQYS
TGQVSVEIEWELQKENSKRWNPEIQYTSNYYKSNNVEFAVNTEGVYSEPRP
IGTRYLTRNL.

In one embodiment, the AAV capsid is derived from the Anc80 AAV VP1 amino acid sequence which is:

(SEQ ID NO: 213)
AADGYLPDWLEDNLSEGIREWDLKPGAPKPKANQQKQDDGRGLVLPGYYL
GPFNGLDKGEPVNAADAAALEHDKAYDQQLKAGDNPYLRYNHADAEFQER
LQEDTSFGGNLGRAVFQAKKRVLEPLGLVEEGAKTAPGKKRPVEQSPQEP
DSSSGIGKKGQQPAXKRLNFGQTGDSESVPDPQPLGEPPAAPSGVGSNTM
XAGGGAPADNNEGADGVGNASGNWHCDSTWLGDRVITTSTRTALPTYNNH
LYKQISSQSGXSTNDNTYFGYSTPWGYFDFNRFHCHFSPRDWQRLINNNW
GFRPKXLNFKLFNIQVKEVTTNDGTTTIANNLTSTVQVFTDSEYQLPYVL
GSAHQGCLPPFPADVFMIPQYGYLTLNNGSQAVGRSSFYCLEYFPSQMLR
TGNNFXFSYTFEDVPFHSSYAHSQSLDRLNPLIDQYLYYLSRTQTTSGTA
GNRXLQFSQAGPSSANQAKNWLPGPCYRQQRVSKTXNQNNNSNFAWTGAT
KYHLNGRDSLVNPGPAMATHKDDEDKFFPMSGVLIFGKQGAGNSNVDLDN
VITXEEEIKTTNPVATEXYGTVATNLQSXNTAPATGTVNSQGALPGVWQX
RDVYLQGPIWAKIPHTDGHFHPSPLMGGFGLKHPPPQILIKNTPVPANPP
TTFSPAKFASFITQYSTGQVSVEIEELQKENSKRWNPEIQYTSNYNKSTN
VDFAVDTNGVYSEPRPIGTRYLTRNL

In one embodiment, the AAV capsid is derived from the AAV12 VP1 amino acid sequence which is:

(SEQ ID NO: 214)
MAADGYLPDWLEDNLSEGIREWWALKPGAPQPKANQQHQDNGRGLVLPGYK
YLGPFNGLDKGEPVNEADAAALEHDKAYDKQLEQGDNPYLKYNHADAEFQQ
RLATDTSFGGNLGRAVFQAKKRILEPLGLVEEGVKTAPGKKRPLEKTPNRP
TNPDSGKAPAKKKQKDGEPADSARRTLDFEDSGAGDGPPEGSSSGEMSHDA
EMRAAPGGNAVEAGQGADGVGNASGDWHCDSTWSEGRVTTTSTRTWVLPTY
NNHLYLRIGTTANSNTYNGFSTPWGYFDFNRFHCHFSPRDWQRLINNNWGL
RPKSMRVKIFNIQVKEVTTSNGETTVANNLTSTVQIFADSTYELPYVMDAG
QEGSFPPFPNDVFMVPQYGYCGVVTGKNQNQTDRNAFYCLEYFPSQMLRTG
NNFEVSYQFEKVPFHSMYAHSQSLDRMMNPLLDQYLWHLQSTTTGNSLNQG
TATTTYGKITTGDFAYYRKNWLPGACIKQQKFSKNANQNYKIPASGGDALL
KYDTHTTLNGRWSNMAPGPPMATAGAGDSDFSNSQLIFAGPNPSGNTTTSS
NNLLFTSEEEIATTNPRDTDMFGQIADNNQNATTAPHIANLDAMGIVPGMV
WQNRDIYYQGPIWAKVPHTDGHFHPSPLMGGFGLKHPPPQIFIKNTPVPAN
PNTTFSAARINSFLTQYSTGQVAVQIDWEIQKEHSKRWNPEVQFTSNYGTQ
NSMLWAPDNAGNYHELRAIGSRFLTHHL

In one embodiment, the AAV capsid is derived from the AAV1 VP1 amino acid sequence which is:

(SEQ ID NO: 215)
MAADGYLPDWLEDNLSEGIREWWDLKPGAPKPKANQQKQDDGRGLVLPGYK
YLGPFNGLDKGEPVNAADAAALEHDKAYDQQLKAGDNPYLRYNHADAEFQE
RLQEDTSFGGNLGRAVFQAKKRVLEPLGLVEEGAKTAPGKKRPVEQSPQEP
DSSSGIGKTGQQPAKKRLNFGQTGDSESVPDPQPLGEPPATPAAVGPTTMA
SGGGAPMADNNEGADGVGNASGNWHCDSTWLGDRVITTSTRTWALPTYNNH
LYKQISSASTGASNDNHYFGYSTPWGYFDFNRFHCHFSPRDWQRLINNNWG
FRPKRLNFKLFNIQVKEVTTNDGVTTIANNLTSTVQVFSDSEYQLPYVLGS
AHQGCLPPFPADVFMIPQYGYLTLNNGSQAVGRSSFYCLEYFPSQMLRTGN
NFTFSYTFEEVPFHSSYAHSQSLDRLMNPLIDQYLYYLNRTQNQSGSAQNK
DLLFSRGSPAGMSVQPKNWLPGPCYRQQRVSKTKTDNNNSNFTWTGASKYN
LNGRESIINPGTAMASHKDDEDKFFPMSGVMIFGKESAGASNTALDNVMIT
DEEEIKATNPVATERFGTVAVNFQSSSTDPATGDVHAMGALPGMVWQDRDV
YLQGPIWAKIPHTDGHFHPSPLMGGFGLKNPPPQILIKNTPVPANPPAEFS
ATKFASFITQYSTGQVSVEIEWELQKENSKRWNPEVQYTSNYAKSANVDFT
VDNNGLYTEPRPIGTRYLTRPL

In one embodiment, the AAV capsid is derived from the AAV2 VP1 amino acid sequence which is:

(SEQ ID NO: 216)
MAADGYLPDWLEDTLSEGIRQWWKLKPGPPPPKPAERHKDDSRGLVLPGYK
YLGPFNGLDKGEPVNEADAAALEHDKAYDRQLDSGDNPYLKYNHADAEFQE
RLKEDTSFGGNLGRAVFQAKKRVLEPLGLVEEPVKTAPGKKRPVEHSPVEP
DSSSGTGKAGQQPARKRLNFGQTGDADSVPDPQPLGQPPAAPSGLGTNTMA
TGSGAPMADNNEGADGVGNSSGNWHCDSTWMGDRVITTSTRTWALPTYNNH
LYKQISSQSGASNDNHYFGYSTPWGYFDFNRFHCHFSPRDWQRLINNNWGF
RPKRLNFKLFNIQVKEVTQNDGTTTIANNLTSTVQVFTDSEYQLPYVLGSA
HQGCLPPFPADVFMVPQYGYLTLNNGSQAVGRSSFYCLEYFPSQMLRTGNN
FTFSYTFEDVPFHSSYAHSQSLDRLMNPLIDQYLYYLSRTNTPSGTTTQSR
LQFSQAGASDIRDQSRNWLPGPCYRQQRVSKTSADNNNSEYSWTGATKYHL
NGRDSLVNPGPAMASHKDDEEKFFPQSGVLIFGKQGSEKTNVDIEKVMITD
EEEIRTTNPVATEQYGSVSTNLQRGNRQAATADVNTQGVLPGMVWQDRDVY
LQGPIWAKIPHTDGHFHPSPLMGGFGLKHPPPQILIKNTPVPANPSTTFSA
AKFASFITQYSTGQVSVEIEWELQKENSKRWNPEIQYTSNYNKSVNVDFTV
DTNGVYSEPRPIGTRYLTRNL

In one embodiment, the AAV capsid is derived from the AAV6 VP1 amino acid sequence which is:

(SEQ ID NO: 217)
MAADGYLPDWLEDNLSEGIREWWDLKPGAPKPKANQQKQDDGRGLVLPGYK
YLGPFNGLDKGEPVNAADAAALEHDKAYDQQLKAGDNPYLRYNHADAEFQE
RLQEDTSFGGNLGRAVFQAKKRVLEPFGLVEEGAKTAPGKKRPVEQSPQEP
DSSSGIGKTGQQPAKKRLNFGQTGDSESVPDPQPLGEPPATPAAVGPTTMA
SGGGAPMADNNEGADGVGNASGNWHCDSTWLGDRVITTSTRTWALPTYNNH
LYKQISSASTGASNDNHYFGYSTPWGYFDFNRFHCHFSPRDWQRLINNNWG
FRPKRLNFKLFNIQVKEVTTNDGVTTIANNLTSTVQVFSDSEYQLPYVLGS
AHQGCLPPFPADVFMIPQYGYLTLNNGSQAVGRSSFYCLEYFPSQMLRTGN
NFTFSYTFEDVPFHSSYAHSQSLDRLMNPLIDQYLYYLNRTQNQSGSAQNK
DLLFSRGSPAGMSVQPKNWLPGPCYRQQRVSKTKTDNNNSNFTWTGASKYN
LNGRESIINPGTAMASHKDDKDKFFPMSGVMIFGKESAGASNTALDNVMIT
DEEEIKATNPVATERFGTVAVNLQSSSTDPATGDVHVMGALPGMVWQDRDV
YLQGPIWAKIPHTDGHFHPSPLMGGFGLKHPPPQILIKNTPVPANPPAEFS
ATKFASFITQYSTGQVSVEIEWELQKENSKRWNPEVQYTSNYAKSANVDFT
VDNNGLYTEPRPIGTRYLTRPL

In one embodiment, the AAV capsid is derived from the AAV8 VP1 amino acid sequence which is:

(SEQ ID NO: 218)
MAADGYLPDWLEDNLSEGIREWWDLKPGAPKPKANQQKQDDGRGLVLPGYK
YLGPFNGLDKGEPVNAADAAALEHDKAYDQQLKAGDNPYLRYNHADAEFQE
RLQEDTSFGGNLGRAVFQAKKRVLEPLGLVEEGAKTAPGKKRPVEQSPQEP
DSSSGIGKTGQQPAKKRLNFGQTGDSESVPDPQPLGEPPAAPSGLGPNTMA
SGGGAPMADNNEGADGVGNSSGNWHCDSTWLGDRVITTSTRTWALPTYNNH
LYKQISNGTSGGSTNDNTYFGYSTPWGYFDFNRFHCHFSPRDWQRLINNNW
GFRPKRLNFKLFNIQVKEVTTNEGTKTIANNLTSTVQVFTDSEYQLPYVLG
SAHQGCLPPFPADVFMVPQYGYLTLNNGSQALGRSSFYCLEYFPSQMLRTG
NNFQFSYTFEDVPFHSSYAHSQSLDRLMNPLIDQYLYYLVRTQTTGTGGTQ
TLAFSQAGPSSMANQARNWVPGPCYRQQRVSTTTNQNNNSNFAWTGAAKFK
LNGRDSLMNPGVAMASHKDDDDRFFPSSGVLIFGKQGAGNDGVDYSQVLIT
DEEEIKATNPVATEEYGAVAINNQAANTQAQTGLVHNQGVIPGMVWQNRDV
YLQGPIWAKIPHTDGNFHPSPLMGGFGLKHPPPQILIKNTPVPADPPLTFN
QAKLNSFITQYSTGQVSVEIEWELQKENSKRWNPEIQYTSNYYKSTNVDFA
VNTEGVYSEPRPIGTRYLTRNL

### RNA-Binding Proteins

An RNA-binding protein, polypeptide, or domain of the disclosure includes, without limitation, an RNA-binding portion or portions of the RNA-binding protein or polypeptide or domain.

In some embodiments of the compositions of the disclosure, the sequence encoding an RNA-binding protein or RNA-binding portion thereof comprises a sequence isolated or derived from a CRISPR Cas protein. In some embodiments, the CRISPR Cas protein comprises a Type II CRISPR Cas protein. In some embodiments, the Type II CRISPR Cas protein comprises a Cas9 protein. Exemplary Cas9 proteins of the disclosure may be isolated or derived from any species, including, but not limited to, a bacteria or an archaea. Exemplary Cas9 proteins of the disclosure may be isolated or derived from any species, including, but not limited to, Streptococcus pyogenes, Haloferax mediteranii, Mycobacterium tuberculosis, Francisella tularensis subsp. novicida, Pasteurella multocida, Neisseria meningitidis, Campylobacter jejune, Streptococcus thermophilus, Campylobacter lari CF89-12, Mycoplasma gallisepticum str. F, Nitratifractor salsuginis str. DSM 16511, Parvibaculum lavamentivorans, Roseburia intestinalis, Neisseria cinerea, a Gluconacetobacter diazotrophicus, an Azospirillum B510, a Sphaerochaeta globus str. Buddy, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Treponema denticola, Legionella pneumophila str. Paris, Sutterella wadsworthensis, Corynebacter diphtherias, Streptococcus aureus, and Francisella novicida.

Exemplary wild type S. pyogenes Cas9 proteins of the disclosure may comprise or consist of the amino acid sequence:

(SEQ ID NO: 147)

| | |
|---|---|
| 1 | MDKKYSIGLD IGTNSVGWAV ITDEYKVPSK KFKVLGNTDR |
| | HSIKKNLIGA LLFDSGETAE |
| 61 | ATRLKRTARR RYTRRKNRIC YLQEIFSNEM AKVDDSFFHR |
| | LEESFLVEED KKHERHPIFG |
| 121 | NIVDEVAYHE KYPTIYHLRK KLVDSTDKAD LRLIYLALAH |
| | MIKFRGHFLI EGDLNPDNSD |
| 181 | VDKLFIQLVQ TYNQLFEENP INASGVDAKA ILSARLSKSR |
| | RLENLIAQLP GEKKNGLFGN |
| 241 | LIALSLGLTP NFKSNFDLAE DAKLQLSKDT YDDDLDNLLA |
| | QIGDQYADLF LAAKNLSDAI |
| 301 | LLSDILRVNT EITKAPLSAS MIKRYDEHHQ DLTLLKALVR |
| | QQLPEKYKEI FFDQSKNGYA |
| 361 | GYIDGGASQE EFYKFIKPIL EKMDGTEELL VKLNREDLLR |
| | KQRTFDNGSI PHQIHLGELH |
| 421 | AILRRQEDFY PFLKDNREKI EKILTFRIPY YVGPLARGNS |
| | RFAWMTRKSE ETITPWNFEE |
| 481 | VVDKGASAQS FIERMTNFDK NLPNEKVLPK HSLLYEYFTV |
| | YNELTKVKYV TEGMRKPAFL |
| 541 | SGEQKKAIVD LLFKTNRKVT VKQLKEDYFK KIECFDSVEI |
| | SGVEDRFNAS LGTYHDLLKI |
| 601 | IKDKDFLDNE ENEDILEDIV LTLTLFEDRE MIEERLKTYA |
| | HLFDDKVMKQ LKRRRYTGWG |
| 661 | RLSRKLINGI RDKQSGKTIL DFLKSDGFAN RNFMQLIHDD |
| | SLTFKEDIQK AQVSGQGDSL |
| 721 | HEHIANLAGS PAIKKGILQT VKVVDELVKV MGRHKPENIV |
| | IEMARENQTT QKGQKNSRER |
| 781 | MKRIEEGIKE LGSQILKEHP VENTQLQNEK LYLYYLQNGR |
| | DMYVDQELDI NRLSDYDVDH |
| 841 | IVPQSFLKDD SIDNKVLTRS DKNRGKSDNV PSEEVVKKMK |
| | NYWRQLLNAK LITQRKFDNL |
| 901 | TKAERGGLSE LDKAGFIKRQ LVETRQITKH VAQILDSRMN |
| | TKYDENDKLI REVKVITLKS |
| 961 | KLVSDFRKDF QFYKVREINN YHHAHDAYLN AVVGTALIKK |
| | YPKLESEFVY GDYKVYDVRK |
| 1021 | MIAKSEQEIG KATAKYFFYS NIMNFFKTEI TLANGEIRKR |
| | PLIETNGETG EIVWDKGRDF |
| 1081 | ATVRKVLSMP QVNIVKKTEV QTGGFSKESI LPKRNSDKLI |
| | ARKKDWDPKK YGGFDSPTVA |
| 1141 | YSVLVVAKVE KGKSKKLKSV KELLGITIME RSSFEKNPID |
| | FLEAKGYKEV KKDLIIKLPK |
| 1201 | YSLFELENGR KRMLASAGEL QKGNELALPS KYVNFLYLAS |
| | HYEKLKGSPE DNEQKQLFVE |
| 1261 | QHKHYLDEII EQISEFSKRV ILADANLDKV LSAYNKHRDK |
| | PIREQAENII HLFTLTNLGA |
| 1321 | PAAFKYFDTT IDRKRYTSTK EVLDATLIHQ SITGLYETRI |
| | DLSQLGGD. |

Nuclease inactivated S. pyogenes Cas9 proteins may comprise a substitution of an Alanine (A) for an Aspartic Acid (D) at position 10 and an alanine (A) for a Histidine (H) at position 840. Exemplary nuclease inactivated S. pyogenes Cas9 proteins of the disclosure may comprise or consist of the amino acid sequence (D10A and H840A bolded and underlined):

(SEQ ID NO: 148)

| | |
|---|---|
| 1 | MDKKYSIGLA IGTNSVGWAV ITDEYKVPSK KFKVLGNTDR |
| | HSIKKNLIGALLFDSGETAE |
| 61 | ATRLKRTARR RYTRRKNRIC YLQEIFSNEM AKVDDSFFHR |
| | LEESFLVEED KKHERHPIFG |
| 121 | NIVDEVAYHE KYPTIYHLRK KLVDSTDKAD LRLIYLALAH |
| | MIKFRGHFLI EGDLNPDNSD |
| 181 | VDKLFIQLVQ TYNQLFEENP INASGVDAKA ILSARLSKSR |
| | RLENLIAQLP GEKKNGLFGN |
| 241 | LIALSLGLTP NFKSNFDLAE DAKLQLSKDT YDDDLDNLLA |
| | QIGDQYADLF LAAKNLSDAI |
| 301 | LLSDILRVNT EITKAPLSAS MIKRYDEHHQ DLTLLKALVR |
| | QQLPEKYKEI FFDQSKNGYA |
| 361 | GYIDGGASQE EFYKFIKPIL EKMDGTEELL VKLNREDLLR |
| | KQRTFDNGSI PHQIHLGELH |
| 421 | AILRRQEDFY PFLKDNREKI EKILTFRIPY YVGPLARGNS |
| | RFAWMTRKSE ETITPWNFEE |
| 481 | VVDKGASAQS FIERMTNFDK NLPNEKVLPK HSLLYEYFTV |
| | YNELTKVKYV TEGMRKPAFL |
| 541 | SGEQKKAIVD LLFKTNRKVT VKQLKEDYFK KIECFDSVEI |
| | SGVEDRFNAS LGTYHDLLKI |
| 601 | IKDKDFLDNE ENEDILEDIV LTLTLFEDRE MIEERLKTYA |
| | HLFDDKVMKQ LKRRRYTGWG |
| 661 | RLSRKLINGI RDKQSGKTIL DFLKSDGFAN RNFMQLIHDD |
| | SLTFKEDIQK AQVSGQGDSL |
| 721 | HEHIANLAGS PAIKKGILQT VKVVDELVKV MGRHKPENIV |
| | IEMARENQTT QKGQKNSRER |
| 781 | MKRIEEGIKE LGSQILKEHP VENTQLQNEK LYLYYLQNGR |
| | DMYVDQELDI NRLSDYDVDA |
| 841 | IVPQSFLKDD SIDNKVLTRS DKNRGKSDNV PSEEVVKKMK |
| | NYWRQLLNAK LITQRKFDNL |
| 901 | TKAERGGLSE LDKAGFIKRQ LVETRQITKH VAQILDSRMN |
| | TKYDENDKLI REVKVITLKS |
| 961 | KLVSDFRKDF QFYKVREINN YHHAHDAYLN AVVGTALIKK |
| | YPKLESEFVY GDYKVYDVRK |
| 1021 | MIAKSEQEIG KATAKYFFYS NIMNFFKTEI TLANGEIRKR |
| | PLIETNGETG EIVWDKGRDF |
| 1081 | ATVRKVLSMP QVNIVKKTEV QTGGFSKESI LPKRNSDKLI |
| | ARKKDWDPKK YGGFDSPTVA |
| 1141 | YSVLVVAKVE KGKSKKLKSV KELLGITIME RSSFEKNPID |
| | FLEAKGYKEV KKDLIIKLPK |
| 1201 | YSLFELENGR KRMLASAGEL QKGNELALPS KYVNFLYLAS |
| | HYEKLKGSPE DNEQKQLFVE |
| 1261 | QHKHYLDEII EQISEFSKRV ILADANLDKV LSAYNKHRDK |
| | PIREQAENII HLFTLTNLGA |
| 1321 | PAAFKYFDTT IDRKRYTSTK EVLDATLIHQ SITGLYETRI |
| | DLSQLGGD. |

Nuclease inactivated S. pyogenes Cas9 proteins may comprise deletion of a RuvC nuclease domain or a portion thereof, an HNH domain, a DNAse active site, a ββα-metal fold or a portion thereof comprising a DNAse active site or any combination thereof.

Other exemplary Cas9 proteins or portions thereof may comprise or consist of the following amino acid sequences.

In some embodiments the Cas9 protein can be S. pyogenes Cas9 and may comprise or consist of the amino acid sequence:

(SEQ ID NO: 149)
MDKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSN
FDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKN
GYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGN
SRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLS
RKLINGIRDKQSGKTILDFLKSDGFANRNFMQLIHDDSLTFKEDIQKAQVS
GQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKS
DNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKD
FQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGE
IVWDKGRDFATVRKVLSMPQVNIVKKTEVQTGGFSKESILPKRNSDKLIAR
KKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAFKY
FDTTIDRKRYTSTKEVLDATLIHQSITGLYETRIDLSQLGGD

In some embodiments the Cas9 protein can be S. aureus Cas9 and may comprise or consist of the amino acid sequence:

(SEQ ID NO: 150)
MKRNYILGLDIGITSVGYGIIDYETRDVIDAGVRLFKEANVENNEGRRSKR
GARRLKRRRRHRIQRVKKLLFDYNLLTDHSELSGINPYEARVKGLSQKLSE
EEFSAALLHLAKRRGVHNVNEVEEDTGNELSTKEQISRNSKALEEKYVAEL
QLERLKKDGEVRGSINRFKTSDYVKEAKQLLKVQKAYHQLDQSFIDTYIDL
LETRRTYYEGPGEGSPFGWKDIKEWYEMLMGHCTYFPEELRSVKYAYNADL
YNALNDLNNLVITRDENEKLEYYEKFQIIENVFKQKKKPTLKQIAKEILVN
EEDIKGYRVTSTGKPEFTNLKVYHDIKDITARKEIIENAELLDQIAKILTI
YQSSEDIQEELTNLNSELTQEEIEQISNLKGYTGTHNLSLKAINLILDELW
HTNDNQIAIFNRLKLVPKKVDLSQQKEIPTTLVDDFILSPVVKRSFIQSIK
VINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPR
SVSFDNSFNNKVLVKQEENSKKGNRTPFQYLSSSDSKISYETFKKHILNLA
KGKGRISKTKKEYLLEERDINRFSVQKDFINRNLVDTRYATRGLMNLLRSY
FRVNNLDVKVKSINGGFTSFLRRKWKFKKERNKGYKHHAEDALIIANADFI
FKEWKKLDKAKKVMENQMFEEKQAESMPEIETEQEYKEIFITPHQIKHIKD
FKDYKYSHRVDKKPNRELINDTLYSTRKDDKGNTLIVNNLNGLYDKDNDKL
KKLINKSPEKLLMYHHDPQTYQKLKLIMEQYGDEKNPLYKYYEETGNYLTK
YSKKDNGPVIKKIKYYGNKLNAHLDITDDYPNSRNKVVKLSLKPYRFDVYL
DNGVYKFVTVKNLDVIKKENYYEVNSKCYEEAKKLKKISNQAEFIASFYNN
DLIKINGELYRVIGVNNDLLNRIEVNMIDITYREYLENMNDKRPPRIIKTI
ASKTQSIKKYSTDILGNLYEVKSKKHPQIIKKG

In some embodiments the Cas9 protein can be S. thermophiles CRISPR1 Cas9 and may comprise or consist of the amino acid sequence:

(SEQ ID NO: 151)
MSDLVLGLDIGIGSVGVGILNKVTGEIIHKNSRIFPAAQAENNLVRRTNR
QGRRLARRKKHRRVRLNRLFEESGLITDFTKISINLNPYQLRVKGLTDEL
SNEELFIALKNMVKHRGISYLDDASDDGNSSVGDYAQIVKENSKQLETKT
PGQIQLERYQTYGQLRGDFTVEKDGKKHRLINVFPTSAYRSEALRILQTQ
QEFNPQITDEFINRYLEILTGKRKYYHGPGNEKSRTDYGRYRTSGETLDN
IFGILIGKCTFYPDEFRAAKASYTAQEFNLLNDLNNLTVP1ETKKLSKEQ
KNQIINYVKNEKAMGPAKLFKYIAKLLSCDVADIKGYRIDKSGKAEIHTF
EAYRKMKTLETLDIEQMDRETLDKLAYVLTLNTEREGIQEALEHEFADGS
FSQKQVDELVQFRKANSSIFGKGWHNFSVKLMMELIPELYETSEEQMTIL
TRLGKQKTTSSSNKTKYIDEKLLTEEIYNPVVAKSVRQAIKIVNAAIKEY
GDFDNIVIEMARETNEDDEKKAIQKIQKANKDEKDAAMLKAANQYNGKAE
LPHSVFHGHKQLATKIRLWHQQGERCLYTGKTISIHDLINNSNQFEVDHI
LPLSITFDDSLANKVLVYATANQEKGQRTPYQALDSMDDAWSFRELKAFV
RESKTLSNKKKEYLLTEEDISKFDVRKKFIERNLVDTRYASRVVLNALQE
HFRAHKIDTKVSVVRGQFTSQLRRHWGIEKTRDTYHHHAVDALIIAASSQ
LNLWKKQKNTLVSYSEDQLLDIETGELISDDEYKESVFKAPYQHFVDTLK
SKEFEDSILFSYQVDSKFNRKISDATIYATRQAKVGKDKADETYVLGKIK
DIYTQDGYDAFMKIYKKDKSKFLMYRHDPQTFEKVIEPILENYPNKQIND
KGKEVPCNPFLKYKEEHGYIRKYSKKGNGPEIKSLKYYDSKLGNHIDITP
KDSNNKVVLQSVSPWRADVYFNKTTGKYEILGLKYADLQFDKGTGTYKIS
QEKYNDIKKKEGVDSDSEFKFTLYKNDLLLVKDIETKEQQLFRFLSRTMP
KQKHYVELKPYDKQKFEGGEALIKVLGNVANSGQCKKGLGKSNISIYKVR
TDVLGNQHIIKNEGDKPKLDF

In some embodiments the Cas9 protein can be N. meningitidis Cas9 and may comprise or consist of the amino acid sequence:

(SEQ ID NO: 152)
MAAFKPNPINYILGLDIGIASVGWAMVEIDEDENPICLIDLGVRVFERAE
VPKTGDSLAMARRLARSVRRLTRRRAHRLLRARRLLKREGVLQAADFDEN
GLIKSLPNTPWQLRAAALDRKLTPLEWSAVLLHLIKHRGYLSQRKNEGET
ADKELGALLKGVADNAHALQTGDFRTPAELALNKFEKESGHIRNQRGDYS
HTFSRKDLQAELILLFEKQKEFGNPHVSGGLKEGIETLLMTQRPALSGDA
VQKMLGHCTFEPAEPKAAKNTYTAERFIWLTKLNNLRILEQGSERPLTDT
ERATLMDEPYRKSKLTYAQARKLLGLEDTAFFKGLRYGKDNAEASTLMEM
KAYHAISRALEKEGLKDKKSPLNLSPELQDEIGTAFSLFKTDEDITGRLK
DRIQPEILEALLKHISFDKFVQISLKALRRIVPLMEQGKRYDEACAEIYG
DHYGKKNIEEKIYLPPIPADEIRNPVVLRALSQARKVINGVVRRYGSPAR
IHIETAREVGKSFKDRKEIEKRQEENRKDREKAAAKFREYFPNFVGEPKS
KDILKLRLYEQQHGKCLYSGKEINLGRLNEKGYVEIDHALPFSRTWDDSF
NNKVLVLGSENQNKGNQTPYEYFNGKDNSREWQEFKARVETSRFPRSKKQ
RILLQKFDEDGFKERNLNDTRYVNRFLCQFVADRMRLTGKGKKRVFASNG
QITNLLRGFWGLRKVRAENDRHHALDAVVVACSTVAMQQKITRFVRYKEM
NAFDGKTIDKETGEVLHQKTHFPQPWEFFAQEVMIRVFGKPDGKPEFEEA
DTPEKLRTLLAEKLSSRPEAVHEYVTPLFVSRAPNRKMSGQGHMETVKSA
KRLDEGVSVLRVPLTQLKLKDLEKMVNREREPKLYEALKARLEAHKDDPA
KAFAEPFYKYDKAGNRTQQVKAVRVEQVQKTGVVVVRNHNGIADNATMVR
VDVFEKGDKYYLVPIYSWQVAKGILPDRAVVQGKDEEDWQLIDDSFNFKF
SLHPNDLVEVITKKARMFGYFASCHRGTGNINIRIHDLDHKIGKNGILEG
IGVKTALSFQKYQIDELGKEIRPCRLKKRPPVR

In some embodiments the Cas9 protein can be Parvibaculum. lavamentivorans Cas9 and may comprise or consist of the amino acid sequence:

(SEQ ID NO: 153)
MERIFGFDIGTTSIGFSVIDYSSTQSAGNIQRLGVRIFPEARDPDGTPLN
QQRRQKRMMRRQLRRRRIRRKALNETLHEAGFLPAYGSADWPVVMADEPY
ELRRRGLEEGLSAYEFGRAIYHLAQHRHFKGRELEESDTPDPDVDDEKEA
ANERAATLKALKNEQTTLGAWLARRPPSDRKRGIHAHRNVVAEEFERLWE
VQSKFHPALKSEEMRARISDTIFAQRPVFWRKNTLGECRFMPGEPLCPKG
SWLSQQRRMLEKLNNLAIAGGNARPLDAEERDAILSKLQQQASMSWPGVR
SALKALYKQRGEPGAEKSLKFNLELGGESKLLGNALEAKLADMFGPDWPA
HPRKQEIRHAVHERLWAADYGETPDKKRVIILSEKDRKAHREAAANSFVA
DFGITGEQAAQLQALKLPTGWEPYSIPALNLFLAELEKGERFGALVNGPD
WEGWRRTNFPHRNQPTGEILDKLPSPASKEERERISQLRNPTVVRTQNEL
RKVVNNLIGLYGKPDRIRIEVGRDVGKSKREREEIQSGIRRNEKQRKKAI
EDLIKNGIANPSRDDVEKWILWKEGQERCPYTGDQIGFNALFREGRYEVE
HIWPRSRSFDNSPRNKTLCRKDVNIEKGNRMPFEAFGHDEDRWSAIQIRL
QGMVSAKGGTGMSPGKVKRFLAKTMPEDFAARQLNDTRYAAKQILAQLKR
LWPDMGPEAPVKVEAVTGQVTAQLRKLWTLNNILADDGEKTRADHRHHAI
DALTVACTHPGMTNKLSRYWQLRDDPRAEKPALTPPWDTIRADAEKAVSE
IVVSHRVRKKVSGPLHKETTYGDTGTDIKTKSGTYRQFVTRKKIESLSKG
ELDEIRDPRIKEIVAAHVAGRGGDPKKAFPPYPCVSPGGPEIRKVRLTSK
QQLNLMAQTGNGYADLGSNHHIAIYRLPDGKADFEIVSLFDASRRLAQRN
PIVQRTRADGASFVMSLAAGEAIMIPEGSKKGIWIVQGVVVASGQVVLER
DTDADHSTTTRPMPNPILKDDAKKVSIDPIGRVRPSND

In some embodiments the Cas9 protein can be Corynebacter diphtheria Cas9 and may comprise or consist of the amino acid sequence:

(SEQ ID NO: 154)
MKYHVGIDVGTFSVGLAAIEVDDAGMPIKTLSLVSHIHDSGLDPDEIKSA
VTRLASSGIARRTRRLYRRKRRRLQQLDKFIQRQGWPVIELEDYSDPLYP
WKVRAELAASYIADEKERGEKLSVALRHIARHRGWRNPYAKVSSLYLPDG
PSDAFKAIREEIKRASGQPVPETATVGQMVTLCELGTLKLRGEGGVLSAR
LQQSDYAREIQEICRMQEIGQELYRKIIDVVFAAESPKGSASSRVGKDPL
QPGKNRALKASDAFQRYRIAALIGNLRVRVDGEKRILSVEEKNLVFDHLV
NLTPKKEPEWVTIAEILGIDRGQLIGTATMTDDGERAGARPPTHDTNRSI
VNSRIAPLVDWWKTASALEQHAMVKALSNAEVDDFDSPEGAKVQAFFADL
DDDVHAKLDSLHLPVGRAAYSEDTLVRLTRRMLSDGVDLYTARLQEFGIE
PSWTPPTPRIGEPVGNPAVDRVLKTVSRWLESATKTWGAPERVIIEHVRE
GFVTEKRAREMDGDMRRRAARNAKLFQEMQEKLNVQGKPSRADLWRYQSV
QRQNCQCAYCGSPITFSNSEMDHIVPRAGQGSTNTRENLVAVCHRCNQSK
GNTPFAIWAKNTSIEGVSVKEAVERTRHWVTDTGMRSTDFKKFTKAVVER
FQRATMDEEIDARSMESVAWMANELRSRVAQHFASHGTTVRVYRGSLTAE
ARRASGISGKLKFFDGVGKSRLDRRHHAIDAAVIAFTSDYVAETLAVRSN
LKQSQAHRQEAPQWREFTGKDAEHRAAWRVVVCQKMEKLSALLTEDLRDD
RVVVMSNVRLRLGNGSAHKETIGKLSKVKLSSQLSVSDIDKASSEALWCA
LTREPGFDPKEGLPANPERHIRVNGTHVYAGDNIGLFPVSAGSIALRGGY
AELGSSFHHARVYKITSGKKPAFAMLRVYTIDLLPYRNQDLFSVELKPQT
MSMRQAEKKLRDALATGNAEYLGWLVVDDELVVDTSKIATDQVKAVEAEL
GTIRRWRVDGFFSPSKLRLRPLQMSKEGIKKESAPELSKIIDRPGWLPAV
NKLFSDGNVTVVRRDSLGRVRLESTAHLPVTWKVQ

In some embodiments the Cas9 protein can be Streptococcus pasteurianus Cas9 and may comprise or consist of the amino acid sequence:

(SEQ ID NO: 155)
MTNGKILGLDIGIASVGVGIIEAKTGKVVHANSRLFSAANAENNAERRGF
RGSRRLNRRKKHRVKRVRDLFEKYGIVTDFRNLNLNPYELRVKGLTEQLK
NEELFAALRTISKRRGISYLDDAEDDSTGSTDYAKSIDENRRLLKNKTPG
QIQLERLEKYGQLRGNFTVYDENGEAHRLINVFSTSDYEKEARKILETQA
DYNKKITAEFIDDYVEILTQKRKYYHGPGNEKSRTDYGRFRTDGTTLENI
FGILIGKCNFYPDEYRASKASYTAQEYNFLNDLNNLKVSIETGKLSTEQK
ESLVEFAKNTATLGPAKLLKEIAKILDCKVDEIKGYREDDKGKPDLHTFE
PYRKLKFNLESINIDDLSREVIDKLADILTLNTEREGIEDAIKRNLPNQF
IEEQISEIIKVRKSQSTAFNKGWHSFSAKLMNELIPELYATSDEQMTILT
RLEKFKVNKKSSKNTKTIDEKEVTDEIYNPVVAKSVRQTIKIINAAVKKY
GDFDKIVIEMPRDKNADDEKKFIDKRNKENKKEKDDALKRAAYLYNSSDK
LPDEVFHGNKQLETKIRLWYQQGERCLYSGKPISIQELVHNSNNFEIDHI
LPLSLSFDDSLANKVLVYAWTNQEKGQKTPYQVIDSMDAAWSFREMKDYV
LKQKGLGKKKRDYLLTIENIDKIEVKKKFIERNLVDTRYASRVVLNSLQS
ALRELGKDTKVSVVRGQFTSQLRRKWKIDKSRETYHHHAVDALIIAASSQ
LKLWEKQDNPMFVDYGKNQVVDKQTGEILSVSDDEYKELVFQPPYQGFVN
TISSKGFEDEILFSYQVDSKYNRKVSDATIYSTRKAKIGKDKKEETYVLG
KIKDIYSQNGFDTFIKKYNKDKTQFLMYQKDSLTWENVIEVILRDYPTTK
KSEDGKNDVKCNPFEEYRRENGLICKYSKKGKGTPIKSLKYYDKKLGNCI
DITPEESRNKVILQSINPWRADVYFNPETLKYELMGLKYSDLSFEKGTGN
YHISQEKYDAIKEKEGIGKKSEFKFTLYRNDLILIKDIASGEQEIYRFLS
RTMPNVNHYVELKPYDKEKFDNVQELVEALGEADKVGRCIKGLNKPNISI
YKVRTDVLGNKYFVKKKGDKPKLDFKNNKK

In some embodiments the Cas9 protein can be Neisseria cinerea Cas9 and may comprise or consist of the amino acid sequence:

(SEQ ID NO: 156)
MAAFKPNPMNYILGLDIGIASVGWAIVEIDEEENPIRLIDLGVRVFERAE
VPKTGDSLAAARRLARSVRRLTRRRAHRLLRARRLLKREGVLQAADFDEN
GLIKSLPNTPWQLRAAALDRKLTPLEWSAVLLHLIKHRGYLSQRKNEGET
ADKELGALLKGVADNTHALQTGDFRTPAELALNKFEKESGHIRNQRGDYS
HTFNRKDLQAELNLLFEKQKEFGNPHVSDGLKEGIETLLMTQRPALSGDA
VQKMLGHCTFEPIEPKAAKNTYTAERFVWLTKLNNLRILEQGSERPLTDI
ERATLMDEPYRKSKLTYAQARKLLDLDDTAFFKGLRYGKDNAEASTLMEM
KAYHAISRALEKEGLKDKKSPLNLSPELQDEIGTAFSLFKTDEDITGRLK
DRVQPEILEALLKHISFDKFVQISLKALRRIVPLMEQGNRYDEACTEIYG
DHYGKKNIEEKIYLPPIPADEIRNPVVLRALSQARKVINGVVRRYGSPAR
IHIETAREVGKSFKDRKEIEKRQEENRKDREKSAAKFREYFPNFVGEPKS
KDILKLRLYEQQHGKCLYSGKEINLGRLNEKGYVEIDHALPFSRTWDDSF
NNKVLALGSENQNKGNQTPYEYFNGKDNSREWQEFKARVETSRFPRSKKQ
RILLQKFDEDGFKERNLNDTRYINRFLCQFVADHMLLTGKGKRRVFASNG
QITNLLRGFWGLRKVRAENDRHHALDAVVVACSTIAMQQKITRFVRYKEM
NAFDGKTIDKETGEVLHQKAHFPQPWEFFAQEVIVIIRVFGKPDGKPEFE
EADTPEKLRTLLAEKLSSRPEAVHKYVTPLFISRAPNRKMSGQGHMETVK
SAKRLDEGISVLRVPLTQLKLKDLEKMVNREREPKLYEALKARLEAHKDD
PAKAFAEPFYKYDKAGNRTQQVKAVRVEQVQKTGVVVVHNHNGIADNATI
VRVDVFEKGGKYYLVPIYSWQVAKGILPDRAVVQGKDEEDWTVMDDSFEF
KFVLYANDLIKLTAKKNEFLGYFVSLNRATGAIDIRTHDTDSTKGKNGIF
QSVGVKTALSFQKYQIDELGKEIRPCRLKKRPPVR

In some embodiments the Cas9 protein can be Campylobacter lari Cas9 and may comprise or consist of the amino acid sequence:

(SEQ ID NO: 157)
MRILGFDIGINSIGWAFVENDELKDCGVRIFTKAENPKNKESLALPRRNA
RSSRRRLKRRKARLIAIKRILAKELKLNYKDYVAADGELPKAYEGSLASV
YELRYKALTQNLETKDLARVILHIAKHRGYMNKNEKKSNDAKKGKILSAL
KNNALKLENYQSVGEYFYKEFFQKYKKNTKNFIKIRNTKDNYNNCVLSSD
LEKELKLILEKQKEFGYNYSEDFINEILKVAFFQRPLKDFSHLVGACTFF
EEEKRACKNSYSAWEFVALTKIINEIKSLEKISGEIVPTQTINEVLNLIL
DKGSITYKKFRSCINLHESISFKSLKYDKENAENAKLIDFRKLVEFKKAL
GVHSLSRQELDQISTHITLIKDNVKLKTVLEKYNLSNEQINNLLEIEFND
YINLSFKALGMILPLMREGKRYDEACEIANLKPKTVDEKKDFLPAFCDST
AHELSNPVVNRAISEYRKVLNALLKKYGKVHKIHLELARDVGLSKKAREK
IEKEQKENQAVNAWALKECENIGLKASAKNILKLKLWKEQKEICIYSGNK
ISIEHLKDEKALEVDHIYPYSRSFDDSFINKVLVFTKENQEKLNKTPFEA
FGKNIEKWSKIQTLAQNLPYKKKNKILDENFKDKQQEDFISRNLNDTRYI
ATLIAKYTKEYLNFLLLSENENANLKSGEKGSKIHVQTISGMLTSVLRHT
WGFDKKDRNNHLHHALDAIIVAYSTNSIIKAFSDFRKNQELLKARFYAKE
LTSDNYKHQVKFFEPFKSFREKILSKIDEIFVSKPPRKRARRALHKDTFH
SENKIIDKCSYNSKEGLQIALSCGRVRKIGTKYVENDTIVRVDIFKKQNK
FYAIPIYAMDFALGILPNKIVITGKDKNNNPKQWQTIDESYEFCFSLYKN
DLILLQKKNMQEPEFAYYNDFSISTSSICVEKHDNKFENLTSNQKLLFSN
AKEGSVKVESLGIQNLKVFEKYIITPLGDKIKADFQPRENISLKTSKKYG
LR

In some embodiments the Cas9 protein can be T. denticola Cas9 and may comprise or consist of the amino acid sequence:

(SEQ ID NO: 158)
MKKEIKDYFLGLDVGTGSVGWAVTDTDYKLLKANRKDLWGMRCFETAETA
EVRRLHRGARRRIERRKKRIKLLQELFSQEIAKTDEGFFQRMKESPFYAE
DKTILQENTLFNDKDFADKTYHKAYPTINHLIKAWIENKVKPDPRLLYLA
CHNIIKKRGHFLFEGDFDSENQFDTSIQALFEYLREDMEVDIDADSQKVK
EILKDSSLKNSEKQSRLNKILGLKPSDKQKKAITNLISGNKINFADLYDN
PDLKDAEKNSISFSKDDFDALSDDLASILGDSFELLLKAKAVYNCSVLSK
VIGDEQYLSFAKVKIYEKHKTDLTKLKNVIKKHFPKDYKKVFGYNKNEKN
NNNYSGYVGVCKTKSKKLIINNSVNQEDFYKFLKTILSAKSEIKEVNDIL
TEIETGTFLPKQISKSNAEIPYQLRKMELEKILSNAEKHFSFLKQKDEKG
LSHSEKIIMLLTFKIPYYIGPINDNHKKFFPDRCWVVKKEKSPSGKTTPW
NFFDHIDKEKTAEAFITSRTNFCTYLVGESVLPKSSLLYSEYTVLNEINN
LQIIIDGKNICDIKLKQKIYEDLFKKYKKITQKQISTFIKHEGICNKTDE
VIILGIDKECTSSLKSYIELKNIFGKQVDEISTKNMLEEIIRWATIYDEG
EGKTILKTKIKAEYGKYCSDEQIKKILNLKFSGWGRLSRKFLETVTSEMP
GFSEPVNIITAMRETQNNLMELLSSEFTFTENIKKINSGFEDAEKQFSYD
GLVKPLFLSPSVKKMLWQTLKLVKEISHITQAPPKKIFIEMAKGAELEPA
RTKTRLKILQDLYNNCKNDADAFSSEIKDLSGKIENEDNLRLRSDKLYLY
YTQLGKCMYCGKPMIGHVFDTSNYDIDHIYPQSKIKDDSISNRVLVCSSC
NKNKEDKYPLKSEIQSKQRGFWNFLQRNNFISLEKLNRLTRATPISDDET
AKFIARQLVETRQATKVAAKVLEKMFPETKIVYSKAETVSMFRNKFDIVK
CREINDFHHAHDAYLNIVVGNVYNTKFTNNPWNFIKEKRDNPKIADTYNY
YKVFDYDVKRNNITAWEKGKTIITVKDMLKRNTPIYTRQAACKKGELFNQ
TIMKKGLGQHPLKKEGPFSNISKYGGYNKVSAAYYTLIEYEEKGNKIRSL
ETIPLYLVKDIQKDQDVLKSYLTDLLGKKEFKILVPKIKINSLLKINGFP
CHITGKTNDSFLLRPAVQFCCSNNEVLYFKKIIRFSEIRSQREKIGKTIS
PYEDLSFRSYIKENLWKKTKNDEIGEKEFYDLLQKKNLEIYDMLLTKHKD
TIYKKRPNSATIDILVKGKEKFKSLIIENQFEVILEILKLFSATRNVSDL
QHIGGSKYSGVAKIGNKISSLDNCILIYQSITGIFEKRIDLLKV

In some embodiments the Cas9 protein can be S. mutans Cas9 and may comprise or consist of the amino acid sequence:

(SEQ ID NO: 159)
MKKPYSIGLDIGTNSVGWAVVTDDYKVPAKKMKVLGNTDKSHIEKNLLGA
LLFDSGNTAEDRRLKRTARRRYTRRRNRILYLQEIFSEEMGKVDDSFFHR
LEDSFLVTEDKRGERHPIFGNLEEEVKYHENFPTIYHLRQYLADNPEKVD
LRLVYLALAHIIKFRGHFLIEGKFDTRNNDVQRLFQEFLAVYDNTFENSS
LQEQNVQVEEILTDKISKSAKKDRVLKLFPNEKSNGRFAEFLKLIVGNQA
DFKKHFELEEKAPLQFSKDTYEEELEVLLAQIGDNYAELFLSAKKLYDSI
LLSGILTVTDVGTKAPLSASMIQRYNEHQMDLAQLKQFIRQKLSDKYNEV
FSDVSKDGYAGYIDGKTNQEAFYKYLKGLLNKIEGSGYFLDKIEREDFLR
KQRTFDNGSIPHQIHLQEMRAIIRRQAEFYPFLADNQDRIEKLLTFRIPY
YVGPLARGKSDFAWLSRKSADKITPWNFDEIVDKESSAEAFINRMTNYDL
YLPNQKVLPKHSLLYEKFTVYNELTKVKYK1EQGKTAFFDANMKQEIFDG
VFKVYRKVTKDKLMDFLEKEFDEFRIVDLTGLDKENKVFNASYGTYHDLC
KILDKDFLDNSKNEKILEDIVLTLTLFEDREMIRKRLENYSDLLTKEQVK
KLERRHYTGWGRLSAELIHGIRNKESRKTILDYLIDDGNSNRNFMQLIND
DALSFKEEIAKAQVIGETDNLNQVVSDIAGSPAIKKGILQSLKIVDELVK
IMGHQPENIVVEMARENQFTNQGRRNSQQRLKGLTDSIKEFGSQILKEHP
VENSQLQNDRLFLYYLQNGRDMYTGEELDIDYLSQYDIDHIIPQAFIKDN
SIDNRVLTSSKENRGKSDDVPSKDVVRKMKSYWSKLLSAKLITQRKFDNL
TKAERGGLTDDDKAGFIKRQLVETRQITKHVARILDERFNTETDENNKKI
RQVKIVTLKSNLVSNFRKEFELYKVREINDYHHAHDAYLNAVIGKALLGV
YPQLEPEFVYGDYPHFHGHKENKATAKKFFYSNIMNFFKKDDVRTDKNGE
IIWKKDEHISNIKKVLSYPQVNIVKKVEEQTGGFSKESILPKGNSDKLIP
RKTKKFYWDTKKYGGFDSPIVAYSILVIADIEKGKSKKLKTVKALVGVTI
MEKMTFERDPVAFLERKGYRNVQEENIIKLPKYSLFKLENGRKRLLASAR
ELQKGNEIVLPNHLGTLLYHAKNIHKVDEPKHLDYVDKHKDEFKELLDVV
SNFSKKYTLAEGNLEKIKELYAQNNGEDLKELASSFINLLTFTAIGAPAT
FKFFDKNIDRKRYTSTlEILNATLIHQSITGLYETRIDLNKLGGD

In some embodiments the Cas9 protein can be S. thermophilus CRISPR 3 Cas9 and may comprise or consist of the amino acid sequence:

(SEQ ID NO: 160)
MTKPYSIGLDIGTNSVGWAVTTDNYKVPSKKMKVLGNTSKKYIKKNLLGV
LLFDSGITAEGRRLKRTARRRYTRRRNRILYLQEIFSTEMATLDDAFFQR
LDDSFLVPDDKRDSKYPIFGNLVEEKAYHDEFPTIYHLRKYLADSTKKAD
LRLVYLALAHMIKYRGHFLIEGEFNSKNNDIQKNFQDFLDTYNAIFESDL
SLENSKQLEEIVKDKISKLEKKDRILKLFPGEKNSGIFSEFLKLIVGNQA
DFRKCFNLDEKASLHFSKESYDEDLETLLGYIGDDYSDVFLKAKKLYDAI
LLSGFLTVTDNElEAPLSSAMIKRYNEHKEDLALLKEYIRNISLKTYNEV
FKDDTKNGYAGYIDGKTNQEDFYVYLKKLLAEFEGADYFLEKIDREDFLR
KQRTFDNGSIPYQIHLQEMRAILDKQAKFYPFLAKNKERIEKILTFRIPY
YVGPLARGNSDFAWSIRKRNEKITPWNFEDVIDKESSAEAFINRMTSFDL
YLPEEKVLPKHSLLYETFNVYNELTKVRFIAESMRDYQFLDSKQKKDIVR
LYFKDKRKVTDKDIIEYLHAIYGYDGIELKGIEKQFNSSLSTYHDLLNII
NDKEFLDDSSNEAIIEEIIHTLTIFEDREMIKQRLSKFENIFDKSVLKKL
SRRHYTGWGKLSAKLINGIRDEKSGNTILDYLIDDGISNRNFMQLIHDDA
LSFKKKIQKAQIIGDEDKGNIKEVVKSLPGSPAIKKGILQSIKIVDELVK
VMGGRKPESIVVEMARENQYTNQGKSNSQQRLKRLEKSLKELGSKILKEN
IPAKLSKIDNNALQNDRLYLYYLQNGKDMYTGDDLDIDRLSNYDIDHIIP
QAFLKDNSIDNKVLVSSASNRGKSDDVPSLEVVKKRKTFWYQLLKSKLIS
QRKFDNLTKAERGGLSPEDKAGFIQRQLVETRQITKHVARLLDEKFNNKK
DENNRAVRTVKIITLKSTLVSQFRKDFELYKVREINDFHHAHDAYLNAVV
ASALLKKYPKLEPEFVYGDYPKYNSFRERKSA1EKVYFYSNIMNIFKKSI
SLADGRVIERPLIEVNEETGESVWNKESDLATVRRVLSYPQVNVVKKVEE
QNHGLDRGKPKGLFNANLSSKPKPNSNENLVGAKEYLDPKKYGGYAGISN
SFTVLVKGTIEKGAKKKITNVLEFQGISILDRINYRKDKLNFLLEKGYKD
IELIIELPKYSLFELSDGSRRMLASILSTNNKRGEIHKGNQIFLSQKFVK
LLYHAKRISNTINENHRKYVENHKKEFEELFYYILEFNENYVGAKKNGKL
LNSAFQSWQNHSIDELCSSFIGPTGSERKGLFELTSRGSAADFEFLGVKI
PRYRDYTPSSLLKDATLIHQSVTGLYETRIDLAKLGEG

In some embodiments the Cas9 protein can be C. jejuni Cas9 and may comprise or consist of the amino acid sequence:

(SEQ ID NO: 161)
MARILAFDIGISSIGWAFSENDELKDCGVRIFTKVENPKTGESLALPRRL
ARSARKRLARRKARLNHLKHLIANEFKLNYEDYQSFDESLAKAYKGSLIS
PYELRFRALNELLSKQDFARVILHIAKRRGYDDIKNSDDKEKGAILKAIK
QNEEKLANYQSVGEYLYKEYFQKFKENSKEFTNVRNKKESYERCIAQSFL
KDELKLIFKKQREFGFSFSKKFEEEVLSVAFYKRALKDFSHLVGNCSFFT
DEKRAPKNSPLAFWVALTRIINLLNNLKNIEGILYTKDDLNALLNEVLKN
GTLTYKQTKKLLGLSDDYEFKGEKGTYFIEFKKYKEFIKALGEHNLSQDD
LNEIAKDITLIKDEIKLKKALAKYDLNQNQIDSLSKLEFKDHLNISFKAL
KLVTPLMLEGKKYDEACNELNLKVAINEDKKDFLPAFNETYYKDEVTNPV
VLRAIKEYRKVLNALLKKYGKVHKINIELAREVGKNHSQRAKIEKEQNEN
YKAKKDAELECEKLGLKINSKNILKLRLFKEQKEFCAYSGEKIKISDLQD
EKMLEIDHIYPYSRSFDDSYMNKVLVFTKQNQEKLNQTPFEAFGNDSAKW
QKIEVLAKNLPTKKQKRILDKNYKDKEQKNFKDRNLNDTRYIARLVLNYT
KDYLDFLPLSDDENTKLNDTQKGSKVHVEAKSGMLTSALRHTWGFSAKDR
NNHLHHAIDAVIIAYANNSIVKAFSDFKKEQESNSAELYAKKISELDYKN
KRKFFEPFSGFRQKVLDKIDEIFVSKPERKKPSGALHEETFRKEEEFYQS
YGGKEGVLKALELGKIRKVNGKIVKNGDMFRVDIFKHKKTNKFYAVPIYT
MDFALKVLPNKAVARSKKGEIKDWILMDENYEFCFSLYKDSLILIQTKDM
QEPEFVYYNAFTSSTVSLIVSKHDNKFETLSKNQKILFKNANEKEVIAKS
IGIQNLKVFEKYIVSALGEVTKAEFRQREDFKK

In some embodiments the Cas9 protein can be P. multocida Cas9 and may comprise or consist of the amino acid sequence:

(SEQ ID NO: 162)
MQTTNLSYILGLDLGIASVGWAVVEINENEDPIGLIDVGVRIFERAEVPK
TGESLALSRRLARSTRRLIRRRAHRLLLAKRFLKREGILSTIDLEKGLPN
QAWELRVAGLERRLSAIEWGAVLLHLIKHRGYLSKRKNESQTNNKELGAL
LSGVAQNHQLLQSDDYRTPAELALKKFAKEEGHIRNQRGAYTHTFNRLDL
LAELNLLFAQQHQFGNPHCKEHIQQYMTELLMWQKPALSGEAILKMLGKC
THEKNEFKAAKHTYSAERFVVVLTKLNNLRILEDGAERALNEEERQLLIN
HPYEKSKLTYAQVRKLLGLSEQAIFKHLRYSKENAESATFMELKAWHAIR
KALENQGLKDTWQDLAKKPDLLDEIGTAFSLYKTDEDIQQYLTNKVPNSV
INALLVSLNFDKFIELSLKSLRKILPLMEQGKRYDQACREIYGHHYGEAN
QKTSQLLPAIPAQEIRNPVVLRTLSQARKVINAIIRQYGSPARVHIETGR
ELGKSFKERREIQKQQEDNRTKRESAVQKFKELFSDFSSEPKSKDILKFR
LYEQQHGKCLYSGKEINIHRLNEKGYVEIDHALPFSRTWDDSFNNKVLVL
ASENQNKGNQTPYEWLQGKINSERWKNFVALVLGSQCSAAKKQRLLTQVI
DDNKFIDRNLNDTRYIARFLSNYIQENLLLVGKNKKNVFTPNGQITALLR
SRWGLIKARENNNRHHALDAIVVACATPSMQQKITRFIRFKEVHPYKIEN
RYEMVDQESGEIISPHFPEPWAYFRQEVNIRVFDNHPDTVLKEMLPDRPQ
ANHQFVQPLFVSRAPTRKMSGQGHMETIKSAKRLAEGISVLRIPLTQLKP
NLLENMVNKEREPALYAGLKARLAEFNQDPAKAFATPFYKQGGQQVKAIR
VEQVQKSGVLVRENNGVADNASIVRTDVFIKNNKFFLVPIYTWQVAKGIL
PNKAIVAHKNEDEWEEMDEGAKFKFSLFPNDLVELKTKKEYFFGYYIGLD
RATGNISLKEHDGEISKGKDGVYRVGVKLALSFEKYQVDELGKNRQICRP
QQRQPVR

In some embodiments the Cas9 protein can be F. novicida Cas9 and may comprise or consist of the amino acid sequence:

(SEQ ID NO: 163)
MNFKILPIAIDLGVKNTGVFSAFYQKGTSLERLDNKNGKVYELSKDSYTL
LMNNRTARRHQRRGIDRKQLVKRLFKLIW1EQLNLEWDKDTQQAISFLFN
RRGFSFITDGYSPEYLNIVPEQVKAILMDIFDDYNGEDDLDSYLKLATEQ
ESKISEIYNKLMQKILEFKLMKLCTDIKDDKVSTKTLKEITSYEFELLAD
YLANYSESLKTQKFSYTDKQGNLKELSYYHHDKYNIQEFLKRHATINDRI
LDTLLTDDLDIWNFNFEKFDFDKNEEKLQNQEDKDHIQAHLHHFVFAVNK
IKSEMASGGRHRSQYFQEITNVLDENNHQEGYLKNFCENLHNKKYSNLSV
KNLVNLIGNLSNLELKPLRKYFNDKIHAKADHWDEQKFTETYCHWILGEW
RVGVKDQDKKDGAKYSYKDLCNELKQKVTKAGLVDFLLELDPCRTIPPYL
DNNNRKPPKCQSLILNPKFLDNQYPNWQQYLQELKKLQSIQNYLDSFETD
LKVLKSSKDQPYFVEYKSSNQQIASGQRDYKDLDARILQFIFDRVKASDE
LLLNEIYFQAKKLKQKASSELEKLESSKKLDEVIANSQLSQILKSQHTNG
IFEQGTFLHLVCKYYKQRQRARDSRLYIMPEYRYDKKLHKYNNTGRFDDD
NQLLTYCNHKPRQKRYQLLNDLAGVLQVSPNFLKDKIGSDDDLFISKWLV
EHIRGFKKACEDSLKIQKDNRGLLNHKINIARNTKGKCEKEIFNLICKIE
GSEDKKGNYKHGLAYELGVLLFGEPNEASKPEFDRKIKKFNSIYSFAQIQ
QIAFAERKGNANTCAVCSADNAHRMQQIKIIEPVEDNKDKIILSAKAQRL
PAIPTRIVDGAVKKMATILAKNIVDDNWQNIKQVLSAKHQLHIPIIlESN
AFEFEPALADVKGKSLKDRRKKALERISPENIFKDKNNRIKEFAKGISAY
SGANLTDGDFDGAKEELDHIIPRSHKKYGTLNDEANLICVTRGDNKNKGN
RIFCLRDLADNYKLKQFETTDDLEIEKKIADTIWDANKKDFKFGNYRSFI
NLTPQEQKAFRHALFLADENPIKQAVIRAINNRNRTFVNGTQRYFAEVLA
NNIYLRAKKENLNTDKISFDYFGIPTIGNGRGIAEIRQLYEKVDSDIQAY
AKGDKPQASYSHLIDAMLAFCIAADEHRNDGSIGLEIDKNYSLYPLDKNT
GEVFTKDIFSQIKITDNEFSDKKLVRKKAIEGFNTHRQMTRDGIYAENYL
PILIHKELNEVRKGYTWKNSEEIKIFKGKKYDIQQLNNLVYCLKFVDKPI
SIDIQISTLEELRNILTTNNIAATAEYYYINLKTQKLHEYYIENYNTALG
YKKYSKEMEFLRSLAYRSERVKIKSIDDVKQVLDKDSNFIIGKITLPFKK
EWQRLYREWQNTTIKDDYEFLKSFFNVKSITKLHKKVRKDFSLPISTNEG
KFLVKRKTWDNNFIYQILNDSDSRADGTKPFIPAFDISKNEIVEAIIDSF
TSKNIFWLPKNIELQKVDNKNIFAIDTSKWFEVETPSDLRDIGIATIQYK
IDNNSRPKVRVKLDYVIDDDSKINYFMNHSLLKSRYPDKVLEILKQSTII
EFESSGFNKTIKEMLGMKLAGIYNETSNN

In some embodiments the Cas9 protein can be Lactobacillus buchneri Cas9 and may comprise or consist of the amino acid sequence:

(SEQ ID NO: 164)
MKVNNYHIGLDIGTSSIGWVAIGKDGKPLRVKGKTAIGARLFQEGNPAAD
RRMFRTTRRRLSRRKWRLKLLEEIFDPYITPVDSTFFARLKQSNLSPKDS
RKEFKGSMLFPDLTDMQYHKNYPTIYHLRHALMTQDKKFDIRMVYLAIHH
IVKYRGNFLNSTPVDSFKASKVDFVDQFKKLNELYAAINPEESFKINLAN
SEDIGHQFLDPSIRKFDKKKQIPKIVPVMMNDKVTDRLNGKIASEIIHAI
LGYKAKLDVVLQCTPVDSKPWALKFDDEDIDAKLEKILPEMDENQQSIVA
ILQNLYSQVTLNQIVPNGMSLSESMIEKYNDHHDHLKLYKKLIDQLADPK
KKAVLKKAYSQYVGDDGKVIEQAEFWSSVKKNLDDSELSKQIMDLIDAEK
FMPKQRTSQNGVIPHQLHQRELDEIIEHQSKYYPWLVEINPNKHDLHLAK
YKIEQLVAFRVPYYVGPMITPKDQAESAETVFSWMERKGIETGQITPWNF
DEKVDRKASANRFIKRMTTKDTYLIGEDVLPDESLLYEKFKVLNELNMVR
VNGKLLKVADKQAIFQDLFENYKHVSVKKLQNYIKAKTGLPSDPEISGLS
DPEHFNNSLGTYNDFKKLFGSKVDEPDLQDDFEKIVEWSTVFEDKKILRE
KLNEITWLSDQQKDVLESSRYQGWGRLSKKLLTGIVNDQGERIIDKLWNT
NKNFMQIQSDDDFAKRIHEANADQMQAVDVEDVLADAYTSPQNKKAIRQV
VKVVDDIQKAMGGVAPKYISIEFTRSEDRNPRRTISRQRQLENTLKDTAK
SLAKSINPELLSELDNAAKSKKGLTDRLYLYFTQLGKDIYTGEPINIDEL
NKYDIDHILPQAFIKDNSLDNRVLVLTAVNNGKSDNVPLRMFGAKMGHFW
KQLAEAGLISKRKLKNLQTDPDTISKYAMHGFIRRQLVETSQVIKLVANI
LGDKYRNDDTKIIEITARMNHQMRDEFGFIKNREINDYHHAFDAYLTAFL
GRYLYHRYIKLRPYFVYGDFKKFREDKVTMRNFNFLHDLTDDTQEKIADA
ETGEVIWDRENSIQQLKDVYHYKFMLISHEVYTLRGAMFNQTVYPASDAG
KRKLIPVKADRPVNVYGGYSGSADAYMAIVRIHNKKGDKYRVVGVPMRAL
DRLDAAKNVSDADFDRALKDVLAPQLTKTKKSRKTGEITQVIEDFEIVLG
KVMYRQLMIDGDKKFMLGSSTYQYNAKQLVLSDQSVKTLASKGRLDPLQE
SMDYNNVYIEILDKVNQYFSLYDMNKFRHKLNLGFSKFISFPNHNVLDGN
TKVSSGKREILQEILNGLHANPTFGNLKDVGITTPFGQLQQPNGILLSDE
TKIRYQSPTGLFERTVSLKDL

In some embodiments the Cas9 protein can be Listeria innocua Cas9 and may comprise or consist of the amino acid sequence:

(SEQ ID NO: 165)
MKKPYTIGLDIGTNSVGWAVLTDQYDLVKRKMKIAGDSEKKQIKKNFWGV
RLFDEGQTAADRRMARTARRRIERRRNRISYLQGIFAEEMSKTDANFFCR
LSDSFYVDNEKRNSRHPFFATIEEEVEYHKNYPTIYHLREELVNSSEKAD
LRLVYLALAHIIKYRGNFLIEGALDTQNTSVDGIYKQFIQTYNQVFASGI
EDGSLKKLEDNKDVAKILVEKVTRKEKLERILKLYPGEKSAGMFAQFISL
IVGSKGNFQKPFDLIEKSDIECAKDSYEEDLESLLALIGDEYAELFVAAK
NAYSAVVLSSIITVAElETNAKLSASMIERFDTHEEDLGELKAFIKLHLP
KHYEEIFSNlEKHGYAGYIDGKTKQADFYKYMKMTLENIEGADYFIAKIE
KENFLRKQRTFDNGAIPHQLHLEELEAILHQQAKYYPFLKENYDKIKSLV
TFRIPYFVGPLANGQSEFAWLTRKADGEIRPWNIEEKVDFGKSAVDFIEK
MTNKDTYLPKENVLPKHSLCYQKYLVYNELTKVRYINDQGKTSYFSGQEK
EQIFNDLFKQKRKVKKKDLELFLRNMSHVESPTIEGLEDSFNSSYSTYHD
LLKVGIKQEILDNPVNIEMLENIVKILTVFEDKRMIKEQLQQFSDVLDGV
VLKKLERRHYTGWGRLSAKLLMGIRDKQSHLTILDYLMNDDGLNRNLMQL
INDSNLSFKSIIEKEQVTTADKDIQSIVADLAGSPAIKKGILQSLKIVDE
LVSVMGYPPQTIVVEMARENQTTGKGKNNSRPRYKSLEKAIKEFGSQILK
EHPTDNQELRNNRLYLYYLQNGKDMYTGQDLDIHNLSNYDIDHIVPQSFI
TDNSIDNLVLTSSAGNREKGDDVPPLEIVRKRKVFWEKLYQGNLMSKRKF
DYLTKAERGGLTEADKARFIHRQLVETRQITKNVANILHQRFNYEKDDHG
NTMKQVRIVTLKSALVSQFRKQFQLYKVRDVNDYHHAHDAYLNGVVANTL
LKVYPQLEPEFVYGDYHQFDWFKANKATAKKQFYTNIMLFFAQKDRIIDE
NGEILWDKKYLDTVKKVMSYRQMNIVKKTEIQKGEFSKATIKPKGNSSKL
IPRKTNWDPMKYGGLDSPNMAYAVVIEYAKGKNKLVFEKKIIRVTIMERK
AFEKDEKAFLEEQGYRQPKVLAKLPKYTLYECEEGRRRMLASANEAQKGN
QQVLPNHLVTLLHHAANCEVSDGKSLDYIESNREMFAELLAHVSEFAKRY
TLAEANLNKINQLFEQNKEGDIKAIAQSFVDLMAFNAMGAPASFKFFETT
IERKRYNNLKELLNSTIIYQSITGLYESRKRLDD

In some embodiments the Cas9 protein can be L. pneumophilia Cas9 and may comprise or consist of the amino acid sequence:

(SEQ ID NO: 166)
MESSQILSPIGIDLGGKFTGVCLSHLEAFAELPNHANTKYSVILIDHNNF
QLSQAQRRATRHRVRNKKRNQFVKRVALQLFQHILSRDLNAKEETALCHY
LNNRGYTYVDTDLDEYIKDETTINLLKELLPSESEHNFIDWFLQKMQSSE
FRKILVSKVEEKKDDKELKNAVKNIKNFITGFEKNSVEGHRHRKVYFENI
KSDITKDNQLDSIKKKIPSVCLSNLLGHLSNLQWKNLHRYLAKNPKQFDE
QTFGNEFLRMLKNFRHLKGSQESLAVRNLIQQLEQSQDYISILEKTPPEI
TIPPYEARTNTGMEKDQSLLLNPEKLNNLYPNWRNLIPGIIDAHPFLEKD
LEHTKLRDRKRIISPSKQDEKRDSYILQRYLDLNKKIDKFKIKKQLSFLG
QGKQLPANLIETQKEMETHFNSSLVSVLIQIASAYNKEREDAAQGIWFDN
AFSLCELSNINPPRKQKILPLLVGAILSEDFINNKDKWAKFKIFWNTHKI
GRTSLKSKCKEIEEARKNSGNAFKIDYEEALNHPEHSNNKALIKIIQTIP
DIIQAIQSHLGHNDSQALIYHNPFSLSQLYTILETKRDGFHKNCVAVTCE
NYWRSQKTEIDPEISYASRLPADSVRPFDGVLARMMQRLAYEIAMAKWEQ
IKHIPDNSSLLIPIYLEQNRFEFEESFKKIKGSSSDKTLEQAIEKQNIQW
EEKFQRIINASMNICPYKGASIGGQGEIDHIYPRSLSKKHFGVIFNSEVN
LIYCSSQGNREKKEEHYLLEHLSPLYLKHQFGTDNVSDIKNFISQNVANI
KKYISFHLLTPEQQKAARHALFLDYDDEAFKTITKFLMSQQKARVNGTQK
FLGKQIMEFLSTLADSKQLQLEFSIKQITAEEVHDHRELLSKQEPKLVKS
RQQSFPSHAIDATLTMSIGLKEFPQFSQELDNSWFINHLMPDEVHLNPVR
SKEKYNKPNISSTPLFKDSLYAERFIPVWVKGETFAIGFSEKDLFEIKPS
NKEKLFTLLKTYSTKNPGESLQELQAKSKAKWLYFPINKTLALEFLHHYF
HKEIVTPDDTTVCHFINSLRYYTKKESITVKILKEPMPVLSVKFESSKKN
VLGSFKHTIALPATKDWERLFNHPNFLALKANPAPNPKEFNEFIRKYFLS
DNNPNSDIPNNGHNIKPQKHKAVRKVFSLPVIPGNAGTMMRIRRKDNKGQ
PLYQLQTIDDTPSMGIQINEDRLVKQEVLMDAYKTRNLSTIDGINNSEGQ
AYATFDNWLTLPVSTFKPEIIKLEMKPHSKTRRYIRITQSLADFIKTIDE
ALMIKPSDSIDDPLNMPNEIVCKNKLFGNELKPRDGKMKIVSTGKIVTYE
FESDSTPQWIQTLYVTQLKKQP

In some embodiments the Cas9 protein can be N. lactamica Cas9 and may comprise or consist of the amino acid sequence:

(SEQ ID NO: 167)
MAAFKPNPMNYILGLDIGIASVGWAMVEVDEEENPIRLIDLGVRVFERAE
VPKTGDSLAMARRLARSVRRLTRRRAHRLLRARRLLKREGVLQDADFDEN
GLVKSLPNTPWQLRAAALDRKLTCLEWSAVLLHLVKHRGYLSQRKNEGET
ADKELGALLKGVADNAHALQTGDFRTPAELALNKFEKESGHIRNQRGDYS
HTFSRKDLQAELNLLFEKQKEFGNPHVSDGLKEDIETLLMAQRPALSGDA
VQKMLGHCTFEPAEPKAAKNTYTAERFIWLTKLNNLRILEQGSERPLTDT
ERATLMDEPYRKSKLTYAQARKLLGLEDTAFFKGLRYGKDNAEASTLMEM
KAYHAISRALEKEGLKDKKSPLNLSTELQDEIGTAFSLFKTDKDITGRLK
DRVQPEILEALLKHISFDKFVQISLKALRRIVPLMEQGKRYDEACAEIYG
DHYCKKNAEEKIYLPPIPADEIRNPVVLRALSQARKVINCVVRRYGSPAR
IHIETAREVGKSFKDRKEIEKRQEENRKDREKAAAKFREYFPNFVGEPKS
KDILKLRLYEQQHGKCLYSGKEINLVRLNEKGYVEIDHALPFSRTWDDSF
NNKVLVLGSENQNKGNQTPYEYFNGKDNSREWQEFKARVETSRFPRSKKQ
RILLQKFDEEGFKERNLNDTRYVNRFLCQFVADHILLTGKGKRRVFASNG
QITNLLRGFWGLRKVRTENDRHHALDAVVVACSTVAMQQKITRFVRYKEM
NAFDGKTIDKETGEVLHQKAHFPQPWEFFAQEVMIRVFGKPDGKPEFEEA
DTPEKLRTLLAEKLSSRPEAVHEYVTPLFVSRAPNRKMSGQGHMETVKSA
KRLDEGISVLRVPLTQLKLKGLEKMVNREREPKLYDALKAQLETHKDDPA
KAFAEPFYKYDKAGSRTQQVKAVRIEQVQKTGVVVVRNHNGIADNATMVR
VDVFEKGGKYYLVPIYSWQVAKGILPDRAVVAFKDEEDWTVMDDSFEFRF
VLYANDLIKLTAKKNEFLGYFVSLNRATGAIDIRTHDTDSTKGKNGIFQS
VGVKTALSFQKNQIDELGKEIRPCRLKKRPPVR

In some embodiments the Cas9 protein can be N. meningitides Cas9 and may comprise or consist of the amino acid sequence:

(SEQ ID NO: 168)
MAAFKPNPINYILGLDIGIASVGWAMVEIDEDENPICLIDLGVRVFERAE
VPKTGDSLAMARRLARSVRRLTRRRAHRLLRARRLLKREGVLQAADFDEN
GLIKSLPNTPWQLRAAALDRKLTPLEWSAVLLHLIKHRGYLSQRKNEGET
ADKELGALLKGVADNAHALQTGDFRTPAELALNKFEKESGHIRNQRGDYS
HTFSRKDLQAELILLFEKQKEFGNPHVSGGLKEGIETLLMTQRPALSGDA
VQKMLGHCTFEPAEPKAAKNTYTAERFIWLTKLNNLRILEQGSERPLTDT
ERATLMDEPYRKSKLTYAQARKLLGLEDTAFFKGLRYGKDNAEASTLMEM
KAYHAISRALEKEGLKDKKSPLNLSPELQDEIGTAFSLFKTDEDITGRLK
DRIQPEILEALLKHISFDKFVQISLKALRRIVPLMEQGKRYDEACAEIYG
DHYGKKNTEEKIYLPPIPADEIRNPVVLRALSQARKVINGVVRRYGSPAR
IHIETAREVGKSFKDRKEIEKRQEENRKDREKAAAKFREYFPNFVGEPKS
KDILKLRLYEQQHGKCLYSGKEINLGRLNEKGYVEIDHALPFSRTWDDSF
NNKVLVLGSENQNKGNQTPYEYFNGKDNSREWQEFKARVETSRFPRSKKQ
RILLQKFDEDGFKERNLNDTRYVNRFLCQFVADRMRLTGKGKKRVFASNG
QITNLLRGFWGLRKVRAENDRHHALDAVVVACSTVAMQQKITRFVRYKEM
NAFDGKTIDKETGEVLHQKTHFPQPWEFFAQEVMIRVFGKPDGKPEFEEA
DTPEKLRTLLAEKLSSRPEAVHEYVTPLFVSRAPNRKMSGQGHMETVKSA
KRLDEGVSVLRVPLTQLKLKDLEKMVNREREPKLYEALKARLEAHKDDPA
KAFAEPFYKYDKAGNRTQQVKAVRVEQVQKTGVVVVRNHNGIADNATMVR
VDVFEKGDKYYLVPIYSWQVAKGILPDRAVVQGKDEEDWQLIDDSFNFKF
SLHPNDLVEVITKKARMFGYFASCHRGTGNINIRIHDLDHKIGKNGILEG
IGVKTALSFQKYQIDELGKEIRPCRLKKRPPVR

In some embodiments the Cas9 protein can be B. longum Cas9 and may comprise or consist of the amino acid sequence:

(SEQ ID NO: 169)
MLSRQLLGASHLARPVSYSYNVQDNDVHCSYGERCFMRGKRYRIGIDVGL
NSVGLAAVEVSDENSPVRLLNAQSVIHDGGVDPQKNKEAITRKNMSGVAR
RTRRMRRRKRERLHKLDMLLGKFGYPVIEPESLDKPFEEWHVRAELATRY
IEDDELRRESISIALRHMARHRGWRNPYRQVDSLISDNPYSKQYGELKEK
AKAYNDDATAAEEESTPAQLVVAMLDAGYAEAPRLRWRTGSKKPDAEGYL
PVRLMQEDNANELKQIFRVQRVPADEWKPLFRSVFYAVSPKGSAEQRVGQ
DPLAPEQARALKASLAFQEYRIANVITNLRIKDASAELRKLTVDEKQSIY
DQLVSPSSEDITWSDLCDFLGFKRSQLKGVGSLTEDGEERISSRPPRLTS
VQRIYESDNKIRKPLVAWWKSASDNEHEAMIRLLSNTVDIDKVREDVAYA
SAIEFIDGLDDDALTKLDSVDLPSGRAAYSVETLQKLTRQMLTTDDDLHE
ARKTLFNVTDSWRPPADPIGEPLGNPSVDRVLKNVNRYLMNCQQRWGNPV
SVNIEHVRSSFSSVAFARKDKREYEKNNEKRSIFRSSLSEQLRADEQMEK
VRESDLRRLEAIQRQNGQCLYCGRTITFRTCEMDHIVPRKGVGSTNTRTN
FAAVCAECNRMKSNTPFAIWARSEDAQTRGVSLAEAKKRVTMFTFNPKSY
APREVKAFKQAVIARLQQTEDDAAIDNRSIESVAWMADELHRRIDWYFNA
KQYVNSASIDDAEAETMKTTVSVFQGRVTASARRAAGIEGKIHFIGQQSK
TRLDRRHHAVDASVIAMMNTAAAQTLMERESLRESQRLIGLMPGERSWKE
YPYEGTSRYESFHLWLDNMDVLLELLNDALDNDRIAVMQSQRYVLGNSIA
HDATIHPLEKVPLGSAMSADLIRRASTPALWCALTRLPDYDEKEGLPEDS
HREIRVHDTRYSADDEMGFFASQAAQIAVQEGSADIGSAIHHARVYRCWK
TNAKGVRKYFYGMIRVFQTDLLRACHDDLFTVPLPPQSISMRYGEPRVVQ
ALQSGNAQYLGSLVVGDEIEMDFSSLDVDGQIGEYLQFFSQFSGGNLAWK
HWVVDGFFNQTQLRIRPRYLAAEGLAKAFSDDVVPDGVQKIVTKQGWLPP
VNTASKTAVRIVRRNAFGEPRLSSAHHMPCSWQWRHE

In some embodiments the Cas9 protein can be A. muciniphila Cas9 and may comprise or consist of the amino acid sequence:

(SEQ ID NO: 170)
MSRSLTFSFDIGYASIGWAVIASASHDDADPSVCGCGTVLFPKDDCQAFK
RREYRRLRRNIRSRRVRIERIGRLLVQAQIITPEMKETSGHPAPFYLASE
ALKGHRTLAPIELWHVLRWYAHNRGYDNNASWSNSLSEDGGNGEDTERVK
HAQDLMDKHGTATMAETICRELKLEEGKADAPMEVSTPAYKNLNTAFPRL
IVEKEVRRILELSAPLIPGLTAEIIELIAQHHPLTTEQRGVLLQHGIKLA
RRYRGSLLFGQLIPRFDNRIISRCPVTWAQVYEAELKKGNSEQSARERAE
KLSKVPTANCPEFYEYRMARILCNIRADGEPLSAEIRRELMNQARQEGKL
TKASLEKAISSRLGKETETNVSNYFTLHPDSEEALYLNPAVEVLQRSGIG
QILSPSVYRIAANRLRRGKSVTPNYLLNLLKSRGESGEALEKKIEKESKK
KEADYADTPLKPKYATGRAPYARTVLKKVVEEILDGEDPTRPARGEAHPD
GELKAHDGCLYCLLDTDSSVNQHQKERRLDTMTNNHLVRHRMLILDRLLK
DLIQDFADGQKDRISRVCVEVGKELTTFSAMDSKKIQRELTLRQKSHTDA
VNRLKRKLPGKALSANLIRKCRIAMDMNWTCPFTGATYGDHELENLELEH
IVPHSFRQSNALSSLVLTWPGVNRMKGQRTGYDFVEQEQENPVPDKPNLH
ICSLNNYRELVEKLDDKKGHEDDRRRKKKRKALLMVRGLSHKHQSQNHEA
MKEIGMTEGMMTQSSHLMKLACKSIKTSLPDAHIDMIPGAVTAEVRKAWD
VFGVFKELCPEAADPDSGKILKENLRSLTHLHHALDACVLGLIPYIIPAH
HNGLLRRVLAMRRIPEKLIPQVRPVANQRHYVLNDDGRMMLRDLSASLKE
NIREQLMEQRVIQHVPADMGGALLKETMQRVLSVDGSGEDAMVSLSKKKD
GKKEKNQVKASKLVGVFPEGPSKLKALKAAIEIDGNYGVALDPKPVVIRH
IKVFKRIMALKEQNGGKPVRILKKGMLIHLTSSKDPKHAGVVVRIESIQD
SKGGVKLDLQRAHCAVPKNKTHECNWREVDLISLLKKYQMKRYPTSYTGT
PR

In some embodiments the Cas9 protein can be O. laneus Cas9 and may comprise or consist of the amino acid sequence:

(SEQ ID NO: 171)
METTLGIDLGTNSIGLALVDQEEHQILYSGVRIFPEGINKDTIGLGEKEE
SRNATRRAKRQMRRQYFRKKLRKAKLLELLIAYDMCPLKPEDVRRWKNWD
KQQKSTVRQFPDTPAFREWLKQNPYELRKQAVTEDVTRPELGRILYQMIQ
RRGFLSSRKGKEEGKIFTGKDRMVGIDETRKNLQKQTLGAYLYDIAPKNG
EKYRFRTERVRARYTLRDMYIREFEIIWQRQAGHLGLAHEQATRKKNIFL
EGSATNVRNSKLITHLQAKYGRGHVLIEDTRITVTFQLPLKEVLGGKIEI
EEEQLKFKSNESVLFWQRPLRSQKSLLSKCVFEGRNFYDPVHQKWIIAGP
TPAPLSHPEFEEFRAYQFINNITYGKNEHLTAIQREAVFELMCTESKDFN
FEKIPKHLKLFEKFNFDDTTKVPACTTISQLRKLFPHPVVVEEKREEIWH
CFYFYDDNTLLFEKLQKDYALQTNDLEKIKKIRLSESYGNVSLKAIRRIN
PYLKKGYAYSTAVLLGGIRNSFGKRFEYFKEYEPEIEKAVCRILKEKNAE
GEVIRKIKDYLVHNRFGFAKNDRAFQKLYHHSQAITTQAQKERLPETGNL
RNPIVQQGLNELRRTVNKLLATCREKYGPSFKFDHIHVEMGRELRSSKIE
REKQSRQIRENEKKNEAAKVKLAEYGLKAYRDNIQKYLLYKEIEEKGGTV
CCPYTGKTLNISHTLGSDNSVQIEHIIPYSISLDDSLANKTLCDATFNRE
KGELTPYDFYQKDPSPEKWGASSWEEIEDRAFRLLPYAKAQRFIRRKPQE
SNEFISRQLNDTRYISKKAVEYLSAICSDVKAFPGQLTAELRHLWGLNNI
LQSAPDITFPLPVSATENHREYYVITNEQNEVIRLFPKQGETPRIEKGEL
LLTGEVERKVFRCKGMQEFQTDVSDGKYWRRIKLSSSVTWSPLFAPKPIS
ADGQIVLKGRIEKGVFVCNQLKQKLKTGLPDGSYWISLPVISQTFKEGES
VNNSKLTSQQVQLFGRVREGIFRCHNYQCPASGADGNFWCTLDTDTAQPA
FTPIKNAPPGVGGGQIILTGDVDDKGIFHADDDLHYELPASLPKGKYYGI
FTVESCDPTLIPIELSAPKTSKGENLIEGNIWVDEHTGEVRFDPKKNRED
QRHHAIDAIVIALSSQSLFQRLSTYNARRENKKRGLDSTEHFPSPWPGFA
QDVRQSVVPLLVSYKQNPKTLCKISKTLYKDGKKIHSCGNAVRGQLHKET
VYGQRTAPGATEKSYHIRKDIRELKTSKHIGKVVDITIRQMLLKHLQENY
HIDITQEFNIPSNAFFKEGVYRIFLPNKHGEPVPIKKIRMKEELGNAERL
KDNINQYVNPRNNHHVMIYQDADGNLKEEIVSFWSVIERQNQGQPIYQLP
REGRNIVSILQINDTFLIGLKEEEPEVYRNDLSTLSKHLYRVQKLSGMYY
TFRHHLASTLNNEREEFRIQSLEAWKRANPVKVQIDEIGRITFLNGPLC.

In some embodiments of the compositions of the disclosure, the sequence encoding the fRNA binding protein comprises a sequence isolated or derived from a CRISPR Cas protein. In some embodiments, the CRISPR Cas protein comprises a Type V CRISPR Cas protein. In some embodiments, the Type V CRISPR Cas protein comprises a Cpf1 protein. Exemplary Cpf1 proteins of the disclosure may be isolated or derived from any species, including, but not limited to, a bacteria or an archaea. Exemplary Cpf1 proteins of the disclosure may be isolated or derived from any species, including, but not limited to, Francisella tularensis subsp. novicida, Acidaminococcus sp. BV3L6 and Lachnospiraceae bacterium sp. ND2006. Exemplary Cpf1 proteins of the disclosure may be nuclease inactivated.

Exemplary wild type Francisella tularensis subsp. Novicida Cpf1 (FnCpf1) proteins of the disclosure may comprise or consist of the amino acid sequence:

| (SEQ ID NO: 172) | |
|---|---|
| 1 | MSIYQEFVNK YSLSKTLRFE LIPQGKTLEN IKARGLILDD EKRAKDYKKA KQIIDKYHQF |
| 61 | FIEEILSSVC ISEDLLQNYS DVYFKLKKSD DDNLQKDFKS AKDTIKKQIS EYIKDSEKFK |
| 121 | NLFNQNLIDA KKGQESDLIL WLKQSKDNGI ELFKANSDIT DIDEALEIIK SFKGWTTYFK |
| 181 | GFHENRKNVY SSNDIPTSII YRIVDDNLPK FLENKAKYES LKDKAPEAIN YEQIKKDLAE |
| 241 | ELTFDIDYKT SEVNQRVFSL DEVFEIANFN NYLNQSGITK FNTIIGGKFV NGENTKRKGI |
| 301 | NEYINLYSQQ INDKTLKKYK MSVLFKQILS DTESKSFVID KLEDDSDVVT TMQSFYEQIA |
| 361 | AFKTVEEKSI KETLSLLFDD LKAQKLDLSK IYFKNDKSLT DLSQQVFDDY SVIGTAVLEY |
| 421 | ITQQIAPKNL DNPSKKEQEL IAKKTEKAKY LSLETIKLAL EEFNKHRDID KQCRFEEILA |
| 481 | NFAAIPMIFD EIAQNKDNLA QISIKYQNQG KKDLLQASAE DDVKAIKDLL DQTNNLLHKL |
| 541 | KIFHISQSED KANILDKDEH FYLVFEECYF ELANIVPLYN KIRNYITQKP YSDEKFKLNF |
| 601 | ENSTLANGWD KNKEPDNTAI LFIKDDKYYL GVMNKKNNKI FDDKAIKENK GEGYKKIVYK |
| 661 | LLPGANKMLP KVFFSAKSIK FYNPSEDILR IRNHSTHTKN GSPQKGYEKF EFNIEDCRKF |
| 721 | IDFYKQSISK HPEWKDFGFR FSDTQRYNSI DEFYREVENQ GYKLTFENIS ESYIDSVVNQ |
| 781 | GKLYLFQIYN KDFSAYSKGR PNLHTLYWKA LFDERNLQDV VYKLNGEAEL FYRKQSIPKK |
| 841 | ITHPAKEAIA NKNKDNPKKE SVFEYDLIKD KRFTEDKFFF HCPITINFKS SGANKFNDEI |
| 901 | NLLLKEKAND VHILSIDRGE RHLAYYTLVD GKGNIIKQDT FNIIGNDRMK TNYHDKLAAI |
| 961 | EKDRDSARKD WKKINNIKEM KEGYLSQVVH EIAKLVIEYN AIVVFEDLNF GFKRGRFKVE |
| 1021 | KQVYQKLEKM LIEKLNYLVF KDNEFDKTGG VLRAYQLTAP FETFKKMGKQ TGIIYYVPAG |
| 1081 | FTSKICPVTG FVNQLYPKYE SVSKSQEFFS KFDKICYNLD KGYFEFSFDY KNFGDKAAKG |
| 1141 | KWTIASFGSR LINFRNSDKN HNWDTREVYP TKELEKLLKD YSIEYGHGEC IKAAICGESD |
| 1201 | KKFFAKLTSV LNTILQMRNS KTGTELDYLI SPVADVNGNF FDSRQAPKNM PQDADANGAY |
| 1261 | HIGLKGLMLL GRIKNNQEGK KLNLVIKNEE YFEFVQNRNN |

Exemplary wild type Lachnospiraceae bacterium sp. ND2006 Cpf1 (LbCpf1) proteins of the disclosure may comprise or consist of the amino acid sequence:

| (SEQ ID NO: 173) | |
|---|---|
| 1 | AASKLEKFTN CYSLSKTLRF KAIPVGKTQE NIDNKRLLVE DEKRAEDYKG VKKLLDRYYL |
| 61 | SFINDVLHSI KLKNLNNYIS LFRKKTRTEK ENKELENLEI NLRKEIAKAF KGAAGYKSLF |
| 121 | KKDIIETILP EAADDKDEIA LVNSENGETT AFTGFFDNRE NMFSEEAKST SIAFRCINEN |
| 181 | LTRYISNMDI FEKVDAIFDK HEVQEIKEKI LNSDYDVEDF FEGEFFNFVL TQEGIDVYNA |
| 241 | IIGGFVTESG EKIKGLNEYI NLYNAKTKQA LPKFKPLYKQ VLSDRESLSF YGEGYTSDEE |
| 301 | VLEVFRNTLN KNSEIFSSIK KLEKLFKNFD EYSSAGIFVK NGPAISTISK DIFGEWNLIR |
| 361 | DKWNAEYDDI HLKKKAVVTE KYEDDRRKSF KKIGSFSLEQ LQEYADADLS VVEKLKEIII |
| 421 | QKVDEIYKVY GSSEKLFDAD FVLEKSLKKN DAVVAIMKDL LDSVKSFENY IKAFFGEGKE |
| 481 | TNRDESFYGD FVLAYDILLK VDHIYDAIRN YVTQKPYSKD KFKLYFQNPQ FMGGWDKDKE |
| 541 | TDYRATILRY GSKYYLAIMD KKYAKCLQKI DKDDVNGNYE KINYKLLPGP NKMLPKVFFS |
| 601 | KKWMAYYNPS EDIQKIYKNG TFKKGDMFNL NDCHKLIDFF KDSISRYPKW SNAYDFNFSE |
| 661 | TEKYKDIAGF YREVEEQGYK VSFESASKKE VDKLVEEGKL YMFQIYNKDF SDKSHGTPNL |
| 721 | HTMYFKLLFD ENNHGQIRLS GGAELFMRRA SLKKEELVVH PANSPIANKN PDNPKKTTTL |
| 781 | SYDVYKDKRF SEDQYELHIP IAINKCPKNI FKINTEVRVL LKHDDNPYVI GIDRGERNLL |
| 841 | YIVVVDGKGN IVEQYSLNEI INNFNGIRIK TDYHSLLDKK EKERFEARQN WTSIENIKEL |
| 901 | KAGYISQVVH KICELVEKYD AVIALEDLNS GFKNSRVKVE KQVYQKFEKM LIDKLNYMVD |
| 961 | KKSNPCATGG ALKGYQITNK FESFKSMSTQ NGFIFYIPAW LTSKIDPSTG FVNLLKTKYT |
| 1021 | SIADSKKFIS SFDRIMYVPE EDLFEFALDY KNFSRTDADY IKKWKLYSYG NRIRIFAAAK |
| 1081 | KNNVFAWEEV CLTSAYKELF NKYGINYQQG DIRALLCEQS DKAFYSSFMA LMSLMLQMRN |
| 1141 | SITGRTDVDF LISPVKNSDG IFYDSRNYEA QENAILPKNA DANGAYNIAR KVLWAIGQFK |
| 1201 | KAEDEKLDKV KIAISNKEWL EYAQTSVK |

Exemplary wild type Acidaminococcus sp. BV3L6 Cpf1 (AsCpf1) proteins of the disclosure may comprise or consist of the amino acid sequence:

| (SEQ ID NO: 174) | |
|---|---|
| 1 | MTQFEGFTNL YQVSKTLRFE LIPQGKTLKH IQEQGFIEED KARNDHYKEL KPIIDRIYKT |
| 61 | YADQCLQLVQ LDWENLSAAI DSYRKEKTEE TRNALIEEQA TYRNAIHDYF IGRIDNLIDA |
| 121 | INKRHAEIYK GLFKAELENG KVLKQLGTVT TTEHENALLR SFDKFTTYFS GFYENRKNVF |
| 181 | SAEDISTAIP HRIVQDNFPK FKENCHIFTR LITAVPSLRE HFENVKKAIG IFVSTSIEEV |
| 241 | FSFPFYNQLL TQTQIDLYNQ LLGGISREAG TEKIKGLNEV LNLAIQKNDE TAHIIASLPH |
| 301 | RFIPLFKQIL SDRNTLSFIL EEFKSDEEVI QSFCKYKTLL RNENVLETAE ALFNELNSID |
| 361 | LTHIFISHKK LETISSALCD HWDTLRNALY ERRISELTGK ITKSAKEKVQ RSLKHEDINL |
| 421 | QEIISAAGKE LSEAFKQKTS EILSHAHAAL DQPLPTTLKK QEEKEILKSQ LDSLLGLYHL |
| 481 | LDWFAVDESN EVDPEFSARL TGIKLEMEPS LSFYNKARNY ATKKPYSVEK FKLNFQMPTL |
| 541 | ASGWDVNKEK NNGAILFVKN GLYYLGIMPK QKGRYKALSF EPTEKTSEGF DKMYYDYFPD |
| 601 | AAKMIPKCST QLKAVTAHFQ THTTPILLSN NFIEPLEITK EIYDLNNPEK EPKKFQTAYA |
| 661 | KKTGDQKGYR EALCKWIDFT RDFLSKYTKT TSIDLSSLRP SSQYKDLGEY YAELNPLLYH |
| 721 | ISFQRIAEKE IMDAVETGKL YLFQIYNKDF AKGHHGKPNL HTLYWTGLFS PENLAKTSIK |
| 781 | LNGQAELFYR PKSRMKRMAH RLGEKMLNKK LKDQKTPIPD TLYQELYDYV NHRLSHDLSD |
| 841 | EARALLPNVI TKEVSHEIIK DRRFTSDKFF FHVPITLNYQ AANSPSKFNQ RVNAYLKEHP |
| 901 | ETPIIGIDRG ERNLIYITVI DSTGKILEQR SLNTIQQFDY QKKLDNREKE RVAARQAWSV |
| 961 | VGTIKDLKQG YLSQVIHEIV DLMIHYQAVV VLENLNFGFK SKRTGIAEKA VYQQFEKMLI |
| 1021 | DKLNCLVLKD YPAEKVGGVL NPYQLTDQFT SFAKMGTQSG FLFYVPAPYT SKIDPLTGFV |
| 1081 | DPFVWKTIKN HESRKHFLEG FDFLHYDVKT GDFILHFKMN RNLSFQRGLP GFMPAWDIVF |
| 1141 | EKNETQFDAK GTPFIAGKRI VPVIENHRFT GRYRDLYPAN ELIALLEEKG IVFRDGSNIL |
| 1201 | PKLLENDDSH AIDTMVALIR SVLQMRNSNA ATGEDYINSP VRDLNGVCFD SRFQNPEWPM |
| 1261 | DADANGAYHI ALKGQLLLNH LKESKDLKLQ NGISNQDWLA YIQELRN |

In some embodiments of the compositions of the disclosure, the sequence encoding the RNA binding protein comprises a sequence isolated or derived from a CRISPR Cas protein or RNA-binding portion thereof. In some embodiments, the CRISPR Cas protein comprises a Type VI CRISPR Cas protein. In some embodiments, the Type VI CRISPR Cas protein comprises a Cas13 protein. Exemplary Cas13 proteins of the disclosure may be isolated or derived from any species, including, but not limited to, a bacteria or an archaea. Exemplary Cas13 proteins of the disclosure may be isolated or derived from any species, including, but not limited to, Leptotrichia wadei, Listeria seeligeri serovar 1/2b (strain ATCC 35967/DSM 20751/CIP 100100/SLCC 3954), Lachnospiraceae bacterium, Clostridium aminophilum DSM 10710, Carnobacterium gallinarum DSM 4847, Paludibacter propionicigenes WB4, Listeria weihenstephanensis FSL R9-0317, Listeria weihenstephanensis FSL R9-0317, bacterium FSL M6-0635 (Listeria newyorkensis), Leptotrichia wadei F0279, Rhodobacter capsulatus SB 1003, Rhodobacter capsulatus R121, Rhodobacter capsulatus DE442 and Corynebacterium ulcerans. Exemplary Cas13 proteins of the disclosure may be DNA nuclease inactivated. Exemplary Cas13 proteins of the disclosure include, but are not limited to, Cas13a, Cas13b, Cas13c, Cas13d and orthologs thereof. Exemplary Cas13b proteins of the disclosure include, but are not limited to, subtypes 1 and 2 referred to herein as Csx27 and Csx28, respectively.

Exemplary Cas13a proteins include, but are not limited to:

| Cas13a | Cas13a | | Accession | |
|---|---|---|---|---|
| number | abbreviation | Organism name | number | Direct Repeat sequence |
| Cas13a1 | LshCas13a | Leptotrichia | WP_018451595.1 | CCACCCCAATATCGAAGGGGACTAA |
| | | shahii | | AAC (SEQ ID NO: 175) |
| Cas13a2 | LwaCas13a | Leptotrichia | WP_021746774.1 | GATTTAGACTACCCCAAAAACGAAG |
| | | wadei | | GGGACTAAAAC (SEQ ID NO: |
| | | | | 176) |
| Cas13a3 | LseCas13a | Listeria seeligeri | WP_012985477.1 | GTAAGAGACTACCTCTATATGAAAG |
| | | | | AGGACTAAAAC (SEQ ID NO: |
| | | | | 177) |
| Cas13a4 | LbmCas13a | Lachnospiraceae | WP_044921188.1 | GTATTGAGAAAAGCCAGATATAGTT |
| | | bacterium | | GGCAATAGAC (SEQ ID NO: 178) |
| | | MA2020 | | |
| Cas13a5 | LbnCas13a | Lachnospiraceae | WP_022785443.1 | GTTGATGAGAAGAGCCCAAGATAG |
| | | bacterium | | AGGGCAATAAC (SEQ ID NO: |
| | | NK4A179 | | 179) |
| Cas13a6 | CamCas13a | [Clostridium] | WP_031473346.1 | GTCTATTGCCCTCTATATCGGGCTGT |
| | | aminophilum | | TCTCCAAAC (SEQ ID NO: 180) |
| | | DSM 10710 | | |
| Cas13a7 | CgaCas13a | Carnobacterium | WP_034560163.1 | ATTAAAGACTACCTCTAAATGTAAG |
| | | gallinarum DSM | | AGGACTATAAC (SEQ ID NO: |
| | | 4847 | | 181) |
| Cas13a8 | Cga2Cas13a | Carnobacterium | WP_034563842.1 | AATATAAACTACCTCTAAATGTAAG |
| | | gallinarum DSM | | AGGACTATAAC (SEQ ID NO: |
| | | 4847 | | 182) |
| Cas13a9 | Pprcas13a | Paludibacter | WP_013443710.1 | CTTGTGGATTATCCCAAAATTGAAG |
| | | propionicigenes | | GGAACTACAAC (SEQ ID NO: |
| | | WB4 | | 183) |
| Cas13a10 | LweCas13a | Listeria | WP_036059185.1 | GATTTAGAGTACCTCAAAATAGAAG |
| | | weihenstephanensis | | AGGTCTAAAAC (SEQ ID NO: |
| | | FSL R9-0317 | | 184) |
| Cas13a11 | LbfCas13a | Listeriaceae | WP_036091002.1 | GATTTAGAGTACCTCAAAACAAAAG |
| | | bacterium FSL | | AGGACTAAAAC (SEQ ID NO: |
| | | M6-0635 | | 185) |
| | | (Listeria | | |
| | | newyorkensis) | | |
| Cas13a12 | Lwa2cas13a | Leptotrichia | WP_021746774.1 | GATATAGATAACCCCAAAAACGAA |
| | | wadei F0279 | | GGGATCTAAAAC (SEQ ID NO: |
| | | | | 186) |
| Cas13a13 | RcsCas13a | Rhodobacter | WP_013067728.1 | GCCTCACATCACCGCCAAGACGACG |
| | | capsulatus SB | | GCGGACTGAAC (SEQ ID NO: |
| | | 1003 | | 187) |
| Cas13a14 | RcrCas13a | Rhodobacter | WP_023911507.1 | GCCTCACATCACCGCCAAGACGACG |
| | | capsulatus R121 | | GCGGACTGAAC (SEQ ID NO: |
| | | | | 188) |
| Cas13a15 | RcdCas13a | Rhodobacter | WP_023911507.1 | GCCTCACATCACCGCCAAGACGACG |
| | | capsulatus | | GCGGACTGAAC (SEQ ID NO: |
| | | DE442 | | 189) |

Exemplary wild type Cas13a proteins of the disclosure may comprise or consist of the amino acid sequence:

| (SEQ ID NO: 190) | |
|---|---|
| 1 | MGNLFGHKRW YEVRDKKDFK IKRKVKVKRN YDGNKYILNI NENNNKEKID NNKFIRKYIN |
| 61 | YKKNDNILKE FTRKFHAGNI LFKLKGKEGI IRIENNDDFL ETEEVVLYIE AYGKSEKLKA |
| 121 | LGITKKKIID EAIRQGITKD DKKIEIKRQE NEEEIEIDIR DEYTNKTLND CSIILRIIEN |
| 181 | DELETKKSIY EIFKNINMSL YKIIEKIIEN ETEKVFENRY YEEHLREKLL KDDKIDVILT |
| 241 | NFMEIREKIK SNLEILGFVK FYLNVGGDKK KSKNKKMLVE KILNINVDLT VEDIADFVIK |
| 301 | ELEFWNITKR IEKVKKVNNE FLEKRRNRTY IKSYVLLDKH EKFKIERENK KDKIVKFFVE |
| 361 | NIKNNSIKEK IEKILAEFKI DELIKKLEKE LKKGNCDTEI FGIFKKHYKV NFDSKKFSKK |
| 421 | SDEEKELYKI IYRYLKGRIE KILVNEQKVR LKKMEKIEIE KILNESILSE KILKRVKQYT |
| 481 | LEHIMYLGKL RHNDIDMITV NTDDFSRLHA KEELDLELIT FFASTNMELN KIFSRENINN |
| 541 | DENIDFFGGD REKNYVLDKK ILNSKIKIIR DLDFIDNKNN ITNNFIRKFT KIGTNERNRI |
| 601 | LHAISKERDL QGTQDDYNKV INIIQNLKIS DEEVSKALNL DVVFKDKKNI ITKINDIKIS |
| 661 | EENNNDIKYL PSFSKVLPEI LNLYRNNPKN EPFDTIETEK IVLNALIYVN KELYKKLILE |
| 721 | DDLEENESKN IFLQELKKTL GNIDEIDENI IENYYKNAQI SASKGNNKAI KKYQKKVIEC |
| 781 | YIGYLRKNYE ELFDFSDFKM NIQEIKKQIK DINDNKTYER ITVKISDKTI VINDDFEYII |
| 841 | SIFALLNSNA VINKIRNRFF ATSVWLNTSE YQNIIDILDE IMQLNTLRNE CITENWNLNL |
| 901 | EEFIQKMKEI EKDFDDFKIQ TKKEIFNNYY EDIKNNILTE FKDDINGCDV LEKKLEKIVI |
| 961 | FDDETKFEID KKSNILQDEQ RKLSNINKKD LKKKVDQYIK DKDQEIKSKI LCRIIFNSDF |
| 1021 | LKKYKKEIDN LIEDMESENE NKFQEIYYPK ERKNELYIYK KNLFLNIGNP NFDKIYGLIS |
| 1081 | NDIKMADAKF LFNIDGKNIR KNKISEIDAI LKNLNDKLNG YSKEYKEKYI KKLKENDDFF |
| 1141 | AKNIQNKNYK SFEKDYNRVS EYKKIRDLVE FNYLNKIESY LIDINWKLAI QMARFERDMH |
| 1201 | YIVNGLRELG IIKLSGYNTG ISRAYPKRNG SDGFYTTTAY YKFFDEESYK KFEKICYGFG |
| 1261 | IDLSENSEIN KPENESIRNY ISHFYIVRNP FADYSIAEQI DRVSNLLSYS TRYNNSTYAS |
| 1321 | VFEVFKKDVN LDYDELKKKF KLIGNNDILE RLMKPKKVSV LELESYNSDY IKNLIIELLT |
| 1381 | KIENTNDTL |

Exemplary Cas13b proteins include, but are not limited to:

| | Cas13b | Cas13b |
|---|---|---|
| Species | Accession | Size (aa) |
| Paludibacter propionicigenes | WP_013446107.1 | 1155 |
| WB4 | | |
| Prevotella sp. P5-60 | WP_044074780.1 | 1091 |
| Prevotella sp. P4-76 | WP_044072147.1 | 1091 |
| Prevotella sp. P5-125 | WP_044065294.1 | 1091 |
| Prevotella sp. P5-119 | WP_042518169.1 | 1091 |
| Capnocytophaga canimorsus Cc5 | WP_013997271.1 | 1200 |
| Phaeodactylibacter xiamenensis | WP_044218239.1 | 1132 |
| Porphyromonas gingivalis W83 | WP_005873511.1 | 1136 |
| Porphyromonas gingivalis F0570 | WP_021665475.1 | 1136 |
| Porphyromonas gingivalis | WP_012458151.1 | 1136 |
| ATCC 33277 | | |
| Porphyromonas gingivalis F0185 | ERJ81987.1 | 1136 |
| Porphyromonas gingivalis F0185 | WP_021677657.1 | 1136 |
| Porphyromonas gingivalis SJD2 | WP_023846767.1 | 1136 |
| Porphyromonas gingivalis F0568 | ERJ65637.1 | 1136 |
| Porphyromonas gingivalis W4087 | ERJ87335.1 | 1136 |
| Porphyromonas gingivalis W4087 | WP_021680012.1 | 1136 |
| Porphyromonas gingivalis F0568 | WP_021663197.1 | 1136 |
| Porphyromonas gingivalis | WP_061156637.1 | 1136 |
| Porphyromonas gulae | WP_039445055.1 | 1136 |
| Bacteroides pyogenes F0041 | ERI81700.1 | 1116 |
| Bacteroides pyogenes JCM 10003 | WP_034542281.1 | 1116 |
| Alistipes sp. ZOR0009 | WP_047447901.1 | 954 |
| Flavobacterium branchiophilum | WP_014084666.1 | 1151 |
| FL-15 | | |
| Prevotella sp. MA2016 | WP_036929175.1 | 1323 |
| Myroides odoratimimus | EHO06562.1 | 1160 |
| CCUG 10230 | | |
| Myroides odoratimimus | EKB06014.1 | 1158 |
| CCUG 3837 | | |
| Myroides odoratimimus | WP_006265509.1 | 1158 |
| CCUG 3837 | | |
| Myroides odoratimimus | WP_006261414.1 | 1158 |
| CCUG 12901 | | |
| Myroides odoratimimus | EHO08761.1 | 1158 |
| CCUG 12901 | | |
| Myroides odoratimimus | WP_058700060.1 | 1160 |
| (NZ_CP013690.1) | | |
| Bergeyella zoohelcum | EKB54193.1 | 1225 |
| ATCC 43767 | | |
| Capnocytophaga cynodegmi | WP_041989581.1 | 1219 |
| Bergeyella zoohelcum | WP_002664492.1 | 1225 |
| ATCC 43767 | | |
| Flavobacterium sp. 316 | WP_045968377.1 | 1156 |
| Psychroflexus torquis | WP_015024765.1 | 1146 |
| ATCC 700755 | | |
| Flavobacterium columnare | WP_014165541.1 | 1180 |
| ATCC 49512 | | |
| Flavobacterium columnare | WP_060381855.1 | 1214 |
| Flavobacterium columnare | WP_063744070.1 | 1214 |
| Flavobacterium columnare | WP_065213424.1 | 1215 |
| Chryseobacterium sp. YR477 | WP_047431796.1 | 1146 |
| Riemerella anatipestifer | WP_004919755.1 | 1096 |
| ATCC 11845 = DSM 15868 | | |
| Riemerella anatipestifer RA-CH-2 | WP_015345620.1 | 949 |
| Riemerella anatipestifer | WP_049354263.1 | 949 |
| Riemerella anatipestifer | WP_061710138.1 | 951 |
| Riemerella anatipestifer | WP_064970887.1 | 1096 |
| Prevotella saccharolytica F0055 | EKY00089.1 | 1151 |
| Prevotella saccharolytica | WP_051522484.1 | 1152 |
| JCM 17484 | | |
| Prevotella buccae ATCC 33574 | EFU31981.1 | 1128 |
| Prevotella buccae ATCC 33574 | WP_004343973.1 | 1128 |
| Prevotella buccae D17 | WP_004343581.1 | 1128 |
| Prevotella sp. MSX73 | WP_007412163.1 | 1128 |
| Prevotella pallens ATCC 700821 | EGQ18444.1 | 1126 |
| Prevotella pallens ATCC 700821 | WP_006044833.1 | 1126 |
| Prevotella intermedia | WP_036860899.1 | 1127 |
| ATCC 25611 = DSM 20706 | | |
| Prevotella intermedia | WP_061868553.1 | 1121 |
| Prevotella intermedia 17 | AFJ07523.1 | 1135 |
| Prevotella intermedia | WP_050955369.1 | 1133 |
| Prevotella intermedia | BAU18623.1 | 1134 |
| Prevotella intermedia ZT | KJJ86756.1 | 1126 |
| Prevotella aurantiaca JCM 15754 | WP_025000926.1 | 1125 |
| Prevotella pleuritidis F0068 | WP_021584635.1 | 1140 |
| Prevotella pleuritidis JCM 14110 | WP_036931485.1 | 1117 |
| Prevotella falsenii | WP_036884929.1 | 1134 |
| DSM 22864 = JCM 15124 | | |
| Porphyromonas gulae | WP_039418912.1 | 1176 |
| Porphyromonas sp. | WP_039428968.1 | 1176 |
| COT-052 OH4946 | | |
| Porphyromonas gulae | WP_039442171.1 | 1175 |
| Porphyromonas gulae | WP_039431778.1 | 1176 |
| Porphyromonas gulae | WP_046201018.1 | 1176 |
| Porphyromonas gulae | WP_039434803.1 | 1176 |
| Porphyromonas gulae | WP_039419792.1 | 1120 |
| Porphyromonas gulae | WP_039426176.1 | 1120 |
| Porphyromonas gulae | WP_039437199.1 | 1120 |
| Porphyromonas gingivalis TDC60 | WP_013816155.1 | 1120 |
| Porphyromonas gingivalis | WP_012458414.1 | 1120 |
| ATCC 33277 | | |
| Porphyromonas gingivalis | WP_058019250.1 | 1176 |
| A7A1-28 | | |
| Porphyromonas gingivalis | EOA10535.1 | 1176 |
| JCVI SC001 | | |
| Porphyromonas gingivalis W50 | WP_005874195.1 | 1176 |
| Porphyromonas gingivalis | WP_052912312.1 | 1176 |
| Porphyromonas gingivalis AJW4 | WP_053444417.1 | 1120 |
| Porphyromonas gingivalis | WP_039417390.1 | 1120 |
| Porphyromonas gingivalis | WP_061156470.1 | 1120 |

Exemplary wild type Bergeyella zoohelcum ATCC 43767 Cas13b (BzCas13b) proteins of the disclosure may comprise or consist of the amino acid sequence:

| (SEQ ID NO: 191) | |
|---|---|
| 1 | menktslgnn iyynpfkpqd ksyfagyfna amentdsvfr elgkrlkgke ytsenffdai |
| 61 | fkenislvey eryvkllsdy fpmarlldkk evpikerken fkknfkgiik avrdlrnfyt |
| 121 | hkehgeveit deifgvldem lkstvltvkk kkvktdktke ilkksiekql dilcqkkley |
| 181 | lrdtarkiee krrnqrerge kelvapfkys dkrddliaai yndafdvyid kkkdslkess |
| 241 | kakyntksdp qqeegdlkip iskngvvfll slfltkqeih afkskiagfk atvideatvs |
| 301 | eatvshgkns icfmatheif shlaykklkr kvrtaeinyg eaenaeqlsv yaketlmmqm |
| 361 | ldelskvpdv vyqnlsedvg ktfiedwney lkenngdvgt meeeqvihpv irkryedkfn |
| 421 | yfairfldef aqfptlrfqv hlgnylhdsr pkenlisdrr ikekitvfgr lselehkkal |
| 481 | fikntetned rehyweifpn pnydfpkeni svndkdfpia gsildrekqp vagkigikvk |
| 541 | llnqqyvsev dkavkahqlk qrkaskpsig niieeivpin esnpkeaivf ggqptaylsm |
| 601 | ndihsilyef fdkwekkkek lekkgekelr keigkelekk ivgkiqaqiq qiidkdtnak |
| 661 | ilkpyqdgns taidkeklik dlkqeqnilq klkdeqtvre keyndfiayq dknreinkvr |
| 721 | drnhkqylkd nlkrkypeap arkevlyyre kgkvavwlan dikrfmptdf knewkgeqhs |
| 781 | llqkslayye qckeelknll pekvfqhlpf klggyfqqky lyqfytcyld krleyisglv |
| 841 | qqaenfksen kvfkkvenec fkflkkqnyt hkeldarvqs ilgypifler gfmdekptii |
| 901 | kgktfkgnea lfadwfryyk eyqnfqtfyd tenyplvele kkqadrkrkt kiyqqkkndv |
| 961 | ftllmakhif ksvfkqdsid qfsledlyqs reerlgnqer arqtgerntn yiwnktvdlk |
| 1021 | lcdgkitven vklknvgdfi kyeydqrvqa flkyeeniew qaflikeske eenypyvver |
| 1081 | eieqyekvrr eellkevhli eeyilekvkd keilkkgdnq nfkyyilngl lkqlknedve |
| 1141 | sykvfnlnte pedvninqlk qeatdleqka fvltyirnkf ahnqlpkkef wdycqekygk |
| 1201 | iekektyaey faevfkkeke alik. |

In some embodiments of the compositions of the disclosure, the sequence encoding the first RNA binding protein comprises a sequence isolated or derived from a CasRX/Cas13d protein. CasRX/Cas13d is an effector of the type VI-D CRISPR-Cas systems. In some embodiments, the CasRX/Cas13d protein is an RNA-guided RNA endonuclease enzyme that can cut or bind RNA. In some embodiments, the CasRX/Cas13d protein can include one or more higher eukaryotes and prokaryotes nucleotide-binding (HEPN) domains. In some embodiments, the CasRX/Cas13d protein can include either a wild-type or mutated HEPN domain. In some embodiments, the CasRX/Cas13d protein includes a mutated HEPN domain that cannot cut RNA but can process guide RNA. In some embodiments, the CasRX/Cas13d protein does not require a protospacer flanking sequence. Also see WO Publication No. WO2019/040664 & US2019/0062724, which is incorporated herein by reference in its entirety, for further examples and sequences of CasRX/Cas13d protein, without limitation, specific reference is made to

Exemplary CasRX/Cas13d proteins may comprise or consist of the sequence: CasRX/Cas13d Gut_metagenome_contig6049000251:

| (SEQ ID NO: 54) | |
|---|---|
| LYLTSFGKGN AAVIEQKIEP ENGYRVTGMQ ITPSITVNKA TDESVRFRVK RKIAQKDEFI | 60 |
| ADNPMHEGRH RIEPSAGSDM LGLKTKLEKY YFGKEFDDNL HIQIIYNILD IEKILAVYST | 120 |
| NITA | 124. |

Exemplary CasRX/Cas13d proteins may comprise or consist of the sequence: CasRX/Cas13d Gut_metagenome_contig546000275:

| (SEQ ID NO: 57) | |
|---|---|
| MDSYRPKLYK LIDFCIFKHY HEYTEISEKN VDTLRAAVSE EQKESFYADE AKRLWGIFDK | 60 |
| QFLGFCKKIN VWVNGSHEKE ILGYIDKDAY RKKSDVSYFS KFLYAMSFFL DGKEINDLLT | 120 |
| TLINKFDNIA SFISTAKELD AEIDRILEKK LDPVTGKPLK GKNSFRNFIA NNVIENKRFI | 180 |
| YVIKFCNPKN VLKLVKNTKV TEFVLKRMPE SQIDRYYSSC IDTEKNPSVD KKISDLAEMI | 240 |
| KKIAFDDFRN VRQKTRTREE SLEKERFKAV IGLYLTVVYL LIKNLVNVNS RYVMAFHCLE | 300 |
| RDAKLYGINI GKNYIELTED LCRENENSRS AYLARNKRLR DCVKQNIDNA KNMKSKEK | 358. |

Exemplary CasRX/Cas13d proteins may comprise or consist of the sequence: CasRX/Cas13d Gut_metagenome_contig4114000374:

| (SEQ ID NO: 61) | |
|---|---|
| DTKINPQTWL YQLENTPDLD NEYRDTLDHF FDERFNEINE HFVTQNATNL CIMKEVFPDE | 60 |
| DFKSIADLYY DFIVVKSYKN IGFSIKKLRE KMLELPEAKR VTSTEMDSVR SKLYKLIDFC | 120 |
| IFKHYHEKPE TVEMIVSMLR AYTSEDMKE | 149. |

Exemplary CasRX/Cas13d proteins may comprise or consist of the sequence: CasRX/Cas13d Gut_metagenome_contig721000619:

| (SEQ ID NO: 67) | |
|---|---|
| KEGSTMAKNE KKKSTAKALG LKSSFVVNND IYMTSFGKGN KAVLEKKITE NTIENKSDTT | 60 |
| YFDVINRDPK GFTLEGRRIA DMTAFSNDPK YHVNVVNGKF LEDQLGARSE LEKKVFGRTF | 120 |
| DDNVHIQLIH NILDIEKIMA QYVSDIVYLL HNTIKRDMND DIMGYISIRN SFDDFCHPER | 180 |
| IPDRKAKDNL QKQHDIFFDE ILKCGRLAYF GNAFFEDGSD NKEIAKLKRY KEIYHIIALM | 240 |
| GSLRQSYFHG ENSDKNFQGP TWAYTLESNL TGKYKEFKDT LDKTFDERYE MISKDFGSTN | 300 |
| MVNLQILEEL LKMLYGNVSP | 320. |

Exemplary CasRX/Cas13d proteins may comprise or consist of the sequence: CasRX/Cas13d Gut_metagenome_contig2002000411:

| (SEQ ID NO: 69) | |
|---|---|
| EKQNKAKYQA IISLYLMVMY QIVKNMIYVN SRYVIAFHCL ERDSNQLLGR FNSRDASMYN | 60 |
| KLTQKFITDK YLNDGAQGCS KKVGNYLSHN ITCCSDELRK EYRNQVDHFA VVRMIGKYAA | 120 |
| DIGKFSTWFE LYHYVMQRII FDKRNPLSET ERTYKQLIAK HHTYCKDLVK ALNTPFGYNL | 180 |
| ARYKNLSIGE LFDRNNYNAK TKET | 204. |

Exemplary CasRX/Cas13d proteins may comprise or consist of the sequence: CasRX/Cas13d Gut_metagenome_contig13552000311:

| (SEQ ID NO: 71) | |
|---|---|
| LIDFLIYDLY YNRKPARIEE IVDKLRESVN DEEKESIYSA ETKYVYEALG KVLVRSLKKY | 60 |
| LNGATIRDLK NRYDAKTANR IWDISEHSKS GHVNCFCKLI YMMTLMLDGK EINDLLTTLV | 120 |
| NKFDNIASFI DVMDELGLEH SFTDNYKMFA DSKAICLDLQ FINSFARMSK IDDEKSKRQL | 180 |
| FRDALVVLDI GDKNEDWIEK YLTSDIFKRD ENGNKIDGEK RDFRNFIANN VIKSARFKYL | 240 |
| VKYSSADGMI KLKKNEKLIS FVLEQLPETQ IDRYYESCGL DCAVADRKVR IEKLTGLIRD | 300 |
| MRFDNFRGVN YSNDACKKDK QAKAKYQAII SLYLMVLYQI VKNMIYVNSR YVIAFHCLER | 360 |
| DLLFFNIELD NSYQYSNCNE LTEKFIKDKY MKEGALGFNM KAGRYLTKNI GNCSNELRKI | 420 |
| YRNQVDHFAV VRKIGNYAAD IASVGSWFE | 449. |

Exemplary CasRX/Cas13d proteins may comprise or consist of the sequence: CasRX/Cas13d Gut_metagenome_contig10037000527:

| (SEQ ID NO: 72) | |
|---|---|
| YMDQNFANSD AWAIHVYRNK IQHLDAVRHA DMYIGDIREF HSWFELYHYI IQRRIIDQYA | 60 |
| YESTPGSSRD GSAIIDEERL NPATRRYFRL ITTYKT | 96. |

Exemplary CasRX/Cas13d proteins may comprise or consist of the sequence: CasRX/Cas13d Gut_metagenome_contig238000329:

| (SEQ ID NO: 73) | |
|---|---|
| RYDKDRSKIY TMMDFVIYRY YIDNNNDSID FINKLRSSID EKSKEKLYNE EANRLWNKLK | 60 |
| EYMLYIKEFN GKLASRTPDR DGNISEFVES LPKIHRLLPR GQKISNFSKL MYLLTMFLDG | 120 |
| KEINDLLTTL INKFENIQGF LDIMPEINVN AKFEPEYVFF NKSHEIAGEL KLIKGFAQMG | 180 |
| EPAATLKLEM TADAIKILGT EKEDAELIKL AESLFKDENG KLLGNKQHGM RNFIGNNVIK | 240 |
| SKRFHYLIRY GDPAHLHKIA TNKNVVRFVL GRIADMQKKQ GQKGKNQIDR YYEVCVGNKD | 300 |
| IKKTIEEKID ALTDIIVNMN YDQFEKKKAV IENQNRGKTF EEKNKYKRDN AEREKFKKII | 360 |
| SLYLTVIYHI LKNIVNVNSR YILGFHCLER DKQLYIEKYN KDKLDGFVAL TKFCLGDEER | 420 |
| YEDLKAKAQA SIQALETANP KLYAKYMNYS DEEKKEEFKK QLNRERVKNA RNAYLKNIKN | 480 |
| YIMIRLQLRD QTDSSGYLCG EFRDKVAHLE VARHAHEYI | 519. |

Exemplary CasRX/Cas13d proteins may comprise or consist of the sequence: CasRX/Cas13d Gut_metagenome_contig2643000492:

| (SEQ ID NO: 84) | |
|---|---|
| NGEIVSLAEK EAFSAKIADK NIGCKIENKQ FRHPKGYDVI ADNPIYKGSP RQDMLGLKET | 60 |
| LEKRYFSPSD SIDNVRVQVA HNILDIEKIL AEYITNAVYS FDNIAGFGKD IIGDDFSPVY | 120 |
| TYDKFEKSDR YEYFKNLLNN SRLGYYGQAF FECDDSKENK KKKDAIKCYN IIALLSGLRH | 180 |
| W | 181. |

Exemplary CasRX/Cas13d proteins may comprise or consist of the sequence: CasRX/Cas13d Gut_metagenome_contig874000057:

| (SEQ ID NO: 85) | |
|---|---|
| MSKNKESYAK GMGLKSALVS GSKVYMTSFE GGNDAKLEKV VENSEIVSLA EKESFSAEIF | 60 |
| KKNIGCKIEN KKFKHPKRYD VIADNPLYKG SVRQDMLGLK ETLEKRYFNS ADGTDNVCIQ | 120 |
| VIHNILDIEK ILAEYITNAV YSFDNIAGFG EDIIGMGGFK PIYTYKQFKE PDKYNKKFDD | 180 |
| ILNNSRLGYY GKAFFEKNDL KHNPNKKKRD KNPYILKYDN ECYYIIALLS GLRHWNIHSH | 240 |
| AKDDLVSYRW LYNLDSILNR EYISTLNYLY DDIADELTES FSKNSSANVN YIAETLNIDP | 300 |
| SEFAQQYFRF SIMKEQKNMG FNVSKLREIM LDRKELSDIR DNHRVFDSIR SKLYTMMDFV | 360 |
| IYRYYIEEAA KTEAENRNLP ENEKKISEKD FFVINLRGSF DENQKEKLYI EEAKRLWEKL | 420 |
| KDIMLKIKEF RGEKVKEYKK | 440. |

Exemplary CasRX/Cas13d proteins may comprise or consist of the sequence: CasRX/Cas13d Gut_metagenome_contig4781000489:

| (SEQ ID NO: 86) | |
|---|---|
| LDKQLDYEYI RTLNYMFNDI ADELTRTFSK NSAANVNYIA ETLNIDPNKF AEQYFRFSIM | 60 |
| KEQKNLGFNL TKLRESMLDR RELSDIRDNH NVFDSIRPKL YTMMDFVIYK HYIDEAKKTE | 120 |
| AENKSLPDDR KNLSEKD | 137. |

Exemplary CasRX/Cas13d proteins may comprise or consist of the sequence: CasRX/Cas13d Gut_metagenome_contig12144000352:

| (SEQ ID NO: 87) | |
|---|---|
| RMGEPVANTK RVMMIDAVKI LGTDLSDDEL KEMADSFFKD SDGNLLKKGK HGMRNFITNN | 60 |
| VIKNKRFHYL IRYGDPAHLH EIAKNEA | 87. |

Exemplary CasRX/Cas13d proteins may comprise or consist of the sequence: CasRX/Cas13d Gut_metagenome_contig5590000448:

| (SEQ ID NO: 88) | |
|---|---|
| VHNNEEKDLI KYTWLYNLDK YLDAEYITTL NYMYNDIGDE LTDSFSKNSA ANINYIAETL | 60 |
| GIDPKTFAEQ YFRFSIMKEQ KNLGFNLTKL REVMLDRKDM SEIRENHNDF DSIRAKVYTM | 120 |
| MDFVIYRYYI EEAAKVNAAN KSLPDNEKSL SEKDIFVISL RGSFNEDQKD RLYYDEAQRL | 180 |
| WSKVGKLMLK IKKFRGKDTR KYKNMGTPRI RRLIPEGRDI STFSKLMYAL TMFLDGKEIN | 240 |
| DLLTTLINKF DNIQSFLKVM PLIGVNAKFA EEYSFFNNSE KIADELRLIK SFARMGEPVA | 300 |
| DARRAMYIDA IRILGTDLSD DELKALADSF SLDENGNKLG KGKHGMRNFI INNVITNKRF | 360 |
| HYLIRYGNPV HLHEIAKNEA VVKFVLGRIA DIQKKQGQNG KNQIDRYYET CIGK | 414. |

Exemplary CasRX/Cas13d proteins may comprise or consist of the sequence: CasRX/Cas13d Gut_metagenome_contig525000349:

| (SEQ ID NO: 89) | |
|---|---|
| MSKKENRKSY VKGLGLKSTL VSDSKVYLTT FADGSNAKLE KCVENNKIIC ISNDKEAFAA | 60 |
| SIANKNVGYK IKNDEKFRHP KGYDIISNNP LLHNNSVQQD MLGLKNVLEK RYFGKSSGGD | 120 |
| NNLCIQIIHN IIDIEKILSE YIPNVVYAFN NIAGFKDEHN NIIDIIGTQT YNSSYTYADF | 180 |
| SKDKSDKKYI EFQKLLKNKR LGYWGKAFFT GQGNNAKVRQ ENQCFHIIAL LISLRNWATH | 240 |
| SNELDKHTKR TWLYKLDDTN ILNAEYVKTL NYLYDTIADE LTKSFSKNGA VNVNYLAKKY | 300 |
| NIKDDLPGFS EQYFRFSIMK EQKNLGFNIS KLRENMLDFK DMSVI | 345. |

Exemplary CasRX/Cas13d proteins may comprise or consist of the sequence: CasRX/Cas13d Gut_metagenome_contig7229000302:

| (SEQ ID NO: 90) | |
|---|---|
| KKISSLTKFC LGESDEKKLK ALAKKSLEEL KTTNSKLYEN YIKYSDERKA EEAKRQINRE | 60 |
| RAKTAMNAHL RNTKWNDIMY GQLKDLADSK SRICSEFRNK AAHLEVARYA HMYINDISEV | 120 |
| KSYFRLYHYI MQRRIIDVIE NNPKAKYEGK VKVYFEDVKK NKKYNKNLLK LMCVPFGYCI | 180 |
| PRFKNLSIEQ MFDMNETDNS DKKKEK | 206. |

Exemplary CasRX/Cas13d proteins may comprise or consist of the sequence: CasRX/Cas13d Gut_metagenome_contig3227000343:

| (SEQ ID NO: 91) | |
|---|---|
| IGDISEVNSY FQLYHYIMQR ILIDKIGSKT TGKAKEYFDS VIVNKKYDDR LLKLLCSPLG | 60 |
| YCLTRYKDLS IEALFDMNEA AKYDKLNKER KNKKK | 95. |

Exemplary CasRX/Cas13d proteins may comprise or consist of the sequence: CasRX/Cas13d Gut_metagenome_contig7030000469:

| (SEQ ID NO: 92) | |
|---|---|
| SIRSKLYTMM DFVIYRYYIE ESAKAAAENK PSESDSFVIR LRGSFNENQK EELYIEEAER | 60 |
| LWKKFGEIML KIKEFRGEKV KEYKKEVPRI ERILPHGKDI SAFSKLMYML SMFLD | 115. |

Exemplary CasRX/Cas13d proteins may comprise or consist of the sequence: CasRX/Cas13d gut_metagenome_P17E0k2120140920, c87000043:

| (SEQ ID NO: 93) | |
|---|---|
| MYFSKMIYML TYFLDGKEIN DLLTTLISKF DNIKEFLKIM KSSAVDVECE LTAGYKLFND | 60 |
| SQRITNELFI VKNIASMRKP AASAKLTMFR DALTILGIDD KITDDRISEI LKLKEKGKGI | 120 |
| HGLRNFITNN VIESSRFVYL IKYANAQKIR EVAKNEKVVM FVLGGIPDTQ IERYYKSCVE | 180 |
| FPDMNSSLEA KRSELARMIK NISFDDFKNV KQQAKGRENV AKERAKAVIG LYLT | 234. |

Exemplary CasRX/Cas13d proteins may comprise or consist of the sequence: CasRX/Cas13d Metagenomic hit (no protein accession): contig emb|OBVH01003037.1, human gut metagenome sequence (also found in WGS contigs emb|OBXZ01000094.1| and emb|OBJF01000033.1|):

| (SEQ ID NO: 94) | |
|---|---|
| MAKKKRITAK ERKQNHRELL MKKADSNAEK EKAKKPVVEN KPDTAISKDN TPKPNKEIKK | 60 |
| SKAKLAGVKW VIKANDDVAY ISSFGKGNNS VLEKRIMGDV SSNVNKDSHM YVNPKYTKKN | 120 |
| YEIKNGFSSG SSLVTYPNKP DKNSGMDALC LKPYFEKDFF GHIFTDNMHI QAIYNIFDIE | 180 |
| KILAKHITNI IYTVNSFDRN YNQSGNDTIG FGLNYRVPYS EYGGGKDSNG EPKNQSKWEK | 240 |
| RDNFIKFYNE SKPHLGYYEN IFYDHGEPIS EEKFYNYLNI LNFIRNNTFH YKDDDIELYS | 300 |
| ENYSEEFVFI NCLNKFVKNK FKNVNKNFIS NEKNNLYIIL NAYGKDTENV EVVKKYSKEL | 360 |
| YKLSVLKTNK NLGVNVKKLR ESAIEYGYCP LPYDKEKEVA KLSSVKHKLY KTYDFVITHY | 420 |
| LNSNDKLLLE IVETLRLSKN DDEKENVYKK YAEKLFKADD VINPIKAISK LFARKGNKLF | 480 |
| KEKIIIKKEY IEDVSIDKNI YDFTKVIFFM TCFLDGKEIN DLLTNIISKL QVIEDHNNVI | 540 |
| KFISNNKDAV YKDYSDKYAI FRNAGKIATE LEAIKSIARM ENKIENAPQE PLLKDALLSL | 600 |
| GVSDDTKVLE NTYNKYFDSK EKTDKQSQKV STFLMNNVIN NNRFKYVIKY INPADINGLA | 660 |
| KNRYLVKFVL SKIPEEQIDS YYKLFSNEEE PGCEEKIKLL TKKISKLNFQ TLFENNKIPN | 720 |
| VEKEKKKAII TLYFTIVYIL VKNLVNINGL YTLALYFVER DGYFYKDICG KKDKKKSYND | 780 |
| VDYLLLPEIF SGSKYREETK NLKLPKEKDR DIMKKYLPND KDREKYNKFF TAYRNNIVHL | 840 |
| NIIAKLSELT KNIDKDINSY FDIYHYCTQR VMFNYCKEKN DVVLAKMKDL AHIKSDCNEF | 900 |
| SSKHTYPFSS AVLRFMNLPF AYNVPRFKNL SYKKFFDKQ. | 939 |

Exemplary CasRX/Cas13d proteins may comprise or consist of the sequence: CasRX/Cas13d Metagenomic hit (no protein accession): contig tpg|DJXD01000002.1| (uncultivated Ruminococcus assembly, UBA7013, from sheep gut metagenome):

| (SEQ ID NO: 95) | |
|---|---|
| MKKQKSKKTV SKTSGLKEAL SVQGTVIMTS FGKGNMANLS YKIPSSQKPQ NLNSSAGLKN | 60 |
| VEVSGKKIKF QGRHPKIATT DNPLFKPQPG MDLLCLKDKL EMHYFGKTFD DNIHIQLIYQ | 120 |
| ILDIEKILAV HVNNIVFTLD NVLHPQKEEL TEDFIGAGGW RINLDYQTLR GQTNKYDRFK | 180 |
| NYIKRKELLY FGEAFYHENE RRYEEDIFAI LTLLSALRQF CFHSDLSSDE SDHVNSFWLY | 240 |
| QLEDQLSDEF KETLSILWEE VTERIDSEFL KTNTVNLHIL CHVFPKESKE TIVRAYYEFL | 300 |
| IKKSFKNMGF SIKKLREIML EQSDLKSFKE DKYNSVRAKL YKLFDFIITY YYDHHAFEKE | 360 |
| ALVSSLRSSL TEENKEEIYI KTARTLASAL GADFKKAAAD VNAKNIRDYQ KKANDYRISF | 420 |
| EDIKIGNTGI GYFSELIYML TLLLDGKEIN DLLTTLINKF DNIISFIDIL KKLNLEFKFK | 480 |
| PEYADFFNMT NCRYTLEELR VINSIARMQK PSADARKIMY RDALRILGMD NRPDEEIDRE | 540 |
| LERTMPVGAD GKFIKGKQGF RNFIASNVIE SSRFHYLVRY NNPHKTRTLV KNPNVVKFVL | 600 |
| EGIPETQIKR YFDVCKGQEI PPTSDKSAQI DVLARIISSV DYKIFEDVPQ SAKINKDDPS | 660 |
| RNFSDALKKQ RYQAIVSLYL TVMYLITKNL VYVNSRYVIA FHCLERDAFL HGVTLPKMNK | 720 |
| KIVYSQLTTH LLTDKNYTTY GHLKNQKGHR KWYVLVKNNL QNSDITAVSS FRNIVAHISV | 780 |
| VRNSNEYISG IGELHSYFEL YHYLVQSMIA KNNWYDTSHQ PKTAEYLNNL KKHHTYCKDF | 840 |
| VKAYCIPFGY VVPRYKNLTI NELFDRNNPN PEPKEEV. | 877 |

An exemplary direct repeat sequence of CasRX/Cas13d Metagenomic hit (no protein accession): contig tpg|DJXD01000002.1| (uncultivated Ruminococcus assembly, UBA7013, from sheep gut metagenome) (SEQ ID NO: 95) comprises or consists of the nucleic acid sequence:

### CasRX/Cas13d DR:

(SEQ ID NO: 96)

| | |
|---|---|
| caactacaac cccgtaaaaa tacggggttc tgaaac. | 36 |

Exemplary CasRX/Cas13d proteins may comprise or consist of the sequence: CasRX/Cas13d Metagenomic hit (no protein accession): contig OGZC01000639.1 (human gut metagenome assembly):

| (SEQ ID NO: 97) | |
|---|---|
| MKKKNIRATR EALKAQKIKK SQENEALKKQ KLAEEAAQKR REELEKKNLA QWEETSAEGR | 60 |
| RSRVKAVGVK SVFVVGDDLY LATFGNGNET VLEKKITPDG KITTFPEEET FTAKLKFAQT | 120 |
| EPTVATSIGI SNGRIVLPEI SVDNPLHTTM QKNTIKRSAG EDILQLKDVL ENRYFDRSFN | 180 |
| DDLHIRLIYN ILDIEKILAE YTTNAVFAID NVSGCSDDFL SNFSTRNQWD EFQNPEQHRE | 240 |
| HFGNKDNVIC SVKKQQDLFF NFFKNNRIGY FGKAFFHAES ERKIVKKTEK EVYHILTLIG | 300 |
| SLRQWITHST EGGISRLWLY QLEDALSREY QETMNNCYNS TIYGLQKDFE KTNAPNLNFL | 360 |
| AEILGKNASE LAEPYFRFII TKEYKNLGFS IKTLREMLLD QPDLQEIREN HNVYDSIRSK | 420 |
| LYKMIDFVLV YAYSNERKSK ADALASNLRS AITEDAKKRI YQNEADQLWT SYQELFKRIR | 480 |
| GFKGAQVKEY SSKNMPIPIQ KQIQNILKPA EQVTYFTKLM YLLTMFLDGK EINDLLTTLI | 540 |
| NKFDNISSLL KTMEQLELQT TFKEDYTFFQ QSSRLCKEIT QLKSFARMGN PISNLKEVMM | 600 |
| VDAIQILGTE KSEQELQSMA CFFFRDKNGK KLNTGEHGMR NFIGNNVISN TRFQYLIRYG | 660 |
| NPQKLHTLSQ NETVVRFVLS RIAKNQRVQG MNGKNQIDRY YETCGGTNSW SVSEEEKINF | 720 |
| LCKILTNMSY DQFQDVKQSG AEITAEEKRK KERYKAIISL YLTVLYQLIK NLVNINARYI | 780 |
| IAFHCLERDA ILYSSKFNTS INLKKRYTAL TEMILGYETD EKARRKDTRT VYEKAEAAKN | 840 |
| RHLKNVKWNC KTRENLENAD KNAIVAFRNI VAHLWIIRDA DRFITGMGAM KRYFDCYHYL | 900 |
| LQRELGYILE KSNQGSEYTK KSLEKVQQYH SYCKDFLHML CLPFAYCIPR YKNLSIAELF | 960 |
| DRHEPEAEPK EEASSVNNSQ FITT. | 984 |

Exemplary CasRX/Cas13d proteins may comprise or consist of the sequence: CasRX/Cas13d Metagenomic hit (no protein accession): contig emb|OHBM01000764.1 (human gut metagenome assembly):

| (SEQ ID NO: 98) | |
|---|---|
| XXXXXXXXXX XXXXXXXXXX XXXXXXXXXX XXXXXXXXXX XXXXXXXXXX XXXXXXXXXX | 60 |
| XXXXXXXXXX XXXXXXXXXX XXXXXXXXXX XXXXXXXXXX XXXXXXXXXX XXXXXXXXXX | 120 |
| XXXXXXXXXX XXXXXXXXXX XXXXXXXXXX XXXXXXXXXX XXXXXXXXXX XXXXXXXXXX | 180 |
| XXXXXXXXXX XXXXXXXXXX XXXXXXXXXX XXXXXXXXXX XXHPLQKRYR YLTSTNLKSF | 240 |
| ETYKNNLVNK KKFDLDRVKK IPQLAYFGSA FYNTPEDTSA KITKTKIKSN EEIYYTFMLL | 300 |
| STARNFSAHY LDRNRAKSSD AEDFDGTSVI MYNLDNEELY KKLYNKKVHM ALTGMKKVLD | 360 |
| ANFNKKVEHL NNSFIKNSAK DFVILCEVLG IKSRDEKTKF VKDYYDFVVR KNYKHLGFSV | 420 |
| KELRELLFAN HDSNKYIKEF DKISNKKFDS VRSRLNRLAD YIIYDYYNKN NAKVSDLVKY | 480 |
| LRAAADDEQK KKIYLNESIN LVKSGILERI KKILPKLNGK IIGNMQPDST ITASMLHNTG | 540 |
| KDWHPISENA HYFTKWIYTL TLFMDGKEIN DLVTTLINKF DNIASFIEVL KSQSVCTHFS | 600 |
| EERKMFIDSA EICSELSAMN SFARMEAPGA SSKRAMFVEA ARILGDNRSK EELEEYFDTL | 660 |
| FDKSASKKEK GFRNFIRNNV VDSNRFKYLT RYTDTSSVKA FSNNKALVKF AIKDIPQEQI | 720 |
| LRYYNSCFGA SERYYNDGMS DKLVEAIGKI NLMQFNGVIQ QADRNMLPEE KKKANAQKEK | 780 |
| YKSIIRLYLT VCYLFFKNLV YVNSRYYSAF YNLEKDRSLF EINGELKPTG KFDEGHYTGL | 840 |
| VKLFIDNGWI NPRASAYLTV NLANSDETAI RTFRNTAEHL EALRNADKYL NDLKQFDSYF | 900 |
| EIYHYITQRN IKEKCEMLKE QTVKYNNDLL KYHGYSKDFV KALCVPFGYN LPRFKNLSID | 960 |
| ALFDKNDKRE KLKKGFED. | 978 |

Exemplary CasRX/Cas13d proteins may comprise or consist of the sequence: CasRX/Cas13d Metagenomic hit (no protein accession): contig emb|OHCP01000044.1 (human gut metagenome assembly):

| (SEQ ID NO: 99) | |
|---|---|
| MAKKITAKQK REEKERLNKQ KWAKNDSVII VPETKEEIKT GEIQDNNRKR SRQKSQAKAM | 60 |
| GLKAVLSFDN KIAIASFVSS KNAKSSHIER ITDKEGTTIS VNSKMFESSV NKRDINIEKR | 120 |
| ITIEEPQQDG TIKKEEKGVK STTCNPYFKV GGKDYIGIKE IAEEHFFGRA FPNENLRVQI | 180 |
| AYNIFDVQKI LGTFVNNIIY SFYNLSRDEV QSDNDVIGML YSISDYDRQK ETETFLQAKS | 240 |
| LLKQTEAYYA YFDDVFKKNK KPDKNKEGDN SKQYQENLRH NFNILRVLSF LRQICMHAEV | 300 |
| HVSDDEGCTR TQNYTDSLEA LFNISKAFGK KMPELKTLID NIYSKGINAI NDEFVKNGKN | 360 |
| NLYILSKVYP NEKREVLLRE YYNFVVCKEG SNIGISTRKL KETMIAQNMP SLKEENTYRN | 420 |
| KLYTVMNFIL VRELKNCATI REQMIKELRA NMDEEEGRDR IYSKYAKEIY LYVKDKLKLM | 480 |
| LNVFKEEAEG IIIPGKEDPV KFSHGKLDKK EIESFCLTTK NTEDITKVIY FLCKFLDGKE | 540 |
| INELCCAMMN KLDGISDLIE TAKQCGEDVE FVDQFKCLSK CATMSNQIRI VKNISRMKKE | 600 |
| MTIDNDTIFL DALELLGRKI EKYQKDKNGD YVKDEKGKKV YTKDYNNFQD MFFEGKNHRV | 660 |
| RNFVSNNVIK SKWFSYVVRY NKPAECQALM RNSKLVKFAL DELPDSQIEK YYISVFGEKS | 720 |
| SSSNEEMRRE LLKKLCDFSV RGFLDEIVLL SEDEMKQKDK FSEKEKKKSL IRLYLTIVYL | 780 |
| ITKSMVKINT RFSIACATYE RDYILLCQSE KAERAWEKGA TAFALTRKFL NHDKPTFEQY | 840 |
| YTREREISAM PQEKRKELRK ENDQLLKKTH YSKHAYCYIV DNVNNLTGAV ANDNGRGLPC | 900 |
| LSEKNDNANL FLEMRNKIVH LNVVHDMVKY INEIKNITSY YAFFCYVLQR MIIGNNSNEQ | 960 |
| NKFKAKYSKT LQEFGTYSKD LMWVLNLPFA YNLPRYKNLS NEQLFYDEEE RMEKIVGRKN | 1020 |
| DSR. | 1023 |

Exemplary CasRX/Cas13d proteins may comprise or consist of the sequence: CasRX/Cas13d Metagenomic hit (no protein accession): contig emb|OGDF01008514.1| (human gut metagenome assembly):

| (SEQ ID NO: 100) | |
|---|---|
| MTETKPKRED IAKTPAAKSR SKAAGLKSTF AVNGSVLLTS FGRGNDAVPE KLITEKAVSE | 60 |
| INTVKPRFSV EKPATSYSSS FGIKSHISAT ADNPLAGRAP VGEDAIHAKE VLEQRVFGKT | 120 |
| FSDDNIHIQL IYNILDIRKI LSTYANNVVF TINSMRRLDE YDREQDYLGY LYTGNSYERL | 180 |
| LDIADKYAVD GEDWRNTAAG ISNDFEKKQF QTINGFWDLL DMIEPYMCYF SEAFFCETTV | 240 |
| KDPDSGRIVP CLEQRSDGDI YNILRILSIV RQTCMHDNAS MRTVMFTLGQ NSVRDRKNGF | 300 |
| DELAELLDYL YDEKIDIVNR DFLRNQKNNI ELLSRIYGSS ADSPERDRLV QNFYDFRVLS | 360 |
| QDKNLGFSIK KLREKLLDSP ALSVVRSKKY DTMRSKIYSL IDFMIYRKFS ENHVAVDDFV | 420 |
| EELRSLLTED EKESAYSRWA ETLINDGFAQ EILVKLLPQT DPAVIGKIKG KKLLNDSIAG | 480 |
| IKLKKDASFF TKIINVLCMF QDGKEINELV SSLVNKFANI QSFVDVMRSQ GIDSGFTADY | 540 |
| AMFAESGRIS RELHILKGIA RMQHSIAGLG DVKIYGSDDK FHGVSRRVYT DAAYILGFGE | 600 |
| RSEDNDGYVD DYVSSKLLGG ADKNLRNFIT NNVIKNRRFL YTVRYMNPKR AKKLVQNDAL | 660 |
| VVLALSGIPE TQIDRYYKSC IEKRSFNPDL NEKIAALSEM ITTLKIDDFE DVKQNPEKNA | 720 |
| NYEAKKNQRI SKERYKACIG LYLTVLYLIC KNLVKINARY SIAIGCLERD TQLHGVDFKG | 780 |
| AAYMTRDVFI AKGWINPKKP TVKSIKEQYA FLTPYIFTTY RNMIAHLAAV TNAYKYIPQM | 840 |
| DRFKSWFHLY HTVIQHSLIQ QYEYDRDYGR KGAPVVSERV LQLLEQCREH SNYSRDLLHI | 900 |
| LNLPFGYNLP RYLNLSSEKY FDANAI. | 926 |

Exemplary CasRX/Cas13d proteins may comprise or consist of the sequence: CasRX/Cas13d Metagenomic hit (no protein accession): contig emb|OGPN01002610.1 (human gut metagenome assembly):

| (SEQ ID NO: 101) | |
|---|---|
| MAKKITAKQK REEKERLNKQ KWAKQDTPVV PKSKTEEKPV AASDDKLLKT TQVKKVQTKS | 60 |
| KAKAMGLKTV LSFDDKIAIA SFVNDKKTKL PHIERITDKS GTTIHENARM FDSSVDEQNV | 120 |
| NIEKRMTIEE KQNDGTFKKD EKDVKATICN PYFKTCGKDY IGIKDVAEKY FFGKTFPNEN | 180 |
| LRVQIAYNVF DIQKILGTYV NNIIYSFYNL RRDGKSDVDI IGSLYAFADF DNQLKDKPAF | 240 |
| REAKDLLKNT EAYFSYFGDV FKKSKKGKKD ENNEDYEKNL RHNFNVLRVL SFLRQICTHA | 300 |
| YVKCTGGAKN NGDSTKVEAE SLDALFNITE YFAKTAPELS KTINEIYKEG IDRINNDFVT | 360 |
| NGKNNLYILS KVYPDMQRNE LVKKYYQFVV CKEGNNVGIN TRKLKESIIS QHPWITTPQD | 420 |
| NNKANDYESC RHKLYTIMCF ILVAELDAHE SIRDNMVAEL RANMDGDDGR DAIYEKYAKD | 480 |
| IYHIVKDKLL AMQKVFDEEL VPVKVEGKND PQQFTHGKLG KKEIESFCLS DKNTSDIAKV | 540 |
| VYFLCNFLDG KEINELCCAM MNKFDGIGDL IDTAKQCGEE VKFIEEFACL SNCRKITNDI | 600 |
| RVAKSISKMK NKVNIDNDII YLDAIELLGR KIEKYQKDEN GKILLGTDGK RLYTQEYKYF | 660 |
| NDMFFNAGNH KVRNFIANNV MQSKWFFYVV RYNKPAECQI IMRNKTLVKF TLDDLPDMQI | 720 |
| QRYYSSVFGD NNMPAVDEMR KRLLDKINQF SVRGFLDELD EIVLMSDEES KRNKSSEKEQ | 780 |
| KKSLIRLYLT IAYLITKSMV KINTRFSIAC AMYERDYALL CQSEMKGGPW DGGAQALAVT | 840 |
| RKFLNHDREV FDRYCAREAE IARLPSEERK PLRKANDKLL KQTHYTNHSY TYIVNNLNSF | 900 |
| TDIDYCAKDV GLPAPNDKND NASILGEMRN DIAHLNIVHD MVKYIEELKD ISSYYAFYCY | 960 |
| VLQRRLVGKD PNCQNKFKAK YAKELNDYGT YNKNLMWMLN LPFAYNLPRY KNLSSEFLFY | 1020 |
| DMEYNKKDDE. | 1030 |

Exemplary CasRX/Cas13d proteins may comprise or consist of the sequence: CasRX/Cas13d Metagenomic hit (no protein accession): from contig emb|OBLI01020244 and emb|OBLI01038679 (from pig gut metagenome):

| (SEQ ID NO: 102) | |
|---|---|
| MAKKITAKQR REERERQNKQ KWAKKQADAT AVFECEADIK PADSKDEDCT NIYIKREKKK | 60 |
| TQAKAMGLKT VLGFDNKIAI ASFMSSKDSK SSHIERITDP NGKTIREDVR MFDSNVDECS | 120 |
| INLEKRMTVE ERQKDGTIKK DEKDVKSTIC NPYSNECGKD YIGIKSVAEE LFFGRTFPND | 180 |
| NLRVQIAYNI FDIQKILGTY INNIIYSFYN LSRDESQSDN DVIGTLYMLK DFDGQKETDT | 240 |
| FRQARALLER TEAYYSYFDN VFKKIDKNKK KSDDCKRERN EILRYNFNVL RVLSFLRQIC | 300 |
| AHAQVKISNE HDREKGGGLV DSLDALFNIS RFFDAVAPEL NEVINSVYSK GIDDINDNFV | 360 |
| KNGKNNFYIL SKIYPEVARE DLLREYYYFV VSKEGNNIGI STKKLKEAII VQDMSYIKSE | 420 |
| DYDTYRNKLY TVLCFILVKE LNERTTIREQ MVADLRANMN GDIGREDIYS KYAKIIYAQV | 480 |
| KPRFDTMKSA FEEEAKDVIV PDKKKPVKFS HGKLDKNEIE RFCITSANTD SVAKIIYFLC | 540 |
| KFLDGKEINE LCCAMMNKLD GINDLIETAE QCGAKVEFVD KFSVLSNCET ISDQIRIVKS | 600 |
| ISKMKKEIAI DNDTIFLDAL ELLGRKIDKY KKDATGKYLK DENGKYLYSK EYDDFQYMFF | 660 |
| KDSHRVRNFI SNSVIKSKWF SYIVRYNQPS ECRAIMKNKT LVKFALDELP DLQIQRYFVA | 720 |
| LYGDEDLPSY GEMRKILLKK LHDFSIKGFL DEIVLLSDLD MESQDKYCEK EQKKSLFRLY | 780 |
| LTIAYLITKS MVKINTRFSI ACATYERDYA LLCASNKQER AWSSGATALA LTRRFLNQDK | 840 |
| LIFEKHYARE GEISKLPKEE RKAMRKVNDQ LLKRTHFSKH SYCYIVDNVN RLTGGECRTD | 900 |
| KRVLPVLNEK NDNAGILLDF RKTIAHLNVV HKMVDYVDEI KGITSYYAFF CYVLQRMLVG | 960 |
| NNLNEKNAIK EKYSATVKSF GTYSKDFMWL INLPFAYNLP RYKNLSNEQL FYDEEERNET | 1020 |
| EEQIDRL. | 1027 |

Exemplary CasRX/Cas13d proteins may comprise or consist of the sequence: CasRX/Cas13d Metagenomic hit (no protein accession): contig OIZX01000427.1:

| (SEQ ID NO: 103) | |
|---|---|
| MAKKKKTARQ LREEMQQQRK QAIQKQQEQR QEKAAAARET AAPEQPAAAP VPKRQRKSLA | 60 |
| KAAGLKSNFI LDPQRRTTVM TAFGQGSTAI LEKQIVDRAI SDLQPVQQFQ VEPASAAKYR | 120 |
| LKNSRVRFPN VTADDPLYRR KDGGFVPGMD ALRRKNVLEQ RFFGKSFADN IHIQMIYSIL | 180 |
| DIHKILAAAS GHIVHLLNIV NGSKDRDFIG MLAAHVLYNE LNEEAKRSIA DFCKSPRLIY | 240 |
| YSAAFYETLD NGKSERRSNE DIFNILALMT CLRNFSSHHS IAIKVKDYSA AGLYNLRRLG | 300 |
| PDMKKMLDTF YTEAFIQLNQ SFQDHNTTNL TCLFDILNIS DSARQKQLAE EFYRYVVFKE | 360 |
| QKNLGFSVRK LREEMLLLPD AAVIADKRYD TCRSKLYNLM DFLILRVYRT GRADRCDKLP | 420 |
| EALRAALTDE EKAVVYHKEA LSLWNEMRTL ILDGLLPQMT PENLSRLSGQ KRKGELSLDD | 480 |
| AMLKECLYEP GPVPEDAAPE EANAEYFCRM IYLATLFMDG KEINTLLTTL ISKFENIAAF | 540 |
| LQTMEQLNIE AELGPEYAMF TRSRAVAEQL RVINSFALMK KPQVNAKQQL YRAAVTLLGT | 600 |
| EDPDGVTDEM LCIDPVTGKM LPPNQRHHGD TGLRNFIANN VVESRRFQYL IRYSDPAQLH | 660 |
| QLASNKKLVR FVLSSIPDTQ INRYYETCGQ TRLAGRAAKV EFLTDMIAAI RFDQFRDVNQ | 720 |
| KERGANTQKE RYKAMLGLYQ TVLYLAVKNL VNINARYVMA FHCVERDMFL YDGELTDPKG | 780 |
| ESVSAFLAVN GKKGVQPQYL LLTQLFIRRD YLKRSACEQI QHNMENISDR LLREYRNAVA | 840 |
| HLNVIAHLAD YSADMREITS YYGLYHYLMQ RHLFKRHAWQ IRQPERPTEE EQKLIEQEQK | 900 |
| QLAWEKALFD KTLQYHSYNK DLVKALNAPF GYNLARYKNL SIEPLFSKEA APAAEIKATH | 960 |
| A. | 961 |

Exemplary CasRX/Cas13d proteins may comprise or consist of the sequence: CasRX/Cas13d Metagenomic hit (no protein accession): contig OCTW011587266.1:

| (SEQ ID NO: 104) | |
|---|---|
| MKQNDRENNN KIKKSAAKAV GVKSLARLSD GSTVVSSFGK GAAAELESLI TGGEIRKLSD | 60 |
| KAILEITDDT QNKNAYNVKS SRIPNLTART DKLSDKSGMD DLGFKRELEL EVFGQCFDDS | 120 |
| IHIQIAHAVF DIQKSLAAVI PNVLYTLNNL DRSYSTDNTS DKKDIIGNTL NYQHSYESFN | 180 |
| VEKRGEFTEY YNAAKDRFSY FPDILCVLEK VNGKDRYQPK SEKDAFNVLS SVNMLRNSLF | 240 |
| HFAPKSNDGK ARIAVFKNQF DSDFSHITST VNKIYSAKIA GVNENFLNNE GNNLYIILKA | 300 |
| TNWDIKKIVP QLYRFSVLKS DKNMGFNMRK LREFAVESKN IDLSRLNDKF LTNNRKKLYK | 360 |
| VIDFIIYYHL NKVLKDSFVD DFVAALRASQ SEEEKEKLYA QYSERLFADE GLKSAIKKAV | 420 |
| DMISDTKSNI FKMKTPLDKA LIENIKVNSD ASDFCKLIYV FTRFLDGKEI NILLNSLIKK | 480 |
| FQDIHSFNTT VKKLSENNLI INADYVDDYS LFEQSGTVAR ELMLIKSISK MDFGLDNINL | 540 |
| SFMYDDALRT LGVSDENLPE VKREYFGKTK NLSAYIRNNV LENRRFKYVI KYIHPSDVQK | 600 |
| IACNKAIAGF VLNRMPDTQI KRYYDSLINK GATDIQAQAK ALLDCITGIS FDAIKDDKHL | 660 |
| HKSKEKSPQR SADRERKKAM LTLYYTIVYI FVKQMLHINS LYTIGFFYLE RDQRFIYSRA | 720 |
| KKENKNPSKN SYLNDFRSVT AYFIPSEIMK RIEKNENKGF LEDFEALWNS CGKTSRLRKE | 780 |
| DVLLYARYIS PDHALKNYKM ILNSYRNKIA HINVIMSAGK YTGGIKRMDS YFSVFQHLVQ | 840 |
| CDILSNPNNK GKCFESESLK PLLLDMKFDG TDEKLYSKRL TRALNIPFGY NVPRYKNLTF | 900 |
| EKIYLKSSIN E. | 911 |

Exemplary CasRX/Cas13d proteins may comprise or consist of the sequence: CasRX/Cas13d Metagenomic hit (no protein accession): contig emb|OGNF01009141.1:

| (SEQ ID NO: 105) | |
|---|---|
| MADIDKKKSS AKAAGLKSTF VLENNKLLMT SFGNGNKAVI EKIIDEKVDS INEPEVFSVT | 60 |
| PCDKKFELQP AKRGLAADSL VDNPLKSKKT AGDDAIHSRK FLERQFFDGN TFNDNIHIQL | 120 |
| IYNILDIEKI LSVHVNDIVY SVNNILSRGE GMEYNDYIGT LNLKSFETYK NNLVNKKKFD | 180 |
| LDRVKKIPQL AYFGSAFYNT PEDTSAKITK TKIKSNEEIY YTFMLLSTAR NFSAHYLDRN | 240 |
| RAKSSDAEDF DGTSVIMYNL DNEELYKKLY NKKVHMALTG MKKVLDANFN KKVEHLNNSF | 300 |
| IKNSAKDFVI LCEVLGIKSR DEKTKFVKDY YDFVVRKNYK HLGFSVKELR ELLFANHDSN | 360 |
| KYIKEFDKIS NKKFDSVRSR LNRLADYIIY DYYNKNNAKV SDLVKYLRAA ADDEQKKKIY | 420 |
| LNESINLVKS GILERIKKIL PKLNGKIIGN MQPDSTITAS MLHNTGKDWH PISENAHYFT | 480 |
| KWIYTLTLFM DGKEINDLVT TLINKFDNIA SFIEVLKSQS VCTHFSEERK MFIDSAEICS | 540 |
| ELSAMNSFAR MEAPGASSKR AMFVEAARIL GDNRSKEELE EYFDTLFDKS ASKKEKGFRN | 600 |
| FIRNNVVDSN RFKYLTRYTD TSSVKAFSNN KALVKFAIKD IPQEQILRYY NSCFGASERY | 660 |
| YNDGMSDKLV EAIGKINLMQ FNGVIQQADR NMLPEEKKKA NAQKEKYKSI IRLYLTVCYL | 720 |
| FFKNLVYVNS RYYSAFYNLE KDRSLFEING ELKPTGKFDE GHYTGLVKLF IDNGWINPRA | 780 |
| SAYLTVNLAN SDETAIRTFR NTAEHLEALR NADKYLNDLK QFDSYFEIYH YITQRNIKEK | 840 |
| CEMLKEQTVK YNNDLLKYHG YSKDFVKALC VPFGYNLPRF KNLSIDALFD KNDKREKLKK | 900 |
| GFED. | 904 |

Exemplary CasRX/Cas13d proteins may comprise or consist of the sequence: CasRX/Cas13d Metagenomic hit (no protein accession): contig emb|OIEN01002196.1:

| (SEQ ID NO: 106) | |
|---|---|
| MERQKRKMKS KSKMAGVKSV FVIGDELLMT SFGDGDDAVL EKDIDENGVV NDCRNPAAYD | 60 |
| AVYGTDSIRV KKTNNNIRAK VNNPLAKSNI RSEESALFRT RVNEYKREQK DKYETLFFGK | 120 |
| TFDDNIHIQL ISKILDIEKT FSVVIGNIVY AINNLSLEQS IDRPIDIFGD KNTQGISLRE | 180 |
| DNDYLKTMLP RCEYLFHNIL NSDSDNNSKM NYNKVNKGKE EKDNRNNENI EKLKKALEVI | 240 |
| KIIRVDSFHG VDGIKGDQKF PRSKYNLAMN YNEEIQKTIS EPFNRKVEEV QQDFYRNSCV | 300 |
| NIDFLKEIMY GSNYTDRGSD SLECSYFNFA ILKQNKNMGF SITSIRECLL DLYELNFESM | 360 |
| QNLRPRANSF CDFLIYDYYC KNESERANLV DCLRSAASEE EKKNIYFQTA ERVKEKFRNA | 420 |
| FNRISRFDAS YIKNSREKNL SGGSSLPKYS FIEGFTKRSK KINDNDEKNA DLFCNMLYYL | 480 |
| AQFLDGKEIN IFLTSIHNIF QNIDSFLKVM KEKGMECKFQ KDFKMFSHAG HVAKKIEIVI | 540 |
| SLAKMKKTLD FYNAQALKDA VTILGVSKKH QYLDMNSYLD FYMFDNRSGA TGKNAGKDHN | 600 |
| LRNFLVSNVI RSRKFNYLSR YSNLAEVKKL AQNPSLVQFV LSRIEPSLIC RYYESSQGIS | 660 |
| SEGITIDEQI KKLTGIIVDM NIDSFENINN GEIGMRYSKA TPQSIERRNQ MRVCVGLYLN | 720 |
| VLYQIEKNLM NVNARYVLAF AFAERDALML NFTLEECKKN KKRSSGGFSF IEMTQFFIDK | 780 |
| KLFKVATEAI KKNVLKYNGN PESLNHIPGE YICKNMEGYH ENTVRNFRNM VAHLTAVARV | 840 |
| PLYISEVTQI DSYYALYHYC MQMNILQGIE QSGKILDNIK LKNALENARV HRTYSKDAVK | 900 |
| YLCLPFAYNI SRYKALTIKD LFDWTEYSCK KDE. | 933 |

Exemplary CasRX/Cas13d proteins may comprise or consist of the sequence: CasRX/Cas13d Metagenomic hit (no protein accession): contig e-k87_11092736:

| (SEQ ID NO: 107) | |
|---|---|
| MKRQKTFAKR IGIKSTVAYG QGKYAITTFG KGSKAEIAVR SADPPEETLP TESDATLSIH | 60 |
| AKFAKAGRDG REFKCGDVDE TRIHTSRSEY ESLISNPAES PREDYLGLKG TLERKFFGDE | 120 |
| YPKDNLRIQI IYSILDIQKI LGLYVEDILH FVDGLQDEPE DLVGLGLGDE KMQKLLSKAL | 180 |
| PYMGFFGSTD VFKVTKKREE RAAADEHNAK VFRALGAIRQ KLAHFKWKES LAIFGANANM | 240 |
| PIRFFQGATG GRQLWNDVIA PLWKKRIERV RKSFLSNSAK NLWVLYQVFK DDTDEKKKAR | 300 |
| ARQYYHFSVL KEGKNLGFNL TKTREYFLDK FFPIFHSSAP DVKRKVDTFR SKFYAILDFI | 360 |
| IYEASVSVAN SGQMGKVAPW KGAIDNALVK LREAPDEEAK EKIYNVLAAS IRNDSLFLRL | 420 |
| KSACDKFGAE QNRPVFPNEL RNNRDIRNVR SEWLEATQDV DAAAFVQLIA FLCNFLEGKE | 480 |
| INELVTALIK KFEGIQALID LLRNLEGVDS IRFENEFALF NDDKGNMAGR IARQLRLLAS | 540 |
| VGKMKPDMTD AKRVLYKSAL EILGAPPDEV SDEWLAENIL LDKSNNDYQK AKKTVNPFRN | 600 |
| YIAKNVITSR SFYYLVRYAK PTAVRKLMSN PKIVRYVLKR LPEKQVASYY SAIWTQSESN | 660 |
| SNEMVKLIEM IDRLTTEIAG FSFAVLKDKK DSIVSASRES RAVNLEVERL KKLTTLYMSI | 720 |
| AYIAVKSLVK VNARYFIAYS ALERDLYFFN EKYGEEFRLH FIPYELNGKT CQFEYLAILK | 780 |
| YYLARDEETL KRKCEICEEI KVGCEKHKKN ANPPYEYDQE WIDKKKALNS ERKACERRLH | 840 |
| FSTHWAQYAT KRDENMAKHP QKWYDILASH YDELLALQAT GWLATQARND AEHLNPVNEF | 900 |
| DVYIEDLRRY PEGTPKNKDY HIGSYFEIYH YIRQRAYLEE VLAKRKEYRD SGSFTDEQLD | 960 |
| KLQKILDDIR ARGSYDKNLL KLEYLPFAYN LPRYKNLTTE ALFDDDSVSG KKRVAEWRER | 1020 |
| EKTREAEREQ RRQR. | 1034 |

An exemplary direct repeat sequence of CasRX/Cas13d Metagenomic hit (no protein accession): contig e-k87_11092736 (SEQ ID NO: 107) comprises or consists of the nucleic acid sequence: CasRX/Cas13d Direct repeat 1: gtgagaagtc tccttatggg gagatgctac (SEQ ID NO: 108).

Exemplary CasRX/Cas13d proteins may comprise or consist of the sequence: CasRX/Cas13d Ga0129306_1000735:

| (SEQ ID NO: 109) | |
|---|---|
| MQKQREQQTV TDESERKKKP LKSGAKAAGL KSVFVLSEGK ELLTSFGRGN EAVPEKRVTG | 60 |
| GTIANARTDN KEAFSAALQN KRFEVFGRTA GSSDDPLAVS RAPGQDLIGA KTALEERYFG | 120 |
| RAFADNIHMQ VIYAIQDINK ILAVHANNIV YTLNNLDREA DPETDDFIGS GYLTLKNTFE | 180 |
| TYCDPAALNE REREKVTVSK QHFDAFMQNP RLAYYGNAFF RKLSKAERLA RGREIFDKES | 240 |
| PERRQEILGS RGKNKSVDDE IRALAPEWVK REERDVYSEL VLMSELRQSC FHGQQKNSAR | 300 |
| IFRLDNDLGP GVDGARELLD RLYAEKINDL RSFDKTSASS NFRLLFNAYH ADNEKKKELA | 360 |
| QEFYRFSVLK VSKNTGFSIR TLREKIIEDH AAQYRDKIYD SMRKKLFSTF DFFLWRFYEE | 420 |
| REDEAEELRA CLRAARSDEE KEQIYAEAAA SCWPSVKPFV ESVAATLCDV VKGRTKLNKL | 480 |
| KLSADESTLV RNAIDGVRIS PRASYFTKLI YLMTLFLDGK EINDLLTTLI HAFENIDSFL | 540 |
| SVLGSERLER TFDANYRIFA DSGVIAQELR AVNSFARMTT EPFNSKLVMF EDAAQLFGMS | 600 |
| GGLVEHAEEL REYLDNKMLD KTKLRLLPDG KVDTGFRNFI ISNVTESRRF RYLVRYCEPR | 660 |
| AVRDYMSCRP LIRLTLRDMP DTILRRYYEQ SVGAATVDRE RILDTLADKL LSLRFTDFEN | 720 |
| VNQRANAERN REKQKMMGII SLYLNVAYQI VKNLVYVNAR YTMAYHCAER DTELLLNAAG | 780 |
| EGNLLRRDRS WPARLHLPRR ALARRRDRVE VMERDVARGP EAYNRDEWLG LVRTLRREKR | 840 |
| VCDNLHNNYA YLCGADAEPG DASLSLLFVY RNKAAHLSVL NKGGRLSGDL KEAKSWFYVY | 900 |
| HFLMQRVLEE EFRNTQALPE RLRELLMMAE RYRGCSKDLI KVLNLTFAYN LPRYKNLSID | 960 |
| GRFDKNHPDP SDE. | 973 |

Exemplary CasRX/Cas13d proteins may comprise or consist of the sequence: CasRX/Cas13d Ga0129317_1008067:

| (SEQ ID NO: 110) | |
|---|---|
| MKKQKKSLVK AAGLKSAFVV GDSVYLTSFG KGNAARLDTK INPDNSTERY VSDSEKHTLK | 60 |
| INSITDTELR LSGPFPKQAE AKNPTHKKDN EQKNTRQDML GLKSTLEKFY FGSTFDDNIH | 120 |
| IQIIHNIQDI AKILAAHSNN AGYALDNMLA YQGVEFSDMI GYMGTSRTFD NYDPNHKNNK | 180 |
| DFFRFLKLPR LGYFGSAFYS QKGKDFEKRS DEEVYNICAL MGQIRQCCFH GKQEKYQLKW | 240 |
| LYNFHNFKSN KPFLDTLDKH FDEMIDRINK NFIKNNTPDL IILSGLYPDM AKKELVRLFY | 300 |
| DFTTVKEYKN MGFSVKKLRE KMLESEEASD FRDKDYDSVR RKLYKLMDFC IYYLYYSDSE | 360 |
| RNENLVSRLR ESLTDENKDI IYSKEAKIVW NELRKKFSTI LDNVKGSNIK KLENVKEKFI | 420 |
| SEDEFDDIKL DIDISYFSKL MYVMCYFLDG KEINDLLTTL VSKFDNIGSI IEAATQIGIN | 480 |
| IEFIDDFKFF DRSKDISVEL NIIRNFARMQ APVPNAKRAM QEDAIRILGG SEEDIFSILD | 540 |
| DMTGYDKSGK KLAQSKKGFR NFIINNVVES SRFKYIVRYS NPQKIRKLAN NSVVVGFVLG | 600 |
| KLPDAQIESY FNSCLPNRVY STPDKARESL RDMLHNISFN DFADVKQDDR RATPEEKVEK | 660 |
| ERYKAIIGLY LTVMYHLVKN LVYVNSRYVM AFHCLERDAM HYDVSLDNYR DLIRHLISEG | 720 |
| DSSCNHFISH NRRMRDCIEE NVKNSEQLIF GKEDAVIRFR NNVAHLSAIR NANEYIGDIR | 780 |
| EITSYFALYH YLMQRKLIDD CKVNDTAHKY FEQLTKYKTY VMDMVKALCS PFGYNLPRFK | 840 |
| NLSIEGKFDM HESK. | 854 |

Exemplary CasRX/Cas13d proteins may comprise or consist of the sequence: CasRX/Cas13d Ga0224415_10048792:

| (SEQ ID NO: 111) | |
|---|---|
| MSKKENRKSY VKGLGLKSTL VSDSKVYLTT FADGSNAKLE KCVENNKIIC ISNDKEAFAA | 60 |
| SIANKNVGYK IKNDEKFRHP KGYDIISNNP LLHNNSVQQD MLGLKNVLEK RYFGKSSGGD | 120 |
| NNLCIQIIHN IIDIEKILSE YIPNVVYAFN NIAGFKDEHN NIIDIIGTQT YNSSYTYADF | 180 |
| SKDKSDKKYI EFQKLLKNKR LGYWGKAFFT GQGNNAKVRQ ENQCFHIIAL LISLRNWATH | 240 |
| SNELDKHTKR TWLYKLDDTN ILNAEYVKTL NYLYDTIADE LTKSFSKNGA VNVNYLAKKY | 300 |
| NIKDDLPGFS EQYFRFSIMK EQKNLGFNIS KLRENMLDFK DMSVIRDDHN RYDKDRSKIY | 360 |
| TMMDFVIYRY YIDNNNDSID FINKLRSSID EKSKEKLYNE EANRLWNKLK EYMLYIKEFN | 420 |
| GKLASRTPDR DGNISEFVES LPKIHRLLPR GQKISNFSKL MYLLTMFLDG KEINDLLTTL | 480 |
| INKFENIQGF LDIMPEINVN AKFEPEYVFF NKSHEIAGEL KLIKGFAQMG EPAATLKLEM | 540 |
| TADAIKILGT EKEDAELIKL AESLFKDENG KLLGNKQHGM RNFIGNNVIK SKRFHYLIRY | 600 |
| GDPAHLHKIA TNKNVVRFVL GRIADMQKKQ GQKGKNQIDR YYEVCVGNKD IKKTIEEKID | 660 |
| ALTDIIVNMN YDQFEKKKAV IENQNRGKTF EEKNKYKRDN AEREKFKKII SLYLTVIYHI | 720 |
| LKNIVNVNSR YILGFHCLER DKQLYIEKYN KDKLDGFVAL TKFCLGDEER FEDLKAKAQA | 780 |
| SIQALETANP KLYAKYMNYS DEEKKEEFKK QLNRERVKNA RNAYLKNIKN YIMIRLQLRD | 840 |
| QTDSSGYLCG EFRDKVAHLE VARHAHEYIG NIKEVNSYFQ LYHYIMQCRL YDVLKNNTKA | 900 |
| EAMVKGKAKE YFEALEKEGT YNDKLLKIAC VPFGYCIPRY KNLSMEELFD MNEEKKFKKK | 960 |
| APENT. | 965 |

Exemplary CasRX/Cas13d proteins may comprise or consist of the sequence CasRX/Cas13d 160582958_gene49834:

| (SEQ ID NO: 112) | |
|---|---|
| MKNSVTFKLI QAQENKEAAR KKAKDIAEQA RIAKRNGVVK KEENRINRIQ IEIQTQKKSN | 60 |
| TQNAYHLKSL AKAAGVKSVF AIGNDLLMTG FGPGNDATIE KRVFQNRAIE TLSSPEQYSA | 120 |
| EFQNKQFKIK GNIKVLNHST QKMEEIQTEL QDNYNRPHFD LLGCKNVLEQ KYFGRTFSDN | 180 |
| IHVQIAYNIM DIEKLLTPYI NNIIYTLNEL MRDNSKDDFF GCDSHFSVAY LYDELKAGYS | 240 |
| DRLKTKPNLS KNIDRIWNNF CNYMNSDSGN TEARLAYFGE LFYKPKETGD AKSDYKTHLS | 300 |
| NNQKEEWELK SDKEVYNIFA ILCDLRHFCT HGESITPSGK PFPYNLEKNL FPEAKQVLNS | 360 |
| LFEEKAESLG AEAFGKTAGK TDVSILLKVF EKEQASQKEQ QALLKEYYDF KVQKTYKNMG | 420 |
| FSIKKLREAI MEIPDAAKFK DDLYSSLRHK LYGLFDFILV KHFLDTSDSE NLQNNDIFRQ | 480 |
| LRACRCEEEK DQVYRSIAVK VWEKVKKKEL NMFKQVVVIP SLSKDELKQM EMTKNTELLS | 540 |
| SIETISTQAS LFSEMIFMMT YLLDGKEINL LCTSLIEKFE NIASFNEVLK SPQIGYETKY | 600 |
| TEGYAFFKNA DKTAKELRQV NNMARMTKPL GGVNTKCVMY NEAAKILGAK PMSKAELESV | 660 |
| FNLDNHDYTY SPSGKKIPNK NFRNFIINNV ITSRRFLYLI RYGNPEKIRK IAINPSIISF | 720 |
| VLKQIPDEQI KRYYPPCIGK RTDDVTLMRD ELGKMLQSVN FEQFSRVNNK QNAKQNPNGE | 780 |
| KARLQACVRL YLTVPYLFIK NMVNINARYV LAFHCLERDH ALCFNSRKLN DDSYNEMANK | 840 |
| FQMVRKAKKE QYEKEYKCKK QETGTAHTKK IEKLNQQIAY IDKDIKNMHS YTCRNYRNLV | 900 |
| AHLNVVSKLQ NYVSELPNDY QITSYFSFYH YCMQLGLMEK VSSKNIPLVE SLKNEANDAQ | 960 |
| SYSAKKTLEY FDLIEKNRTY CKDFLKALNA PFSYNLPRFK NLSIEALFDK NIVYEQADLK | 1020 |
| KE. | 1022 |

An exemplary direct repeat sequence of CasRX/Cas13d proteins may comprise or consist of the sequence CasRX/Cas13d 160582958_gene49834 (SEQ ID NO: 112) comprises or consists of the

| nucleic acid sequence: CasRX/Cas13d DR: | |
|---|---|
| (SEQ ID NO: 113) | |
| gaactacacc cctctgttct tgtaggggtc taacac. | 36 |

Exemplary CasRX/Cas13d proteins may comprise or consist of the sequence: CasRX/Cas13d 250twins_35838_GL0110300:

| (SEQ ID NO: 114) | |
|---|---|
| MGNKQRVSAQ KRRENAKLCN QQKARQAESQ RDKIKNMNVE KMKNINTNDI KHTKTTAKKL | 60 |
| GLKSTIIADK KIILTSFINE QSSKTANIEK VAGFKGDTID TISYTPRMFR SEINPGEIVI | 120 |
| SKGDDLSEFA NPANFPIGRD YVKIRSALEK QYFGKEFPED NLHVQIAYNV ADIKKILSVY | 180 |
| INNIIYMFYN LARSEEYDIF YNSQSENSGR DCDVIGSLYY QASYRNQDAN RFEKDGKKKA | 240 |
| IDSLLDDTRA YYTYFDGLFS VPKREDDGKI KESEKEKAKD QNFDVLRLLS VGRQLTFHSD | 300 |
| KSNNEAYLFD LSKLTRAAQD ENRRQDIQSL LNILNSTCRS NLEGVNGDFV KHAKNNLYVL | 360 |
| NQLYPSLKAN DLIGEYYNFI VKKENRNIGI RLITVRELII EHNYTNLKDS KYDTYRNKIY | 420 |
| TVLNFILFRE IQENSIAIKN FREKLRSTEK AEQPALYQAF ANKIYPMVQA KFAKAIDLFE | 480 |
| EQYKTKFKSE FKGGISIENM QQQNILLQTE NIDYFSKYVL FLTKFLDGKE INELLCALIN | 540 |
| KFDNIADLLD ISKQIGTPVV FCADYESLND AAKIAENIRL IKNIAHLRPA IQEAQSSKDN | 600 |
| ADAAGTPATL LIDAYNMLNT DIQLVYGEAA YEELRKDLFE RKNGTKYNKK GKKVDVYDHK | 660 |
| FRNFLINNVI KSKWFFYIAK YVKPADCAKM MSNKKMIEFA LRDLPETQIK RYYYTITGNE | 720 |
| ALGDAESLKG VIIEQLHAFS IKNTLLSIKN MGEGEYKIQQ IGSSKEKLKA IVNLYLTVAY | 780 |
| LLTKSLVKVN IRFSIAFGCL ERDLVLQKKS EKKFDAIINE ILLEDDKIRK ECDKERAQAK | 840 |
| TLPRELAQER FAQIKRRESG CYFKSYHVYD YLSKNSNEFK QNHIDFAVTS YRNNVEHLNV | 900 |
| VHCMTKYFSE VKDVKSYYGV YCYIMQRMLC DELIIKNQDK PDVRQTFEEY NRLLKDHGTY | 960 |
| SKNLMWLLNF PFAYNLARYK NLSNEDLFNA KNNDQKSK. | 998 |

Exemplary CasRX/Cas13d proteins may comprise or consist of the sequence: CasRX/Cas13d 250twins_36050_GL0158985:

| (SEQ ID NO: 115) | |
|---|---|
| MKKKHQSAAE KRQVKKLKNQ EKAQKYASEP SPLQSDTAGV ECSQKKTVVS HIASSKTLAK | 60 |
| AMGLKSTLVM GDKLVITSFA ASKAVGGAGY KSANIEKITD LQGRVIEEHE RMFSADVGEK | 120 |
| NIELSKNDCH TNVNNPVVTN IGKDYIGLKS RLEQEFFGKT FENDNLHVQL AYNILDIKKI | 180 |
| LGTYVNNIIY IFYNLNRAGT GRDERMYDDL IGTLYAYKPM EAQQTYLLKG DKDMRRFEEV | 240 |
| KQLLQNTSAY YVYYGTLFEK VKAKSKKEQR AKEAEIDACT AHNYDVLRLL SLMRQLCMHS | 300 |
| VAGTAFKLAE SALFNIEDVL SADLKEILDE AFSGAVNKLN DGFVQHSGNN LYVLQQLYPN | 360 |
| ETIERIAEKY YRLTVRKEDL NMGVNIKKLR ELIVGQYFPE VLDKEYDLSK NGDSVVTYRS | 420 |
| KIYTVMNYIL LYYLEDHDSS RESMVEALRQ NREGDEGKEE IYRQFAKKVW NGVSGLFGVC | 480 |
| LNLFKTEKRN KFRSKVALPD VSGAAYMLSS ENIDYFVKML FFVCKFLDGK EINELLCALI | 540 |
| NKFDNIADIL DAAAQCGSSV WFVDSYRFFE RSRRISAQIR IVKNIASKDF KKSKKDSDES | 600 |
| YPEQLYLDAL ALLGDVISKY KQNRDGSVVI DDQGNAVLTE QYKRFRYEFF EEIKRDESGG | 660 |
| IKYKKSGKPE YNHQRRNFIL NNVLKSKWFF YVVKYNRPSS CRELMKNKEI LRFVLRDIPD | 720 |
| SQVRRYFKAV QGEEAYASAE AMRTRLVDAL SQFSVTACLD EVGGMTDKEF ASQRAVDSKE | 780 |
| KLRAIIRLYL TVAYLITKSM VKVNTRFSIA FSVLERDYYL LIDGKKKSSD YTGEDMLALT | 840 |
| RKFVGEDAGL YREWKEKNAE AKDKYFDKAE RKKVLRQNDK MIRKMHFTPH SLNYVQKNLE | 900 |
| SVQSNGLAAV IKEYRNAVAH LNIINRLDEY IGSARADSYY SLYCYCLQMY LSKNFSVGYL | 960 |
| INVQKQLEEH HTYMKDLMWL LNIPFAYNLA RYKNLSNEKL FYDEEAAAEK ADKAENERGE. | 1020 |

Yan et al. (2018) Mol Cell. 70(2):327-339 (doi: 10.1016/j.molcel.2018.02.2018) and Konermann et al. (2018) Cell 173(3):665-676 (doi: 10.1016/j.cell/2018.02.033) have described CasRX/Cas13d proteins and both of which are incorporated by reference herein in their entireties. Also see WO Publication Nos. WO2018/183703 (CasM) and WO2019/006471 (Cas13d), which are incorporated herein by reference in their entirety.

Exemplary wild type Cas13d proteins of the disclosure may comprise or consist of the amino acid sequence:

Cas13d (Ruminococcus flavefaciens XPD3002) sequence:

(SEQ ID NO: 45)

| | |
|---|---|
| 1 | IEKKKSFAKG MGVKSTLVSG SKVYMTTFAE GSDARLEKIV |
| | EGDSIRSVNE GEAFSAEMAD |
| 61 | KNAGYKIGNA KFSHPKGYAV VANNPLYTGP VQQDMLGLKE |
| | TLEKRYFGES ADGNDNICIQ |
| 121 | VIHNILDIEK ILAEYITNAA YAVNNISGLD KDIIGFGKFS |
| | TVYTYDEFKD PEHHRAAFNN |
| 181 | NDKLINAIKA QYDEFDNFLD NPRLGYFGQA FFSKEGRNYI |
| | INYGNECYDI LALLSGLAHW |
| 241 | VVANNEEESR ISRTWLYNLD KNLDNEYIST LNYLYDRITN |
| | ELTNSFSKNS AANVNYIAET |
| 301 | LGINPAEFAE QYFRFSIMKE QKNLGFNITK LREVMLDRKD |
| | MSEIRKNHKV FDSIRTKVYT |
| 361 | MMDFVIYRYY IEEDAKVAAA NKSLPDNEKS LSEKDIFVIN |
| | LRGSFNDDQK DALYYDEANR |
| 421 | IWRKLENIMH NIKEFRGNKT REYKKKDAPR LPRILPAGRD |
| | VSAFSKLMYA LTMFLDGKEI |
| 481 | NDLLTTLINK FDNIQSFLKV MPLIGVNAKF VEEYAFFKDS |
| | AKIADELRLI KSFARMGEPI |
| 541 | ADARRAMYID AIRILGTNLS YDELKALADT FSLDENGNKL |
| | KKGKHGMRNF IINNVISNKR |
| 601 | FHYLIRYGDP AHLHEIAKNE AVVKFVLGRI ADIQKKQGQN |
| | GKNQIDRYYE TCIGKDKGKS |
| 661 | VSEKVDALTK IITGMNYDQF DKKRSVIEDT GRENAEREKF |
| | KKIISLYLTV IYHILKNIVN |
| 721 | INARYVIGFH CVERDAQLYK EKGYDINLKK LEEKGFSSVT |
| | KLCAGIDETA PDKRKDVEKE |
| 781 | MAERAKESID SLESANPKLY ANYIKYSDEK KAEEFTRQIN |
| | REKAKTALNA YLRNTKWNVI |
| 841 | IREDLLRIDN KTCTLFANKA VALEVARYVH AYINDIAEVN |
| | SYFQLYHYIM QRIIMNERYE |
| 901 | KSSGKVSEYF DAVNDEKKYN DRLLKLLCVP FGYCIPRFKN |
| | LSIEALFDRN EAAKFDKEKK |
| 961 | SGNS. |

Exemplary wild type Cas13d proteins of the disclosure may comprise or consist of the amino acid sequence:

Cas13d (contig e-k87_11092736):

(SEQ ID NO: 46)
MKRQKTFAKRIGIKSTVAYGQGKYAITTFGKGSKAEIAVRSADPPEETLP
TESDATLSIHAKFAKAGRDGREFKCGDVDETRIHTSRSEYESLISNPAES
PREDYLGLKGTLERKFFGDEYPKDNLRIQIIYSILDIQKILGLYVEDILH
FVDGLQDEPEDLVGLGLGDEKMQKLLSKALPYMGFFGSTDVFKVTKKREE
RAAADEHNAKVFRALGAIRQKLAHFKWKESLAIFGANANMPIRFFQGATG
GRQLWNDVIAPLWKKRIERVRKSFLSNSAKNLWVLYQVFKDDTDEKKKAR
ARQYYHFSVLKEGKNLGFNLTKTREYFLDKFFPIFHSSAPDVKRKVDTFR
SKFYAILDFIIYEASVSVANSGQMGKVAPWKGAIDNALVKLREAPDEEAK
EKIYNVLAASIRNDSLFLRLKSACDKFGAEQNRPVFPNELRNNRDIRNVR
SEWLEATQDVDAAAFVQLIAFLCNFLEGKEINELVTALIKKFEGIQALID
LLRNLEGVDSIRFENEFALFNDDKGNMAGRIARQLRLLASVGKMKPDMTD
AKRVLYKSALEILGAPPDEVSDEWLAENILLDKSNNDYQKAKKTVNPFRN
YIAKNVITSRSFYYLVRYAKPTAVRKLMSNPKIVRYVLKRLPEKQVASYY
SAIWTQSESNSNEMVKLIEMIDRLTTEIAGFSFAVLKDKKDSIVSASRES
RAVNLEVERLKKLTTLYMSIAYIAVKSLVKVNARYFIAYSALERDLYFFN
EKYGEEFRLHFIPYELNGKTCQFEYLAILKYYLARDEETLKRKCEICEEI
KVGCEKHKKNANPPYEYDQEWIDKKKALNSERKACERRLHFSTHWAQYAT
KRDENMAKHPQKWYDILASHYDELLALQATGWLATQARNDAEHLNPVNEF
DVYIEDLRRYPEGTPKNKDYHIGSYFEIYHYIRQRAYLEEVLAKRKEYRD
SGSFTDEQLDKLQKILDDIRARGSYDKNLLKLEYLPFAYNLPRYKNLTTE
ALFDDDSVSGKKRVAEWREREKTREAEREQRRQR.

An exemplary direct repeat sequence of Cas13d (contig e-k87_11092736) (SEQ ID NO: 46) comprises or consists of the nucleic acid sequence: Cas13d (contig e-k87_11092736)

Direct Repeat Sequence):
(SEQ ID NO: 47)
GTGAGAAGTCTCCTTATGGGGAGATGCTAC.

Exemplary wild type Cas13d proteins of the disclosure may comprise or consist of the amino acid sequence:

Cas13d (160582958_gene49834):

(SEQ ID NO: 48)
MKNSVTFKLIQAQENKEAARKKAKDIAEQARIAKRNGVVKKEENRINRIQ
IEIQTQKKSNTQNAYHLKSLAKAAGVKSVFAIGNDLLMTGFGPGNDATIE
KRVFQNRAIETLSSPEQYSAEFQNKQFKIKGNIKVLNHSTQKMEEIQTEL
QDNYNRPHFDLLGCKNVLEQKYFGRTFSDNIHVQIAYNIMDIEKLLTPYI
NNIIYTLNELMRDNSKDDFFGCDSHFSVAYLYDELKAGYSDRLKTKPNLS
KNIDRIWNNFCNYMNSDSGNTEARLAYFGELFYKPKETGDAKSDYKTHLS
NNQKEEWELKSDKEVYNIFAILCDLRHFCTHGESITPSGKPFPYNLEKNL
FPEAKQVLNSLFEEKAESLGAEAFGKTAGKTDVSILLKVFEKEQASQKEQ
QALLKEYYDFKVQKTYKNMGFSIKKLREAIMEIPDAAKFKDDLYSSLRHK
LYGLFDFILVKHFLDTSDSENLQNNDIFRQLRACRCEEEKDQVYRSIAVK
VWEKVKKKELNMFKQVVVIPSLSKDELKQMEMTKNTELLSSIETISTQAS
LFSEMIFMMTYLLDGKEINLLCTSLIEKFENIASFNEVLKSPQIGYETKY
TEGYAFFKNADKTAKELRQVNNMARMTKPLGGVNTKCVMYNEAAKILGAK
PMSKAELESVFNLDNHDYTYSPSGKKIPNKNFRNFIINNVITSRRFLYLI
RYGNPEKIRKIAINPSIISFVLKQIPDEQIKRYYPPCIGKRTDDVTLMRD
ELGKMLQSVNFEQFSRVNNKQNAKQNPNGEKARLQACVRLYLTVPYLFIK
NMVNINARYVLAFHCLERDHALCFNSRKLNDDSYNEMANKFQMVRKAKKE
QYEKEYKCKKQETGTAHTKKIEKLNQQIAYIDKDIKNMHSYTCRNYRNLV
AHLNVVSKLQNYVSELPNDYQITSYFSFYHYCMQLGLMEKVSSKNIPLVE
SLKNEANDAQSYSAKKTLEYFDLIEKNRTYCKDFLKALNAPFSYNLPRFK
NLSIEALFDKNIVYEQADLKKE.

An exemplary direct repeat sequence of Cas13d (160582958_gene49834) (SEQ ID NO: 48) comprises or consists of the nucleic acid sequence:

Cas13d (160582958_gene49834) Direct Repeat Sequence:

(SEQ ID NO: 49)
GAACTACACCCCTCTGTTCTTGTAGGGGTCTAACAC.

Exemplary wild type Cas13d proteins of the disclosure may comprise or consist of the amino acid sequence:

Cas13d (contig tpg|DJXD01000002.1|; uncultivated Ruminococcus assembly, UBA7013, from sheep gut metagenome):

(SEQ ID NO: 50)
MKKQKSKKTVSKTSGLKEALSVQGTVIMTSFGKGNMANLSYKIPSSQKPQ
NLNSSAGLKNVEVSGKKIKFQGRHPKIATTDNPLFKPQPGMDLLCLKDKL
EMHYFGKTFDDNIHIQLIYQILDIEKILAVHVNNIVFTLDNVLHPQKEEL
TEDFIGAGGWRINLDYQTLRGQTNKYDRFKNYIKRKELLYFGEAFYHENE
RRYEEDIFAILTLLSALRQFCFHSDLSSDESDHVNSFWLYQLEDQLSDEF
KETLSILWEEVTERIDSEFLKTNTVNLHILCHVFPKESKETIVRAYYEFL
IKKSFKNMGFSIKKLREIMLEQSDLKSFKEDKYNSVRAKLYKLFDFIITY
YYDHHAFEKEALVSSLRSSLTEENKEEIYIKTARTLASALGADFKKAAAD
VNAKNIRDYQKKANDYRISFEDIKIGNTGIGYFSELIYMLTLLLDGKEIN
DLLTTLINKFDNIISFIDILKKLNLEFKFKPEYADFFNMTNCRYTLEELR
VINSIARMQKPSADARKIMYRDALRILGMDNRPDEEIDRELERTMPVGAD
GKFIKGKQGFRNFIASNVIESSRFHYLVRYNNPHKTRTLVKNPNVVKFVL
EGIPETQIKRYFDVCKGQEIPPTSDKSAQIDVLARIISSVDYKIFEDVPQ
SAKINKDDPSRNFSDALKKQRYQAIVSLYLTVMYLITKNLVYVNSRYVIA
FHCLERDAFLHGVTLPKMNKKIVYSQLTTHLLTDKNYTTYGHLKNQKGHR
KWYVLVKNNLQNSDITAVSSFRNIVAHISVVRNSNEYISGIGELHSYFEL
YHYLVQSMIAKNNWYDTSHQPKTAEYLNNLKKHHTYCKDFVKAYCIPFGY
VVPRYKNLTINELFDRNNPNPEPKEEV.

An exemplary direct repeat sequence of Cas13d (contig tpg|DJXD01000002.1|; uncultivated Ruminococcus assembly, UBA7013, from sheep gut metagenome) (SEQ ID NO: 50) comprises or consists of the nucleic acid sequence: Cas13d (contig tpg|DJXD01000002.1|; uncultivated Ruminococcus assembly, UBA7013, from sheep gut metagenome) Direct Repeat Sequence:

(SEQ ID NO: 51)
CAACTACAACCCCGTAAAAATACGGGGTTCTGAAAC.

In some embodiments of the compositions of the disclosure, the sequence comprising the gRNA further comprises a spacer sequence that specifically binds to the target RNA sequence. In some embodiments, the spacer sequence has at least 50%, 55%, 60%, 65%, 70%, 75%, 80%, 87%, 90%, 95%, 97%, 99% or any percentage in between of complementarity to the target RNA sequence. In some embodiments, the spacer sequence has 100% complementarity to the target RNA sequence. In some embodiments, the spacer sequence comprises or consists of 20 nucleotides. In some embodiments, the spacer sequence comprises or consists of 21 nucleotides. In some embodiments, the spacer sequence comprises or consists of the sequence UGGAGCGAGCAUCCCCCAAA (SEQ ID NO: 1), GUUUGGGGGAUGCUCGCUCCA (SEQ ID NO: 2), CCCUCACUGCUGGGGAGUCC (SEQ ID NO: 3), GGACUCCCCAGCAGUGAGGG (SEQ ID NO: 4), GCAACUGGAUCAAUUUGCUG (SEQ ID NO: 5), GCAGCAAAUUGAUCCAGUUGC (SEQ ID NO: 6), GCAUUCUUAUCUGGUCAGUGC (SEQ ID NO: 7), GCACUGACCAGAUAAGAAUG (SEQ ID NO: 8), GAGCAGCAGCAGCAGCAGCAG (SEQ ID NO: 9), GCAGGCAGGCAGGCAGGCAGG (SEQ ID NO: 10), GCCCCGGCCCCGGCCCCGGC (SEQ ID NO: 11), or GCTGCTGCTGCTGCTGCTGC (SEQ ID NO: 12), GGGGCCGGGGCCGGGGCCGG (SEQ ID NO: 74), GGGCCGGGGCCGGGGCCGGG (SEQ ID NO: 75), GGCCGGGGCCGGGGCCGGGG (SEQ ID NO: 76), GCCGGGGCCGGGGCCGGGGC (SEQ ID NO: 77), CCGGGGCCGGGGCCGGGGCC (SEQ ID NO: 78), or CGGGGCCGGGGCCGGGGCCG (SEQ ID NO: 79).

In some embodiments of the compositions of the disclosure, the sequence comprising the gRNA further comprises a spacer sequence that specifically binds to the target RNA sequence. In some embodiments, the spacer sequence has at least 50%, 55%, 60%, 65%, 70%, 75%, 80%, 87%, 90%, 95%, 97%, 99% or any percentage in between of complementarity to the target RNA sequence.

In some embodiments, the spacer sequence has 100% complementarity to the target RNA sequence. In some embodiments, the spacer sequence comprises or consists of 20 nucleotides. In some embodiments, the spacer sequence comprises or consists of 21 nucleotides. In some embodiments, the spacer sequence comprises or consists of the sequence GUGAUAAGUGGAAUGCCAUG (SEQ ID NO: 14), CUGGUGAACUUCCGAUAGUG (SEQ ID NO: 15), or GAGATATAGCCTGGTGGTTC (SEQ ID NO: 16).

In some embodiments of the compositions of the disclosure, the sequence comprising the gRNA further comprises a spacer sequence that specifically binds to the target RNA sequence. In some embodiments, the spacer sequence has at least 50%, 55%, 60%, 65%, 70%, 75%, 80%, 87%, 90%, 95%, 97%, 99% or any percentage in between of complementarity to the target RNA sequence. In some embodiments, the spacer sequence has 100% complementarity to the target RNA sequence. In some embodiments, the spacer sequence comprises or consists of 20 nucleotides. In some embodiments, the spacer sequence comprises or consists of 21 nucleotides. In some embodiments, the spacer sequence comprises or consists of a sequence comprising at least 1, 2, 3, 4, 5, 6, or 7 repeats of the sequence CUG (SEQ ID NO: 18), CCUG (SEQ ID NO: 19), CAG (SEQ ID NO: 80), GGGGCC (SEQ ID NO: 81) or any combination thereof.

In some embodiments of the compositions of the disclosure, the sequence comprising the gRNA further comprises a scaffold sequence that specifically binds to the first RNA binding protein. In some embodiments, the scaffold sequence comprises a stem-loop structure. In some embodiments, the scaffold sequence comprises or consists of 90 nucleotides. In some embodiments, the scaffold sequence comprises or consists of 93 nucleotides. In some embodiments, the scaffold sequence comprises or consists of the sequence

(SEQ ID NO: 13)
GUUUAAGAGCUAUGCUGGAAACAGCAUAGCAAGUUUAAAUAAGGCUAGUC
CGUUAUCAACUUGAAAAAGUGGCACCGAGUCGGUGCUUUUUUU.

In some embodiments, the scaffold sequence comprises or consists of the sequence

(SEQ ID NO: 17)
GGACAGCAUAGCAAGUUAAAAUAAGGCUAGUCCGUUAUCAACUUGAAAAA
GUGGCACCGAGUCGGUGCUUUUU.

In some embodiments, the scaffold sequence comprises or consists of the sequence

(SEQ ID NO: 82)
GUUUAAGAGCUAUGCUGGAAACAGCAUAGCAAGUUUAAAUAAGGCUAGUC
CGUUAUCAACUUGAAAAAGUGGCACCGAGUCGGUGCUUUUUUU
or
(SEQ ID NO: 83)
GUUUUAGAGCUAGAAAUAGCAAGUUAAAAUAAGGCUAGUCCGUUAUCAAC
UUGAAAAAGUGGCACCGAGUCGGUGCUUUUUUU.

In some embodiments of the compositions of the disclosure, the gRNA does not bind or does not selectively bind to a second sequence within the RNA molecule.

In some embodiments of the compositions of the disclosure, an RNA genome or an RNA transcriptome comprises the RNA molecule.

In some embodiments of the compositions of the disclosure, the sequence encoding the RNA-binding protein encodes a CRISPR-Cas protein or RNA-binding portion thereof. In some embodiments, the RNA-binding protein is a fusion protein. In some embodiments, the CRISPR-Cas protein is a Type II CRISPR-Cas protein. In some embodiments, the RNA-binding protein comprises a Cas9 polypeptide or an RNA-binding portion thereof. In some embodiments, the CRISPR-Cas protein comprises a native RNA nuclease activity. In some embodiments, the native RNA nuclease activity is reduced or inhibited. In some embodiments, the native RNA nuclease activity is increased or induced. In some embodiments, the CRISPR-Cas protein comprises a native DNA nuclease activity and the native DNA nuclease activity is inhibited. In some embodiments, the CRISPR-Cas protein comprises a mutation. In some embodiments, a nuclease domain of the CRISPR-Cas protein comprises the mutation. In some embodiments, the mutation occurs in a nucleic acid encoding the CRISPR-Cas protein. In some embodiments, the mutation occurs in an amino acid encoding the CRISPR-Cas protein. In some embodiments, the mutation comprises a substitution, an insertion, a deletion, a frameshift, an inversion, or a transposition. In some embodiments, the mutation comprises a deletion of a nuclease domain, a binding site within the nuclease domain, an active site within the nuclease domain, or at least one essential amino acid residue within the nuclease domain.

In some embodiments of the compositions of the disclosure, the RNA binding protein comprises a CRISPR-Cas protein or RNA-binding portion thereof. In some embodiments, the CRISPR-Cas protein is a Type V CRISPR-Cas protein. In some embodiments, the first RNA binding protein comprises a Cpf1 polypeptide or an RNA-binding portion thereof. In some embodiments, the CRISPR-Cas protein comprises a native RNA nuclease activity. In some embodiments, the native RNA nuclease activity is reduced or inhibited. In some embodiments, the native RNA nuclease activity is increased or induced. In some embodiments, the CRISPR-Cas protein comprises a native DNA nuclease activity and the native DNA nuclease activity is inhibited. In some embodiments, the CRISPR-Cas protein comprises a mutation. In some embodiments, a nuclease domain of the CRISPR-Cas protein comprises the mutation. In some embodiments, the mutation occurs in a nucleic acid encoding the CRISPR-Cas protein. In some embodiments, the mutation occurs in an amino acid encoding the CRISPR-Cas protein. In some embodiments, the mutation comprises a substitution, an insertion, a deletion, a frameshift, an inversion, or a transposition. In some embodiments, the mutation comprises a deletion of a nuclease domain, a binding site within the nuclease domain, an active site within the nuclease domain, or at least one essential amino acid residue within the nuclease domain.

In some embodiments of the compositions of the disclosure, the RNA binding protein comprises a CRISPR-Cas protein or RNA-binding portion thereof. In some embodiments, the CRISPR-Cas protein is a Type VI CRISPR-Cas protein. In some embodiments, the RNA binding protein comprises a Cas13 polypeptide or an RNA-binding portion thereof. In some embodiments, the RNA binding protein comprises a Cas13d polypeptide or an RNA-binding portion thereof. In some embodiments, the CRISPR-Cas protein comprises a native RNA nuclease activity. In some embodiments, the native RNA nuclease activity is reduced or inhibited. In some embodiments, the native RNA nuclease activity is increased or induced. In some embodiments, the CRISPR-Cas protein comprises a native DNA nuclease activity and the native DNA nuclease activity is inhibited. In some embodiments, the CRISPR-Cas protein comprises a mutation. In some embodiments, a nuclease domain of the CRISPR-Cas protein comprises the mutation. In some embodiments, the mutation occurs in a nucleic acid encoding the CRISPR-Cas protein. In some embodiments, the mutation occurs in an amino acid encoding the CRISPR-Cas protein. In some embodiments, the mutation comprises a substitution, an insertion, a deletion, a frameshift, an inversion, or a transposition. In some embodiments, the mutation comprises a deletion of a nuclease domain, a binding site within the nuclease domain, an active site within the nuclease domain, or at least one essential amino acid residue within the nuclease domain.

In some embodiments, a target RNA-binding fusion protein is not an RNA-guided target RNA-binding fusion protein and as such comprises at least one RNA-binding polypeptide which is capable of binding a target RNA without a corresponding gRNA sequence. Such non-guided RNA-binding polypeptides include, without limitation, at least one RNA-binding protein or RNA-binding portion thereof which is a PUF (Pumilio and FBF homology family). This type RNA-binding polypeptide can be used in place of a gRNA-guided RNA binding protein such as CRISPR/Cas. In some embodiments of the compositions of the disclosure, the RNA-binding protein or RNA-binding portion thereof is a PUF (Pumilio and FBF homology family). The unique RNA recognition mode of PUF proteins (named for Drosophila Pumilio and C. elegans fem-3 binding factor) that are involved in mediating mRNA stability and translation are well known in the art. The PUF domain of human Pumiliol, also known in the art, binds tightly to cognate RNA sequences and its specificity can be modified. It contains eight PUF repeats that recognize eight consecutive RNA bases with each repeat recognizing a single base. Since two amino acid side chains in each repeat recognize the Watson-Crick edge of the corresponding base and determine the specificity of that repeat, a PUF domain can be designed to specifically bind most 8-nt RNA. Wang et al., Nat Methods. 2009; 6(11): 825-830. See also WO2012/068627 which is incorporated by reference herein in its entirety.

In some embodiments of the compositions of the disclosure, the RNA-binding protein or RNA-binding portion thereof is a PUMBY (Pumilio-based assembly) protein. RNA-binding protein PumHD (Pumilio homology domain, a member of the PUF family), which has been widely used in native and modified form for targeting RNA, has been engineered to yield a set of four canonical protein modules, each of which targets one RNA base. These modules (i.e., Pumby, for Pumilio-based assembly) can be concatenated in chains of varying composition and length, to bind desired target RNAs. The specificity of such Pumby-RNA interactions is high, with undetectable binding of a Pumby chain to RNA sequences that bear three or more mismatches from the target sequence. Katarzyna et al., PNAS, 2016; 113(19): E2579-E2588.

In some embodiments of the compositions of the disclosure, the first RNA binding protein comprises a Pumilio and FBF (PUF) protein. In some embodiments, the first RNA binding protein comprises a Pumilio-based assembly (PUMBY) protein. In some embodiments, a PUF1 protein of the disclosure comprises or consists of the amino acid sequence of

| SEQ ID NO: 219) | |
|---|---|
| MDKSKQMNIN NLSNIPEVID PGITIPIYEE EYENNGESNS QLQQQPQKLG SYRSRAGKFS | 60 |
| NTLSNLLPSI SAKLHHSKKN SHGKNGAEFS SSNNSSQSTV ASKTPRASPS RSKMMESSID | 120 |
| GVTMDRPGSL TPPQDMEKLV HFPDSSNNFL IPAPRGSSDS FNLPHQISRT RNNTMSSQIT | 180 |
| SISSIAPKPR TSSGIWSSNA SANDPMQQHL LQQLQPTTSN NTTNSNTLND YSTKTAYFDN | 240 |
| MVSTSGSQMA DNKMNTNNLA IPNSVWSNTR QRSQSNASSI YTDAPLYEQP ARASISSHYT | 300 |
| IPTQESPLIA DEIDPQSINW VTMDPTVPSI NQISNLLPTN TISISNVFPL QHQQPQLNNA | 360 |
| INLTSTSLAT LCSKYGEVIS ARTLRNLNMA LVEFSSVESA VKALDSLQGK EVSMIGAPSK | 420 |
| ISFAKILPMH QQPPQFLLNS QGLPLGLENN NLQPQPLLQE QLFNGAVTFQ QQGNVSIPVF | 480 |
| NQQSQQSQHQ NHSSGSAGFS NVLHGYNNNN SMHGNNNNSA NEKEQCPFPL PPPNVNEKED | 540 |
| LLREIIELFF ANSDEYQINS LIKKSLNHKG TSDTQNFGPL PEPLSGREFD PPKLRELRKS | 600 |
| IDSNAESDLE IEQLATAMLD ELPELSSDYL GNTIVQKLFE HSSDIIKDIM LRKTSKYLTS | 660 |
| MGVHKNGTWA CQKMITMAHT PRQIMQVTQG VKDYCTPLIN DQFGNYVIQC VLKFGFPWNQ | 720 |
| FIFESIIANF WVIVQNRYGA RAVRACLEAH DIVTPEQSIV LSAMIVTYAE YLDTNSNGAL | 780 |
| LVTWFLDTSV LPNRHSILAP RLTKRIVELC GHRLASLTIL KVLNYRGDDN ARKIILDSLF | 840 |
| GNVNAHDSSP PKELTKLLCE TNYGPTFVHK VLAMPLLEDD LRAHIIKQVR KVLTDSTQIQ | 900 |
| PSRRLLEEVG LASPSSTHNK TKQQQQQHHN SSISHMFATP DTSGQHMRGL SVSSVKSGGS | 960 |
| KHTTMNTTTT NGSSASTLSP GQPLNANSNS SMGYFSYPGV FPVSGFSGNA SNGYAMNNDD | 1020 |
| LSSQFDMLNF NNGTRLSLPQ LSLTNHNNTT MELVNNVGSS QPHTNNNNNN NNTNYNDDNT | 1080 |
| VFETLTLHSA N | 1091. |

In some embodiments, a PUF3 protein of the disclosure comprises or consists of the amino acid sequence of

| (SEQ ID NO: 220) | |
|---|---|
| 1 | MEMNMDMDMD MELASIVSSL SALSHSNNNG GQAAAAGIVN GGAAGSQQIG GFRRSSFTTA |
| 61 | NEVDSEILLL HGSSESSPIF KKTALSVGTA PPFSTNSKEF FGNGGNYYQY RSTDTASLSS |
| 121 | ASYNNYHTHH TAANLGKNNK VNHLLGQYSA SIAGPVYYNG NDNNNSGGEG FFEKFGKSLI |
| 181 | DGTRELESQD RPDAVNTQSQ FISKSVSNAS LDTQNTFEQN VESDKNFNKL NRNTTNSGSL |
| 241 | YHSSSNSGSS ASLESERAHY PKRNIWNVAN TPVFRPSNNP AAVGATNVAL PNQQDGPANN |
| 301 | NFPPYMNGFP PNQFHQGPHY QNFPNYLIGS PSNFISQMIS VQIPANEDTE DSNGKKKKKA |
| 361 | NRPSSVSSPS SPPNNSPFPF AYPNPMMFMP PPPLSAPQQQ QQQQQQQQQE DQQQQQQQEN |
| 421 | PYIYYPTPNP IPVKMPKDEK TFKKRNNKNH PANNSNNANK QANPYLENSI PTKNTSKKNA |
| 481 | SSKSNESTAN NHKSHSHSHP HSQSLQQQQQ TYHRSPLLEQ LRNSSSDKNS NSNMSLKDIF |
| 541 | GHSLEFCKDQ HGSRFIQREL ATSPASEKEV IFNEIRDDAI ELSNDVFGNY VIQKFFEFGS |
| 601 | KIQKNTLVDQ FKGNMKQLSL QMYACRVIQK ALEYIDSNQR IELVLELSDS VLQMIKDQNG |
| 661 | NHVIQKAIET IPIEKLPFIL SSLTGHIYHL STHSYGCRVI QRLLEFGSSE DQESILNELK |
| 721 | DFIPYLIQDQ YGNYVIQYVL QQDQFTNKEM VDIKQETIET VANNVVEYSK HKFASNVVEK |
| 781 | SILYGSKNQK DLIISKILPR DKNHALNLED DSPMILMIKD QFANYVIQKL VNVSEGEGKK |
| 841 | LIVIAIRAYL DKLNKSNSLG NRHLASVEKL AALVENAEV. |

In some embodiments, a PUF4 protein of the disclosure comprises or consists of the amino acid sequence of

(SEQ ID NO: 221)

| | |
|---|---|
| 1 | MSTKGLKEEI DDVPSVDPVV SETVNSALEQ LQLDDPEENA |
| | TSNAFANKVS QDSQFANGPP |
| 61 | SQMFPHTQMM GGMGFMPYSQ MMQVPHNPCP FFPPPDFNDP |
| | TAPLSSSPLN AGGPPMLFKN |
| 121 | DSLPFQMLSS GSSVATQGGQ NLNPLINDNS MKVIPIASAD |
| | PLWTHSNVTG SASVAIEETT |
| 181 | ATLQESLPSK GRESNNKASS FPRQTFHALS PTDLINAANN |
| | VTLSKDFQSD MQNFSKAKKP |
| 241 | SVGANNTAKT RTQSISFDNT PSSTSFIPPT NSVSEKLSDF |
| | KIETSKEDLI NKTAPAKKES |
| 301 | PTTYGAAYPY GGPLLQPNPI MPGHPHNISS PIYGIRSPFP |
| | NSYEMGAQFQ PFSPILNPTS |
| 361 | HSLNANSPIP LTQSPIHLAP VLNPSSNSVA FSDMKNDGGK |
| | PTTDNDKAGP NVRMDLINPN |
| 421 | LGPSMQPFHI LPPQQNTPPP PWLYSTPPPF NAMVPPHLLA |
| | QNHMTLMNSA NNKHHGRNNN |
| 481 | SMSSHNDNDN IGNSNYNNKD TGRSNVGKMK NMKNSYHGYY |
| | NNNNNNNNNN NYNNNSNATN |
| 541 | SNSAEKQRKI EESSRFADAV LDQYIGSIHS LCKDQHGCRF |
| | LQKQLDILGS FAADAIFEET |
| 601 | KDYTVELMTD SFGNYLIQKL LEEVTTEQRI VLTKISSPHF |
| | VEISLNPHGT RALQKLIECI |
| 661 | KTDEEAQIVV DSIRTYTVQL SKDLNGNHVI QKCLQRLKPE |
| | NFQFIFDAIS DSCIDIATHR |
| 721 | HGCCVLQRCL DHGTTEQCDN LCDKLLALVD KLTLDPFGNY |
| | VVQYIITKEA EKNKYDYTHK |
| 781 | IVHLLKPRAI ELSIHKFGSN VIEKILKTAI VSEPMILEIL |
| | NNGGETGIQS LLNDSYGNYV |
| 841 | LQTALDISHK QNDYLYKRLS EIVAPLLVGP IRNTPHGKRI |
| | IGMLHLDS. |

In some embodiments, a PUF5 protein of the disclosure comprises or consists of the amino acid sequence of

| (SEQ ID NO: 222) | |
|---|---|
| 1 | MSDSTGRINS KASDSSSISD HQTADLSIFN GSFDGGAFSS SNIPLFNFMG TGNQRFQYSP |
| 61 | HPFAKESDPC RLAALTPSTP KGPLNLTPAD FGLADFSVGN ESFADFTANN TSFVGNVQSN |
| 121 | VRSTRLLPAW AVDNSGNIRD DLTLQDVVSN GSLIDFAMDR TGVKFLERHF PEDHDNEMHF |
| 181 | VLFDKLTEQG AVFTSLCRSA AGNFIIQKFV EHATLDEQER LVRKMCDNGL IEMCLDKFAC |
| 241 | PVVQMSIQKF DVSIAMKLVE KISSLDFLPL CTDQCAIHVL QKVVKLLPIS AWSFFVKFLC |
| 301 | RDDNLMTVCQ DKYGCRLVQQ TIDKLSDNPK LHCFNTRLQL LHGLMTSVAR NCFRLSSNEF |
| 361 | ANYVVQYVIK SSGVMEMYRD TIIEKCLLRN ILSMSQDKYA SHVVEGAFLF APPLLLSEMM |
| 421 | DEIFDGYVKD QETNRDALDI LLFHQYGNYV VQQMISICIS ALLGKEERKM VASEMRLYAK |
| 481 | WFDRIKNRVN RHSGRLERFS SGKKIIESLQ KLNVPMTMTN EPMPYWAMPT PLMDISAHFM |
| 541 | NKLNFQKNSV FDE. |

In some embodiments, a PUF6 protein of the disclosure comprises or consists of the amino acid sequence of

| (SEQ ID NO: 223) | |
|---|---|
| 1 | MTPNRRSTDS YNMLGASEDF DPDFSLLSNK THKNKNTKPP VKLLPYRHGS NTTSSDLDNY |
| 61 | IFNSGSGSSD DETPPPAAPI FISLEEVLLN GLLIDFAIDP SGVKFLEANY PLDSEDQIRK |
| 121 | AVFEKLTEST TLFVGICHSR NGNFIVQKLV ELATPAEQRE LLRQMIDGGL LVMCKDKFAC |
| 181 | RVVQLALQKF DHSNVFQLIQ ELSTFDLAAM CTDQISIHVI QRVVKQLPVD MWTFFVHFLS |
| 241 | SGDSLMAVCQ DKYGCRLVQQ VIDRLAENPK LPCFKFRIQL LHSLMTCIVR NCYRLSSNEF |
| 301 | ANYVIQYVIK SSGIMEMYRD TIIDKCLLRN LLSMSQDKYA SHVIEGAFLF APPALLHEMM |
| 361 | EEIFSGYVKD VELNRDALDI LLFHQYGNYV VQQMISICTA ALIGKEERQL PPAILLLYSG |
| 421 | WYEKMKQRVL QHASRLERFS SGKKIIDSVM RHGVPTAAAI NAQAAPSLME LTAQFDAMFP |
| 481 | SFLAR. |

In some embodiments, a PUF7 protein of the disclosure comprises or consists of the amino acid sequence of

| (SEQ ID NO: 224) | |
|---|---|
| 1 | MTPNRRSTDS YNMLGASFDF DPDFSLLSNK THKNKNPKPP VKLLPYRHGS NTTSSDSDSY |
| 61 | IFNSGSGSSD AETPAPVAPI FISLEDVILN GQLIDFAIDP SGVKFLEANY PLDSEDQIRK |
| 121 | AVFEKFTEST TLFVGLCHSR NGNFIVQKLV ELATPAEQRE LLRQMIDGGL LAMCKDKFAC |
| 181 | RVVQLALQKF DHSNVFQLIQ ELSTFDLAAM CTDQISIHVI QRVVKQLPVD MWTFFVHFLS |
| 241 | SGDSLMAVCQ DKYGCRLVQQ VIDRLAENPK LPCFKFRIQL LHSLMTCIVR NCYRLSSNEF |
| 301 | ANYVIQYVIK SSGIMEMYRD TIIDKCLLRN LLSMSQDKYA SHVIEGAFLF APPALLHEMM |
| 361 | EEIFSGYVKD VESNPDALDI LLFHQYGNYV VQQMISICTA ALIGKEEREL PPAILLLYSG |
| 421 | WYEKMKQRVL QHASRLERFS SGKKIIDSVM RHGVPTAAAV NAQAAPSLME LTAQFDAMFP |
| 481 | SFLAR. |

In some embodiments, a PUF8 protein of the disclosure comprises or consists of the amino acid sequence of

| (SEQ ID NO: 225) | |
|---|---|
| 1 | MSRPISIGNT CTFDPSASPI ESLGRSIGAQ KIVDSVCGSP IRSYGRHIST NPKNERLPDT |
| 61 | PEFQFATYMH QGGKVIGQNT LHMFGTPPSC YCAQENIPIS SNVGHVLSTI NNNYMNHQYN |
| 121 | GSNMFSNQMT QMLQAQAYND LQMHQAHSQS IRVPVQPSAT GIFSNTYREP TTTDDLLTRY |
| 181 | RANPAMMKNL KLSDIRGALL KFAKDQVGSR FIQQELkSSK DRFEKDSIFD EVVSNADELV |
| 241 | DDIFGNYVVQ KFFEYGEERH WARLVDAIID RVPEYAFQMY ACRVLQKALE KINEPLQIKI |
| 301 | LSQIRHVIHR CMKDQNGNHV VQKAIEKVSP QYVQFIVDTL LESSNTIYEM SVDPYGCRVV |
| 361 | QRCLEHCSPS QTKPVIGQIH KRFDEIANNQ YGNYVVQHVI EHGSEEDRMV IVTRVSNNLF |
| 421 | EFATHKYSSN VIEKCLEQGA VYHKSMIVGA ACHHQEGSVP IVVQMMKDQY ANYVVQKMFD |
| 481 | QVTSEQRREL ILTVRPHIPV LRQFPHGKHI LAKLEKYFQK PAVMSYPYQD MQGSH. |

In some embodiments, a PUF9 protein of the disclosure comprises or consists of the amino acid sequence of

| (SEQ ID NO: 226) | |
|---|---|
| 1 | MADPNWAYAP PTNYYADHSI AKPIMISGGH PSQDQGHSPK SESFGQSVTT AFNGMVDNLV |
| 61 | GSPSSSVQQR NYFTTTPFPI SRSPNDRNDD KIMGNGSYGV PIPIPQDGVP QGTPDFQMTP |
| 121 | FLQQGGHLIG GSPNGPVQVS GNWYSGGAGI FSTMQQADPS NGMPGMAAEF VNNENGMPGP |
| 181 | NGMHQQAMIS GSPPFPYQNM MNLTTSFGAM GLGPQQIQQR DPQMFQQPIL HEPIQGMAQN |
| 241 | GFGQQVFFTQ MQNQQHPQGQ AQQQLQQLAQ QHQQQQNSQQ FFGQGPNGMG NGGVMNDWSQ |
| 301 | RSFGMPQQQA QQNGLPPNFS QNPPRRRGPE DPNGQTPKTL QDIKNNVIEF AKDQHGSRFI |
| 361 | QQKLERASLR DKAAIFTPVL ENAEELMTDV FGNYVIQKFF EFGNNEQRNQ LVGTIRGNVM |
| 421 | KLALQMYGCR VIQKALEYVE EKYQHEILGE MEGQVLKCVK DQNGNHVIQK VIERVEPERL |
| 481 | QFIIDAFTKN NSDNVYTLSV HPYGCRVIQR VLEYCNEEQK QPVLDAIQIH LKQLVLDQYG |
| 541 | NYVIQHVIEH GSPSDKEQIV QDVISDDLLK FAQHKFASNV IEKCLTFGGH AERNLIIDKV |
| 601 | CGDPNDPSPP LLQMMKDPFA NYVVQKMLDV ADPQHRKKIT LTIKPHIATL RKYNFGKHIL |
| 661 | LKLEKYFAKQ APANSSNSSS NDQIYEHSPF DIPLGADFSN HPF. |

In some embodiments of the compositions of the disclosure, the RNA-binding protein or RNA-binding portion thereof is a PPR protein. PPR proteins (proteins with pentatricopeptide repeat (PPR) motifs derived from plants) are nuclear-encoded and exclusively controlled at the RNA level organelles (chloroplasts and mitochondria), cutting, translation, splicing, RNA editing, genes specifically acting on RNA stability. PPR proteins are typically a motif of 35 amino acids and have a structure in which a PPR motif is about 10 contiguous amino acids. The combination of PPR motifs can be used for sequence-selective binding to RNA. PPR proteins are often comprised of PPR motifs of about 10 repeat domains. PPR domains or RNA-binding domains may be configured to be catalytically inactive. WO 2013/058404 incorporated herein by reference in its entirety.

In some embodiments of the compositions of the disclosure, a fusion protein comprises the RNA-binding polypeptide. In some embodiments, the fusion protein comprises a sequence encoding a first RNA-binding polypeptide and a sequence encoding a second RNA-binding polypeptide, wherein neither the first RNA-binding polypeptide nor the second RNA-binding polypeptide comprises a significant DNA-nuclease activity, wherein the first RNA-binding polypeptide and the second RNA-binding polypeptide are not identical, and wherein the second RNA-binding polypeptide comprises an RNA-nuclease activity.

In some embodiments of the compositions of the disclosure, including those wherein a fusion protein comprises a sequence encoding a first RNA-binding polypeptide and a sequence encoding a second RNA-binding polypeptide, the first RNA binding protein comprises a CRISPR-Cas protein. In some embodiments, the CRISPR-Cas protein is a Type II CRISPR-Cas protein. In some embodiments, the first RNA binding protein comprises a Cas9 polypeptide or an RNA-binding portion thereof. In some embodiments, the CRISPR-Cas protein is a Type V CRISPR-Cas protein. In some embodiments, the first RNA binding protein comprises a Cpf1 polypeptide or an RNA-binding portion thereof. In some embodiments, the CRISPR-Cas protein is a Type VI CRISPR-Cas protein. In some embodiments, the first RNA binding protein comprises a Cas13 polypeptide or an RNA-binding portion thereof. In some embodiments, the CRISPR-Cas protein comprises a native RNA nuclease activity.

In some embodiments of the compositions of the disclosure, including those wherein a fusion protein comprises a sequence encoding a first RNA-binding polypeptide and a sequence encoding a second RNA-binding polypeptide, the native RNA nuclease activity is reduced or inhibited. In some embodiments, the native RNA nuclease activity is increased or induced. In some embodiments, the CRISPR-Cas protein comprises a native DNA nuclease activity and wherein the native DNA nuclease activity is inhibited. In some embodiments, the CRISPR-Cas protein comprises a mutation. In some embodiments, a nuclease domain of the CRISPR-Cas protein comprises the mutation. In some embodiments, the mutation occurs in a nucleic acid encoding the CRISPR-Cas protein. In some embodiments, the mutation comprises a substitution, an insertion, a deletion, a frameshift, an inversion, or a transposition. In some embodiments, the mutation comprises a deletion of a nuclease domain, a binding site within the nuclease domain, an active site within the nuclease domain, or at least one essential amino acid residue within the nuclease domain.

In some embodiments of the compositions of the disclosure, including those wherein a fusion protein comprises a sequence encoding a first RNA-binding polypeptide or RNA-binding portion thereof and a sequence encoding a second RNA-binding polypeptide or RNA-binding portion thereof, the first RNA binding protein comprises a Pumilio and FBF (PUF) protein. In some embodiments, the first RNA binding protein comprises a Pumilio-based assembly (PUMBY) protein. In some embodiments, the first RNA binding protein comprises a PPR (pentatricopeptide repeat) protein.

In some embodiments of the compositions of the disclosure, including those wherein a fusion protein comprises a sequence encoding a first RNA-binding polypeptide and a sequence encoding a second RNA-binding polypeptide, the first RNA binding protein does not require multimerization for RNA-binding activity. In some embodiments, the first RNA binding protein is not a monomer of a multimer complex. In some embodiments, a multimer protein complex does not comprise the first RNA binding protein.

In some embodiments of the compositions of the disclosure, including those wherein a fusion protein comprises a sequence encoding a first RNA-binding polypeptide and a sequence encoding a second RNA-binding polypeptide, the first RNA binding protein selectively binds to a target sequence within the RNA molecule. In some embodiments, the first RNA binding protein does not comprise an affinity for a second sequence within the RNA molecule. In some embodiments, the first RNA binding protein does not comprise a high affinity for or selectively bind a second sequence within the RNA molecule.

In some embodiments of the compositions of the disclosure, including those wherein a fusion protein comprises a sequence encoding a first RNA-binding polypeptide and a sequence encoding a second RNA-binding polypeptide, an RNA genome or an RNA transcriptome comprises the RNA molecule.

In some embodiments of the compositions of the disclosure, including those wherein a fusion protein comprises a sequence encoding a first RNA-binding polypeptide and a sequence encoding a second RNA-binding polypeptide, the first RNA binding protein comprises between 2 and 1300 amino acids, inclusive of the endpoints.

In some embodiments of the compositions of the disclosure, including those wherein a fusion protein comprises a sequence encoding a first RNA-binding polypeptide and a sequence encoding a second RNA-binding polypeptide, the sequence encoding the first RNA binding protein further comprises a sequence encoding a nuclear localization signal (NLS). In some embodiments, the sequence encoding a nuclear localization signal (NLS) is positioned 3′ to the sequence encoding the first RNA binding protein. In some embodiments, the first RNA binding protein comprises an NLS at a C-terminus of the protein. In some embodiments, the sequence encoding the first RNA binding protein further comprises a first sequence encoding a first NLS and a second sequence encoding a second NLS. In some embodiments, the sequence encoding the first NLS or the second NLS is positioned 3′ to the sequence encoding the first RNA binding protein. In some embodiments, the first RNA binding protein comprises the first NLS or the second NLS at a C-terminus of the protein.

### RNA-Binding Endonucleases

In some embodiments of the compositions of the disclosure, the second RNA binding protein comprises or consists of a nuclease domain. In some embodiments, the second RNA binding protein binds RNA in a manner in which it associates with RNA. In some embodiments, the second RNA binding protein associates with RNA in a manner in which it cleaves RNA.

In some embodiments of the compositions of the disclosure, the second RNA binding protein comprises or consists of an RNAse.

In some embodiments of the compositions of the disclosure, including those wherein a fusion protein comprises a sequence encoding a first RNA-binding polypeptide and a sequence encoding a second RNA-binding polypeptide, the second RNA binding protein comprises or consists of a nuclease domain. In some embodiments, the sequence encoding the second RNA binding protein comprises or consists of an RNAse. In some embodiments, the second RNA binding protein comprises or consists of an RNAse1. In some embodiments, the sequence encoding the RNAse1 comprises or consists of:

(SEQ ID NO: 20)
KESRAKKFQRQHMDSDSSPSSSSTYCNQMMRRRNMTQGLCKPVNTFVHE
PLVDVQNVCFQEKVTCKNGQGNCYKSNSSMHITDCRLTNGSRYPNCAYR
TSPKERHIIVACEGSPYVPVHFDASVEDST.

In some embodiments, the second RNA binding protein comprises or consists of an RNAse4. In some embodiments, the sequence encoding the RNAse4 comprises or consists of:

(SEQ ID NO: 21)
QDGMYQRFLRQHVHPEETGGSDRYCDLMMQRRKMTLYHCKRENTFIHED
IWNIRSICSTTNIQCKNGKMNCHEGVVKVTDCRDTGSSRAPNCRYRAIA
STRRVVIACEGNPQVPVHFDG.

In some embodiments, the second RNA binding protein comprises or consists of an RNAse6. In some embodiments, the sequence encoding the RNAse6 comprises or consists of:

(SEQ ID NO: 22)
WPKRLTKAHWFEIQHIQPSPLQCNRAMSGINNYTQHCKHQNTFLHDSFQ
NVAAVCDLLSIVCKNRRHNCHQSSKPVNMTDCRLTSGKYPQCRYSAAAQ
YKFFIVACDPPQKSDPPYKLVPVHLDSIL.

In some embodiments, the second RNA binding protein comprises or consists of an RNAse7. In some embodiments, the sequence encoding the RNAse7 comprises or consists of:

(SEQ ID NO: 23)
APARAGFCPLLLLLLLGLWVAEIPVSAKPKGMTSSQWFKIQHMQPSPQA
CNSAMKNINKHTKRCKDLNTFLHEPFSSVAATCQTPKIACKNGDKNCHQ
SHGPVSLTMCKLTSGKYPNCRYKEKRQNKSYVVACKPPQKKDSQQFHLV
PVHLDRVL.

In some embodiments, the second RNA binding protein comprises or consists of an RNAse8. In some embodiments, the sequence encoding the RNAse8 comprises or consists of:

(SEQ ID NO: 24)
TSSQWFKTQHVQPSPQACNSAMSIINKYTERCKDLNTFLHEPFSSVAIT
CQTPNIACKNSCKNCHQSHGPMSLTMGELTSGKYPNCRYKEKHLNTPYI
VACDPPQQGDPGYPLVPVHLDKVV.

In some embodiments, the second RNA binding protein comprises or consists of an RNAse2. In some embodiments, the sequence encoding the RNAse2 comprises or consists of:

(SEQ ID NO: 25)
KPPQFTWAQWFETQHINMTSQQCTNAMQVINNYQRRCKNQNTFLLTTFA
NVVNVCGNPNMTCPSNKTRKNCHHSGSQVPLIHCNLTTPSPQNISNCRY
AQTPANMFYIVACDNRDQRRDPPQYPVVPVHLDRII.

In some embodiments, the second RNA binding protein comprises or consists of an RNAse6PL. In some embodiments, the sequence encoding the RNAse6PL comprises or consists of:

(SEQ ID NO: 26)
DKRLRDNHEWKKLIMVQHWPETVCEKIQNDCRDPPDYWTIHGLWPDKSE
GCNRSWPFNLEEIKKNWMEITDSSLPSPSMGPAPPRWMRSTPRRSTLAE
AWNSTGSWTSTGGCALPPAALPSGDLCCRPSLTAGSRGVGVDLTALHQL
LHVHYSATGIIPEECSEPTKPFQIILHHDHTEWVQSIGMPIWGTISSSE
SAIGKNEESQPACAVLSHDS.

In some embodiments, the second RNA binding protein comprises or consists of an RNAseL. In some embodiments, the sequence encoding the RNAseL comprises or consists of:

(SEQ ID NO: 27)
AAVEDNHLLIKAVQNEDVDLVQQLLEGGANVNFQEEEGGWTPLHNAVQMS
REDIVELLLRHGADPVLRKKNGATPFILAAIAGSVKdLLKLFLSKGADVN
ECDFYGFTAFMEAAVYGKVKALKFLYKRGANVNLRRKTKEDQERLRKGGA
TALMDAAEKGHVEVLKILLDEMGADVNACDNMGRNALIHALLSSDDSDVE
AITHLLLDHGADVNVRGERGKTPLILAVEKKHLGLVQRLLEQEHIEINDT
DSDGKTALLLAVELKLKKIAELLCKRGASTDCGDLVMTARRNYDHSLVKV
LLSHGAKEDFHPPAEDWKPQSSHWGAALKDLHRIYRPMIGKLKFFIDEKY
KIADTSEGGIYLGFYEKQEVAVKTFCEGSPRAQREVSCLQSSRENSHLVT
FYGSESHRGHLFVCVTLCEQTLEACLDVHRGEDVENEEDEFARNVLSSIF
KAVQELHLSCGYTHQDLQPQNILIDSKKAAHLADFDKSIKWAGDPQEVKR
DLEDLGRLVLYVVKKGSISFEDLKAQSNEEVVQLSPDEETKDLIHRLFHP
GEHVRDCLSDLLGHPFFWTWESRYRTLRNVGNESDIKTRKSESEILRLLQ
PGPSEHSKSFDKWTTKINECVMKKMNKFYEKRGNFYQNTVGDLLKFIRNL
GEHIDEEKHKKMKLKIGDPSLYFQKTFPDLVIYVYTKLQNTEYRKHFPQT
HSPNKPQCDGAGGASGLASPGC.

In some embodiments, the second RNA binding protein comprises or consists of an RNAseT2. In some embodiments, the sequence encoding the RNAseT2 comprises or consists of:

(SEQ ID NO: 28)
VQHWPETVCEKIQNDCRDPPDYWTIHGLWPDKSEGCNRSWPFNLEEIKD
LLPEIVIRAYWFIDVIHSFPNRSRFWKHEWEKHGTCAAQVDALNSQKKY
FGRSLELYRELDLNSVLLKLGIKPSINYYQVADFKDALARVYGVIPKIQ
CLPPSQDEEVQTIGQIELCLTKQDQQLQNCTEPGEQPSPKQEVWLANGA
AESRGLRVCEDGPVFYPPPKKTKH.

In some embodiments, the second RNA binding protein comprises or consists of an RNAse11. In some embodiments, the sequence encoding the RNAse11 comprises or consists of:

(SEQ ID NO: 29)
EASESTMKIIKEEFTDEEMQYDMAKSGQEKQTIEILMNPILLVKNTSLS
MSKDDMSSTLLTFRSLHYNDPKGNSSGNDKECCNDMTVWRKVSEANGSC
KWSNNFIRSSTEVMRRVHRAPSCKFVQNPGISCCESLELENTVCQFTTG
KQFPRCQYHSVTSLEKILTVLTGHSLMSWLVCGSKL.

In some embodiments, the second RNA binding protein comprises or consists of an RNAseT2-like. In some embodiments, the sequence encoding the RNAseT2-like comprises or consists of:

(SEQ ID NO: 30)
XLGGADKRLRDNHEWKKLIMVQHWPETVCEKIQNDCRDPPDYWTIHGLW
PDKSEGCNRSWPFNLEEIKDLLPEMRAYWPDVIHSFPNRSRFWKHEWEK
HGTCAAQVDALNSQKKYFGRSLELYRELDLNSVLLKLGIKPSINYYQTT
EEDLNLDVEPTTEDTAEEVTIHVLLHSALFGEIGPRRW.

In some embodiments of the compositions of the disclosure, the second RNA binding protein comprises or consists of a mutated RNAse.

In some embodiments, the second RNA binding protein comprises or consists of a mutated Rnase1 (Rnase1(K41R)) polypeptide. In some embodiments, the Rnase1(K41R) polypeptide comprises or consists of:

(SEQ ID NO: 116)
KESRAKKFQRQHMDSDSSPSSSSTYCNQMMRRRNIVITQGRCRPVNTFV
HEPLVDVQNVCFQEKVTCKNGQGNCYKSNSSMHITDCRLTNGSRYPNCA
YRTSPKERHIIVACEGSPYVPVHFDASVEDS.

In some embodiments, the second RNA binding protein comprises or consists of a mutated Rnase1 (Rnase1(K41R, D121E)) polypeptide. In some embodiments, the Rnase1 (Rnase1(K41R, D121E)) polypeptide comprises or consists of:

(SEQ ID NO: 117)
KESRAKKFQRQHMDSDSSPSSSSTYCNQMMRRRNIVITQGRCRPVNTFV
HEPLVDVQNVCFQEKVTCKNGQGNCYKSNSSMHITDCRLTNGSRYPNCA
YRTSPKERHIIVACEGSPYVPVHFEASVEDST.

In some embodiments, the second RNA binding protein comprises or consists of a mutated Rnase1 (Rnase1(K41R, D121E, H119N)) polypeptide. In some embodiments, the Rnase1 (Rnase1(K41R, D121E, H119N)) polypeptide comprises or consists of:

(SEQ ID NO: 118)
KESRAKKFQRQHMDSDSSPSSSSTYCNQMMRRRNIVITQGRCRPVNTFV
HEPLVDVQNVCFQEKVTCKNGQGNCYKSNSSMHITDCRLTNGSRYPNCA
YRTSPKERHIIVACEGSPYVPVNFEASVEDST.

In some embodiments, the second RNA binding protein comprises or consists of a mutated Rnase1. In some embodiments, the second RNA binding protein comprises or consists of a mutated Rnase1 (Rnase1(H119N)) polypeptide. In some embodiments, the Rnase1 (Rnase1(H119N)) polypeptide comprises or consists of:

(SEQ ID NO: 119)
KESRAKKFQRQHMDSDSSPSSSSTYCNQMMRRRNMTQGRCKPVNTFVHEP
LVDVQNVCFQEKVTCKNGQGNCYKSNSSMHITDCRLTNGSRYPNCAYRTS
PKERHIIVACEGSPYVPVNFDASVEDST.

In some embodiments, the second RNA binding protein comprises or consists of a mutated Rnase1 (Rnase1(R39D, N67D, N88A, G89D, R91D, H119N)) polypeptide. In some embodiments, the Rnase1 (Rnase1(R39D, N67D, N88A, G89D, R91D, H119N)) polypeptide comprises or consists of:

(SEQ ID NO: 120)
KESRAKKFQRQHMDSDSSPSSSSTYCNQMMRRRNMTQGDCKPVNTFVHE
PLVDVQNVCFQEKVTCKDGQGNCYKSNSSMHITDCRLTADSDYPNCAYR
TSPKERHIIVACEGSPYVPVNFDASVEDST.

In some embodiments, the second RNA binding protein comprises or consists of a mutated Rnase1 (Rnase1(R39D, N67D, N88A, G89D, R91D, H119N)) polypeptide.

In some embodiments, the Rnase1 (Rnase1(R39D, N67D, N88A, G89D, R91D, H119N, K41R, D121E)) polypeptide comprises or consists of:

(SEQ ID NO: 121)
KESRAKKFQRQHMDSDSSPSSSSTYCNQMMRRRNMTQGDCRPVNTFVHEP
LVDVQNVCFQEKVTCKDGQGNCYKSNSSMHITDCRLTADSDYPNCAYRTS
PKERHIIVACEGSPYVPVNFEASVEDST.

In some embodiments, the second RNA binding protein comprises or consists of a mutated Rnase1 (Rnase1(R39D, N67D, N88A, G89D, R91D, H119N)) polypeptide. In some embodiments, the Rnase1 (Rnase1(R39D, N67D, N88A, G89D, R91D)) polypeptide comprises or consists of:

(SEQ ID NO: 122)
KESRAKKFQRQHMDSDSSPSSSSTYCNQMMRRRNMTQGDCKPVNTFVHEP
LVDVQNVCFQEKVTCKDGQGNCYKSNSSMHITDCRLTADSDYPNCAYRTS
PKERHIIVACEGSPYVPVHFDASVEDST.

In some embodiments, the second RNA binding protein comprises or consists of a mutated Rnase1 (Rnase1 (R39D, N67D, N88A, G89D, R91D, H119N, K41R, D121E)) polypeptide that comprises or consists of:

(SEQ ID NO: 208)
KESRAKKFQRQHMDSDSSPSSSSTYCNQMMRRRNMTQGDCRPVNTFVHEP
LVDVQNVCFQEKVTCKDGQGNCYKSNSSMHITDCRLTADSDYPNCAYRTS
PKERHIIVACEGSPYVPVNFEASVEDST.

In some embodiments, the sequence encoding the second RNA binding protein comprises or consists of a NOB1 polypeptide. The composition of claim 101, wherein the sequence encoding the NOB1 polypeptide comprises or consists of:

(SEQ ID NO: 31)
APVEHVVADAGAFLRHAALQDIGKNIYTIREVVTEIRDKATRRRLAVLP
YELRFKEPLPEYVRLVTEFSKKTGDYPSLSATDIQVLALTYQLEAEFVG
VSHLKQEPQKVKVSSSIQHPETPLHISGFHLPYKPKPPQETEKGHSACE
PENLEFSSFNIFWRNPLPNIDHELQELLIDRGEDVPSEEEEEEENGFED
RKDDSDDDGGGWITPSNIKQIQQELEQCDVPEDVRVGCLTTDFAMQNVL
LQMGLHVLAVNGMLIREARSYILRCHGCFKTTSDMSRVFCSHCGNKTLK
KVSVTV.

In some embodiments, the sequence encoding the second RNA binding protein comprises or consists of an endonuclease. In some embodiments, the sequence encoding the second RNA binding protein comprises or consists of an endonuclease V (ENDOV). In some embodiments, the sequence encoding the ENDOV comprises or consists of:

(SEQ ID NO: 32)
AFSGLQRVGGVDVSFVKGDSVRACASLVVLSFPELEVVYEESRMVSLTAP
YVSGFLAFREVPFLLELVQQLREKEPGLMPQVLLVDGNGVLHHRGFGVAC
HLGVLTDLPCVGVAKKLLQVDGLENNALHKEKIRLLQTRGDSFPLLGDSG
TVLGMALRSHDRSTRPLYISVGHRMSLEAAVRLTCCCCRFRIPEPVRQAD
ICSREHIRKS.

In some embodiments, the sequence encoding the second RNA binding protein comprises or consists of an endonuclease G (ENDOG). In some embodiments, the sequence encoding the ENDOG comprises or consists of:

(SEQ ID NO: 33)
AELPPVPGGPRGPGELAKYGLPGLAQLKSRESYVLCYDPRTRGALWVVEQ
LRPERLRGDGDRRECDFREDDSVHAYHRATNADYRGSGFDRGHLAAAANH
RWSQKAMDDTFYLSNVAPQVPHLNQNAWNNLEKYSRSLTRSYQNVYVCTG
PLFLPRTEADGKSYVKYQVIGKNHVAVPTHFEKVLILEAAGGQIELRTYV
MPNAPVDEAIPLERFLVPIESIERASGLLFVPNILARAGSLKAITAGSK.

In some embodiments, the sequence encoding the second RNA binding protein comprises or consists of an endonuclease D1 (ENDOD1). In some embodiments, sequence encoding the ENDOD1 comprises or consists of:

(SEQ ID NO: 34)
RLVGEEEAGFGECDKFFYAGTPPAGLAADSHVKICQRAEGAERFATLYST
RDRIPVYSAFRAPRPAPGGAEQRWLVEPQIDDPNSNLEEAINEAEAITSV
NSLGSKQALNTDYLDSDYQRGQLYPFSLSSDVQVATFTLTNSAPMTQSFQ
ERWYVNLHSLMDRALTPQCGSGEDLYILTGTVPSDYRVKDKVAVPEFVWL
AACCAVPGGGWAMGFVKHTRDSDIIEDVMVKDLQKLLPFNPQLFQNNCGE
TEQDTEKMKKILEVVNQIQDEERMVQSQKSSSPLSSTRSKRSTLLPPEAS
EGSSSFLGKLMGFIATPFIKLFQLIYYLVVAILKNIVYFLWCVTKQVING
IESCLYRLGSATISYFMAIGEELVSIPWKVLKVVAKVIRALLRILCCLLK
AICRVLSIPVRVLVDVATFPVYTMGAIPIVCKDIALGLGGTVSLLFDTAF
GTLGGLFQVVFSVCKRIGYKVTFDNSGEL.

In some embodiments, the sequence encoding the second RNA binding protein comprises or consists of a Human flap endonuclease-1 (hFEN1). In some embodiments, the sequence encoding the hFEN1 comprises or consists of:

(SEQ ID NO: 35)
MGIQGLAKLIADVAPSAIRENDIKSYFGRKVAIDASMSIYQFLIAVRQGG
DVLQNEEGETTSHLMGMFYRTIRMMENGIKPVYVFDGKPPQLKSGELAKR
SERRAEAEKQLQQAQAAGAEQEVEKFTKRLVKVTKQHNDECKHLLSLMGI
PYLDAPSEAEASCAALVKAGKVYAAATEDMDCLTFGSPVLMRHLTASEAK
KLPIQEFHLSRILQELGLNQEQFVDLCILLGSDYCESIRGIGPKRAVDLI
QKHKSIEEIVRRLDPNKYPVPENWLHKEAHQLFLEPEVLDPESVELKWSE
PNEEELIKFMCGEKQFSEERIRSGVKRLSKSRQGSTQGRLDDFFKVTGSL
SSAKRKEPEPKGSTKKKAKTGAAGKFKRGK.

In some embodiments, the sequence encoding the second RNA binding protein comprises or consists of a human Schlafen 14 (hSLFN14) polypeptide. In some embodiments, the sequence encoding the hSLFN14 comprises or consists of:

(SEQ ID NO: 36)
ESTHVEFKRFTTKKVIPRIKEMLPHYVSAFANTQGGYVLIGVDDKSKEVV
GCKWEKVNPDLLKKEIENCIEKLPTFHFCCEKPKVNFTTKILNVYQKDVL
DGYVCVIQVEPFCCVVFAEAPDSWIMKDNSVTRLTAEQWVVMMLDTQSAP
PSLVTDYNSCLISSASSARKSPGYPIKVHKFKEALQ.

In some embodiments, the sequence encoding the second RNA binding protein comprises or consists of a human beta-lactamase-like protein 2 (hLACTB2) polypeptide. In some embodiments, the sequence encoding the hLACTB2 comprises or consists of:

(SEQ ID NO: 37)
TLQGTNTYLVGTGPRRILIDTGEPAIPEYISCLKQALTEFNTAIQEIVVT
HWHRDHSGGIGDICKSINNDTTYCIKKLPRNPQREDIGNGEQQYVYLKDG
DVIKTEGATLRVLYTPGHTDDHMALLLEEENAIFSGDCILGEGTTVFEDL
YDYMNSLKELLKIKADIIYPGHGPVIHNAEAKIQQYISHRNIREQQILTL
FRENFEKSFTVMELVKIIYKNTPENLHEMAKHNLLLHLKKLEKEGKIFSN
TDPDKKWKAHL.

In some embodiments, the sequence encoding the second RNA binding protein comprises or consists of an apurinic/apyrimidinic (AP) endodeoxyribonuclease (APEX2) polypeptide. In some embodiments, the sequence encoding the APEX2 comprises or consists of:

(SEQ ID NO: 38)
MLRVVSWNINGIRRPLQGVANQEPSNCAAVAVGRILDELDADIVCLQETK
VTRDALTEPLAIVEGYNSYFSFSRNRSGYSGVATFCKDNATPVAAEEGLS
GLFATQNGDVGCYGNMDEFTQEELRALDSEGRALLTQHKIRTWEGKEKTL
TLINVYCPHADPGRPERLVFKMRFYRLLQIRAEALLAAGSHVIILGDLNT
AHRPIDHWDAVNLECFEEDPGRKWMDSLLSNLGCQSASHVGPFIDSYRCF
QPKQEGAFTCWSAVTGARHLNYGSRLDYVLGDRTLVIDTFQASFLLPEVI
VIGSDHCPVGAVLSVSSVPAKQCPPLCTRFLPEFAGTQLKILRFLVPLEQ
SPVLEQSTLQHNNQTRVQTCQNKAQVRSTRPQPSQVGSSRGQKNLKSYFQ
PSPSCPQASPDIELPSLPLMSALMTPKTPEEKAVAKVVKGQAKTSEAKDE
KELRTSFWKSVLAGPLRTPLCGGHREPCVMRTVKKPGPNLGRRFYMCARP
RGPPTDPSSRCNFFLWSRPS.

In some embodiments, the sequence encoding the APEX2 comprises or consists of:

(SEQ ID NO: 39)
MLRVVSWNINGIRRPLQGVANQEPSNCAAVAVGRILDELDADIVCLQETK
VTRDALTEPLAIVEGYNSYFSFSRNRSGYSGVATFCKDNATPVAAEEGLS
GLFATQNGDVGCYGNMDEFTQEELRALDSEGRALLTQHKIRTWEGKEKTL
TLINVYCPHADPGRPERLVFKMRFYRLLQIRAEALLAAGSHVIILGDLNT
AHRPIDHWDAVNLECFEEDPGRKWMDSLLSNLGCQSASHVGPFIDSYRCF
QPKQEGAFTCWSAVTGARHLNYGSRLDYVLGDRTLVIDTFQASFLLPEVG
SDHCPVGAVLSVSSVPAKQCPPLCTRFLPEFAGTQLKILRFLVPLEQSP.

In some embodiments, the sequence encoding the second RNA binding protein comprises or consists of an angiogenin (ANG) polypeptide. In some embodiments, the sequence encoding the ANG comprises or consists of:

(SEQ ID NO: 40)
QDNSRYTHFLTQHYDAKPQGRDDRYCESIMRRRGLTSPCKDINTFIHGNK
RSIKAICENKNGNPHRENLRISKSSFQVTTCKLHGGSPWPPCQYRATAGF
RNVVVACENGLPVHLDQSIFRRP.

In some embodiments, the sequence encoding the second RNA binding protein comprises or consists of a heat responsive protein 12 (HRSP12) polypeptide. In some embodiments, the sequence encoding the HRSP12 comprises or consists of:

(SEQ ID NO: 41)
SSLIRRVISTAKAPGAIGPYSQAVLVDRTIYISGQIGMDPSSGQLVSGGV
AEEAKQALKNMGEILKAAGCDFTNVVKTTVLLADINDFNTVNEIYKQYFK
SNFPARAAYQVAALPKGSRIEIEAVAIQGPLTTASL.

In some embodiments, the sequence encoding the second RNA binding protein comprises or consists of a Zinc Finger CCCH-Type Containing 12A (ZC3H12A) polypeptide. In some embodiments, the sequence encoding the ZC3H12A comprises or consists of:

(SEQ ID NO: 42)
GGGTPKAPNLEPPLPEEEKEGSDLRPVVIDGSNVAMSHGNKEVFSCRGIL
LAVNWFLERGHTDITVFVPSWRKEQPRPDVPITDQHILRELEKKKILVFT
PSRRVGGKRVVCYDDRFIVKLAYESDGIVVSNDTYRDLQGERQEWKRFIE
ERLLMYSFVNDKFMPPDDPLGRHGPSLDNFLRKKPLTLE.

In some embodiments, the sequence encoding the ZC3H12A comprises or consists of:

(SEQ ID NO: 43)
SGPCGEKPVLEASPTMSLWEFEDSHSRQGTPRPGQELAAEEASALELQM
KVDFFRKLGYSSTEIHSVLQKLGVQADTNTVLGELVKHGTATERERQTS
PDPCPQLPLVPRGGGTPKAPNLEPPLPEEEKEGSDLRPVVIDGSNVAMS
HGNKEVFSCRGILLAVNWFLERGHTDITVFVPSWRKEQPRPDVPITDQH
ILRELEKKKILVFTPSRRVGGKRVVCYDDRFIVKLAYESDGIVVSNDTY
RDLQGERQEWKRFIEERLLMYSFVNDKFMPPDDPLGRHGPSLDNFLRKK
PLTLEHRKQPCPYGRKCTYGIKCRFFHPERPSCPQRSVADELRANALLS
PPRAPSKDKNGRRPSPSSQSSSLLTESEQCSLDGKKLGAQASPGSRQEG
LTQTYAPSGRSLAPSGGSGSSFGPTDWLPQTLDSLPYVSQDCLDSGIGS
LESQMSELWGVRGGGPGEPGPPRAPYTGYSPYGSELPATAAFSAFGRAM
GAGHFSVPADYPPAPPAFPPREYWSEPYPLPPPTSVLQEPPVQSPGAGR
SPWGRAGSLAKEQASVYTKLCGVFPPHLVEAVMGRFPQLLDPQQLAAEI
LSYKSQHPSE.

In some embodiments, wherein the sequence encoding the second RNA binding protein comprises or consists of a Reactive Intermediate Imine Deaminase A (RIDA) polypeptide. In some embodiments, the sequence encoding the RIDA comprises or consists of:

(SEQ ID NO: 44)
SSLIRRVISTAKAPGAIGPYSQAVLVDRTIYISGQIGMDPSSGQLVSGGV
AEEAKQALKNMGEILKAAGCDFTNVVKTTVLLADINDFNTVNEIYKQYFK
SNFPARAAYQVAALPKGSRIEIEAVAIQGPLTTASL.

In some embodiments, the sequence encoding the second RNA binding protein comprises or consists of a Phospholipase D Family Member 6 (PDL6) polypeptide. In some embodiments, the sequence encoding the PDL6 comprises or consists of:

(SEQ ID NO: 126)
EALFFPSQVTCTEALLRAPGAELAELPEGCPCGLPHGESALSRLLRALLA
ARASLDLCLFAFSSPQLGRAVQLLHQRGVRVRVVTDCDYMALNGSQIGLL
RKAGIQVRHDQDPGYMHHKFAIVDKRVLITGSLNWTTQAIQNNRENVLIT
EDDEYVRLFLEEFERIWEQFNPTKYTFFPPKKSHGSCAPPVSRAGGRLLS
WHRTCGTSSESQT.

In some embodiments, the sequence encoding the second RNA binding protein comprises or consists of a Endonuclease III-like protein 1 (NTHL) polypeptide. In some embodiments, the sequence encoding the NTHL comprises or consists of:

(SEQ ID NO: 123)
CSPQESGMTALSARMLTRSRSLGPGAGPRGCREEPGPLRRREAAAEARKS
HSPVKRPRKAQRLRVAYEGSDSEKGEGAEPLKVPVWEPQDWQQQLVNIRA
MRNKKDAPVDHLGTEHCYDSSAPPKVRRYQVLLSLMLSSQTKDQVTAGAM
QRLRARGLTVDSILQTDDATLGKLIYPVGFWRSKVKYIKQTSAILQQHYG
GDIPASVAELVALPGVGPKMAHLAMAVAWGTVSGIAVDTHVHRIANRLRW
TKKATKSPEETRAALEEWLPRELWHEINGLLVGFGQQTCLPVHPRCHACL
NQALCPAAQGL.

In some embodiments, the sequence encoding the second RNA binding protein comprises or consists of a Mitochondrial ribonuclease P catalytic subunit (KIAA0391) polypeptide. In some embodiments, the sequence encoding the KIAA0391 comprises or consists of:

(SEQ ID NO: 127)
KARYKTLEPRGYSLLIRGLIHSDRWREALLLLEDIKKVITPSKKNYNDCI
QGALLHQDVNTAWNLYQELLGHDIVPMLETLKAFFDFGKDIKDDNYSNKL
LDILSYLRNNQLYPGESFAHSIKTWFESVPGKQWKGQFTTVRKSGQCSGC
GKTIESIQLSPEEYECLKGKIMRDVIDGGDQYRKTTPQELKRFENFIKSR
PPFDVVIDGLNVAKMFPKVRESQLLLNVVSQLAKRNLRLLVLGRKHMLRR
SSQWSRDEMEEVQKQASCFFADDISEDDPFLLYATLHSGNHCRFITRDLM
RDHKACLPDAKTQRLFFKWQQGHQLAIVNRFPGSKLTFQRILSYDTVVQT
TGDSWHIPYDEDLVERCSCEVPTKWLCLHQKT.

In some embodiments, the sequence encoding the second RNA binding protein comprises or consists of an apurinic or apyrimidinic site lyase (APEX1) polypeptide. In some embodiments, the sequence encoding the APEX1 comprises or consists of:

(SEQ ID NO: 125)
PKRGKKGAVAEDGDELRTEPEAKKSKTAAKKNDKEAAGEGPALYEDPPDQ
KTSPSGKPATLKICSWNVDGLRAWIKKKGLDWVKEEAPDILCLQETKCSE
NKLPAELQELPGLSHQYWSAPSDKEGYSGVGLLSRQCPLKVSYGIGDEEH
DQEGRVIVAEFDSFVLVTAYVPNAGRGLVRLEYRQRWDEAFRKFLKGLAS
RKPLVLCGDLNVAHEEIDLRNPKGNKKNAGFTPQERQGFGELLQAVPLAD
SFRHLYPNTPYAYTFWTYMMNARSKNVGWRLDYFLLS.

In some embodiments, the sequence encoding the second RNA binding protein comprises or consists of an argonaute 2 (AGO2) polypeptide. In some embodiments, the sequence encoding the AGO2 comprises or consists of:

(SEQ ID NO: 128)
SVEPMFRHLKNTYAGLQLVVVILPGKTPVYAEVKRVGDTVLGMATQCVQM
KNVQRTTPQTLSNLCLKINVKLGGVNNILLPQGRPPVFQQPVIFLGADVT
HPPAGDGKKPSIAAVVGSMDAHPNRYCATVRVQQHRQEIIQDLAAMVREL
LIQFYKSTRFKPTRIIFYRDGVSEGQFQQVLHHELLAIREACIKLEKDYQ
PGITFIVVQKRHHTRLFCTDKNERVGKSGNIPAGTTVDTKITHPTEFDFY
LCSHAGIQGTSRPSHYHVLWDDNRFSSDELQILTYQLCHTYVRCTRSVSI
PAPAYYAHLVAFRARYHLVDKEHDSAEGSHTSGQSNGRDHQALAKAVQVH
QDTLRTMYFA.

In some embodiments, the sequence encoding the second RNA binding protein comprises or consists of a mitochondrial nuclease EXOG (EXOG) polypeptide. In some embodiments, the sequence encoding the EXOG comprises or consists of:

(SEQ ID NO: 129)
QGAEGALTGKQPDGSAEKAVLEQFGFPLTGTEARCYTNHALSYDQAKRVP
RWVLEHISKSKIMGDADRKHCKFKPDPNIPPTFSAFNEDYVGSGWSRGHM
APAGNNKFSSKAMAETFYLSNIVPQDFDNNSGYWNRIEMYCRELTERFED
VWVVSGPLTLPQTRGDGKKIVSYQVIGEDNVAVPSHLYKVILARRSSVST
EPLALGAFVVPNEAIGFQPQLTEFQVSLQDLEKLSGLVFFPHLDRTSDIR
NICSVDTCKLLDFQEFTLYLSTRKIEGARSVLRLEKIMENLKNAEIEPDD
YFMSRYEKKLEELKAKEQSGTQIRKPS.

In some embodiments, the sequence encoding the second RNA binding protein comprises or consists of a Zinc Finger CCCH-Type Containing 12D (ZC3H12D) polypeptide. In some embodiments, the sequence encoding the ZC3H12D comprises or consists of:

(SEQ ID NO: 130)
EHPSKMEFFQKLGYDREDVLRVLGKLGEGALVNDVLQELIRTGSRPGALE
HPAAPRLVPRGSCGVPDSAQRGPGTALEEDFRTLASSLRPIVIDGSNVAM
SHGNKETFSCRGIKLAVDWFRDRGHTYIKVFVPSWRKDPPRADTPIREQH
VLAELERQAVLVYTPSRKVHGKRLVCYDDRYIVKVAYEQDGVIVSNDNYR
DLQSENPEWKWFIEQRLLMFSFVNDRFMPPDDPLGRHGPSLSNFLSRKPK
PPEPSWQHCPYGKKCTYGIKCKFYHPERPHHAQLAVADELRAKTGARPGA
GAEEQRPPRAPGGSAGARAAPREPFAHSLPPARGSPDLAALRGSFSRLAF
SDDLGPLGPPLPVPACSLTPRLGGPDWVSAGGRVPGPLSLPSPESQFSPG
DLPPPPGLQLQPRGEHRPRDLHGDLLSPRRPPDDPWARPPRSDRFPGRSV
WAEPAWGDGATGGLSVYATEDDEGDARARARIALYSVFPRDQVDRVMAAF
PELSDLARLILLVQRCQSAGAPLGKP.

In some embodiments, the sequence encoding the second RNA binding protein comprises or consists of an endoplasmic reticulum to nucleus signaling 2 (ERN2) polypeptide. In some embodiments, the sequence encoding the ERN2 comprises or consists of:

(SEQ ID NO: 131)
RQQQPQVVEKQQETPLAPADFAHISQDAQSLHSGASRRSQKRLQSPSKQA
QPLDDPEAEQLTVVGKISFNPKDVLGRGAGGTFVFRGQFEGRAVAVKRLL
RECFGLVRREVQLLQESDRHPNVLRYFCTERGPQFHYIALELCRASLQEY
VENPDLDRGGLEPEVVLQQLMSGLAHLHSLHIVHRDLKPGNILITGPDSQ
GLGRVVLSDFGLCKKLPAGRCSFSLHSGIPGTEGWMAPELLQLLPPDSPT
SAVDIFSAGCVFYYVLSGGSHPFGDSLYRQANILTGAPCLAHLEEEVHDK
VVARDLVGAMLSPLPQPRPSAPQVLAHPFFWSRAKQLQFFQDVSDWLEKE
SEQEPLVRALEAGGCAVVRDNWHEHISMPLQTDLRKFRSYKGTSVRDLLR
AVRNKKHHYRELPVEVRQALGQVPDGFVQYFTNRFPRLLLHTHRAMRSCA
SESLFLPYYPPDSEARRPCPGATGR.

In some embodiments, the sequence encoding the second RNA binding protein comprises or consists of a pelota mRNA surveillance and ribosome rescue factor (PELO) polypeptide. In some embodiments, the sequence encoding the PELO comprises or consists of:

(SEQ ID NO: 132)
KLVRKNIEKDNAGQVTLVPEEPEDMWHTYNLVQVGDSLRASTIRKVQTES
STGSVGSNRVRTTLTLCVEAIDFDSQACQLRVKGTNIQENEYVKMGAYHT
IELEPNRQFTLAKKQWDSVVLERIEQACDPAWSADVAAVVMQEGLAHICL
VTPSMTLTRAKVEVNIPRKRKGNCSQHDRALERFYEQVVQAIQRHIHFDV
VKCILVASPGFVREQFCDYLFQQAVKTDNKLLLENRSKFLQVHASSGHKY
SLKEALCDPTVASRLSDTKAAGEVKALDDFYKMLQHEPDRAFYGLKQVEK
ANEAMAIDTLLISDELFRHQDVATRSRYVRLVDSVKENAGTVRIFSSLHV
SGEQLSQLTGVAAILRFPVPELSDQEGDSSSEED.

In some embodiments, wherein the sequence encoding the second RNA binding protein comprises or consists of a YBEY metallopeptidase (YBEY) polypeptide. In some embodiments, the sequence encoding the YBEY comprises or consists of:

(SEQ ID NO: 133)
SLVIRNLQRVIPIRRAPLRSKIEIVRRILGVQKFDLGIICVDNKNIQHIN
RIYRDRNVPTDVLSFPFHEHLKAGEFPQPDFPDDYNLGDIFLGVEYIFHQ
CKENEDYNDVLTVTATHGLCHLLGFTHGTEAEWQQMFQKEKAVLDELGRR
TGTRLQPLTRGLFGGS.

In some embodiments, the sequence encoding the second RNA binding protein comprises or consists of a cleavage and polyadenylation specific factor 4 like (CPSF4L) polypeptide. In some embodiments, the sequence encoding the CPSF4L comprises or consists of:

(SEQ ID NO: 134)
QEVIAGLERFTFAFEKDVEMQKGTGLLPFQGMDKSASAVCNFFTKGLCEK
GKLCPFRHDRGEKMVVCKHWLRGLCKKGDHCKFLHQYDLTRMPECYFYSK
FGDCSNKECSFLHVKPAFKSQDCPWYDQGFCKDGPLCKYRHVPRIMCLNY
LVGFCPEGPKCQFAQKIREFKLLPGSKI.

In some embodiments, the sequence encoding the second RNA binding protein comprises or consists of an hCG_2002731polypeptide. In some embodiments, the sequence encoding the hCG_2002731 comprises or consists of:

(SEQ ID NO: 135)
KLVRKNIEKDNAGQVTLVPEEPEDMWHTYNLVQVGDSLRASTIRKVQTES
STGSVGSNRVRTTLTLCVEAIDFDSQACQLRVKGTNIQENEYVKMGAYHT
IELEPNRQFTLAKKQWDSVVLERIEQACDPAWSADVAAVVMQEGLAHICL
VTPSMTLTRAKVEVNIPRKRKGNCSQHDRALERFYEQVVQAIQRHIHFDV
VKCILVASPGFVREQFCDYMFQQAVKTDNKLLLENRSKFLQVHASSGHKY
SLKEALCDPTVASRLSDTKAAGEVKALDDFYKMLQHEPDRAFYGLKQVEK
ANEAMAIDTLLISDELFRHQDVATRSRYVRLVDSVKENAGTVRIFSSLHV
SGEQLSQLTGVAAILRFPVPELSDQEGDSSSEED.

In some embodiments, the sequence encoding the hCG 2002731 comprises or consists of:

(SEQ ID NO: 136)
DPAWSADVAAVVMQEGLAHICLVTPSMTLTRAKVEVNIPRKRKGNCSQHD
RALERFYEQVVQAIQRHIHFDVVKCILVASPGFVREQFCDYMFQQAVKTD
NKLLLENRSKFLQVHASSGHKYSLKEALCDPTVASRLSDTKAAGEVKALD
DFYKMLQHEPDRAFYGLKQVEKANEAMAIDTLLISDELFRHQDVATRSRY
VRLVDSVKENAGTVRIFSSLHVSGEQLSQLTGVAAILRFPVPELSDQEGD
SSSEED.

In some embodiments, the sequence encoding the second RNA binding protein comprises or consists of an Excision Repair Cross-Complementation Group 1 (ERCC1) polypeptide. In some embodiments, the sequence encoding the ERCC1 comprises or consists of:

(SEQ ID NO: 137)
MDPGKDKEGVPQPSGPPARKKFVIPLDEDEVPPGVRGNPVLKFVRNVPWE
FGDVIPDYVLGQSTCALFLSLRYHNLHPDYIHGRLQSLGKNFALRVLLVQ
VDVKDPQQALKELAKMCILADCTLILAWSPEEAGRYLETYKAYEQKPADL
LMEKLEQDFVSRVTECLTTVKSVNKTDSQTLLTTFGSLEQLIAASREDLA
LCPGLGPQK.

In some embodiments, the sequence encoding the second RNA binding protein comprises or consists of a ras-related C3 botulinum toxin substrate 1 isoform (RAC1) polypeptide. In some embodiments, the sequence encoding the RAC1 comprises or consists of:

(SEQ ID NO: 138)
KESRAKKFQRQHMDSDSSPSSSSTYCNQMMRRRNMTQGRCKPVNTFVHEP
LVDVQNVCFQEKVTCKNGQGNCYKSNSSMHITDCRLTNGSRYPNCAYRTS
PKERHIIVACEGSPYVPVHFDASVEDST.

In some embodiments, the sequence encoding the second RNA binding protein comprises or consists of a Ribonuclease A A1 (RAA1) polypeptide. In some embodiments, the sequence encoding the RAA1 comprises or consists of:

(SEQ ID NO: 139)
QDNSRYTHFLTQHYDAKPQGRDDRYCESIMRRRGLTSPCKDINTFIHGNK
RSIKAICENKNGNPHRENLRISKSSFQVTTCKLHGGSPWPPCQYRATAGF
RNVVVACENGLPVHLDQSIFRRP.

In some embodiments, the sequence encoding the second RNA binding protein comprises or consists of a Ras Related Protein (RAB1) polypeptide. In some embodiments, the sequence encoding the RAB1 comprises or consists of:

(SEQ ID NO: 140)
GLGLVQPSYGQDGMYQRFLRQHVHPEETGGSDRYCNLMMQRRKMTLYHC
KRFNTFIHEDIWNIRSICSTTNIQCKNGKMNCHEGVVKVTDCRDTGSSR
APNCRYRAIASTRRVVIACEGNPQVPVHFDG.

In some embodiments, the sequence encoding the second RNA binding protein comprises or consists of a DNA Replication Helicase/Nuclease 2 (DNA2) polypeptide. In some embodiments, the sequence encoding the DNA2 comprises or consists of:

(SEQ ID NO: 141)
XSAVDNILLKLAKFKIGFLRLGQIQKVHPAIQQFTEQEICRSKSIKSLA
LLEELYNSQLIVATTCMGINHPIFSRKIFDFCIVDEASQISQPICLGPL
FFSRRFVLVGDHQQLPPLVLNREARALGMSESLFKRLEQNKSAVVQLTV
QYRIVINSKIMSLSNKLTYEGKLECGSDKVANAVINLRHFKDVKLELEF
YADYSDNPWLMGVFEPNNPVCFLNTDKVPAPEQVEKGGVSNVTEAKLIV
FLTSIFVKAGCSPSDIGIIAPYRQQLKIINDLLARSIGMVEVNTVDKYQ
GRDKSIVLVSFVRSNKDGTVGELLKDWRRLNVAITRAKHKLILLGCVPS
LNCYPPLEKLLNHLNSEKLISFFFCIWSHLIALL.

In some embodiments, the sequence encoding the second RNA binding protein comprises or consists of a FLJ35220 polypeptide. In some embodiments, the sequence encoding the FLJ35220 comprises or consists of:

(SEQ ID NO: 142)
MALRSHDRSTRPLYISVGHRIVISLEAAVRLTCCCCRFRIPEPVRQADI
CSREHIRKSLGLPGPPTPRSPKAQRPVACPKGDSGESSALC.

In some embodiments, wherein the sequence encoding the second RNA binding protein comprises or consists of a FLJ13173 polypeptide. In some embodiments, the sequence encoding the FLJ13173 comprises or consists of:

(SEQ ID NO: 143)
CYTNHALSYDQAKRVPRWVLEHISKSKIMGDADRKHCKFKPDPNIPPTF
SAFNEDYVGSGWSRGHMAPAGNNKFSSKAMAETFYLSNIVPQDFDNNSG
YWNRIEMYCRELTERFEDVWVVSGPLTLPQTRGDGKKIVSYQVIGEDNV
AVPSHLYKVILARRSSVSTEPLALGAFVVPNEAIGFQPQLTEFQVSLQD
LEKLSGLVFFPHLDRT.

In some embodiments, the sequence encoding the second RNA binding protein comprises or consists of a DNA repair endonuclease XPF (ERCC4) polypeptide. In some embodiments, the sequence encoding the ERCC4 comprises or consists of:

(SEQ ID NO: 64)
MESGQPARRIAMAPLLEYERQLVLELLDTDGLVVCARGLGADRLLYHFL
QLHCHPACLVLVLNTQPAEEEYFINQLKIEGVEHLPRRVTNEITSNSRY
EVYTQGGVIFATSRILVVDFLTDRIPSDLITGILVYRAHRIIESCQEAF
ILRLFRQKNKRGFIKAFTDNAVAFDTGFCHVERVMRNLFVRKLYLWPRF
HVAVNSFLEQHKPEVVEIHVSMTPTMLAIQTAILDILNACLKELKCHNP
SLEVEDLSLENAIGKPFDKTIRHYLDPLWHQLGAKTKSLVQDLKILRTL
LQYLSQYDCVTFLNLLESLRATEKAFGQNSGWLFLDSSTSMFINARARV
YHLPDAKMSKKEKISEKMEIKEGEGILWG.

In some embodiments, the sequence encoding the second RNA binding protein comprises or consists of Teneurin Transmembrane Protein 1 (TENM1) polypeptide. In some embodiments, the sequence encoding the TENM1 comprises or consists of:

(SEQ ID NO: 144)
VTVSQMTSVLNGKTRRFADIQLQHGALCFNIRYGTTVEEEKNHVLEIAR
QRAVAQAWTKEQRRLQEGEEGIRAWTEGEKQQLLSTGRVQGYDGYFVLS
VEQYLELSDSANNIHFMRQSEIGRR.

In some embodiments, the sequence encoding the second RNA binding protein comprises or consists of Teneurin Transmembrane Protein 2 (TENM2) polypeptide. In some embodiments, the sequence encoding the TENM2 comprises or consists of:

(SEQ ID NO: 145)
TVSQPTLLVNGKTRRFTNIEFQYSTLLLSIRYGLTPDTLDEEKARVLDQ
ARQRALGTAWAKEQQKARDGREGSRLWTEGEKQQLLSTGRVQGYEGYYV
LPVEQYPELADSSSNIQFLRQNEMGKR.

In some embodiments, the second RNA binding protein comprises or consists of a transcription activator-like effector nuclease (TALEN) polypeptide or a nuclease domain thereof. In some embodiments, the sequence encoding the TALEN polypeptide comprises or consists of:

| (SEQ ID NO: 205) | |
|---|---|
| 1 | MRIGKSSGWL NESVSLEYEH VSPPTRPRDT RRRPRAAGDG GLAHLHRRLA VGYAEDTPRT |
| 61 | EARSPAPRRP LPVAPASAPP APSLVPEPPM PVSLPAVSSP RFSAGSSAAI TDPFPSLPPT |
| 121 | PVLYAMAREL EALSDATWQP AVPLPAEPPT DARRGNTVFD EASASSPVIA SACPQAFASP |
| 181 | PRAPRSARAR RARTGGDAWP APTFLSRPSS SRIGRDVFGK LVALGYSREQ IRKLKQESLS |
| 241 | EIAKYHTTLT GQGFTHADIC RISRRRQSLR VVARNYPELA AALPELTRAH IVDIARQRSG |
| 301 | DLALQALLPV ATALTAAPLR LSASQIATVA QYGERPAIQA LYRLRRKLTR APLHLTPQQV |
| 361 | VAIASNTGGK RALEAVCVQL PVLRAAPYRL STEQVVAIAS NKGGKQALEA VKAHLLDLLG |
| 421 | APYVLDTEQV VAIASHNGGK QALEAVKADL LDLRGAPYAL STEQVVAIAS HNGGKQALEA |
| 481 | VKADLLELRG APYALSTEQV VAIASHNGGK QALEAVKAHL LDLRGVPYAL STEQVVAIAS |
| 541 | HNGGKQALEA VKAQLLDLRG APYALSTAQV VAIASNGGGK QALEGIGEQL LKLRTAPYGL |
| 601 | STEQVVAIAS HDGGKQALEA VGAQLVALRA APYALSTEQV VAIASNKGGK QALEAVKAQL |
| 661 | LELRGAPYAL STAQVVAIAS HDGGNQALEA VGTQLVALRA APYALSTEQV VAIASHDGGK |
| 721 | QALEAVGAQL VALRAAPYAL NTEQVVAIAS SHGGKQALEA VRALFPDLRA APYALSTAQL |
| 781 | VAIASNPGGK QALEAVRALF RELRAAPYAL STEQVVAIAS NHGGKQALEA VRALFRGLRA |
| 841 | APYGLSTAQV VAIASSNGGK QALEAVWALL PVLRATPYDL NTAQIVAIAS HDGGKPALEA |
| 901 | VWAKLPVLRG APYALSTAQV VAIACISGQQ ALEAIEAHMP TLRQASHSLS PERVAAIACI |
| 961 | GGRSAVEAVR QGLPVKAIRR IRREKAPVAG PPPASLGPTP QELVAVLHFF RAHQQPRQAF |
| 1021 | VDALAAFQAT RPALLRLLSS VGVTEIEALG GTIPDATERW QRLLGRLGFR PATGAAAPSP |
| 1081 | DSLQGFAQSL ERTLGSPGMA GQSACSPHRK RPAETAIAPR SIRRSPNNAG QPSEPWPDQL |
| 1141 | AWLQRRKRTA RSHIRADSAA SVPANLHLGT RAQFTPDRLR AEPGPIMQAH TSPASVSFGS |
| 1201 | HVAFEPGLPD PGTPTSADLA SFEAEPFGVG PLDFHLDWLL QILET. |

In some embodiments, the sequence encoding the TALEN polypeptide comprises or consists of:

| (SEQ ID NO: 206) | |
|---|---|
| 1 | mdpirsrtps parellpgpq pdrvqptadr ggappaggpl dglparrtms rtrlpsppap |
| 61 | spafsagsfs dllrqfdpsl ldtslldsmp avgtphtaaa paecdevqsg lraaddpppt |
| 121 | vrvavtaarp prakpaprrr aausdaspa aqvdlrtlgy sqqqqekikp kvgstvaqhh |
| 181 | ealvghgfth ahivalsrhp aalgtvavky qdmiaalpea thedivgvgk qwsgaralea |
| 241 | lltvagelrg pplqldtgql vkiakrggvt aveavhasrn altgapinit paqvvaiasn |
| 301 | nggkgaletv grllpvlcqa hgltpaqvva iashdggkqa letmqrllpv logahglppd |
| 361 | qvvaiasnig gkgaletvqr llpvlogahg ltpdqvvaia shgggkgale tvqrllpvlc |
| 421 | qahgltpdqv vaiashdggk galetvqrll pvlogahglt pdqvvaiasn gggkgaletv |
| 481 | grllpvlcqa hgltpdqvva iasnggkgal etvqrllpvl cgahgltpdg vvaiashdgg |
| 541 | kgaletvqrl lpvlcgthgl tpaqvvaias hdggkgalet vqqllpvlcq ahgltpdqvv |
| 601 | aiasniggkq alatvqrllp vlogahgltp dqvvaiasng ggkgaletvg rllpvlogah |
| 661 | gltpdqvvai asngggkgal etvqrllpvl cgahgltqvg vvaiasnigg kgaletvqrl |
| 721 | lpvlogahgl tpaqvvaias hdggkgalet vqrllpvlcq ahgltpdqvv aiasngggkq |
| 781 | aletvqrllp vlogahgltq eqvvaiasnn ggkgaletvg rllpvlogah gltpdqvvai |
| 841 | asngggkgal etvqrllpvl cqahgltpaq vvaiasnigg kgaletvqrl lpvlcqdhgl |
| 901 | tlaqvvaias niggkgalet vqrllpvlcq ahgltqdqvv aiasniggkq aletvqrllp |
| 961 | vlcqdhgltp dqvvaiasni ggkgaletvg rllpvlcqdh gltldqvvai asnggkgale |
| 1021 | tvqrllpvlc qdhgltpdqv vaiasnsggk galetvqrll pvlcqdhglt pnqvvaiasn |
| 1081 | ggkgalesiv aqlsrpdpal aaltndhlva laclggrpam davkkglpha pelirrvnrr |
| 1141 | igertshrva dyaqvvrvle ffqchshpay afdeamtqfg msrnglvqlf rrvgvtelea |
| 1201 | rggtlppasq rwdrilqasg mkrakpspts aqtpdgaslh afadslerdl dapspmhegd |
| 1261 | qtgassrkrs rsdravtgps aqhsfevrvp eqrdalhlpl swrvkrprtr iggglpdpgt |
| 1321 | piaadlaass tvmwegdaap fagaaddfpa fneeelawlm ellpqsgsvg gti. |

In some embodiments, the second RNA binding protein comprises or consists of a zinc finger nuclease polypeptide or a nuclease domain thereof. In some embodiments, the sequence encoding the zinc finger nuclease polypeptide comprises or consists of:

| (SEQ ID NO: 52) | |
|---|---|
| 1 | MSRPRFNPRG DFPLQRPRAP NPSGMRPPGP FMRPGSMGLP RFYPAGRARG IPHRFAGHES |
| 61 | YQNMGPQRMN VQVTQHRTDP RLTKEKLDFH EAQQKKGKPH GSRWDDEPHI SASVAVKQSS |
| 121 | VTQVTEQSPK VQSRYTKESA SSILASFGLS NEDLEELSRY PDEQLTPENM PLILRDIRMR |
| 181 | KMGRRLPNLP SQSRNKETLG SEAVSSNVID YGHASKYGYT EDPLEVRIYD PEIPTDEVEN |
| 241 | EFQSQQNISA SVPNPNVICN SMFPVEDVFR QMDFPGESSN NRSFFSVESG TKMSGLHISG |
| 301 | GQSVLEPIKS VNQSINQTVS QTMSQSLIPP SMNQQPFSSE LISSVSQQER IPHEPVINSS |
| 361 | NVHVGSRGSK KNYQSQADIP IRSPFGIVKA SWLPKFSHAD AQKMKRLPTP SMMNDYYAAS |
| 421 | PRIFPHLCSL CNVECSHLKD WIQHQNTSTH IESCRQLRQQ YPDWNPEILP SRRNEGNRKE |
| 481 | NETPRRRSHS PSPRRSRRSS SSHRFRRSRS PMHYMYRPRS RSPRICHRFI SRYRSRSRSR |
| 541 | SPYRIRNPFR GSPKCFRSVS PERMSRRSVR SSDRKKALED VVQRSGHGTE FNKQKHLEAA |
| 601 | DKGHSPAQKP KTSSGTKPSV KPTSATKSDS NLGGHSIRCK SKNLEDDTLS ECKQVSDKAV |
| 661 | SLQRKLRKEQ SLHYGSVLLI TELPEDGCTE EDVRKLFQPF GKVNDVLIVP YRKEAYLEME |
| 721 | FKEAITAIMK YIETTPLTIK GKSVKICVPG KKKAQNKEVK KKTLESKKVS ASTLKRDADA |
| 781 | SKAVEIVTST SAAKTGQAKA SVAKVNKSTG KSASSVKSVV TVAVKGNKAS IKTAKSGGKK |
| 841 | SLEAKKTGNV KNKDSNKPVT IPENSEIKTS IEVKATENCA KEAISDAALE ATENEPLNKE |
| 901 | TEEMCVMLVS NLPNKGYSVE EVYDLAKPFG GLKDILILSS HKKAYIEINR KAAESMVKFY |
| 961 | TCFPVLMDGN QLSISMAPEN MNIKDEEAIF ITLVKENDPE ANIDTIYDRF VHLDNLPEDG |
| 1021 | LQCVLCVGLQ FGKVDHHVFI SNRNKAILQL DSPESAQSMY SFLKQNPQNI GDHMLTCSLS |
| 1081 | PKIDLPEVQI EHDPELEKES PGLKNSPIDE SEVQTATDSP SVKPNELEEE STPSIQTETL |
| 1141 | VQQEEPCEEE AEKATCDSDF AVETLELETQ GEEVKEEIPL VASASVSIEQ FTENAEECAL |
| 1201 | NQQMFNSDLE KKGAEIINPK TALLPSDSVF AEERNLKGIL EESPSEAEDF ISGITQTMVE |
| 1261 | AVAEVEKNET VSEILPSTCI VTLVPGIPTG DEKTVDKKNI SEKKGNMDEK EEKEFNTKET |
| 1321 | RMDLQIGTEK AEKNEGRMDA EKVEKMAAMK EKPAENTLFK AYPNKGVGQA NKPDETSKTS |
| 1381 | ILAVSDVSSS KPSIKAVIVS SPKAKATVSK TENQKSFPKS VPRDQINAEK KLSAKEFGLL |
| 1441 | KPTSARSGLA ESSSKFKPTQ SSLTRGGSGR ISALQGKLSK LDYRDITKQS QETEARPSIM |
| 1501 | KRDDSNNKTL AEQNTKNPKS TTGRSSKSKE EPLFPFNLDE FVTVDEVIEE VNPSQAKQNP |
| 1561 | LKGKRKETLK NVPFSELNLK KKKGKTSTPR GVEGELSFVT LDEIGEEEDA AAHLAQALVT |
| 1621 | VDEVIDEEEL NMEEMVKNSN SLFTLDELID QDDCISHSEP KDVTVLSVAE EQDLLKQERL |
| 1681 | VTVDEIGEVE ELPLNESADI TFATLNTKGN EGDTVRDSIG FISSQVPEDP STLVTVDEIQ |
| 1741 | DDSSDLHLVT LDEVTEEDED SLADFNNLKE ELNFVTVDEV GEEEDGDNDL KVELAQSKND |
| 1801 | HPTDKKGNRK KRAVDTKKTK LESLSQVGPV NENVMEEDLK TMIERHLTAK TPTKRVRIGK |
| 1861 | TLPSEKAVVT EPAKGEEAFQ MSEVDEESGL KDSEPERKRK KTEDSSSGKS VASDVPEELD |
| 1921 | FLVPKAGFFC PICSLFYSGE KAMTNHCKST RHKQNTEKFM AKQRKEKEQN EAEERSSR. |

### Guide RNA

The terms guide RNA (gRNA) and single guide RNA (sgRNA) are used interchangeably throughout the disclosure.

Guide RNAs (gRNAs) of the disclosure may comprise of a spacer sequence and a scaffolding sequence. In some embodiments, a guide RNA is a single guide RNA (sgRNA) comprising a contiguous spacer sequence and scaffolding sequence. In some embodiments, the spacer sequence and the scaffolding sequence are not contiguous. In some embodiments, a sequence encoding a guide RNA or single guide RNA of the disclosure comprises or consists of a spacer sequence and a scaffolding sequence, that are separated by a linker sequence. In some embodiments, the linker sequence may comprise or consist of 1, 2, 3, 4, 5, 6, 7, 8, 9, 10, 15, 20, 25, 30, 35, 40, 45, 50 or any number of nucleotides in between. In some embodiments, the linker sequence may comprise at least 1, 2, 3, 4, 5, 6, 7, 8, 9, 10, 15, 20, 25, 30, 35, 40, 45, 50 or any number of nucleotides in between.

Guide RNAs (gRNAs) of the disclosure may comprise non-naturally occurring nucleotides. In some embodiments, a guide RNA of the disclosure or a sequence encoding the guide RNA comprises or consists of modified or synthetic RNA nucleotides. Exemplary modified RNA nucleotides include, but are not limited to, pseudouridine (4), dihydrouridine (D), inosine (I), and 7-methylguanosine (m7G), hypoxanthine, xanthine, xanthosine, 7-methylguanine, 5, 6-Dihydrouracil, 5-methylcytosine, 5-methylcytidine, 5-hydropxymethylcytosine, isoguanine, and isocytosine.

Guide RNAs (gRNAs) of the disclosure may bind modified RNA within a target sequence. Within a target sequence, guide RNAs (gRNAs) of the disclosure may bind modified RNA. Exemplary epigenetically or post-transcriptionally modified RNA include, but are not limited to, 2′-O-Methylation (2′-OMe) (2′-O-methylation occurs on the oxygen of the free 2′-OH of the ribose moiety), N6-methyladenosine (m6A), and 5-methylcytosine (m5C).

In some embodiments of the compositions of the disclosure, a guide RNA of the disclosure comprises at least one sequence encoding a non-coding C/D box small nucleolar RNA (snoRNA) sequence. In some embodiments, the snoRNA sequence comprises at least one sequence that is complementary to the target RNA, wherein the target sequence of the RNA molecule comprises at least one 2′-OMe. In some embodiments, the snoRNA sequence comprises at least one sequence that is complementary to the target RNA, wherein the at least one sequence that is complementary to the target RNA comprises a box C motif (RUGAUGA) and a box D motif (CUGA).

Spacer sequences of the disclosure bind to the target sequence of an RNA molecule. Spacer sequences of the disclosure may comprise a CRISPR RNA (crRNA). Spacer sequences of the disclosure comprise or consist of a sequence having sufficient complementarity to a target sequence of an RNA molecule to bind selectively to the target sequence. Upon binding to a target sequence of an RNA molecule, the spacer sequence may guide one or more of a scaffolding sequence and a fusion protein to the RNA molecule. In some embodiments, a sequence having sufficient complementarity to a target sequence of an RNA molecule to bind selectively to the target sequence has at least 50%, 55%, 60%, 65%, 70%, 75%, 80%, 85%, 90%, 95%, 96, 97%, 98%, 99%, or any percentage identity in between to the target sequence. In some embodiments, a sequence having sufficient complementarity to a target sequence of an RNA molecule to bind selectively to the target sequence has 100% identity the target sequence.

Scaffolding sequences of the disclosure bind the first RNA-binding polypeptide of the disclosure. Scaffolding sequences of the disclosure may comprise a trans acting RNA (tracrRNA). Scaffolding sequences of the disclosure comprise or consist of a sequence having sufficient complementarity to a target sequence of an RNA molecule to bind selectively to the target sequence. Upon binding to a target sequence of an RNA molecule, the scaffolding sequence may guide a fusion protein to the RNA molecule. In some embodiments, a sequence having sufficient complementarity to a target sequence of an RNA molecule to bind selectively to the target sequence has at least 50%, 55%, 60%, 65%, 70%, 75%, 80%, 85%, 90%, 95%, 96, 97%, 98%, 99%, or any percentage identity in between to the target sequence. In some embodiments, a sequence having sufficient complementarity to a target sequence of an RNA molecule to bind selectively to the target sequence has 100% identity the target sequence. Alternatively or in addition, in some embodiments, scaffolding sequences of the disclosure comprise or consist of a sequence that binds to a first RNA binding protein or a second RNA binding protein of a fusion protein of the disclosure. In some embodiments, scaffolding sequences of the disclosure comprise a secondary structure or a tertiary structure. Exemplary secondary structures include, but are not limited to, a helix, a stem loop, a bulge, a tetraloop and a pseudoknot. Exemplary tertiary structures include, but are not limited to, an A-form of a helix, a B-form of a helix, and a Z-form of a helix. Exemplary tertiary structures include, but are not limited to, a twisted or helicized stem loop. Exemplary tertiary structures include, but are not limited to, a twisted or helicized pseudoknot. In some embodiments, scaffolding sequences of the disclosure comprise at least one secondary structure or at least one tertiary structure. In some embodiments, scaffolding sequences of the disclosure comprise one or more secondary structure(s) or one or more tertiary structure(s).

In some embodiments of the compositions of the disclosure, a guide RNA or a portion thereof selectively binds to a tetraloop motif in an RNA molecule of the disclosure. In some embodiments, a target sequence of an RNA molecule comprises a tetraloop motif. In some embodiments, the tetraloop motif is a “GRNA” motif comprising or consisting of one or more of the sequences of GAAA, GUGA, GCAA or GAGA.

In some embodiments of the compositions of the disclosure, a guide RNA or a portion thereof that binds to a target sequence of an RNA molecule hybridizes to the target sequence of the RNA molecule. In some embodiments, a guide RNA or a portion thereof that binds to a first RNA binding protein or to a second RNA binding protein covalently binds to the first RNA binding protein or to the second RNA binding protein. In some embodiments, a guide RNA or a portion thereof that binds to a first RNA binding protein or to a second RNA binding protein non-covalently binds to the first RNA binding protein or to the second RNA binding protein.

In some embodiments of the compositions of the disclosure, a guide RNA or a portion thereof comprises or consists of between 10 and 100 nucleotides, inclusive of the endpoints. In some embodiments, a spacer sequence of the disclosure comprises or consists of between 10 and 30 nucleotides, inclusive of the endpoints. In some embodiments, a spacer sequence of the disclosure comprises or consists of 15, 16, 17, 18, 19, 20, 21, 22, 23, 24, 25, 26, 27, 28, 29 or 30 nucleotides. In some embodiments, the spacer sequence of the disclosure comprises or consists of 20 nucleotides. In some embodiments, the spacer sequence of the disclosure comprises or consists of 21 nucleotides. In some embodiments, a scaffold sequence of the disclosure comprises or consists of between 10 and 100 nucleotides, inclusive of the endpoints. In some embodiments, a spacer sequence of the disclosure comprises or consists of 30, 35, 40, 45, 50, 55, 60, 65, 70, 76, 80, 87, 90, 95, 100 or any number of nucleotides in between. In some embodiments, the scaffold sequence of the disclosure comprises or consists of between 85 and 95 nucleotides, inclusive of the endpoints. In some embodiments, the scaffold sequence of the disclosure comprises or consists of 85 nucleotides. In some embodiments, the scaffold sequence of the disclosure comprises or consists of 90 nucleotides. In some embodiments, the scaffold sequence of the disclosure comprises or consists of 93 nucleotides.

In some embodiments of the compositions of the disclosure, a guide RNA or a portion thereof does not comprise a nuclear localization sequence (NLS).

In some embodiments of the compositions of the disclosure, a guide RNA or a portion thereof does not comprise a sequence complementary to a protospacer adjacent motif (PAM).

Therapeutic or pharmaceutical compositions of the disclosure do not comprise a PAMmer oligonucleotide. In other embodiments, optionally, non-therapeutic or non-pharmaceutical compositions may comprise a PAMmer oligonucleotide.

In some embodiments of the compositions of the disclosure, a guide RNA or a portion thereof comprises a sequence complementary to a protospacer flanking sequence (PFS). In some embodiments, including those wherein a guide RNA or a portion thereof comprises a sequence complementary to a PFS, the first RNA binding protein may comprise a sequence isolated or derived from a Cas13 protein. In some embodiments, including those wherein a guide RNA or a portion thereof comprises a sequence complementary to a PFS, the first RNA binding protein may comprise a sequence encoding a Cas13 protein or an RNA-binding portion thereof. In some embodiments, the guide RNA or a portion thereof does not comprise a sequence complementary to a PFS.

In some embodiments of the compositions of the disclosure, a sequence encoding a guide RNA of the disclosure further comprises a sequence encoding a promoter to drive expression of the guide RNA. In some embodiments, a vector comprising a sequence encoding a guide RNA of the disclosure further comprises a sequence encoding a promoter to drive expression of the guide RNA. In some embodiments, a sequence encoding a promoter to drive expression of the guide RNA comprises a sequence encoding a constitutive promoter. In some embodiments, a sequence encoding a promoter to drive expression of the guide RNA comprises a sequence encoding an inducible promoter. In some embodiments, a sequence encoding a promoter to drive expression of the guide RNA comprises a sequence encoding a hybrid or a recombinant promoter. In some embodiments, a sequence encoding a promoter to drive expression of the guide RNA comprises a sequence encoding a promoter capable of expressing the guide RNA in a mammalian cell. In some embodiments, a sequence encoding a promoter to drive expression of the guide RNA comprises a sequence encoding a promoter capable of expressing the guide RNA in a human cell. In some embodiments, a sequence encoding a promoter to drive expression of the guide RNA comprises a sequence encoding a promoter capable of expressing the guide RNA and restricting the guide RNA to the nucleus of the cell. In some embodiments, a sequence encoding a promoter to drive expression of the guide RNA comprises a sequence encoding a human RNA polymerase promoter or a sequence isolated or derived from a sequence encoding a human RNA polymerase promoter. In some embodiments, a sequence encoding a promoter to drive expression of the guide RNA comprises a sequence encoding a U6 promoter or a sequence isolated or derived from a sequence encoding a U6 promoter. In some embodiments, a sequence encoding a promoter to drive expression of the guide RNA comprises a sequence encoding a human tRNA promoter or a sequence isolated or derived from a sequence encoding a human tRNA promoter. In some embodiments, a sequence encoding a promoter to drive expression of the guide RNA comprises a sequence encoding a human valine tRNA promoter or a sequence isolated or derived from a sequence encoding a human valine tRNA promoter.

In some embodiments of the compositions of the disclosure, a sequence encoding a promoter to drive expression of the guide RNA further comprises a regulatory element. In some embodiments, a vector comprising a sequence encoding a promoter to drive expression of the guide RNA further comprises a regulatory element. In some embodiments, a regulatory element enhances expression of the guide RNA. Exemplary regulatory elements include, but are not limited to, an enhancer element, an intron, an exon, or a combination thereof.

In some embodiments of the compositions of the disclosure, a vector of the disclosure comprises one or more of a sequence encoding a guide RNA, a sequence encoding a promoter to drive expression of the guide RNA and a sequence encoding a regulatory element. In some embodiments of the compositions of the disclosure, the vector further comprises a sequence encoding a fusion protein of the disclosure.

### Fusion Proteins

Fusion proteins in the context of the compositions of the disclosure may comprise a first RNA binding protein and a second RNA binding protein. In some embodiments, along a sequence encoding the fusion protein, the sequence encoding the first RNA binding protein is positioned 5′ of the sequence encoding the second RNA binding protein. In some embodiments, along a sequence encoding the fusion protein, the sequence encoding the first RNA binding protein is positioned 3′ of the sequence encoding the second RNA binding protein.

In some embodiments of the compositions of the disclosure, the sequence encoding the first RNA binding protein comprises a sequence isolated or derived from a protein capable of binding an RNA molecule. In some embodiments, the sequence encoding the first RNA binding protein comprises a sequence isolated or derived from a protein capable of selectively binding an RNA molecule and not binding a DNA molecule, a mammalian DNA molecule or any DNA molecule. In some embodiments, the sequence encoding the first RNA binding protein comprises a sequence isolated or derived from a protein capable of binding an RNA molecule and inducing a break in the RNA molecule. In some embodiments, the sequence encoding the first RNA binding protein comprises a sequence isolated or derived from a protein capable of binding an RNA molecule, inducing a break in the RNA molecule, and not binding a DNA molecule, a mammalian DNA molecule or any DNA molecule. In some embodiments, the sequence encoding the first RNA binding protein comprises a sequence isolated or derived from a protein capable of binding an RNA molecule, inducing a break in the RNA molecule, and neither binding nor inducing a break in a DNA molecule, a mammalian DNA molecule or any DNA molecule.

In some embodiments of the compositions of the disclosure, the sequence encoding the first RNA binding protein comprises a sequence isolated or derived from a protein with no DNA nuclease activity.

In some embodiments of the compositions of the disclosure, the sequence encoding the first RNA binding protein comprises a sequence isolated or derived from a protein having DNA nuclease activity, wherein the DNA nuclease activity does not induce a break in a DNA molecule, a mammalian DNA molecule or any DNA molecule when a composition of the disclosure is contacted to an RNA molecule or introduced into a cell or into a subject of the disclosure.

In some embodiments of the compositions of the disclosure, the sequence encoding the first RNA binding protein comprises a sequence isolated or derived from a protein having DNA nuclease activity, wherein the DNA nuclease activity is inactivated and wherein the DNA nuclease activity does not induce a break in a DNA molecule, a mammalian DNA molecule or any DNA molecule when a composition of the disclosure is contacted to an RNA molecule or introduced into a cell or into a subject of the disclosure. In some embodiments, the sequence encoding the first RNA binding protein comprises a mutation that inactivates or decreases the DNA nuclease activity to a level at which the DNA nuclease activity does not induce a break in a DNA molecule, a mammalian DNA molecule or any DNA molecule when a composition of the disclosure is contacted to an RNA molecule or introduced into a cell or into a subject of the disclosure. In some embodiments, the sequence encoding the first RNA binding protein comprises a mutation that inactivates or decreases the DNA nuclease activity and the mutation comprises one or more of a substitution, inversion, transposition, insertion, deletion, or any combination thereof to a nucleic acid sequence or amino acid sequence encoding the first RNA binding protein or a nuclease domain thereof.

In some embodiments, the fusion protein disclosed herein comprises a linker between the at least two RNA-binding polypeptides. In some embodiments, the linker is a peptide linker. In some embodiments, the peptide linker comprises one or more repeats of the tri-peptide GGS. In other embodiments, the linker is a non-peptide linker. In some embodiments, the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.

In some embodiments, the at least one RNA-binding protein does not require multimerization for RNA-binding activity. In some embodiments, the at least one RNA-binding protein is not a monomer of a multimer complex. In some embodiments, a multimer protein complex does not comprise the RNA binding protein. In some embodiments, the at least one of RNA-binding protein selectively binds to a target sequence within the RNA molecule. In some embodiments, the at least one RNA-binding protein does not comprise an affinity for a second sequence within the RNA molecule. In some embodiments, the at least one RNA-binding protein does not comprise a high affinity for or selectively bind a second sequence within the RNA molecule. In some embodiments, the at least one RNA-binding protein comprises between 2 and 1300 amino acids, inclusive of the endpoints.

In some embodiments, the at least one RNA-binding protein of the fusion proteins disclosed herein further comprises a sequence encoding a nuclear localization signal (NLS). In some embodiments, a nuclear localization signal (NLS) is positioned 3′ to the RNA binding protein. In some embodiments, the at least one RNA-binding protein comprises an NLS at a C-terminus of the protein. In some embodiments, the at least one RNA-binding protein further comprises a first sequence encoding a first NLS and a second sequence encoding a second NLS. In some embodiments, the first NLS or the second NLS is positioned 3′ to the RNA-binding protein. In some embodiments, the at least one RNA-binding protein comprises the first NLS or the second NLS at a C-terminus of the protein. In some embodiments, the at least one RNA-binding protein further comprises an NES (nuclear export signal) or other peptide tag or secretory signal.

In some embodiments, a fusion protein disclosed herein comprises the at least one RNA-binding protein as a first RNA-binding protein together with a second RNA-binding protein comprising or consisting of a nuclease domain.

In some embodiments, the second RNA-binding polypeptide is operably configured to the first RNA-binding polypeptide at the C-terminus of the first RNA-binding polypeptide. In some embodiments, the second RNA-binding polypeptide is operably configured to the first RNA-binding polypeptide at the N-terminus of the first RNA-binding polypeptide. For example, one such exemplary fusion protein is E99 which is configured so that RNAse1(R39D, N67D, N88A, G89D, R19D, H119N, K41R) is located at the N-terminus of SpyCas9 whereas another exemplary fusion protein, E100, is configured so that RNAse1(R39D, N67D, N88A, G89D, R19D, H119N, K41R) is located at the C-terminus of SpyCas9.

gRNA Target Sequences

In some embodiments of the compositions of the disclosure, a target sequence of an RNA molecule comprises a sequence motif corresponding to the RNA binding protein and/or the RNA binding proteins and/or fusion protein thereof.

In some embodiments of the compositions and methods of the disclosure, the sequence motif is a signature of a disease or disorder.

A sequence motif of the disclosure may be isolated or derived from a sequence of foreign or exogenous sequence found in a genomic sequence, and therefore translated into an mRNA molecule of the disclosure or a sequence of foreign or exogenous sequence found in an RNA sequence of the disclosure.

A sequence motif of the disclosure may comprise or consist of a mutation in an endogenous sequence that causes a disease or disorder. The mutation may comprise or consist of a sequence substitution, inversion, deletion, insertion, transposition, or any combination thereof.

A sequence motif of the disclosure may comprise or consist of a repeated sequence. In some embodiments, the repeated sequence may be associated with a microsatellite instability (MSI). MSI at one or more loci results from impaired DNA mismatch repair mechanisms of a cell of the disclosure. A hypervariable sequence of DNA may be transcribed into an mRNA of the disclosure comprising a target sequence comprising or consisting of the hypervariable sequence.

A sequence motif of the disclosure may comprise or consist of a biomarker. The biomarker may indicate a risk of developing a disease or disorder. The biomarker may indicate a healthy gene (low or no determinable risk of developing a disease or disorder. The biomarker may indicate an edited gene. Exemplary biomarkers include, but are not limited to, single nucleotide polymorphisms (SNPs), sequence variations or mutations, epigenetic marks, splice acceptor sites, exogenous sequences, heterologous sequences, and any combination thereof.

A sequence motif of the disclosure may comprise or consist of a secondary, tertiary or quaternary structure. The secondary, tertiary or quaternary structure may be endogenous or naturally occurring. The secondary, tertiary or quaternary structure may be induced or non-naturally occurring. The secondary, tertiary or quaternary structure may be encoded by an endogenous, exogenous, or heterologous sequence.

In some embodiments of the compositions and methods of the disclosure, a target sequence of an RNA molecule comprises or consists of between 2 and 100 nucleotides or nucleic acid bases, inclusive of the endpoints. In some embodiments, the target sequence of an RNA molecule comprises or consists of between 2 and 50 nucleotides or nucleic acid bases, inclusive of the endpoints. In some embodiments, the target sequence of an RNA molecule comprises or consists of between 2 and 20 nucleotides or nucleic acid bases, inclusive of the endpoints.

In some embodiments of the compositions and methods of the disclosure, a target sequence of an RNA molecule is continuous. In some embodiments, the target sequence of an RNA molecule is discontinuous. For example, the target sequence of an RNA molecule may comprise or consist of one or more nucleotides or nucleic acid bases that are not contiguous because one or more intermittent nucleotides are positioned in between the nucleotides of the target sequence.

In some embodiments of the compositions and methods of the disclosure, a target sequence of an RNA molecule is naturally occurring. In some embodiments, the target sequence of an RNA molecule is non-naturally occurring. Exemplary non-naturally occurring target sequences may comprise or consist of sequence variations or mutations, chimeric sequences, exogenous sequences, heterologous sequences, chimeric sequences, recombinant sequences, sequences comprising a modified or synthetic nucleotide or any combination thereof.

In some embodiments of the compositions and methods of the disclosure, a target sequence of an RNA molecule binds to a guide RNA of the disclosure.

In some embodiments of the compositions and methods of the disclosure, a target sequence of an RNA molecule binds to a first RNA binding protein of the disclosure.

In some embodiments of the compositions and methods of the disclosure, a target sequence of an RNA molecule binds to a second RNA binding protein of the disclosure.

### RNA Molecules

In some embodiments of the compositions and methods of the disclosure, an RNA molecule of the disclosure comprises a target sequence. In some embodiments, the RNA molecule of the disclosure comprises at least one target sequence. In some embodiments, the RNA molecule of the disclosure comprises one or more target sequence(s). In some embodiments, the RNA molecule of the disclosure comprises two or more target sequences.

In some embodiments of the compositions and methods of the disclosure, an RNA molecule of the disclosure is a naturally occurring RNA molecule. In some embodiments, the RNA molecule of the disclosure is a non-naturally occurring molecule. Exemplary non-naturally occurring RNA molecules may comprise or consist of sequence variations or mutations, chimeric sequences, exogenous sequences, heterologous sequences, chimeric sequences, recombinant sequences, sequences comprising a modified or synthetic nucleotide or any combination thereof.

In some embodiments of the compositions and methods of the disclosure, an RNA molecule of the disclosure comprises or consists of a sequence isolated or derived from a virus.

In some embodiments of the compositions and methods of the disclosure, an RNA molecule of the disclosure comprises or consists of a sequence isolated or derived from a prokaryotic organism. In some embodiments, an RNA molecule of the disclosure comprises or consists of a sequence isolated or derived from a species or strain of archaea or a species or strain of bacteria.

In some embodiments of the compositions and methods of the disclosure, the RNA molecule of the disclosure comprises or consists of a sequence isolated or derived from a eukaryotic organism. In some embodiments, an RNA molecule of the disclosure comprises or consists of a sequence isolated or derived from a species of protozoa, parasite, protist, algae, fungi, yeast, amoeba, worm, microorganism, invertebrate, vertebrate, insect, rodent, mouse, rat, mammal, or a primate. In some embodiments, an RNA molecule of the disclosure comprises or consists of a sequence isolated or derived from a human.

In some embodiments of the compositions and methods of the disclosure, the RNA molecule of the disclosure comprises or consists of a sequence derived from a coding sequence from a genome of an organism or a virus. In some embodiments, the RNA molecule of the disclosure comprises or consists of a primary RNA transcript, a precursor messenger RNA (pre-mRNA) or messenger RNA (mRNA). In some embodiments, the RNA molecule of the disclosure comprises or consists of a gene product that has not been processed (e.g. a transcript). In some embodiments, the RNA molecule of the disclosure comprises or consists of a gene product that has been subject to post-transcriptional processing (e.g. a transcript comprising a 5′ cap and a 3′ polyadenylation signal). In some embodiments, the RNA molecule of the disclosure comprises or consists of a gene product that has been subject to alternative splicing (e.g. a splice variant). In some embodiments, the RNA molecule of the disclosure comprises or consists of a gene product that has been subject to removal of non-coding and/or intronic sequences (e.g. a messenger RNA (mRNA)).

In some embodiments of the compositions and methods of the disclosure, the RNA molecule of the disclosure comprises or consists of a sequence derived from a non-coding sequence (e.g. a non-coding RNA (ncRNA)). In some embodiments, the RNA molecule of the disclosure comprises or consists of a ribosomal RNA. In some embodiments, the RNA molecule of the disclosure comprises or consists of a small ncRNA molecule. Exemplary small RNA molecules of the disclosure include, but are not limited to, microRNAs (miRNAs), small interfering (siRNAs), piwi-interacting RNAs (piRNAs), small nucleolar RNAs (snoRNAs), small nuclear RNAs (snRNAs), extracellular or exosomal RNAs (exRNAs), and small Cajal body-specific RNAs (scaRNAs). In some embodiments, the RNA molecule of the disclosure comprises or consists of a long ncRNA molecule. Exemplary long RNA molecules of the disclosure include, but are not limited to, X-inactive specific transcript (Xist) and HOX transcript antisense RNA (HOTAIR).

In some embodiments of the compositions and methods of the disclosure, the RNA molecule of the disclosure contacted by a composition of the disclosure in an intracellular space. In some embodiments, the RNA molecule of the disclosure contacted by a composition of the disclosure in a cytosolic space. In some embodiments, the RNA molecule of the disclosure contacted by a composition of the disclosure in a nucleus. In some embodiments, the RNA molecule of the disclosure contacted by a composition of the disclosure in a vesicle, membrane-bound compartment of a cell, or an organelle.

In some embodiments of the compositions and methods of the disclosure, the RNA molecule of the disclosure contacted by a composition of the disclosure in an extracellular space. In some embodiments, the RNA molecule of the disclosure contacted by a composition of the disclosure in an exosome. In some embodiments, the RNA molecule of the disclosure contacted by a composition of the disclosure in a liposome, a polymersome, a micelle or a nanoparticle. In some embodiments, the RNA molecule of the disclosure contacted by a composition of the disclosure in an extracellular matrix. In some embodiments, the RNA molecule of the disclosure contacted by a composition of the disclosure in a droplet. In some embodiments, the RNA molecule of the disclosure contacted by a composition of the disclosure in a microfluidic droplet.

In some embodiments of the compositions and methods of the disclosure, a RNA molecule of the disclosure comprises or consists of a single-stranded sequence. In some embodiments, the RNA molecule of the disclosure comprises or consists of a double-stranded sequence. In some embodiments, the double-stranded sequence comprises two RNA molecules. In some embodiments, the double-stranded sequence comprises one RNA molecule and one DNA molecule. In some embodiments, including those wherein the double-stranded sequence comprises one RNA molecule and one DNA molecule, compositions of the disclosure selectively bind and, optionally, selectively cut the RNA molecule.

### Vectors

In some embodiments of the compositions and methods of the disclosure, a vector comprises a guide RNA of the disclosure. In some embodiments, the vector comprises at least one guide RNA of the disclosure. In some embodiments, the vector comprises one or more guide RNA(s) of the disclosure. In some embodiments, the vector comprises two or more guide RNAs of the disclosure. In some embodiments, the vector further comprises a fusion protein of the disclosure. In some embodiments, the fusion protein comprises a first RNA binding protein and a second RNA binding protein.

In some embodiments of the compositions and methods of the disclosure, a first vector comprises a guide RNA of the disclosure and a second vector comprises a fusion protein of the disclosure. In some embodiments, the first vector comprises at least one guide RNA of the disclosure. In some embodiments, the first vector comprises one or more guide RNA(s) of the disclosure. In some embodiments, the first vector comprises two or more guide RNA(s) of the disclosure. In some embodiments, the fusion protein comprises a first RNA binding protein and a second RNA binding protein. In some embodiments, the first vector and the second vector are identical. In some embodiments, the first vector and the second vector are not identical.

In some embodiments of the compositions and methods of the disclosure, the vector is or comprises a component of a “2-component RNA targeting system” comprising (a) nucleic acid sequence encoding a RNA-targeted fusion protein of the disclosure; and (b) a single guide RNA (sgRNA) sequence comprising: on its 5′ end, an RNA sequence (or spacer sequence) that hybridizes to or binds to a target RNA sequence; and on its 3′ end, an RNA sequence (or scaffold sequence) capable of binding to or associating with the CRISPR/Cas protein of the fusion protein; and wherein the 2-component RNA targeting system recognizes and alters the target RNA in a cell in the absence of a PAMmer. In some embodiments, the sequences of the 2-component system are in a single vector. In some embodiments, the spacer sequence of the 2-component system targets a repeat sequence selected from the group consisting of CUG, CCUG, CAG, and GGGGCC.

In some embodiments of the compositions and methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments of the compositions and methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11, or AAV12, or the vector and/or components are derived from a synthetic AAV serotype, such as, without limitation, Anc80 AAV (an ancestor of AAV 1, 2, 6, 8 and 9). In some embodiments, the viral vector is replication incompetent. In some embodiments, the viral vector is isolated or recombinant (rAAV). In some embodiments, the viral vector is self-complementary (scAAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term “recombinant expression system” refers to a genetic construct for the expression of certain genetic material formed by recombination.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. Non-limiting exemplary promoters include CMV, CBA, CAG, Cbh, EF-1a, PGK, UBC, GUSB, UCOE, hAAT, TBG, Desmin, MCK, C5-12, NSE, Synapsin, PDGF, MecP2, CaMKII, mGluR2, NFL, NFH, nβ2, PPE, ENK, EAAT2, GFAP, MBP, and U6 promoters. An “enhancer” is a region of DNA that can be bound by activating proteins to increase the likelihood or frequency of transcription. Non-limiting exemplary enhancers and posttranscriptional regulatory elements include the CMV enhancer and WPRE.

In some embodiments of the compositions and methods of the disclosure, an expression vector, viral vector or non-viral vector provided herein, includes without limitation, vector elements such as an IRES or 2A peptide sites for configuration of “multicistronic” or “polycistronic” or “bicistronic” or tricistronic” constructs, i.e., having double or triple or multiple coding areas or exons, and as such will have the capability to express from mRNA two or more proteins from a single construct. Multicistronic vectors simultaneously express two or more separate proteins from the same mRNA. The two strategies most widely used for constructing multicistronic configurations are through the use of an IRES or a 2A self-cleaving site. An “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” or “2A self-cleaving site” refer to linking sequences which are used within vector constructs to incorporate sites to promote ribosomal skipping and thus to generate two polypeptides from a single promoter, such self-cleaving peptides include without limitation, T2A, and P2A peptides or sequences encoding the self-cleaving peptides.

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers. In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 4.5 kb to 4.75 kb. In some embodiments, exemplary AAV vectors that may be used in any of the herein described compositions, systems, methods, and kits can include an AAV1 vector, a modified AAV1 vector, an AAV2 vector, a modified AAV2 vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh64R1 vector, and a modified AAV.rh64R1 vector and any combinations or equivalents thereof. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Lentiviral vectors are well-known in the art (see, e.g., Trono D. (2002) Lentiviral vectors, New York: Spring-Verlag Berlin Heidelberg and Durand et al. (2011) Viruses 3(2):132-159 doi: 10.3390/v3020132). In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIV_{SM}) vector, a modified sooty mangabey simian immunodeficiency virus (SIV_{SM}) vector, a African green monkey simian immunodeficiency virus (SIV_{AGM}) vector, a modified African green monkey simian immunodeficiency virus (SIV_{AGM}) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer.

### Nucleic Acids

Provided herein are the nucleic acid sequences encoding the fusion proteins disclosed herein for use in gene transfer and expression techniques described herein. It should be understood, although not always explicitly stated that the sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

The nucleic acid sequences (e.g., polynucleotide sequences) disclosed herein may be codon-optimized which is a technique well known in the art. In some embodiments disclosed herein, exemplary Cas sequences, such as e.g., SEQ ID NO: 46 (Cas13d), are codon optimized for expression in human cells. Codon optimization refers to the fact that different cells differ in their usage of particular codons. This codon bias corresponds to a bias in the relative abundance of particular tRNAs in the cell type. By altering the codons in the sequence to match with the relative abundance of corresponding tRNAs, it is possible to increase expression. It is also possible to decrease expression by deliberately choosing codons for which the corresponding tRNAs are known to be rare in a particular cell type. Codon usage tables are known in the art for mammalian cells, as well as for a variety of other organisms. Based on the genetic code, nucleic acid sequences coding for, e.g., a Cas protein, can be generated. In some embodiments, such a sequence is optimized for expression in a host or target cell, such as a host cell used to express the Cas protein or a cell in which the disclosed methods are practiced (such as in a mammalian cell, e.g., a human cell). Codon preferences and codon usage tables for a particular species can be used to engineer isolated nucleic acid molecules encoding a Cas protein (such as one encoding a protein having at least 80%, at least 85%, at least 90%, at least 92%, at least 95%, at least 96%, at least 97%, at least 98%, at least 99%, or 100% sequence identity to its corresponding wild-type protein) that takes advantage of the codon usage preferences of that particular species. For example, the Cas proteins disclosed herein can be designed to have codons that are preferentially used by a particular organism of interest. In one example, an Cas nucleic acid sequence is optimized for expression in human cells, such as one having at least 70%, at least 80%, at least 85%, at least 90%, at least 92%, at least 95%, at least 98%, or at least 99% sequence identity to its corresponding wild-type or originating nucleic acid sequence. In some embodiments, an isolated nucleic acid molecule encoding at least one Cas protein (which can be part of a vector) includes at least one Cas protein coding sequence that is codon optimized for expression in a eukaryotic cell, or at least one Cas protein coding sequence codon optimized for expression in a human cell. In one embodiment, such a codon optimized Cas coding sequence has at least 80%, at least 85%, at least 90%, at least 92%, at least 95%, at least 96%, at least 97%, at least 98%, at least 99%, or 100% sequence identity to its corresponding wild-type or originating sequence. In another embodiment, a eukaryotic cell codon optimized nucleic acid sequence encodes a Cas protein having at least 85%, at least 90%, at least 92%, at least 95%, at least 96%, at least 97%, at least 98%, at least 99%, or 100% sequence identity to its corresponding wild-type or originating protein. In another embodiment, a variety of clones containing functionally equivalent nucleic acids may be routinely generated, such as nucleic acids which differ in sequence but which encode the same Cas protein sequence. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W.H. 5 Freeman and Co., NY).

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

### Cells

In some embodiments of the compositions and methods of the disclosure, a cell of the disclosure is a prokaryotic cell.

In some embodiments of the compositions and methods of the disclosure, a cell of the disclosure is a eukaryotic cell. In some embodiments, a cell of the disclosure is a somatic cell. In some embodiments, a cell of the disclosure is a germline cell. In some embodiments, a germline cell of the disclosure is not a human cell.

In some embodiments of the compositions and methods of the disclosure, a cell of the disclosure is a stem cell. In some embodiments, a cell of the disclosure is an embryonic stem cell. In some embodiments, an embryonic stem cell of the disclosure is not a human cell. In some embodiments, a cell of the disclosure is a multipotent stem cell or a pluripotent stem cell. In some embodiments, a cell of the disclosure is an adult stem cell. In some embodiments, a cell of the disclosure is an induced pluripotent stem cell (iPSC). In some embodiments, a cell of the disclosure is a hematopoietic stem cell (HSC).

In some embodiments of the compositions and methods of the disclosure, a somatic cell of the disclosure is an immune cell. In some embodiments, an immune cell of the disclosure is a lymphocyte. In some embodiments, an immune cell of the disclosure is a T lymphocyte (also referred to herein as a T-cell). Exemplary T-cells of the disclosure include, but are not limited to, naïve T cells, effector T cells, helper T cells, memory T cells, regulatory T cells (Tregs) and Gamma delta T cells. In some embodiments, an immune cell of the disclosure is a B lymphocyte. In some embodiments, an immune cell of the disclosure is a natural killer cell. In some embodiments, an immune cell of the disclosure is an antigen-presenting cell.

In some embodiments of the compositions and methods of the disclosure, a somatic cell of the disclosure is a muscle cell. In some embodiments, a muscle cell of the disclosure is a myoblast or a myocyte. In some embodiments, a muscle cell of the disclosure is a cardiac muscle cell, skeletal muscle cell or smooth muscle cell. In some embodiments, a muscle cell of the disclosure is a striated cell.

In some embodiments of the compositions and methods of the disclosure, a somatic cell of the disclosure is an epithelial cell. In some embodiments, an epithelial cell of the disclosure forms a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the disclosure forms a gland including, but not limited to, a pineal gland, a thymus gland, a pituitary gland, a thyroid gland, an adrenal gland, an apocrine gland, a holocrine gland, a merocrine gland, a serous gland, a mucous gland and a sebaceous gland. In some embodiments, an epithelial cell of the disclosure contacts an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the disclosure contacts an outer surface of a blood vessel or a vein.

In some embodiments of the compositions and methods of the disclosure, a somatic cell of the disclosure is a neuronal cell. In some embodiments, a neuron cell of the disclosure is a neuron of the central nervous system. In some embodiments, a neuron cell of the disclosure is a neuron of the brain or the spinal cord. In some embodiments, a neuron cell of the disclosure is a neuron of the retina. In some embodiments, a neuron cell of the disclosure is a neuron of a cranial nerve or an optic nerve. In some embodiments, a neuron cell of the disclosure is a neuron of the peripheral nervous system. In some embodiments, a neuron cell of the disclosure is a neuroglial or a glial cell. In some embodiments, a glial of the disclosure is a glial cell of the central nervous system including, but not limited to, oligodendrocytes, astrocytes, ependymal cells, and microglia. In some embodiments, a glial of the disclosure is a glial cell of the peripheral nervous system including, but not limited to, Schwann cells and satellite cells.

In some embodiments of the compositions and methods of the disclosure, a somatic cell of the disclosure is a primary cell.

In some embodiments of the compositions and methods of the disclosure, a somatic cell of the disclosure is a cultured cell.

In some embodiments of the compositions and methods of the disclosure, a somatic cell of the disclosure is in vivo, in vitro, ex vivo or in situ.

In some embodiments of the compositions and methods of the disclosure, a somatic cell of the disclosure is autologous or allogeneic.

### Methods of Use

The disclosure provides a method of modifying level of expression of an RNA molecule of the disclosure or a protein encoded by the RNA molecule comprising contacting the composition and the RNA molecule under conditions suitable for binding of one or more of the guide RNA or the RNA-binding protein or fusion protein thereof (or a portion thereof) to the RNA molecule and providing immune masking activity specific to the RNA-binding protein.

The disclosure provides a method of modifying an activity of a protein encoded by an RNA molecule comprising contacting the composition and the RNA molecule under conditions suitable for binding of one or more of the guide RNA or the fusion protein (or a RNA-binding portion thereof) to the RNA molecule and providing immune masking activity specific to the RNA-binding protein.

The disclosure provides a method of modifying level of expression of an RNA molecule of the disclosure or a protein encoded by the RNA molecule comprising contacting the composition and a cell comprising the RNA molecule under conditions suitable for binding of one or more of the guide RNA or the RNA-binding protein or fusion protein thereof (or a portion thereof) to the RNA molecule and providing immune masking activity specific to the RNA-binding protein. In some embodiments, the cell is in vivo, in vitro, ex vivo or in situ. In some embodiments, the composition comprises a vector comprising composition comprising a guide RNA of the disclosure and a fusion protein of the disclosure. In some embodiments, the vector is an AAV.

The disclosure provides a method of modifying an activity of a protein encoded by an RNA molecule comprising contacting the composition and a cell comprising the RNA molecule under conditions suitable for binding of one or more of the guide RNA or the RNA-binding protein or fusion protein thereof (or a portion thereof) to the RNA molecule and providing immune masking activity specific to the RNA-binding protein. In some embodiments, the cell is in vivo, in vitro, ex vivo or in situ. In some embodiments, the composition comprises a vector comprising composition comprising a guide RNA or a single guide RNA of the disclosure and a fusion protein of the disclosure. In some embodiments, the vector is an AAV.

The disclosure provides a method of modifying level of expression of an RNA molecule of the disclosure or a protein encoded by the RNA molecule comprising contacting the composition and the RNA molecule under conditions suitable for RNA nuclease activity wherein the RNA-binding protein or fusion protein thereof or portion thereof induces a break in the RNA molecule and provides immune masking activity specific to the RNA-binding protein.

The disclosure provides a method of modifying an activity of a protein encoded by an RNA molecule comprising contacting the composition and the RNA molecule under conditions suitable for RNA nuclease activity wherein the RNA-binding protein or fusion protein thereof (or a portion thereof) induces a break to the RNA molecule and provides immune masking activity specific to the RNA-binding protein.

The disclosure provides a method of modifying a level of expression of an RNA molecule of the disclosure or a protein encoded by the RNA molecule and provides immune masking activity specific to the RNA-binding protein comprising contacting the composition and a cell comprising the RNA molecule under conditions suitable for RNA nuclease activity wherein the RNA-binding protein or fusion protein thereof induces a break in the RNA molecule. In some embodiments, the cell is in vivo, in vitro, ex vivo or in situ. In some embodiments, the composition comprises a vector comprising composition comprising a guide RNA of the disclosure and an RNA-binding protein of the disclosure and a mutated non-cleavable FasL of the disclosure. In some embodiments, the vector is an AAV.

The disclosure provides a method of modifying an activity of a protein encoded by an RNA molecule comprising contacting the composition and a cell comprising the RNA molecule under conditions suitable for RNA nuclease activity wherein the RNA-binding protein or fusion protein thereof or portion thereof induces a break in the RNA molecule. In some embodiments, the cell is in vivo, in vitro, ex vivo or in situ. In some embodiments, the composition comprises a vector comprising composition comprising a guide RNA sequence or a single guide RNA of the disclosure and a sequence encoding an RNA-binding protein of the disclosure and sequence encoding a mutated non-cleavable FasL of the disclosure. In some embodiments, the vector is an AAV.

The disclosure provides a method of treating a disease or disorder comprising administering to a subject a therapeutically effective amount of a composition of the disclosure.

The disclosure provides a method of treating a disease or disorder comprising administering to a subject a therapeutically effective amount of a composition of the disclosure, wherein the composition comprises a vector comprising a guide RNA sequence of the disclosure, a sequence encoding an RNA-binding protein of the disclosure, and a sequence encoding a mutated non-cleavable FasL of the disclosure, and wherein the composition modifies a level of expression of an RNA molecule of the disclosure or a protein encoded by the RNA molecule and provides immune masking activity specific to the RNA-binding protein.

The disclosure provides a method of treating a disease or disorder comprising administering to a subject a therapeutically effective amount of a composition of the disclosure, wherein the composition comprises a vector comprising composition comprising a compositions of the disclosure.

In some embodiments of the compositions and methods of the disclosure, a disease or disorder of the disclosure includes, but is not limited to, a genetic disease or disorder. In some embodiments, the genetic disease or disorder is a single-gene disease or disorder. In some embodiments, the single-gene disease or disorder is an autosomal dominant disease or disorder, an autosomal recessive disease or disorder, an X-chromosome linked (X-linked) disease or disorder, an X-linked dominant disease or disorder, an X-linked recessive disease or disorder, a Y-linked disease or disorder or a mitochondrial disease or disorder. In some embodiments, the genetic disease or disorder is a multiple-gene disease or disorder. In some embodiments, the genetic disease or disorder is a multiple-gene disease or disorder. In some embodiments, the single-gene disease or disorder is an autosomal dominant disease or disorder including, but not limited to, Huntington's disease, neurofibromatosis type 1, neurofibromatosis type 2, Marfan syndrome, hereditary nonpolyposis colorectal cancer, hereditary multiple exostoses, Von Willebrand disease, and acute intermittent porphyria. In some embodiments, the single-gene disease or disorder is an autosomal recessive disease or disorder including, but not limited to, Albinism, Medium-chain acyl-CoA dehydrogenase deficiency, cystic fibrosis, sickle-cell disease, Tay-Sachs disease, Niemann-Pick disease, spinal muscular atrophy, and Roberts syndrome. In some embodiments, the single-gene disease or disorder is X-linked disease or disorder including, but not limited to, muscular dystrophy, Duchenne muscular dystrophy, Hemophilia, Adrenoleukodystrophy (ALD), Rett syndrome, and Hemophilia A. In some embodiments, the single-gene disease or disorder is a mitochondrial disorder including, but not limited to, Leber's hereditary optic neuropathy.

In some embodiments of the compositions and methods of the disclosure, a disease or disorder of the disclosure includes, but is not limited to, an immune disease or disorder. In some embodiments, the immune disease or disorder is an immunodeficiency disease or disorder including, but not limited to, B-cell deficiency, T-cell deficiency, neutropenia, asplenia, complement deficiency, acquired immunodeficiency syndrome (AIDS) and immunodeficiency due to medical intervention (immunosuppression as an intended or adverse effect of a medical therapy). In some embodiments, the immune disease or disorder is an autoimmune disease or disorder including, but not limited to, Achalasia, Addison's disease, Adult Still's disease, Agammaglobulinemia, Alopecia areata, Amyloidosis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, or Wegener's granulomatosis.

In some embodiments of the compositions and methods of the disclosure, a disease or disorder of the disclosure includes, but is not limited to, an inflammatory disease or disorder.

In some embodiments of the compositions and methods of the disclosure, a disease or disorder of the disclosure includes, but is not limited to, a metabolic disease or disorder.

In some embodiments of the compositions and methods of the disclosure, a disease or disorder of the disclosure includes, but is not limited to, a degenerative or a progressive disease or disorder. In some embodiments, the degenerative or a progressive disease or disorder includes, but is not limited to, amyotrophic lateral sclerosis (ALS), Huntington's disease, Alzheimer's disease, and aging.

In some embodiments of the compositions and methods of the disclosure, a disease or disorder of the disclosure includes, but is not limited to, an infectious disease or disorder.

In some embodiments of the compositions and methods of the disclosure, a disease or disorder of the disclosure includes, but is not limited to, a pediatric or a developmental disease or disorder.

In some embodiments of the compositions and methods of the disclosure, a disease or disorder of the disclosure includes, but is not limited to, a cardiovascular disease or disorder.

In some embodiments of the compositions and methods of the disclosure, a disease or disorder of the disclosure includes, but is not limited to, a proliferative disease or disorder. In some embodiments, the proliferative disease or disorder is a cancer. In some embodiments, the cancer includes, but is not limited to, Acute Lymphoblastic Leukemia (ALL), Acute Myeloid Leukemia (AML), Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Gastrointestinal Carcinoid Tumors, Astrocytomas, Atypical Teratoid/Rhabdoid Tumor, Central Nervous System (Brain Cancer), Basal Cell Carcinoma, Bile Duct Cancer, Bladder Cancer, Bone Cancer, Ewing Sarcoma, Osteosarcoma, Malignant Fibrous Histiocytoma, Brain Tumors, Breast Cancer, Burkitt Lymphoma, Carcinoid Tumor, Carcinoma, Cardiac (Heart) Tumors, Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CIVIL), Chronic Myeloproliferative Neoplasms, Colorectal Cancer, Craniopharyngioma, Cutaneous T-Cell Lymphoma, Ductal Carcinoma In Situ, Embryonal Tumors, Endometrial Cancer (Uterine Cancer), Ependymoma, Esophageal Cancer, Esthesioneuroblastoma (Head and Neck Cancer), Ewing Sarcoma (Bone Cancer), Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST) (Soft Tissue Sarcoma), Childhood Gastrointestinal Stromal Tumors, Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis, Hodgkin Lymphoma, Hypopharyngeal Cancer (Head and Neck Cancer), Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Leukemia, Lip and Oral Cavity Cancer (Head and Neck Cancer), Liver Cancer, Lung Cancer (Non-Small Cell and Small Cell), Childhood Lung Cancer, Lymphoma, Male Breast Cancer, Malignant Fibrous Histiocytoma of Bone and Osteosarcoma, Melanoma, Merkel Cell Carcinoma (Skin Cancer), Mesothelioma, Metastatic Squamous Neck Cancer with Occult Primary (Head and Neck Cancer), Midline Tract Carcinoma With NUT Gene Changes, Mouth Cancer (Head and Neck Cancer), Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer, Lip and Oral Cavity Cancer and Oropharyngeal Cancer, Osteosarcoma and Malignant Fibrous Histiocytoma of Bone, Ovarian Cancer, Pancreatic Cancer, Pancreatic Neuroendocrine Tumors (Islet Cell Tumors), Papillomatosis, Paraganglioma, Parathyroid Cancer, Penile Cancer, Pharyngeal Cancer (Head and Neck Cancer), Pheochromocytoma, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Childhood (Soft Tissue Sarcoma), Salivary Gland Cancer (Head and Neck Cancer), Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Uterine Sarcoma, Sézary Syndrome, Lymphoma, Skin Cancer, Small Cell Lung Cancer, Small Intestine Cancer, Soft Tissue Sarcoma, Squamous Cell Carcinoma of the Skin, Squamous Neck Cancer, Stomach (Gastric) Cancer, T-Cell Lymphoma, Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter, Renal Cell Cancer, Urethral Cancer, Uterine Sarcoma, Vaginal Cancer, Vascular Tumors (Soft Tissue Sarcoma), Vulvar Cancer, Wilms Tumor and Other Childhood Kidney Tumors.

In some embodiments of the methods of the disclosure, a subject of the disclosure has been diagnosed with the disease or disorder. In some embodiments, the subject of the disclosure presents at least one sign or symptom of the disease or disorder. In some embodiments, the subject has a biomarker predictive of a risk of developing the disease or disorder. In some embodiments, the biomarker is a genetic mutation.

In some embodiments of the methods of the disclosure, a subject of the disclosure is female. In some embodiments of the methods of the disclosure, a subject of the disclosure is male. In some embodiments, a subject of the disclosure has two XX or XY chromosomes. In some embodiments, a subject of the disclosure has two XX or XY chromosomes and a third chromosome, either an X or a Y.

In some embodiments of the methods of the disclosure, a subject of the disclosure is a neonate, an infant, a child, an adult, a senior adult, or an elderly adult. In some embodiments of the methods of the disclosure, a subject of the disclosure is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days old. In some embodiments of the methods of the disclosure, a subject of the disclosure is at least 1, 2, 3, 4, 5, 6, 7, 8, 9, 10, 11 or 12 months old. In some embodiments of the methods of the disclosure, a subject of the disclosure is at least 1, 2, 3, 4, 5, 6, 7, 8, 9, 10, 15, 20, 25, 30, 35, 40, 45, 50, 55, 60, 65, 70, 75, 80, 85, 90, 95, 100 or any number of years or partial years in between of age.

In some embodiments of the methods of the disclosure, a subject of the disclosure is a mammal. In some embodiments, a subject of the disclosure is a non-human mammal.

In some embodiments of the methods of the disclosure, a subject of the disclosure is a human.

In some embodiments of the methods of the disclosure, a therapeutically effective amount comprises a single dose of a composition of the disclosure. In some embodiments, a therapeutically effective amount comprises a therapeutically effective amount comprises at least one dose of a composition of the disclosure. In some embodiments, a therapeutically effective amount comprises a therapeutically effective amount comprises one or more dose(s) of a composition of the disclosure.

In some embodiments of the methods of the disclosure, a therapeutically effective amount eliminates a sign or symptom of the disease or disorder. In some embodiments, a therapeutically effective amount reduces a severity of a sign or symptom of the disease or disorder.

In some embodiments of the methods of the disclosure, a therapeutically effective amount eliminates the disease or disorder.

In some embodiments of the methods of the disclosure, a therapeutically effective amount prevents an onset of a disease or disorder. In some embodiments, a therapeutically effective amount delays the onset of a disease or disorder. In some embodiments, a therapeutically effective amount reduces the severity of a sign or symptom of the disease or disorder. In some embodiments, a therapeutically effective amount improves a prognosis for the subject.

In some embodiments of the methods of the disclosure, a composition of the disclosure is administered to the subject systemically. In some embodiments, the composition of the disclosure is administered to the subject by an intravenous route. In some embodiments, the composition of the disclosure is administered to the subject by an injection or an infusion.

In some embodiments of the methods of the disclosure, a composition of the disclosure is administered to the subject locally. In some embodiments, the composition of the disclosure is administered to the subject by an intraosseous, intraocular, intracerebrospinal or intraspinal route. In some embodiments, the composition of the disclosure is administered directly to the cerebral spinal fluid of the central nervous system. In some embodiments, the composition of the disclosure is administered directly to a tissue or fluid of the eye and does not have bioavailability outside of ocular structures. In some embodiments, the composition of the disclosure is administered to the subject by an injection or an infusion.

### Numbered Embodiments

1. A composition comprising:

(a) a sequence encoding a non-self polypeptide of interest (POI), and

(b) a sequence encoding a non-cleavable Fas Ligand (FASL),

wherein expression of the non-cleavable FASL eliminates MHC-mediated immunogenic peptides and helper T cells specific to the expression of the POI.

2. A composition comprising:

(a) a sequence encoding a non-self polypeptide, and
(b) a sequence encoding a non-cleavable FASL,
wherein expression of the non-cleavable FASL selectively eliminates a T-cell that recognizes a MHC-peptide complex, wherein the peptide is derived from the non-self polypeptide.

3. A composition comprising:

(a) a sequence encoding a therapeutic polypeptide, and
(b) a sequence encoding a non-cleavable FASL,
wherein expression of the non-cleavable FASL selectively eliminates a T-cell that recognizes a MHC-peptide complex, wherein the peptide is derived from the therapeutic polypeptide.

4. A composition comprising an adeno-associated virus (AAV) vector comprising:

a sequence encoding an AAV capsid polypeptide, and
a composition comprising
(a) a sequence encoding a human polypeptide, and
(b) a sequence encoding a non-cleavable FASL,
wherein expression of the non-cleavable FASL selectively eliminates a T-cell that recognizes a MHC-peptide complex, wherein the peptide is derived from the human polypeptide and/or the AAV capsid polypeptide.

5. The composition of embodiment 4, wherein the human polypeptide is a self polypeptide and wherein the peptide is derived from the AAV capsid polypeptide.

6. A composition comprising:

(a) a sequence comprising a guide RNA (gRNA) that specifically binds a target sequence within an RNA molecule,
(b) a sequence encoding an RNA-binding polypeptide, and
(c) a sequence encoding a non-cleavable FASL,

wherein expression of the non-cleavable FASL selectively eliminates a T-cell that recognizes a MHC-peptide complex, wherein the peptide is derived from the RNA-binding polypeptide.

7. The composition of any one of embodiments 1-5, wherein a vector comprises the sequence of (a) and the sequence of (b).

8. The composition of embodiment 6, wherein a vector comprises the sequence of (a), the sequence of (b) and the sequence of (c).

9. The composition of embodiment 7 or 8, wherein the vector is an expression vector.

10. The composition of embodiment 9, wherein the expression vector is a plasmid.

11. The composition of any one of embodiments 1-10, wherein a promoter drives expression of the sequence of (a).

12. The composition of any one of embodiments 1-5 and 7-11, wherein the promoter drives expression of the sequence of (b).

13. The composition of embodiment 6, wherein a first promoter drives expression of the sequence of (a) and a second promoter drives expression of the sequence of (b).

14. The composition of embodiment 13, wherein the second promoter drives expression of the sequence of (b) and the sequence of (c).

15. The composition of embodiment 11, wherein a first promoter drives expression of the sequence of (a) and a second promoter drives expression of the sequence of (b).

16. The composition of any one of embodiments 1-15, wherein one or more sequence(s) encoding the promoter comprises a sequence isolated or derived from a U6 promoter.

17. The composition of any one of embodiments 1-15, wherein one or more sequence(s) encoding the promoter comprises a sequence isolated or derived from a promoter capable of diving expression of a transfer RNA (tRNA).

18. The composition of embodiment 17, wherein the sequence encoding the promoter comprises a sequence isolated or derived from an alanine tRNA promoter, an arginine tRNA promoter, an asparagine tRNA promoter, an aspartic acid tRNA promoter, a cysteine tRNA promoter, a glutamine tRNA promoter, a glutamic acid tRNA promoter, a glycine tRNA promoter, a histidine tRNA promoter, an isoleucine tRNA promoter, a leucine tRNA promoter, a lysine tRNA promoter, a methionine tRNA promoter, a phenylalanine tRNA promoter, a proline tRNA promoter, a serine tRNA promoter, a threonine tRNA promoter, a tryptophan tRNA promoter, a tyrosine tRNA promoter, or a valine tRNA promoter.

19. The composition of embodiment 17, wherein the sequence encoding the promoter comprises a sequence isolated or derived from a valine tRNA promoter.

20. The composition of any one of embodiment 1-3 or 6-19, wherein a delivery vector comprises the composition.

21. The composition of embodiment 20, wherein the delivery vector is an adeno-associated viral (AAV) vector.

22. The composition of embodiment 20, wherein the AAV comprises a sequence isolated or derived from an AAV of serotype AAV1, AAV2, AAV3, AAV4, AAV5, AAV6, AAV7, AAV8, AAV9, AAV10, AAV11, or AAV12.

23. The composition of embodiment 4 or 5, wherein the AAV comprises a sequence isolated or derived from an AAV of serotype AAV1, AAV2, AAV3, AAV4, AAV5, AAV6, AAV7, AAV8, AAV9, AAV10, AAV11, or AAV12.

24. The composition of any one of embodiments 1-5, 7-14 or 16-23, wherein the sequence of (a) or the sequence of (b) further comprises a sequence encoding an Internal Ribosomal Entry Site (IRES) or a sequence encoding a self-cleaving peptide.

25. The composition of embodiment 6, wherein the sequence of (b) or the sequence of (c) further comprises a sequence encoding IRES or a sequence encoding a self-cleaving peptide.

26. The composition of any one of embodiments 8-23, wherein the vector comprises a sequence encoding IRES or a sequence encoding a self-cleaving peptide.

27. The composition of embodiment 24 or 26, wherein the sequence encoding IRES or the sequence encoding a self-cleaving peptide is positioned between the sequence of (a) and the sequence of (b).

28. The composition of embodiment 25 or 26, wherein the sequence encoding IRES or the sequence encoding a self-cleaving peptide is positioned between the sequence of (b) and the sequence of (c).

29. The composition of any one of embodiments 24-28, wherein the self-cleaving peptide comprises a 2A self-cleaving peptide.

30. The composition of any one of embodiments 1-29, wherein the non-cleavable FASL comprises a mutation in a metalloproteinase cleavage site.

31. The composition of embodiment 30, wherein the metalloproteinase cleavage site comprises the amino acid sequence ELAELR.

32. The composition of embodiment 31, wherein the mutation comprises one or more of a substitution, an insertion, a deletion, a frameshift, an inversion, or a transposition of the amino acid sequence ELAELR.

33. The composition of any one of embodiments 30-32, wherein the non-cleavable FASL comprises the amino acid sequence of:

(SEQ ID NO: 210)
MQQPFNYPYPQIYWVDSSASSPWAPPGTVLPCPTSVPRRPGQRRPPPPP
PPPPLPPPPPPPPLPPLPLPPLKKRGNHSTGLCLLVMFFMVLVALVGLG
LGMFQLFHLQKX1X2X3X4X5X6ESTSQMHTASSLEKQIGHPSPPPEKK
ELRKVAHLTGKSNSRSMPLEWEDTYGIVLLSGVKYKKGGLVINETGLYF
VYSKVYFRGQSCNNLPLSHKVYMRNSKYPQDLVMMEGKMMSYCTTGQMW
ARSSYLGAVFNLTSADHLYVNVSELSLVNFEESQTFFGLYKL,

wherein X1 is not a glutamic acid (E), X2 is not an leucine (L), X3 is not an alanine (A), X4 is not an glutamic acid (E), X5 is not an leucine (L) or X6 is not an arginine (R).

34. The composition of any one of embodiments 30-32, wherein the non-cleavable FASL comprises the amino acid sequence of:

(SEQ ID NO: 210)
MQQPFNYPYPQIYWVDSSASSPWAPPGTVLPCPTSVPRRPGQRRPPPPP
PPPPLPPPPPPPPLPPLPLPPLKKRGNHSTGLCLLVMFFMVLVALVGLG
LGMFQLFHLQKX1X2X3X4X5X6ESTSQMHTASSLEKQIGHPSPPPEKK
ELRKVAHLTGKSNSRSMPLEWEDTYGIVLLSGVKYKKGGLVINETGLYF
VYSKVYFRGQSCNNLPLSHKVYMRNSKYPQDLVMMEGKMMSYCTTGQMW
ARSSYLGAVFNLTSADHLYVNVSELSLVNFEESQTFFGLYKL,

wherein X1 is not a glutamic acid (E), X2 is not an leucine (L), X3 is not an alanine (A), X4 is not an glutamic acid (E), X5 is not an leucine (L) and X6 is not an arginine (R).

35. The composition of embodiment 6, wherein the sequence comprising the gRNA further comprises a spacer sequence that specifically binds to the target RNA sequence.

36. The composition of embodiment 35, wherein the spacer sequence has at least 50%, 55%, 60%, 65%, 70%, 75%, 80%, 87%, 90%, 95%, 97%, 99% or any percentage in between of complementarity to the target RNA sequence.

37. The composition of embodiment 35, wherein the spacer sequence has 100% complementarity to the target RNA sequence.

38. The composition of any one of embodiments 35-37, wherein the spacer sequence comprises or consists of 20 nucleotides.

39. The composition of any one of embodiments 35-37, wherein the spacer sequence comprises or consists of 21 nucleotides.

40. The composition of embodiment 39, wherein the spacer sequence comprises the sequence UGGAGCGAGCAUCCCCCAAA (SEQ ID NO: 1), GUUUGGGGGAUGCUCGCUCCA (SEQ ID NO: 2), CCCUCACUGCUGGGGAGUCC (SEQ ID NO: 3), GGACUCCCCAGCAGUGAGGG (SEQ ID NO: 4), GCAACUGGAUCAAUUUGCUG (SEQ ID NO: 5), GCAGCAAAUUGAUCCAGUUGC (SEQ ID NO: 6), GCAUUCUUAUCUGGUCAGUGC (SEQ ID NO: 7), GCACUGACCAGAUAAGAAUG (SEQ ID NO: 8), GAGCAGCAGCAGCAGCAGCAG (SEQ ID NO: 9), GCAGGCAGGCAGGCAGGCAGG (SEQ ID NO: 10), GCCCCGGCCCCGGCCCCGGC (SEQ ID NO: 11), or GCTGCTGCTGCTGCTGCTGC (SEQ ID NO: 12), GGGGCCGGGGCCGGGGCCGG (SEQ ID NO: 74), GGGCCGGGGCCGGGGCCGGG (SEQ ID NO: 75), GGCCGGGGCCGGGGCCGGGG (SEQ ID NO: 76), GCCGGGGCCGGGGCCGGGGC (SEQ ID NO: 77), CCGGGGCCGGGGCCGGGGCC (SEQ ID NO: 78), CGGGGCCGGGGCCGGGGCCG (SEQ ID NO: 79).

41. The composition of any one of embodiments 6, 11, 13-14, 17-23, 25, and 28-40, wherein the sequence comprising the gRNA further comprises a scaffold sequence that specifically binds to the RNA binding protein.

42. The composition of embodiment 41, wherein the scaffold sequence comprises a stem-loop structure.

43. The composition of embodiment 41 or 42, wherein the scaffold sequence comprises or consists of 90 nucleotides.

44. The composition of embodiment 41 or 42, wherein the scaffold sequence comprises or consists of 93 nucleotides.

45. The composition of embodiment 44, wherein the scaffold sequence comprises the sequence

(SEQ ID NO: 13)
GUUUAAGAGCUAUGCUGGAAACAGCAUAGCAAGUUUAAAUAAGGCUAGUC
CGUUAUCAACUUGAAAAAGUGGCACCGAGUCGGUGCUUUUUUU.

46. The composition of embodiment 45, wherein the spacer sequence comprises the sequence GUGAUAAGUGGAAUGCCAUG (SEQ ID NO: 14), CUGGUGAACUUCCGAUAGUG (SEQ ID NO: 15), or GAGATATAGCCTGGTGGTTC (SEQ ID NO: 16).

47. The composition of embodiment 41 or 42, wherein the scaffold sequence comprises or consists of 85 nucleotides.

48. The composition of embodiment 47, wherein the scaffold sequence comprises the sequence

(SEQ ID NO: 17)
GGACAGCAUAGCAAGUUAAAAUAAGGCUAGUCCGUUAUCAACUUGAAAAA
GUGGCACCGAGUCGGUGCUUUUU.

49. The composition of embodiment 48, wherein the spacer sequence comprises the sequence at least 1, 2, 3, 4, 5, 6, or 7 repeats of the sequence CUG (SEQ ID NO: 18), CCUG (SEQ ID NO: 19), CAG (SEQ ID NO: 80), GGGGCC (SEQ ID NO: 81) or any combination thereof.

50. The composition of embodiment 41 or 42, wherein the scaffold sequence comprises the sequence

(SEQ ID NO: 82)
GUUUAAGAGCUAUGCUGGAAACAGCAUAGCAAGUUUAAAUAAGGCUAGUC
CGUUAUCAACUUGAAAAAGUGGCACCGAGUCGGUGCUUUUUUU
or
(SEQ ID NO: 83)
GUUUUAGAGCUAGAAAUAGCAAGUUAAAAUAAGGCUAGUCCGUUAUCAAC
UUGAAAAAGUGGCACCGAGUCGGUGCUUUUUUU.

51. The composition of any one of embodiments 6, 11, 13-14, 17-23, 25, and 28-50, wherein the gRNA does not bind or does not selectively bind to a second sequence within the RNA molecule.

52. The composition of any one of embodiments 6, 11, 13-14, 17-23, 25, and 28-51, wherein an RNA genome or an RNA transcriptome comprises the RNA molecule.

53. The composition of any one of embodiments 6, 11, 13-14, 17-23, 25, and 28-52, wherein the RNA-binding polypeptide is selected from the group consisting of CRISPR-Cas, PUF, Pumilio, and PPR.

54. The composition of embodiment 53, wherein a fusion protein comprises the RNA-binding polypeptide.

55. The composition of embodiment 54, wherein the fusion protein comprises a sequence encoding a first RNA-binding polypeptide and a sequence encoding a second RNA-binding polypeptide,

wherein neither the first RNA-binding polypeptide nor the second RNA-binding polypeptide comprises a significant DNA-nuclease activity,
wherein the first RNA-binding polypeptide and the second RNA-binding polypeptide are not identical, and
wherein the second RNA-binding polypeptide comprises an RNA-nuclease activity.

56. The composition embodiment 55, wherein the first RNA binding protein comprises a CRISPR-Cas protein.

57. The composition of embodiment 56, wherein the CRISPR-Cas protein is a Type II CRISPR-Cas protein.

58. The composition of embodiment 57, wherein the first RNA binding protein comprises a Cas9 polypeptide or an RNA-binding portion thereof.

59. The composition of embodiment 56, wherein the CRISPR-Cas protein is a Type V CRISPR-Cas protein.

60. The composition of embodiment 59, wherein the first RNA binding protein comprises a Cpf1 polypeptide or an RNA-binding portion thereof.

61. The composition of embodiment 56, wherein the CRISPR-Cas protein is a Type VI CRISPR-Cas protein.

62. The composition of embodiment 61, wherein the first RNA binding protein comprises a Cas13 polypeptide or an RNA-binding portion thereof.

63. The composition of any one of embodiments 56-62, wherein the CRISPR-Cas protein comprises a native RNA nuclease activity.

64. The composition of embodiment 63, wherein the native RNA nuclease activity is reduced or inhibited.

65. The composition of embodiment 63, wherein the native RNA nuclease activity is increased or induced.

66. The composition of any one of embodiments 56-63, wherein the CRISPR-Cas protein comprises a native DNA nuclease activity and wherein the native DNA nuclease activity is inhibited.

67. The composition of any one of embodiments 56-66, wherein the CRISPR-Cas protein comprises a mutation.

68. The composition of embodiment 67, wherein a nuclease domain of the CRISPR-Cas protein comprises the mutation.

69. The composition of embodiment 67 or 68, wherein the mutation occurs in a nucleic acid encoding the CRISPR-Cas protein.

70. The composition of any one of embodiments 67-69, wherein the mutation comprises a substitution, an insertion, a deletion, a frameshift, an inversion, or a transposition.

71. The composition of any one of embodiments 67-69, wherein the mutation comprises a deletion of a nuclease domain, a binding site within the nuclease domain, an active site within the nuclease domain, or at least one essential amino acid residue within the nuclease domain.

72. The composition of embodiment 55, wherein the first RNA binding protein comprises a Pumilio and FBF (PUF) protein.

73. The composition of embodiment 72, wherein the first RNA binding protein comprises a Pumilio-based assembly (PUMBY) protein.

74. The composition of any one of embodiments 55-73, wherein the first RNA binding protein does not require multimerization for RNA-binding activity.

75. The composition of any one of embodiments 55-74, wherein the first RNA binding protein is not a monomer of a multimer complex

76. The composition of any one of embodiments 55-75, wherein a multimer protein complex does not comprise the first RNA binding protein.

77. The composition of any one of embodiments 55-76, wherein the first RNA binding protein selectively binds to a target sequence within the RNA molecule.

78. The composition of any one of embodiments 55-77, wherein the first RNA binding protein does not comprise an affinity for a second sequence within the RNA molecule.

79. The composition of any one of embodiments 55-78, wherein the first RNA binding protein does not comprise a high affinity for or selectively bind a second sequence within the RNA molecule.

80. The composition of any one of embodiments 55-79, wherein an RNA genome or an RNA transcriptome comprises the RNA molecule.

81. The composition of any one of embodiments 55-80, wherein the first RNA binding protein comprises between 2 and 1300 amino acids, inclusive of the endpoints.

82. The composition of any one of embodiments 55-81, wherein the sequence encoding the first RNA binding protein further comprises a sequence encoding a nuclear localization signal (NLS).

83. The composition of embodiment 82, wherein the sequence encoding a nuclear localization signal (NLS) is positioned 3′ to the sequence encoding the first RNA binding protein.

84. The composition of embodiment 82, wherein the first RNA binding protein comprises an NLS at a C-terminus of the protein.

85. The composition of any one of embodiments 55-81, wherein the sequence encoding the first RNA binding protein further comprises a first sequence encoding a first NLS and a second sequence encoding a second NLS.

86. The composition of embodiment 85, wherein the sequence encoding the first NLS or the second NLS is positioned 3′ to the sequence encoding the first RNA binding protein.

87. The composition of embodiment 85, wherein the first RNA binding protein comprises the first NLS or the second NLS at a C-terminus of the protein.

88. The composition of any one of embodiments 55-87, wherein the second RNA binding protein comprises or consists of a nuclease domain.

89. The composition of embodiment 88, wherein the sequence encoding the second RNA binding protein comprises or consists of an RNAse.

### EXAMPLES

### Example 1: Preventing Adaptive Immune Response to a Non-Self Therapeutic Transgene

A non-self therapeutic transgene is delivered to a target issue via viral or nonviral means. In order to prevent adaptive immune response to this therapeutic, vector with DNA encoding mutant FASL (mFASL) is co-delivered by AAV. mFASL expression is driven by a promoter that is activated by TNFα or IL-6 signaling (FIG. 3A). This regulated expression of mFASL induces expression of mFASL only in the presence of activated T cells. In turn, T cells become sensitive to mFASL-mediated death only when activated. Two AAV-9 transfer vectors were produced that 1) encode Cas13d and guide RNA, and 2) encode mFASL driven by an IL-6-regulated promoter. The following IL-6-regulated promoters were compared:

i. CALCB promoter:
(SEQ ID NO: 192)
tcctggtgtggtactacaaaggtcctggaagtcctcctgccttacttcgagtccgccacaggccgga
gctacttctagaacagttctgactgccttcccaggggagggggcgactgcaggtcaccctgcctcag
tgcacgctgcgcatctgagttgcaggccgggcggcagtcacccgagtcagcggtcccgattcttcg
tgggtgttacctggagtttccattgcacgggtgctggaggccaacccgccctagtcccaccagctgg
gtcctgccacaagaaaaggacccagagggcctagacagatatcccctgaacttttttctgacccgca
gaaacagtgagcatcacctgcaggggttccgccagcctctggctttgcttcctgcccggcagcctcc
agcaagttttcccgaatgctctccaggagtgcagtgggcgccggcggacttatttagggtcgctgct
gccaagcgtcagagagcgaaccggaggcccggggctgggggctgtggtgagaggcgccccttt
ctttcttgccatcgcccctacccccagctccttcctagcctattctgccaaagctgggatcttctcctgg
aacccgggagcaagaagagctggcagtgccgactcccagaacccggctttccttcagaaaagatg
gataggccgagtttgtgtgcgtggacgtgcatgtgtcggtgtgcatgtttaggggacagtatgtaccc
ccaattgcataaaacacgcctgtttttggaaacagaaacacacggggttgctttcgggtatgggtgtg
gttagtttgggttcctgcgcacgctcccttttcggagcagctggtgagggcgcatctgttccagtgagt
gctggctgtctgatggactgtctgtgaatgcagaaggcagagcgtgtggtgggggtcctgccatgtg
agtgatgatgcgcgcactggctggtctgtagtcgcgagtggaacttgtgtgaaaattctcaggctgac
tctcgcgctgcagcacctgctcccctccgtgggtggcagctggaccccagcgcgctcagctctcag
gcgcttcagcgaagtggggtaggggtgtggaacgagagagatagagacccgggcatagaccatc
tccgccaggcagcttggcaaacaggtggcagagttgcagggcagctgtgtaagccaacttcggcg
cagcagtggagggtcctggcttggcgtgggggatgctggacccgcggtcgaggatttggggatat
aggggaagagggaggaggtggatgctgagccttgtatgcaggctatgtcagtttagccctctcccc
aacctctcttcggctcctgcccgtcccagaggagtgaggtggagaagggctggctgccagactgg
caccaaaacagccttctttgggtgcccaggttgccagggctcgaggggtcggaggatatccaggg
aagcaccccaggtggtccaaaaagatcaaattttgaggacccctccctccccttttccctcccccccc
ctccttccctgccgtgggctctttcagctgtggtccctttagaacccaggactactactgctcaacctcg
ctgggggttcgggtggctggattcgggtccctcactggcgtgacaggagggagtgcgaggcagg
aatttaggagccaaggaggtgagagcagctctggcccctcactgtaggtgacgccaaactctcctc
gacttgccccgactcttagttgaaaaatctctgtcctctcccaggctctccagcttcccaagcaatgac
ctcaatgaaaaaaatgacagcggggcggactgcccccgctccagagtaccagtgccggcagtgc
gagctatgacgcaatcggagctcggtcggtcctttgattggctagtcctggccactttggattggccg
cgcgggctggtggggaccccccccctccagctatctctgtaataagagcggggtctccgcgggga
aggcGCCCACAGCAGGTGTGGTGTTCATCCCGGGTCGACCGGC
CGCTCGCGCTGCCCTGAAACTCTAGTCGCCAGGTGAGGAAC
TTCCCATTCCCCATTCCGCTC
ii. BCAR3 promoter:
(SEQ ID NO: 193)
taaatagaatattatcagcatgcagaagtcacctctgggatccctttctgtcataatccacagctcaata
taaccactgtcttcacttttaacatcacagatatgctttgactatttttgaagtttatgtaaatggaatcatat
agtatgtacccatttgcggccagcttctttcactcaatatcctatttgtaagattcactcatcttgttgagtg
taaatgagttcatgcattcttattactatgttgtatgttacacagtttatctctcttctgctgatggacatttgg
gttgttttctttttggccgtcataaagagatacattataaacatttttcaacatcttttggtgaatatgtgtata
catctctgttgggtgcacacttaggggtggaattgctgggtcataggatacatgttaatttagcttttcta
gataatgccaagcaggtttttaaagtggttgtaaaaagttcattttgacgagcagtatggtcaggccctt
taattttagtcattctgggtggtatgaagttagatcacattgaaatatcaatttgcatttttgggatggttaa
taaattgagatattttccatatgtttattggcttttttgagaaatgcctgtttgtcctctatgaggacagttg
aaatattctgcctgtttttaattggattttctgccttattctggctgatccgtaggtgttctttatatatctgtaa
acaaaccttttttggatgtatgtgttgcaattatgttttctcactctgcggtttgccttttcactcgtggtgtct
tttcatgaacagaagctataattttaatcttgtccaatgatgagttttttttctttatattagtactacatgtgt
ttaagacatcgtctttgtctaccccaaggtaatgaagatttttttggacattttctttggatcgtgttattggt
ttactttttacctttaaaatccatctggaggccgggcgcggtggctcacacctgtaatcccagcactttg
ggaggccgaggtgggtggatcacctgaggtcaggagttcgagaccagcctgaccaaaatggtga
acccgtctctactaaaaatacaaaaattagccgggcatagtggcgtgcgcctgtaatcccagctactt
gggaagctgaggcaagataattgcttgaacccgggaggcggaggttgcagtgagccaagatcatg
ccgttgcacaacagcttgggcaacaagaccgaaactctgtctcgaaaagaaaaattccatctggaat
tgactggggatggggggattcagttgatccagcatcatttactggaaagagagagctaatctgaccc
ccaatacagcacagcagcacctgacataaatcaagtgaccacatattgtaaatctgtttttggactttat
tccattggtcagtttgtctatccttctactatttctatgactttataatggatcttgatataagatagtgttaag
tcttttccagtgggctccaatttttcaccccaccaaatcatctagtgctgaagtgccaccgccaactcgc
actttgctttcaatacgtccatacgagtgcaattgatttgcctgttcccgtggatggttcatttgtggttac
ctggggctttgtctcatcaaagcccttcacattgaaagccagccaggtgtctgcagacgagcaaagc
agatctgttgggtaattaaataacgcggggaaggggaaggagtggattcgacgaggctgtctctgg
agagagcttcagaaaggaagtgatcgagctatacttgaggactggctccttggggagatacgaaga
gagccaaactctaaaatccgggcagaggagggcctggctaggtcaccgaatgtaaatgtctcggg
gggttccgcaaggcagcgcgaatcggctcgccggggtggggccgcggagccgcaaatcaccag
ttgagggccggagtgcgcgccgccggctcAGAGCTGCGCCTGCTGCTGGCC
GGGCGGGGGACGGGGCCGGGACCGGAGCCGGAGCTGCGGG
GCGCACCGGCTAGACGCGCGCGGGATTCTCGGCGGGCAC
iii. CCAR6 promoter:
(SEQ ID NO: 194)
ccacattcctcgccttttatgcacctcacagtgtctatgcaaatgaacagtgtgattttaaatttgaggaa
gtttcaataagagtgagatctaagaggatctttaatataggggcatttttagagagctaaaaaccaaat
aggtctcagctttctcagggtgacatataatttagatttgacttggaactacagaaggaatatggggca
aaggacatgagaaacattgcagaggtcagtgccatatggagcttccacctcagctctcgaagtaaag
gcaaccagcccaggctgggagccagtcagcagcagggtctcaatctccatccactaccttcccctct
ggggcagggagggtgttgcagggaggaggccactcttggagacctccagatccctgcctctgtgc
agtctaacagaaggggcccccacagtggccatgaatctctttgttctgcaaggaagggggtgtcatt
gggcctcctgggtccctcagcatctgcgtggccacacaacgcggttttgttcaacacgttacatttctg
cttaattaatgttcatttttggtccccagcaaagtccaatctgtttttcgttttctttcttttctttccaggcagg
cattgccagggctaatcatctataaaagggcttactttcttaccttcacgctaagcaagaccatccaag
ggcagtgttagagggcacctgagaacgcaggaggggtgtgttacctggtggcctgtttcttcctccta
tgggcagcccttctacaggaagcagaagcctcaggggcgatggtagaaatgaggaggaggggat
ggagctgaggtagcaggggaggggtcgtaagagcagaggggagggtcttgctttgttcgccatcc
aataatcagcaggtttcgggttggggtgaactactggggaggagcgccccagggcttctctcagat
ggaggagggacaggtcacccagagtgaaggaagttgtgagctctgttagaggaaaacagccagg
acttagtcaggagaggctttgtttggaaggattgttgtaaggcagggagaggaatgatggcagcaag
aggaccccgtgaccataggtctgcaagcacttcaaacagaaaaggcccttcttttatccagtaaggg
ggagccactggggccagcagagtctttggaggggaccctggacaaacccgggaaaatggccagt
ggggttgagcaggacacaggtcctgctgtgtctagctggttccccagagagatgataaggggtgcg
ctccagcttctcaggctcactcaggcgtgaggacgtggagctcagggctctgcaggaaggagcga
cccaggtgaggtgtggtcaagatagagcagagctgggcagcgggcagtggagcctcgtgggcag
cctgggggtggggaggcacagtgcactgggaagtggagaaagtgtgagtccatcaggctggctg
agaattgatcacgaacctattgtctgtaaaacttttgttatttcctgagacgtggttcacagcaacccag
gtgcgaacagccttgtgattctagggttcttttctattttttaagcacttgcatctacaaataaatttctgag
tgacttgtcgtcagctgctttccttgatatgtctaaagacagggcagtgacccgcatcgtcacccaga
gattctgtctctgtgccacatgaagattaggtgcccgcttttgattgaggagctcctctgttgctctcaaa
gtatcttgtaataatagctgagatgcatggagaaccacctctccttcaggcgctgctcctcggcttccg
tggacgggcatggctatttctcggaaccctctgaggttagagctgtcatggtctttcttctgaaagagg
aaaccgaggcttgctggggctcagtggcccttctgtggctgcacagctttcggggtggggccagga
ctgactgactccacacaaaagtgctcccggcccatgtctttAACTCACACGGCCTCTT
GCAAACGTTCCCAAATCTTCCCAGTCGGCTTGCAGAGACTCC
TTGCTCCCAGGAGATAACCAGGTAAAGGAGTATGAAAGTTT
G
iv. COL6A3 promoter:
(SEQ ID NO: 195)
agtgtatttgtatttgaaagaaaccgtggagtggaaacacctaaaacgtgcttgttcagttaacctcagt
ttgctgagctgatggagcatggggttgtaatcaaagattgtcttcttcgcagcagacaatgttctggg
agaaatagtcctccttagtgctgaagttgcagactctaaccaagggggtggcagcaagtatccccgg
tctctgtaggggcttgagtcaacgcctgcactgtgcagagaggatgagggcagagaattggatgcc
gctggaggggcgtgttgtccttctacatgtcatgcaggcagcgcggttctatactcggagcctctgct
cagcgtgtcttcacctaagaaccccataattcaggttccatccttgttccctactccagtgctctgcaag
tgagccctttggtttagagatgggttgggcttctttatgggagaggaagggagccctggagctgcag
aggggagcaggcattctctctggggtgctgtctcctttcttccctaaattggagggagataatccatgg
aaaggagttaatgatttctttgctcttcaaacttggtttggaaggatctctcagtcaaaaagaacctttcg
gatgtctcttgatatttcacattaatggacttttcataaggaccacatgatgggagagcagtgagaagtt
tggggatggccaaagctgggttgtcatttgagctctgttactaacccagactggacaatgacgatgtc
acttactctctcggaacttcttttcttttcttcttttcttttctttttttcttttttctcttctcttctcttctc
ttctcttctcttctcttcttttcttttcatttcttttctttctttcctttttctcttttttggagtctcactctg
ttgcccaggctagagtgcagtggcgccatctcggctcactgcaacctctgcctcctgggttcaagcaattgtcct
gcctcagcctcccaaatagctgggactacaagcgcctgccaccatgcctggctaatttttatatttttagtagag
atttagtaggggtttcaccatgttggccaggctggtctccaactcctgaccttgtgatctgcctgccttagc
ctcccgaagtgctgagattacaggcataagccaccgtgcccagcctgtttctttcatctgtaaaatggg
accacaatttcacctaataaaagaagacatctttctatttaaaagggcttagggtgttgatgtttgtgata
aaggagagaatgtatattgaagtgttttgaaatgtgcaaagctttctagaaacagaagttcttactcaag
tattttcccgaagctttggcaagataaccatttttattaccccgtctgtgcctagaatgggcctataagcg
ccacaatcagaatcattagatatagaaattaagagaaatgtagcctccttttttttgccggtgaacagag
ctttggttaacagaaaaccaaggcgattttaattgctggtttttctatttgaagggggaagttattagtag
aagtctcaattcagaaacttcaagaagaaatgggagggtgtggtgagggtaagcgggggactgcat
ttcctgttttcctttcagatggtgttggaaaacattgcaggaaaaccatggatacccacgaagaaattcc
aaaatttattctttttgacgccaagggcccagcccaaaaggtgacgagtaggagtggtcaatttttttttt
taagagttggggcttgcaggagtccagctaaacgcttgtagggtgaagacagaattcagagggtga
catcagcctgagcgggggccagaagaaacagagtggaggagtctggtttcatttacagttttgggtc
agttctgcagtgaggagggggagaggaggggtccgggagggaggaggaggaggaggaggag
ctggaggaagccctgactggtatccctggccccagtccagtttggagctcAGTCTTCCACC
AAAGGCCGTTCAGTTCTCCTGGGCTCCAGCCTCCTGCAAGGA
CTGCAAGAGTTTTCCTCCGCAGCTCTGAGTCTCCACTTTTTTG
GTGGA
v. CXCR5 promoter:
(SEQ ID NO: 196)
atggaatcttttttttttttttttttgagtgtcaccaaggctagagggcagtggtgtgatcatggctcattgc
agcctcaacctcctgggctcaagcgatccgcctatctcagcctcctgagtagctgggactacaggtg
tgtgccaccatgcccagctaatttttgaattttttgtagagacgaggtttcaccatgttgcccaggctagt
ctcaaattcctgggctcaaaggattctcccatcttgacctcccaaagtgctgggattacaggcatgag
caaccacgccctgccaagtatagagtcttgaacaaggaaatgcatatcgtcctatattttttcctagtca
gataatatctagaccattaaccagaaatcacccagaggtcaaaaaacagggcgtcaaaggacccag
aaaccaagtctgcaaataacgactgaagacactgtggaagtgtgtttgggagacaacaagactctca
ggatgtgctggctgtatcagaggatgcttattggaagaggagtcagacagtccagacagaaggca
cagccaggacctctggagaggagttacaggaagacatattttgactcatcataaggaataagtttcta
atcatgaaaccatcctccactgaaacatgatctattgaaaggagcaaatgtctcaccttcattgatgttc
gtattcattgattctgggtgatcatctgataaggatgcagtgacgagaatcttgcatttgctgggggtgg
gggtgtggttgaggatagtctggtttatattccaaagttcctttcaattcctctatgattctatacgctgtac
tccttcctgatcaatgtccctagccagggtggccaaggctaagtcaagtatgctaagggattggagg
ggcagggatattgagaatagggtgaatggaaggatgaggagttcccagcaagcttgggacacagg
aaaccttggggcagcttcctcctggaggtttcaggactgtacgtgctggagaagaagtgtgatgcctt
gtcctgaaagccgtcttctttgaaagcagcttctaaaggcagtgaatggagaagagcgaggaaacg
accccaataccaccaacagaggctggaaactcctcaggctgtttaatcctaagaatgatgcatctgtt
ggccgggcacggtggttcacatctgtaatcctagcactttgggaggctgaggcaggcggatcacctt
aggtcaggaattcaagaccagactgaccaacatggagaaaccccatttctactaaaaatacaaaatt
agccgggcgtggtagcgcatgcctgtaatcccagctactcgggaggctgaagcaggagaattgctt
gaatccgggaggtggagtttgtggtgagctgagatctcgccattgcactccagcctgggcaacgag
agcaaaattctgtctcaaaataaataaataaaaatacaaaattagccgggcgtggtggtgcatgcctg
taatcccagatgctcgggaggctgaggcaggagaatcgcttgaacctgggaggtggaggttgtggt
gagccaagatcatgccattgcactccagcctgggcaagaagtgcaaaactctgtctcaaaaaaaaa
aaaaaaaaaaaaaaaaaagaatgatgcatctgttggggatgcagtggggtaagcatcttcagtaagc
aaggtgtgaagaggggaaagagggaaggtgaatatggaggagagggtgaaggagggcactgg
aaagggtagtaggatcccagcaaagggcgatttggctgaaagggagcgtgataacaagggtggg
ggtggggccaagaagcagccaccatgtgtgggtgcctctgtgcgtgcagtcatctttctcacatcatt
gtggatcaagagaggaaatgcccacttctggaagaaaaagccacaaaatgagacttggaagggaa
attgatcaacatctacaaaacggcttcttaaaggaagcggccctcAGACAGGACAGAG
TTGAGGGAAAGGACAGAGGTTATGAGTGCCTGCAAGAGTGG
CAGCCTGGAGTAGAGAAAACACTAAAGGTGGAGTCAAAAG
ACCTGAG

AAV-9 preparations were generated according to standard techniques (triple-transfection method) and purified by IDX gradient ultracentrifugation. AAV was titered by qPCR after dialysis against PBS. One of the three AAV versions described above is next injected into the tibialis anterior muscles of wildtype FVB strain mice (304, total volume, 2*10\hat{ }10 vg, 1*10\hat{ }11 vg or 4*10\hat{ }12 vg) and subjected to daily clinical observation subsequently. (Contralateral injection of vector 1 and either vector 2, 3, or PBS. 4 mice for each combination, 1/2, 1/3, 1/PBS). Mice are sacrificed at 1 w, 4 w, and 6 w after injection. For each animal, the proximal half of the tibialis anterior muscle (injection site), heart, spleen, liver (representative portion, i.e. piece of a lobe) and kidneys are collected, placed individually (except pair organs) into cryovials and flash frozen in liquid nitrogen for RNA/protein assessment and changes in gene expressions. The other half of the tibialis anterior muscle is embedded in OCT and frozen. The tibialis anterior muscle is cut in a transverse fashion.

RNA isolations from frozen tissue is carried out with RNAeasy columns (Qiagen) according to the manufacturer's protocol. RNA quality and concentrations are estimated using the Nanodrop spectrophotometer. cDNA preparation is done using Superscript III (Thermo) with random primers according to the manufacturer's protocol. qPCR is carried out to assess the levels of Cas9 in tissue among the three mouse groups (vector 1/2, 1/3, 1/PBS).

Immunofluorescence with sectioned tibialis anterior muscle is conducted to measure infiltration of immune cells (CD3 and CD45 staining).

### Example 2: Preventing Adaptive Immune Response to a Non-Self Therapeutic Transgene

A non-self therapeutic transgene is delivered to a target issue via viral or nonviral means. In order to prevent adaptive immune response to this therapeutic, vector with DNA encoding mutant FASL (mFASL) is co-delivered by viral or nonviral means. The mFASL mRNA contains an intron that splits the coding sequence of FASL (FIG. 3B). This intron is bound by an RNA-binding protein Cas13d with a single guide RNA that is partially complementary to the intron which prevents splicing of the adjacent exons. The Cas13d guide RNA is perfectly complementary to genes whose expression is regulated by TNFα or IL-6 signaling so that mFASL splicing is released from blockage upon TNFα or IL-6 signaling. Systems where the guide RNA is perfectly complementary to mRNAs encoded by the following genes were constructed: BCAR3, CALCB, CCR6, COL6A3, CXCR5, DHRS9, FLT1, FNBP1L, FNDC9, GBP4, GPR87, GZMB, HOPX, HSD11B1, IFIT2, IFNL1, IGFBP6, IL12RB2, IL1R1, IL1R2, IL23R, IL24, KCNK18, MAF, NAPSA, PALLD, PRG4, PSD3, RORA, TNFSF1, TNFSF13B, TSHZ2. Two AAV-9 transfer vectors were produced that 1) encode Cas13d and guide RNA, and 2) encode the mFASL construct with the intervening intron.

AAV-9 preparations were generated according to standard techniques (triple-transfection method) and purified by IDX gradient ultracentrifugation. AAV was titered by qPCR after dialysis against PBS. The AAV encoding the non-self transgene along with a vector containing the engineered mFASL construct and Cas13d were next injected into the tibialis anterior muscles of wildtype FVB strain mice (304, total volume, 2*10\hat{ }10 vg, 1*10\hat{ }11 vg or 4*10\hat{ }12 vg) and subjected to daily clinical observation subsequently. (Contralateral injection of vector 1 and either vector 2, 3, or PBS. 4 mice for each combination, 1/2, 1/3, 1/PBS). Mice are sacrificed at 1 w, 4 w, and 6 w after injection. For each animal, the proximal half of the tibialis anterior muscle (injection site), heart, spleen, liver (representative portion, i.e. piece of a lobe) and kidneys are collected, placed individually (except pair organs) into cryovials and flash frozen in liquid nitrogen for RNA/protein assessment and changes in gene expressions. The other half of the tibialis anterior muscle is embedded in OCT and frozen. The tibialis anterior muscle is cut in a transverse fashion.

RNA isolations from frozen tissue is carried out with RNAeasy columns (Qiagen) according to the manufacturer's protocol. RNA quality and concentrations are estimated using the Nanodrop spectrophotometer. cDNA preparation is done using Superscript III (Thermo) with random primers according to the manufacturer's protocol. qPCR is carried out to assess the levels of Cas9 in tissue among the three mouse groups (vector 1/2, 1/3, 1/PBS).

Immunofluorescence with sectioned tibialis anterior muscle is conducted to measure infiltration of immune cells (CD3 and CD45 staining).

### Example 3: Treatment of Myotonic Dystrophy Type I (DM1)

Compositions of the disclosure are used for the treatment of myotonic dystrophy type I (DM1) wherein an RNA-targeting CRISPR system composed of a therapeutic transgene (Cas9 or Cas13d and corresponding single guide RNA targeting the CUG repeats that cause DM1) is delivered to patient muscle or the central nervous system. The presence of mFASL causes the elimination of T cells that are specific to Cas9 or Cas13d and potentially cytotoxic against treated cells.

### Example 4: Treatment of Hemophilia

Compositions of the disclosure are used for the treatment of hemophilia. A secreted transgene such as Factor IX is used for the treatment of hemophilia. A vector carrying an expression cassette for factor IX along with mFASL reduces, eliminates, or prevents an adaptive immune response to Factor IX-expressing cells.

### Example 5: Preventing Adaptive Immune Response to a Non-Self Therapeutic Transgene while Simultaneously Preventing Immune Response to Repeated AAV Administrations

Compositions of the disclosure may comprise an AAV vector containing an expressed polypeptide composed of all or part of AAV viral capsid protein. The AAV capsid polypeptide is identical to the serotype used to deliver the system. Co-expression of this AAV capsid polypeptide causes the elimination of T cells that are specific to the AAV capsid in a manner described above. This causes depletion of T cells that can regulate both cellular and humoral immunity to the AAV capsid. This allows repeated dosing of the same AAV serotype. In the absence of the compositions of the disclosure, and using the standard of care prior to development of the compositions of the disclosure, an individual AAV serotype could not be used in more than once in a patient due to the formation of adaptive immune response against the viral capsid.

The compositions of the disclosure may be useful in situations wherein incomplete therapeutic transfer occurs during the first administration of a gene therapy or wherein a second dose is desired. In this case, the second dose of the gene therapy does not require the presence of the mFASL and AAV capsid polypeptide unless subsequent doses beyond the second dose are desired. One situation could be during the treatment of large organs such as skeletal muscle where the volume of virus required to transduce muscle in a single dose is prohibitively high. Another situation could be during treatment involving complicated administration methods in the brain or spine where initial treatments do not provide satisfactory infection of targeted cells.

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### OTHER EMBODIMENTS

While particular embodiments of the disclosure have been illustrated and described, various other changes and modifications can be made without departing from the spirit and scope of the disclosure. The scope of the appended claims includes all such changes and modifications that are within the scope of this disclosure.

1. A composition comprising:
(a) a sequence encoding a non-self polypeptide of interest (POI), and
(b) a sequence encoding a non-cleavable Fas Ligand (FASL),
wherein expression of the non-cleavable FASL eliminates MHC-mediated immunogenic peptides and helper T cells specific to the expression of the POI.

2. The composition of claim 1, wherein expression of the non-cleavable FASL selectively eliminates a T-cell that recognizes a MHC-peptide complex, wherein the peptide is derived from the non-self polypeptide, and wherein expression of FASL is in the presence of IL-6 or TNF-alpha.

3. The composition of claim 1, wherein the non-self POI is a nucleoprotein complex encoded by (i) a sequence comprising a guide RNA (gRNA) that specifically binds a target sequence within an RNA molecule, and (ii) a sequence encoding an RNA-binding polypeptide.

4. The composition of claim 1, wherein a vector comprises the sequence of (a) and the sequence of (b).

5.-6. (canceled)

7. The composition of claim 1, wherein a promoter drives expression of the sequence of (a).

8. The composition of claim 1, wherein the promoter drives expression of the sequence of (b).

9. The composition of claim 8, wherein the promoter is a promoter regulated by the presence of IL-6 receptor or TNF-alpha receptor.

10. (canceled)

11. The composition of claim 3, wherein a first promoter drives expression of the sequences encoding the nucleoprotein complex and a second promoter drives expression of the sequence of (b).

12. The composition of claim 11, wherein the first promoter comprises a sequence isolated or derived from a U6 promoter or wherein the first promoter comprises a sequence isolated or derived from a promoter capable of driving expression of a transfer RNA (tRNA).

13.-15. (canceled)

16. The composition of claim 1, wherein a delivery vector comprises the composition.

17. The composition of claim 16, wherein the delivery vector is an adeno-associated viral (AAV) vector.

18. (canceled)

19. The composition of any one of claim 1, wherein the sequence of (a) or the sequence of (b) further comprises an Internal Ribosomal Entry Site (IRES) or sequence encoding a self-cleaving peptide.

20. The composition of claim 19, wherein the IRES or the sequence encoding a self-cleaving peptide is positioned between the sequence of (a) and the sequence of (b).

21. The composition of claim 19, wherein the self-cleaving peptide comprises a 2A self-cleaving peptide.

22. The composition of claim 1, wherein the non-cleavable FASL comprises a mutation in a metalloproteinase cleavage site, wherein the mutation comprises one or more of a substitution, an insertion, a deletion, a frameshift, an inversion, or a transposition of the amino acid sequence ELAELR.

23.-24. (canceled)

25. The composition of claim 22, wherein the non-cleavable FASL comprises the amino acid sequence of:
(SEQ ID NO: 210)
MQQPFNYPYPQIYWVDSSASSPWAPPGTVLPCPTSVPRRPGQRRPPPPPP
PPPLPPPPPPPPLPPLPLPPLKKRGNHSTGLCLLVMFFMVLVALVGLGLG
MFQLFHLQKX₁X₂X₃X₄X₅X₆ESTSQMHTASSLEKQIGHPSPPPEKKELR
KVAHLTGKSNSRSMPLEWEDTYGIVLLSGVKYKKGGLVINETGLYFVYSK
VYFRGQSCNNLPLSHKVYMRNSKYPQDLVMMEGKMMSYCTTGQMWARSSY
LGAVFNLTSADHLYVNVSELSLVNFEESQTFFGLYKL,
wherein X₁ is not a glutamic acid (E), X₂ is not an leucine (L), X₃ is not an alanine (A), X₄ is not an glutamic acid (E), X₅ is not an leucine (L) or X₆ is not an arginine (R).

26. (canceled)

27. The composition of claim 1, wherein the non-cleavable FASL comprises an intron, wherein the intron blocks FASL splicing in the absence of IL-6 or TNF-alpha.

28. The composition of claim 27, further comprising synthetic mRNA target sites which are expressed in the presence of IL-6 or TNF-alpha.

29. The composition of claim 1, further comprising 1) a synthetic notch system, 2) microRNA target sites, or a 3) split intein and engineered IL-6 or TNF-alpha receptors for regulating expression of FASL in the presence of IL-6 or TNF-alpha.

30. The composition of claim 3, wherein the RNA-binding polypeptide is a CRISPR/Cas polypeptide selected from the group consisting of Cas9, Cpf1, Cas13a, Cas13b, Cas13c, and Cas13d.
